SEQUENCE LISTING (12 sequences)

SEQ ID NO: 1 - protein, 122 aa, Homo sapiens [sequence omitted]

SEQ ID NO: 2 - DNA, 369 bp, Homo sapiens
atgaagcttctcacgggcctggttttctgctccttggtcctgggtgtcagcagccgaagcttcttttcgttccttggcgaggcttttgatggggctcgggacatgtggagagcctactctgacatgagagaagccaattacatcggctcagacaaatacttccatgctcgggggaactatgatgctgccaaaaggggacctgggggtgtctgggctgcagaagcgatcagcgatgccagagagaatatccagagattctttggccatggtgcggaggactcgctggctgatcaggctgccaatgaatggggcaggagtggcaaagaccccaatcacttccgacctgctggcctgtctgagaaatactga

SEQ ID NO: 3 - DNA, 10717 bp, Homo sapiens
ttttcacagttcatttacggccctctttccaacacattcactgccaatactcttattgacaataactgtattgttgaaccttccagtatcctgcattcccggatcaaggccccctcaaagccctgatatgcaaatatctgggaaaagaatgttccagaggaaaggaacagctaatccgaggcccctagggtaagatgtgcctgggggtttggagaccagtgtggccagagcaaaatgagcaggaggagagaattggatgatgaggtacgagaggaaggagttaggacagtttgagtaaagtttgaaaaccattataagggctttgacttcaactatgagtggaagtggaatcctccggagagttttgaatggagagtgatagaagttgtcttgtgttgtaacagtctggctgctatactgaaaagagactagttggcggcaaagggggaaatgtggaagccagttaagaagccatcataacccagaaggtgatgcctaataacatctctctgggagcagcggagagatgataagggtttgccttctgaatatgttttttgacaattaatgtaaacatttcaagtaggctgagattttattgcatattaacaatgtccatgttcactcgcggcagccgcccccttctgcgcggtcatgccgagccagcacctgggcctggaactgggccgcagcccccagcttcacccaccacctccctaccatggacccctgcaaagtgaacgagcttcgggcctttgtgaaaatgtgtaagcaggatccgagcgttctgcacaccgaggaaatgcgcttcctgagagagtgggtggagagcatgggaggtaaagtaccacctgctactcagaaggctaaatcagaagaaaataccaaggaagaaaaacctgatagtaagaaggtggaggaagacttaaaggcagacgaaccatcaactgaggaaagtgatctagaaattgataaagaaggtgtgattgaaccagacactgatgctcctcaagaaatgggagatgaaaatgtggagataacggaggagatgatggatcaggcaaatgataaaaaagtggctgctattgaagtcctaaatgatggtgaactccagaaagccattgacttattcacagatgccatcaagctgaatcctcgcttggccattttgtatgcaaagagggccagtgtcttcgtcaaattacagaagccaaatgctgccatccaagactgtgacagagccattgaaataaatcctgattcagctcagccttacaagtggcgggggaaagcacacagacttctaggccactgggaagaagcagcccatgatcttgcctttgcctgtaaattggattatgatgaagatgctagtgcaatgctgaaagaagttcaacctagggcacagaaaattgcagaacattggagaaagtatgagcgaaaacatgaagagcgagagatcaaagaaagaatagaacgagttaagaaggctcaagaagagcaggagagagcccagagggaggaagaagccagacgacagtcaggagctcactatggcccttttccaggtggctttcctggtggaatgcctggtaattttcccggaggaatgcctggaatgggaggggacatgcctggaatggccggaatgcctggactcaatgaaattcttagtgatccagaggctcttgcagccatgcaggatccagaagttatggtggccttccaggatgtggctcagaacccagcaaatatgtcaaaataccagagcaacccaaaggttatgaatctcatcagtaaattgtcagccaaatttggaggtcaagcataatgcccttctgataaataaagccctgctgaaggaaaagcaacctagatcaccttatggatgtcgcaataatacaaaccaacgtacctctgaccttctcatcaagagagctggggtgctttgaagataatccctacccctctcccccaaatgcagctgaagcattttacagtggtttgccattagggtattcattcagataatgttttcctactaggaattacaaactttaaacactttttaaatcttcaaatatttaaaacaaatttaaagggtctgttaattcttatatttttctttactaatcattgtggatttttccttaaattattgggcagggaatatacttatttatggaagattactgctctaatttgagtgaaataaaagttattagtgcgaggcaaacataaaaaaaaaaagtccatgttcatctctaaatgacatcattgttccaaagcttttccattcttcttaaccttccacctgtcaatctataggagatgacttctcctacttcactcatgcattgactccttcaatcaataaaagtgactaagaacctgctacaggtgaggtgctgtgtttggtgttaaagtgacaacagttatctgtcaataagcctgacaaggttcctatccctgtgttttgtgcactctgggtcaaactcagaaatgcaaacaggtggagagcgatgagttctatgactggtaaagaaaagggcctgctggtttccctcaggatctctgtccttcatctcaaaatgcatcttccttgttatcgttcctctccttcctgtctcagaggaagacctgctcctgctacactctgggcaaccttgtccccgtggccctgtggccccttggttgttgaagtctatgttatgccctatcttttaccctcagtcactctctctgttaacattctccctgtgccctgtaaccctccctcatctttaaataaatcctcctcctttgaccttcgcatgtattcagtcatgcaactcaacaagcatttattgcacagtgatattcaatttgccacttgctaaaagtctgaaccttggcagctgaatgtgatcagaaaaaaagcacgactgctatgactagtctcactttaaattcatggtcgttgaccaagagctaccatacaatccactacctttctcaagttcagtcacattcttcctttcctagatgtctgctttctacttctcttctcttctgaaacttcccacaactcctcgttcattctcttctcagttgacaactttgcttcctatttcactgaaaaatagaagcaatcagatatgaacttctggctgggcatggtagctcatgcctataatctcagcactttgggaggccaaggcaggaggactgcaggttaggaatttgagaccagcctgggcaacatggtgaaactcccactgtactaaaaattttaaaaattactcaaacatattggcaaacaactgcagtcccagctacttgggaggttgagatgcaaggatcacttaaacctgggaggctgaggctgcagtgagccatgattgcaccactgcactccagctcaggcaacagagcaagaccctgtcttgagaggagaggagaagagaggaggggaggggagggcaggggaggggaggggaggggaagggagaggggaggggagaggggaggagagaggggaggggaggggaggggaggggaggggaggagaggaggatcaggtgaggagtatgccaaggagtgtttttaagacttactgttttctctttcccaacaagattgtcatttcctttaaaaagtagttatcctgaggcctatattcatagcattctgaaagaaagaaaagaaaagaggaaagaaagagagaggaaggaaggaaggagaaagagagaggaaggaaggagaaagagagaggaaggaagggaggaagagaagaagggaggaagaaaagaaggaaggaaggagggagggagggaagggagggagggaaagaggaagaaaggagggaaagaaggaaggaagagagagaggaaggaaggaggaagagagaagaaggaaggaggaagacagagagggagtaaggaaggaaggaaggagaaagagagaggaaggaagaaatgaaggaaggaaggaaagaaagaaaaaataaaagagtgaaaacggactggagaagaagaaaccacagttgctgctatatccaccagcctctctgcatgtcctggcctcagccctgctgggctctggtactgaccacttccttccttcctaatttcctaattgactaggccagctgagcagggcttttctgtgctgaggaggtaaatctctggatatctagactgaggggtggaaggagccttccagggcacacatgagacatggcaggggtaggctgctagttttattttgttttcttttagacacagggtcttgctctgttaaccaggctggagtgcagtggcgtgattatagctcactgcagccttgacctcctgggtctcccacaatccttccgcttcagcctcttgagtagctgggactgcaggtgcacactaccacacccggtccatttatttttatatttcgtagagacaagatcttacagttttgcacagagtgatcttaaactcttgaccccaagtgatcctcctgccttggcctccaaaagcattgggattataggagtgagccactgtgctggacctagtctgtcagctttgaagctttagatatgaactcagagggacttcatttcagaggcatctgccatgtggcccagcagagcccatcctgaggaaatgactggtagagtcaggagctggcttcaaagctgccctcacttcacaccttccagcagcccaggtgccgccatcacggggctcccactctcaactccgcagcctcagccccctcaatgctgaggagcagagctggtctcctgccctgacagctgccaggcacatcttgttccctcaggttgcacaactgggataaatgacccgggatgaagaaaccactggcatccaggaacttgtcttagaccgttttgtaggggaaatgacctgcagggactttccccagggaccacatccagcttttcttcgctcccaagaaaccagcagggaaggctcagtataaatagcagccaccgctccctggcaggcagggacccgcagctcagctacagcacagatcaggtgaggagcacaccaaggagtgatttttaaaacttactctgttttctctttcccaacaagattatcatttcctttaaaaaaaatagttatcctggggcatacagccataccattctgaaggtgtcttatctcctctgatctagagaggtaagcagggtcgggcctggtagtacttggatgggagaacacctgggaataccaggtgctaaaggctttaagaataaaaaataatgatcctgctttgtgtttatcccatgttgagttctgtgcggggcagagggaacacacggtaaatgcgttatggggaattataggctacttgagggagtgacagtctggtggtaactcctgccttcctccatcagtgccacgttggcatcctcttatgcagtcaggcttcagggctgatgggttcagaaccgagggcttctggctctgagtgaggtcctgctgcaaggtttcctagatgagccactgagactctaataagatccagtggaaataaccaggctctcgtcggaatataagtcccaagggaagctgtgccagtcttgtgggcgactgcctgacttctcctttcatttcagcaccatgaagcttctcacgggcctggttttctgctccttggtcctgggtgtcagcagccgaagcttcttttcgttccttggcgaggcttttgatggtaaggcttcagaaggtttgcaggatttctgaagagaaacatcaccctggacctgataaactggggaaaatgatgctttcggaaggctgcttttgaaccacagagttgctagtgtctgcgttgctgaggcctgccaggaactagggtttgctgggttgcctgtctcgagtctttcagagctgctgggaatatcccctttccccgtagtgcagcttctcaggatgtgttaagtggatggatcacatttcagaagccgctgcaaggtgtatcaaaaacacatctcctgagccgtaagggacggggcatccagtaacaacgcacacggggtatttttgggcttccttaagatttgagccgctgccttaggttgtgctgcccaatgtgcctggggagctgctaaacagattagagagtcgaggattgttgtcagttactcagagaaagaacaatcatcctttccaggagcacctgagctgtttgttttgcgtagaagatgcaaaataaggcctgcaatgggtataaaatgtccctcagcataaatcgcataggagtatgactaaggctgttgactcttctgtcttctttctccttcctccttcgatttcctagttggataatgtacagggctctttagcctcgctctgtcaggggctcccttcctggtttgttctgtttccattcttccttctccagccttcttgacaagagctgggaactaacgtgcctcaagcccccacaaggaccacagcattttctcatttagtttcagaatgactctgtgacgcaatcttcctctcttggaaggtgagaaagctgatcttggaaggtgagaaagctgagacttagagcagctgaagccaatgcccagggacttactgccagtcagcaggtggcagggcagaggtttgagcccggctgtgcttgaggtcagggctcttgccaggtagacgcatcactgaccacctcctagaggttgatggttatgaatctcaggcacaccttggcatcacctgaaatacccatgccttcaactccccagcagagtctgcagaaactggcctggggtgtggcctgggcactgggactttcagtttctctctgggtgattagaaagtgcagccaaggctcacgcctgtaattccagcactttgggaggccaaggtggatgaatcacttgaggtcatgagttccggagcagcctggccaacatggtgaaaccccgtctctactaaaaatactaaaatgtagccaggcgtggtggcaggcacctgtaatcccagctactcaggaggctgaagcacgagaatcacttgaacccgagaagcagaggttgcagtgactagagatcgcaccagtgtcctccaacctgggtgacagagcgagactccatctaaaaaaaatgaaaaagaaagtgcagccaaggcagagcaccactgccctattgcttcctcaagcaacccacagcatcagtacagcctactaagaaagtatttagggacttttatgctcctaacagtcactggaactcacgtcacaatgacgtgtattccatttgcaagaatatatactttaggtcggggtgcggtggctcacgcctgtaatcccagcactttgggaggccaaggcagggggatcacgaggtcaggagttcgagaccagcctgaccaacatggtgaaatccccgtctctactaaaaatacaaaaattagccaggcgtgatggcgcatgcctgtaatctcagctactcaggaggctgaggcagaagaatctcttgaacctgggaggtggaggttgcgatgagctgagatagcaccactgcactccagcctgggcgacagagcaagactctgtctaaaaaaaaaaaaaaaaaaaaaaaaaaaaaaaagaatataaactttagtagtcagggcagaagtactctgtgtctgccacctttctcagcatcagtattccatgtcactacctcattcatacacactcctggatcttatcataggcagcttcattctatagcagtggctcttcaccagggcacttgaagaagccaactaggataaaggaatgtgcttctcaacccatggtatccaaggctgctatgatcacaggctgaaagcttgaagtcagtggaagatttgtccttcctcattcccctctaaggtgttgttggagtctttatgttctcctgatgtcccttctgcctttcctttcctttccaggggctcgggacatgtggagagcctactctgacatgagagaagccaattacatcggctcagacaaatacttccatgctcgggggaactatgatgctgccaaaaggggacctgggggtgcctgggctgcagaagtgatcaggtaactggagctcctgggacgttagggctgggtgagcagagcttgcctgccttggacagtcaggagggagacgagctccttgtggagaagttagaggctgcggcccctcctcctcttgccctctctctgcctctgtgctcagtgtgaggtctgagtggatggtaggagtgagtgattcctcatcctccctctctgggtgctgttcatccagcctaggggtgcccagcctggctgaatggggtggtgcccagtgttttcatccctccttccttggcctttctgggctcctctctgagccctcccttggaacagggagaatgggagggtgggctattgctcactggcctgattattaatctccttcttgcctgccttgattacagcgatgccagagagaatatccagagattctttggccatggtgcggaggactcgctggctgatcaggctgccaatgaatggggcaggagtggcaaagaccccaatcacttccgacctgctggcctgcctgagaaatactgagcttcctcttcactctgctctcaggagatctggctgtgaggccctcagggcagggatacaaagcggggagagggtacacaatgggtatctaataaatacttaagaggtggaatttgtggaaactgggtgttatactttgtggtatagactgcctgtttagtatgaaggggcgatccatgcacatctaagtgaacgtggaggctgggtgggtgggagacgactcctgggcacacagggcatcctgggcatccctgaggcaaggacatgatgagttcagtggccacccccacaggatcccaggggcttcagcagatcccaccccttaccccatgtgagcagctgcccagtgagtctgtaggaacccgagccacattcccagtgagttcaactgcaccccggcacgttttgctagcacctcaatggagagctccttgcttgcagctttggcttgtggcacccagcaaaagcttcctgccacccagtggctacagccacacactctccagcaagatttaatctcagccttgtgaggagccctttcccaaatttatttctttctgtgttttttatcccttagtagctaatctcatgttagccattaataactctctatgttaaacccttccttttgtatctgcggctacattgatcaattgtctcacaccgctcacccaccccctctccctggtcatgcagaggcctcaccagtcattttattgctattcccaggcctctgtgtgcccagatctcttcactgccctgtcagttgtgtcctgtccccttctcgacctcctggccttgtcctcaatgatgtttctatgaggctttggaaagcctcatcccaagagtcctggcaactgatacattagtctcaccaacactagcccctactcctgatctcattttaaaattttattttcttattttattattattatttttagggacatcaccctgcttctgactgacccaatttttaaagtttctctttattctccttaaagaatggctctcccatttgtttccctcatctttcttttcactgacttagaattcaggtccagacaaaaattccacttccttgaagagccttcccagtagccatgaaccacactctggggcagagtttgtggctcccaagcactttgttcacacctgctcgttcatctttaccccctcctctcagaattggttttgtatgtcagttcccctgctggactggaagcccctgtaacataactagcatttgaacagttcataaaaacactttcatttctattgtcctgtgtaactgttcacaagatacattagtctcactcattgtcgtttgacaatgtttattgttctaaagagaaagcgagatatgtaggtagacacaacacaataagagcctcaatacaggcacacactggtggcagtccaaactctgctggaatatggaaggccagtgaatagtaactagaatcgctgccatcttttacactgcaattgcaagactgtgtcctatactttacatgctttatcggattgacactgcagagaagcctttgatgttctcggttggataaacaggtttagaggtatgtgttcaggccttctccaaggtcacacatgtaacaggtgcagagccaagactgcaggctggtttgtctgactccagaacctgtgatacactgaacaaggccaagagattcagcagtggagacctcctctcttcttcaggaagatagggaacatggtaagagcagtgaattccataagcatgggaccccgttatacttcattttttgtaaaattagttccttgatcagaagcaacgcctttgggtctctcgctattgagttaagtgtggatgtgccactctccctagtaccccagagtgtagttggaggcttgcctagagattagaggttgaggatccttcagtagttcctataccaagccataagttgtgctgcatctgcaggtagggtcagggagctccgctgaacagatgtggaggacatcagagtgggaggaaaaggaagcaagtcgtgtggggagagaagacccagcctgcaatgatgactggttaactactgtttcttacctaatacacactcagataacagactaagtctag

SEQ ID NO: 4 - protein, 224 aa, Homo sapiens
MEKLLCFLVLTSLSHAFGQTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 5 - DNA, 1307 bp, Homo sapiens
aaggcaagagatctaggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggcccagctgtgggtcctgaagtgctttcttaattttatggctcttctgggaaactcctccccttttccacacgaaccttgtggggctgtgaattctttcttcatccccgcattcccaatatacccaggccacaagagtggacgtgaaccacagggtgtcctgtcagaggagcccatctcccatctccccagctccctatctggaggatagttggatagttacgtgttcctagcaggaccaactacagtcttcccaaggattgagttatggactttgggagtgagacatcttcttgctgctggatttccaagctgagaggacgtgaacctgggaccaccagtagccatcttgtttgccacatggagagagactgtgaggacagaagccaaactggaagtggaggagccaagggattgacaaacaacagagccttgaccacgtggagtctctgaatcagccttgtctggaaccagatctacacctggactgcccaggtctataagccaataaagcccctgtttacttga

SEQ ID NO: 6 - DNA, 2480 bp, Homo sapiens
tttgcttcccctcttcccgaagctctgacacctgccccaacaagcaatgttggaaaattatttacatagtggcgcaaactcccttactgctttggatataaatccaggcaggaggaggtagctctaaggcaagagatctgggacttctagcccctgaactttcagccgaatacatcttttccaaaggagtgaattcaggcccttgtatcactggcagcaggacgtgaccatggagaagctgttgtgtttcttggtcttgaccagcctctctcatgcttttggccagacaggtaagggccaccccaggctatgggagagttttgatctgaggtatgggggtggggtctaagactgcatgaacagtctcaaaaaaaaaaaaaaaagactgtatgaacagaacagtggagcatccttcatggtgtgtgtgtgtgtgtgtgtgtgtgtgtgtggtgtgtaactggagaaggggtcagtctgtttctcaatcttaaattctatacgtaagtgaggggatagatctgtgtgatctgagaaacctctcacatttgcttgtttttctggctcacagacatgtcgaggaaggcttttgtgtttcccaaagagtcggatacttcctatgtatccctcaaagcaccgttaacgaagcctctcaaagccttcactgtgtgcctccacttctacacggaactgtcctcgacccgtgggtacagtattttctcgtatgccaccaagagacaagacaatgagattctcatattttggtctaaggatataggatacagttttacagtgggtgggtctgaaatattattcgaggttcctgaagtcacagtagctccagtacacatttgtacaagctgggagtccgcctcagggatcgtggagttctgggtagatgggaagcccagggtgaggaagagtctgaagaagggatacactgtgggggcagaagcaagcatcatcttggggcaggagcaggattccttcggtgggaactttgaaggaagccagtccctggtgggagacattggaaatgtgaacatgtgggactttgtgctgtcaccagatgagattaacaccatctatcttggcgggcccttcagtcctaatgtcctgaactggcgggcactgaagtatgaagtgcaaggcgaagtgttcaccaaaccccagctgtggccctgaggccagctgtgggtcctgaaggtacctcccggttttttacaccgcatgggccccacgtctctgtctctggtacctcccgcttttttacactgcatggttcccacgtctctgtctctgggcctttgttcccctatatgcattgaggcctgctccaccctcctcagcgcctgagaatggaggtaaagtgtctggtctgggagctcgttaactatgctgggaaatggtccaaaagaatcagaatttgaggtgttttgttttcatttttatttcaagttggacagatcttggagataatttcttacctcacatagatgagaaaactaacacccagaaaggagaaatgatgttataaaaaactcataaggcaagagctgagaaggaagcgctgatcttctatttaattccccacccatgacccccagaaagcaggagcattgcccacattcacagggctcttcagtctcagaatcaggacactggccaggtgtctggtttgggtccagagtgctcatcatcatgtcatagaactgctgggcccaggtctcctgaaatgggaagcccagcaataccacgcagtccctccactttctcaaagcacactggaaaggccattagaattgccccagcagagcagatctgctttttttccagagcaaaatgaagcactaggtataaatatgttgttactgccaagaacttaaatgactggtttttgtttgcttgcagtgctttcttaattttatggctcttctgggaaactcctccccttttccacacgaaccttgtggggctgtgaattctttcttcatccccgcattcccaatatacccaggccacaagagtggacgtgaaccacagggtgtcctgtcagaggagcccatctcccatctccccagctccctatctggaggatagttggatagttacgtgttcctagcaggaccaactacagtcttcccaaggattgagttatggactttgggagtgagacatcttcttgctgctggatttccaagctgagaggacgtgaacctgggaccaccagtagccatcttgtttgccacatggagagagactgtgaggacagaagccaaactggaagtggaggagccaagggattgacaaacaacagagccttgaccacgtggagtctctgaatcagccttgtctggaaccagatctacacctggactgcccaggtctataagccaataaagcccctgtttacttgagtgagtccaagctgttttctgatagttgctttagaagttgtgactaacttctctatgacctttgaa

SEQ ID NO: 7 - protein, 406 aa, Homo sapiens
MSALGAVIALLLWGQLFAVDSGNDVTDIADDGCPKPPEIAHGYVEHSVRYQCKNYYKLRTEGDGVYTLNDKKQWINKAVGDKLPECEADDGCPKPPEIAHGYVEHSVRYQCKNYYKLRTEGDGVYTLNNEKQWINKAVGDKLPECEAVCGKPKNPANPVQRILGGHLDAKGSFPWQAKMVSHHNLTTGATLINEQWLLTTAKNLFLNHSENATAKDIAPTLTLYVGKKQLVEIEKVVLHPNYSQVDIGLIKLKQKVSVNERVMPICLPSKDYAEVGRVGYVSGWGRNANFKFTDHLKYVMLPVADQDQCIRHYEGSTVPEKKTPKSPVGVQPILNEHTFCAGMSKYQEDTCYGDAGSAFAVHDLEEDTWYATGILSFDKSCAVAEYGVYVKVTSIQDWVQKTIAEN

SEQ ID NO: 8 - DNA, 38542 bp, Homo sapiens
tctagaagctgccatcatactgaaggtaatcttctgaaacttgcggttttctttcaaaaattatgtttataagatgatccatgttcttgcagagtttattcattttatgctgtataatattccattatatccacatacaatgcagtattgacccttcctcctgttgatgggcatttgtcttgtttctagttactttgctattatatcagtgtcaccatgattatccaaaagtaattcttttgtacactctaatttaagaacaactaaccctttttaatgaataaatcaaccttgtattgagttgctactaagtttcagttgactagtacctgggatacacacaggtgcagacatttgactgagacatattgatttttctcatctgcctatttaggctaatcaccagactataaaaccatgagaaccactgccattgagtatagtctgtgtcagtctacactatagctttaactagttgtgtgatttcttgcaaagagcaatcagagaagacacaataaacacatttactgatttcaggctggagagcttttaagcaatagggagatggccacacacaaggtggagaaaattactgtgaaaaggaagtactttctttagagccccacctaagctaggctgcagaaatgtctacaatgggtttgaaaaaactcaaaatgagcctttctgcagtgtgaaaatcctccaagataaagagacagattgatggttcctgccgccgccctgtcctgcccagttgctgatttcaggaaatactttggcaggtttgtgggtcatagagttgccaggtttcttgggatttgtaatagaacatcacaagaaaatcaagtgtgaagcaagagctcaactcttaacaggggtattgtttgtggttttgttactggaaaagatagtgaccttaccagggccaaagtttgtagacacaggaattacgaaatggagaagggggagaagtgagctagtggcagcataaaaagaccagcagatgccccacagcactgctcttccagaggcaagaccaaccaagatgaggtgggtccacagctttccctcctgcctttcctctggttctttatttcagtcttttttgcatacatcggtagagatgcagaaatagaacaaagaaacgggcaaatgggctaaattatagtgaaccaaagggcttagtgtgttaaatcttctccttttctgcatccatagaagacagtgctgctgtctttcccaggagataagatttactctcaggagtgtctttttccttcaggttacatttttgactttatagggtatgtcatcagctcccgtggtaggcttcctggcatcctgagtatatttattagcagatattttcctctttaaaaatgtacaataaggaagactaatagtaacacatttgaatgacacaattaattgactagtacctgggatacacactaatacctgggatacatctaattaaggcacttagatcttataaaaataaacactttttgaaatgttgaaataataagactagaaactttttttttttttgagatggagtctcgctttgtcaccaggctgcagtgcagtggcatgatctcggctcactgcaacctccacctcccaggttcaagtgattctcctgcctcagcctcccaggtagctgggactacaggcgtgcgccactatgcccacctaatttttgtatttttagtagagacgggctttcaccatgttggccaggatgacctcgatctcttgacctcgtgatctgcctgccttggcctcccaaagtgctgggattacaggcatgagccaccgtgtctggcctagaaactattttaatagaagcaagtagtgcccgaatggttggcatttgttagtgagatggtgaactggcagacggcacctgtgggtcaatgcccatggccaccgtcctgcttttggacaccagttctcttccaggtaaccttctggcattttggggtttcagataccatttcctaaaggtgaattattataaaatactagaaatacccacgtgtttaaaattatatatttaaggaaccttttattacggaaaatatcaagaagtagaggaatagcacagtaagcccaaagttcccatctctgagttatctacattttattaccactatctttgcggtgtctgagggaggtttctctttcctggagggctcctgtattattgccaatgtactttcctgaatgcagccagaaactgagcccacccctccacctatgtgcctttctatccctctctgaagcttctgcagaattcccagcaggacagggcttgctggaagcttggtatgctcagaagctgctaaagtgtgtatgggcaggtgtgggggcaatttcttggtcctagcacttccatatatcgactttcttttctggctgctaagtgggaggagtgtgtgtgtatgcatgtgtgtgtgtgtgtgtgtacatgcatgtgtgtgtggatgcatgcatgtgctgtgaagcagggagactagctttccactcctccttgtcttctctctgcagtgccctgggagctgtcattgccctcctgctctggggacagctttttgcagtggactcaggcaatgatgtcacggatatcgcaggtcagtctttggttgggtaggagtgtgcatcccactctgaccctctcgggtctgcactctctctgagaacacccaattcccccttcttatctcgacctctgggctttcaggaccataaagaacattggggttcctgccagaaatgaggggagcttgcctttccattggcttctattcggggtggaaggagattgatgtgcagagcagctcccgctcatctgacttttcacggttcactgggaacaatttccaaatagcaaactctctggcttctctctctttgcagatgacggctgcccgaagccccccgagattgcacatggctatgtggagcactcggttcgctaccagtgtaagaactactacaaactgcgcacagaaggagatggtaagatgtggacaactgtctccatgccctacatacaacccccttctctgacatttccatgatgggtggtgctgaggtgattcgccagaaagttcgttgctctccttggagccaggagatttagattctaataagcgttttgtcgccagtagccatggccctttgggcagactaacttttgtcagcctcaagttttctgttttgttaaggggaggcgatgccatgcagcctacctcatgtaaatctcagagtcagatttacatctccagcagatgtgggaaaagaaggaatgctgatgatgatgtcaccctcacctagtgagtcttgctgtcctggcactgctctaagggctttatacttatttgctcacttagtcctcacagtatccctctgaacagagtttattgttttcactttgctgataaggaaactgaggcacagacaggttgagtatcttgcccaaattcaggcagcctgtaagaggcagagtcaggatttgaaccctgagccctccctgtactgcttggctgtgaccgccatgaccacagtgtgttctgctgggcttaactggtgtccaggcacttggcttccagcacagcactctttcccttcctccttctcatattctctctcctttctcccttcctgtctgcctcctttcttcttcttctttttaattcttctccttaaatgccttctcactctgctctgggtgcagacttgacttttcctttggctcatttcttgccttttgtttcaggagtatacaccttaaatgataagaagcagtggataaataaggctgttggagataaacttcctgaatgtgaagcaggtgggtgctgagcactgagcacttaagagagcaggcaggcgtccagcggggaacgtcctagaggcacagccttccagtgcggcttcctctgagcacacaagagccaggaggagggatgtgggagaaccgcagctggccagggagagacttaagcagttaggtgatgactccctaagggtcaccaagggtcttgttcattggggcctgaagggcactggctgaatccactgtcggcactgcccacagatcaggagagcctgtgcatacagagagcctgctagaaagccctgggtctaaggagaagcaagctccagggagaacaagtcaaggaatgacataaaatcttaatccatggaagcctagcaggaggctggacatgggctggaactcctgcttctcgttattaggaggagctgttgctctctcctttcattctcagaaccagaggcaaagacccagcctcttctgctcttactggtgtggaaatgccaacctgcctcgtattaactgcaccatctacaaaatctgagctccagccagtgctgctctagattcatctttctttagagagaatgaattattgtagcccctagccctttcaatgaatttcagggaattgtggaaattcctttattgggataattgtttaaatataatacagttcgcgagcttctattcggggtggaaggagattgatgtgcagagcagctcccgctcatctgacttttcacggttcactgggaacaatttccaaatagcaaactctctggcttctctctctttgcagatgacggctgcccgaagccccccgagattgcacatggctatgtggagcactcggttcgctaccagtgtaagaactactacaaactgcgcacagaaggagatggtaagatgtggacaactgtctccatgccctacatacaacccccttctctgacatttccatgatgggtggtgctgaggtgattcgccagaaagttcgttgctctccttggagccaggagatttagattctaataagcgttttgtcgccagtagccatggccctttgggcagactaacttttgtcagcctcaagttttctgttttgttaaggggaggcgatgccatgcagcctacctcatgtaaatctcagagtcagatttacatctccagcagatgtgggaaaagaaggaatgctgatgatgatgtcaccctcacctagtgagtcttgctgtcctggcactgctctaagggctttatacttatttgctcacttagtcctcacagtatccctctgaacagagtttattgttttcactttgctgataaggaaactgaggcacagacaggttgagtatcttgcccaaattcaggcagcctgtaagaggcagagtcaggatttgaaccctgagccctccctgtactgcttggctgtgaccgccatgaccacagtgtgttctgctgggcttaactggcatccaggcacttggcttccagcacagcactctttcccttcctccttctcatatactctctccttttccccttcctttttgtccccttttcctcttccttttagttcttctctttaaatgccttctcactctgcacggggtctagacttgacttctcctttggctcacttcttgccttttgtttcaggagtgtacaccttaaacaatgagaagcagtggataaataaggctgttggagataaacttcctgaatgtgaagcaggtgggtgctgagcacttaagagagcaggcaggcgtccagcggggaacgtcctagaggcacagccttccagtgcggcttcctctgagcacacaagagccaggaggagggatgtgggagaaccgcagctggccagggagagacttaagcagttaggtgatgactccctaagggtcaccaagggtcttgttcattagggcctgaagggcactggctgaatccattgtctacatcgcccacagattaggagagcctgtgcatacagagagcctgctagagagccctgggtctaaggagaagcaagctccagggagaacaagtcaaggaatgacataaaatcttaatccatggaagcctagcaggaggctggacatgggctggaactcctgcttctcgttattaggaggagctgttgctctctcctttcattctcagaacaagaggcaaaggcccagcctcttctgctcttactggtgtggaaatgccaacctgcctcgtattaactgcaccatctacaaaatctgagctccagccagtgctgctctagattcatctttctttagagagaatgaattattgtagcccctagccctttcaatgaatttcagggaattgtggaaattcctttattgggataattgtttaaatataatacagttcaccagccagggctcaaaaatctcagtatttcccacttcctttgttagaaaagtgggaaatagagctttttgtaatgtaaacaatttaaaaaacagaattattttaaaactgcaactattggaaatgagatcagcaggtggtaagggcaaagcatttaaatctttctactttacgcagcagtgacagccgcccatgctttcacccctttctcagatggaaaggctcttgcacatttccactcacgagtgtcttgctctccttgacagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccatttctgtgttgtgttcagtcttgagtcttttttatttgctcctttatggtccagggtagtcagaaggtatagagtctactgggagtatggcagaaaacaccctaaacccactggaaatcccgaaggtgatacaaactcttccaccttagggaatcatgctcactgattgagtgcctattgaatgctaggtcccagaaagttaactgttgtccttgttttacagacaaggaaacagagactcagagatggtaagtgagttgcttaaggttacatagctatgaaacagggaagcagaactttgaacccaggtctgtttgatacaaactcagaggtcctttcactgcatgctgttgcctcctcaaagtgaattaggagaaaaggcatgggcctggttgaggaagaggctagctcaaaatgggatggggaaaaagtgttttaacacagacagtaattcagagtttgggatctaacctaccagcactccagaaaacaaaagacaatgatgtaaaggcaggatctctggacttactgagtccaaatcacagaatggcccataaatttgctaggtaatttaggtttcctggccatcagttcctggtctcttccatgaaggacttgagccaggcgatggctaatgtccctatcagcaataaccttctggtattccataaagagtggggaggtagagtccctgcatcagtagaagctgcttttcctactgggaacaaacccaccctgcaccaggaatcaaatgcaggctgtctgagcctcctctcctgatggcaggtcatgctattaagggtctcccagaagaaaggttctggaactctgagaaacagaagctcttctaacagaactttttcttgctataagctttcaataccaccaatcaccagtgcaccttcttctcatgccaaaatacggcccctcctttgtagggtgtaaaacgacactgatgtcactctctctcaccaaggtctggccatctctgtgaagctctcctcacaggcaccctgcagattcatcaataaacttcaacaggttagcgccagtaggccaataagatgcacacttatattctagaacctgtgaaagaagtaatagccagaaactggaaaataacagtcaaactcttaaaaaaatgcctctcagaacacagtcacttacaccttttcctctggtagttctcttcagcaccaagtttcctggaagaacaagctactccccagttttcttaaagaattgtctgattgtattaagtaagtggcttctggtaaaataatggtaagagaaaagctctggcattctcttgatcactcatcttctctgccttcctccctagcaacatctcctggcctcagcctgtgggcttcagcaacaccaaactccatgttgctctctgacaagcccggccgtctccagttcttgggctttatttcctcgggggccctctgctgaaattcccttccccttgctgtattagcacctcctacccgccttcaacatgcaggcactgcctcctctaaggagccatctgtacttacgtagctgtgagcataggatggggcatacagcaggcacttaacaaatacttgtggaatggaaagaaactaagataggatcaaaaatacaaatcaagatgtgagagaaagacgatcaaaagccctcacgagggggtcgggcgtgatggctcacacctgtaatctcaacactttgggaggctaaggcgggtggatcacttgagggtgggagtttgagatcagccagagcaacatggagaaacctcgtctctactaaaaatacaaaaaattagccaggcatggtgatatatgcctgtaatcccagctactcgggaggctgaggcaggagaattgcttgaacctcggaggtagaagttgtggtgagctgagatcatgccattgtactccagcctgggcaacaggagtgaaactctgtctcaaaaaaaacaaaaaacaaaaaagaacaccacgggaaaatcaagtgtgaagcaagagctcaactcttaacaggggcaatgtttggagttttgttactggaaaagatagagaccttaccaggtccaaagtttgtagacacaggaattacgaaatggagaagggggagaagtgagctagtggcagcataaaaagaccagcagatgccccacagcactgctcttccagaggcaagaccaaccaagatgaggtgggtccacagctttccctcctgcctttcctctggttctttatttcagtcttttttgcatacattggtacagatgcagaaatagaacaaagaaacagggcaaatgggctaaattatagtgaaccaaagggcttagtgtgttaaatcttctccttttctgcatccatagaagacagtgctgctgtctttcccaggagataagatttactctcaggagtgtctttttccttgaggttacgtttttgtctttgtagggtatgtcatcagctcccgtggtaggcttcctggcattcggaatatatttactagcagatattttcctctttaaaaatgtataataagaaagactaatagtaacacatttgaatgacacaattaattgactagtacttgggatacacactagtacctgggatacatctaattaagacacttagatcttaaaaaataaaaagactttttgaaatgttgaaataataagactagaaacttttttttttgtgagacagagtctcactctgtcaccaggatggagtgagtggcgcgatctcagctcatcgcaacctccccctccccggttcaagcaattctcctgcctcagcctcccaagtagctaggactacaggcgtgtgccaccacgcccagctaatttttgtatttttagtagcgatgtggttcaccatgttggcccggatgatctcgatctcttgacctcgtgatccacctgtctcggcctcccaaagtgttgggattacaggtgtgagccaccgcaccctgcctagaaactattttaatagaagcaaatagtgcctgaatggtgggcgtctgttagcgagatggtgaactggcagatggcgcctgtgtgtcaatgcccatggccaccgtcctgctttcagacaccagttctcttccaggtaaccttctggcattttggggtttgagataccatttcctaaaggtgaattatcacaaaatactagaaataaccacataagtgtttaaaattattgttaaatacagtaagaaattcttcttcaaaagtttagcctgcttaagtttccttgtcctttgtttcctgctttcaaggccagacttccttactctctgtgtttcccctaccctggtaaacaaccttcctgccagtccttacccatacagcccacattccacatctgctacccactctgtgatttacctctcccgtcgcaatagcctctcccaccaaaactgatcttcccgccttcccaccagtgcaaccacattcctgcactttgcaagttagccaaccgggttcggattgtgcagtccaactccagccaatggagtcaggacacagtagcagggacaagctgcgttagacataaaaacctctgctttcctttgtttagggtgctctcgtggcaaccagacttaccaggagctctattctgcaaaagtaaatttgccttgctgagagaccctttgtcctttggctcagtgttggttcttctttgcagcaccgagcatttgtttccaacaaatttggtggcccatacagggaaaacattgtcctccgggaaagggttctttgatcatcctcttgagaggagaacacatcccactgtccttgttgcggtggcctcatgagtaggaatcgagacccacctgtctgatgaataaccccagactctcaacaacgtggggagaaaaagacttgcaacactatggtggccaggtaactctgtgcgcagaccaaggtaagaaatgtcgcaggagtgacaaagtacttccttggtggtcactatattctggtggctgaaagttcatgaatggtaacaagtgctactgctgtgtggagtgaatgagtccaatctgtgggtctatggttacctcatacggcttagccttctctgaaggatcctgatgttggggtttatatagtcctcccaatgctaagcgggacttaaaatattcctaggaggaaagtggccagagtggatgaagcaaaaggagaagagtgtgaagaacctccaggaggtggggctaaaagataggcaagaaatctctaatacgagggattgagccacagaaagctccagacagataaaaaagaaattcctaatatgaggaactgagccacagcaagcctccagcaggcaacaaatccctaatatgagggattgagcctagctaagacccaatatgggaaataccccaagaaagacagagaataagaaggatgaaaatagtaacaaggatataccccttgatagtcccctaggtttcatgttaaaatactggaaagataatgagaggactaagcataagaagaaggagcaaatgataaaatattgctgtttcatttggacccaaggtcccatcctcaaaccctcaatcttctggccaaagtatgggtcgaatgagggtgtaatgagtcaactcctaatccaatatgttaatgataaaagtctggtttctcaagaagaactagactatgctctttgttggaagcagggacctgtcctcctctttcccttaaagacaactagggaagaatccgatccagcatctcaaattgagaagtcagacaagccgactcccacacctaaagtcagcacatgggatcccctagactgtcttcctttgcttactgccccaatcctaacccccctactcctcaggcagctgctgctgccccagatcccatcccagattcttccccaactcatgatgttcctcccgcttacaaccctgactctcaggggcagtcgtcccaagagcctgttcattgccaacctaaatatccttccttaaaagggctccaacatgaaatagagcagtgtaaaaaggacattcagaactccccttttctttccacacctaaggagtcagccataactttcttccctttaaaaggggtatcacaaggaggggaagtcattggttttgtaaatgctctcttgactatttcagaagtctgaggtctgaagaaagaacttaagccactgctagatgacccctatggagtggcagatcaagttgatcaattcttagggacctcagttatacacttgggtcgagcaaatgtctatcctaggctcttcttttcaggggaggaaagaaagcatgatccgtagggctgctatggcaatttgggaacatgaacaccctcctggtcaaaacgttcctactgtggaccaaacatttgctgcccaagcaagactcctggtgggacaacagcaatgcagcccactgagaaaacatgcaggacctaagggaaataataataaaaggaatcagtgaatccatacccagaactcaaaagctctctaaagcatttgatatacaacaggagaaagatgagggacctataagattcctagacagactaaaggagcaaatgaagcaatatacaggtctgaatttagaagatccccttgggcaaaggatgttaaagatccattttgtcactaaaggctggccagatgtttcaaaaaagttacaaaaattggaggactgagaaaaccgacctctaagagaacttctcagagaagctcaaaaggtgtatgtgaggagggacagggaaaaacaaaaacagaagtcaaaaccaatgttatctactttccagcaggtggctccaaacccatatgctactaaatgaggcttccagggagccagaaactataaaaggtcccaagcctcccaaacccagtttagagaaaccaaaccttcagctagaggacccaagtctacatttcccaggccccctaaagagcatagaaaagcaagaccaaaaaatcccaaaactgagagaggggaaggacaagataagtgttacaaatgtggaaggacagcccccttcaaaagagaatgtcccaaattagaaaaggagagagaagcccttccactcacgacctttgaagaggaacagggaagtcaggggctctgtctatattatcttgagtcccaccaggagcccttgataaatttggaggtgggacctacacatgagcttatcacatttttggttgattcaggagcggcctgttcctctgtttgtttcccctcatctaatgttgcctgctcttcagaagaacttatagtctctgagataaaaggggaaggatttacggtgagaatcttagaaaatacagaagtcaagtaccaagactaaacaacccaggttcaatttttgttaatccctgaagcagcaactagtttgttaggaagagacttaatgttaaagttaggcataggcctacaagtcagcccaaagggattccttacttcattaaacttactcaccacggcggatgagaaatacattcatcctgatgtttggtcaagggaagaaaactgaggaaagcttcgaattctcccaatccacatcaagctaaacaccccgcactgggaagtagtgaggaggaagcaattccccattcccttagagggcatgctagggctaaaacctataattgaaagtctcattaatgatgggcttcttgaaccctgtatgtctccttataacaccccaatactgcctgtcaaaaaatcagatgggtcataccggctggtgaaagacctcagagccattaaccaaacagtccagaccactaaccctgttgtccccaacccttacaccattctcagcaaaattccatataatcatcaatggtttactgtaatagatttaaaggatgctttttgggcatgtccctggctgaagagagccgagacacatttgcctttgagtgggaagatccccagttagggtgaaaacaatggtatcaatggacagtcttgcctcaggggttcatggattcacccaacctttttggtcaaattttagaacaagtgctagacaaagtttctgttccaaaacaattatgcctgcttcaatatgtcgatgatattctcatatctggtgaggatatagagaaagaagctggcttctctacacatatttttgaccatctacagttcgaggggttacgggtctcaaagggaaagcttcagtgtatggaacctaaagttaaatatttaggccgcttaataagtgcgggcaagcgaaggatagggcctgaatgggtcgaaggaatcgtgtccttacccttgcctcagactaaacaggaactcaggaaatttttagggttagttggatactgccgcttaaggattaactcacatgccctaaacagtaaacttttataccaaaaacttgcccagggaaaacctgagcatctcctgtggacttctaaagaggtcgatcaggtcaaagagctaaaaggaatagcttataactgctcttgccctagccttaccttccctagaaaatacacttcaccttttcgtcagcgtgaaaaatggggtggctttaggggtgcttatccaagagcacagaggctgctggcagcccatggccttcctgtcaaaaattttggacctggtcacctgtggatggcctcagtgcatccaatccattgcagctacagcagtattagttgaagagagtagaaaattaacctttgggtggagattaacagtaagcacaccccaccaagttagagagctattttaaataaaaaagcaggaaggcgactaactgactccagaatcttaaaatatggggctattctactaaaaaaagatgagagaacacctatgtctagatttaattgactaccaaacaaaactcaggccagatctaggaaagatccctttcaaaacaggacggcacttatttatagatggttcctcccagctgattgagggaaaaagacacaacgggtatccagtaatcgatggagaaattctctatgtataagaacagagtcaagaaaattgcctaataattggtctgcccaaacttgtgaactgtttgcactcagccaagccttaaagcacttgcaaaaccaggaaggaaccatctatactgattctaagtatgcctttggagtggctcatacatttggaaaaagttggactgaatgtggcctcactaatagtaaaggtcatgaccttgttcataaggagttaatcatccaagtactggataaccttcagttgccagaagaaatagctattgtccgtgtacccgggcaccagaaaagcctttcttttgaaagtcgaggaaataacctaacaaaccagatagccaaacaagctgctgtttcctccgaaacacctatgtttcacttaactccttgtcttccttcccctactgcaatttcctttttctcttccattgaaaaagaagaataaagataggagccaaaggagaagaccagaatgaaaacggctgttactagaccaaaaggaaatgttatccaagcccgttatgtggaagatctagtctcaactatatctgaggacacactggggatcccaagccatgtgcgatgcagttctcagggtctatggatgtataagaatttacaccctagccaaacaagttacagatagttgcttaatatgtaaaaagactagtaagcagattctaagaaaaccgccccttggagaaagagattcagggctaagaccatttccaagtgttcaattaattatactgaaatgcccccaattggtcatttaaaatacttattagtaataatagaccactttacctactgggtagagactatcccacactcaaatgcaaccaccagtaacgtagttaaggcattaattgaaaacattgtacccagatttggactaatacaaagcactgattcagacaatggaacccatttcactgcatatgtcattaaaaagttagcccaggtactagacataaaatggaaaaaccatatcccttggcatctctcctcctcaagaagagtagaaaggatgaatcagactctaaaaagccacttaactaaattagttctagaaacttgattgccatggactaaatgtcttcctattgccttgttaagaatccgaactgctcctcagagagatactggcctttccccttatgagatgctctatggattgccctatttatactccactgctaacattcctacattcaaaataaaagatcagttcctcaaaaattatatacttggtccatactctactttctcttcccttaagactaaaggtctcctagcacaggcgccaccgctggagtttccagcacattagcgtcagcctggagaccatgtcctcataaaagggtggaaggaaggcaaactcaaaccagcttgggaaggaccctacttggtgctcctaaatactaagactgcagtccgaacagcagaatgaggatggactgatcacacccgcgtcaaaaaggcgctgccacctccaggatcataaaccgtcactccagggctcaccccaaccaaattaactctaaaaagggcttaataatcacttgtttattttttcttttctttccaacagaaggtcatcttgtcatcaatgtaacttgggctaaccatcctttaatccttcagtttgatgcttgttcagtcatcctgtgtggagacaagcaagctcaaaggaagctgtctcatgtagataagtacctatgtccataccataaaaagtcaaccaagtataagtatagaaccttaaaaagtccctgtggtgactggacagatgtttggaaaccactcagtatggagggtggacagccaggcccccttttcaaataagttatggggactaaaacagaaactccaactaattcatggtcccaccccaccaaactgtaagccactgcagtgtaacccctcattgctaattatagctaacccccaaacaatggcccaagaacccactatattcaaacagtatggattaggagcaaatgttacaaaacaaaatcccataaaaatcttcaaccactggttaacaatgataattaaaaaaaaatccacacccagagtctgggaaacgagtggaatctaacactctctccaagtatagcgagctcagcgtcctcctcccatctccaaaacaacccaactaaagtaacggttgtaaaggtaaaaaatttaaagcagactatagcccaactaaagtaacggttgtaaaggtaaaaaatttaaagcagactatagccctagagacaggatatcaagatgcaaagtcttggctaaaatggattaaatattccatctgcactttaaacaaatatgaatgttatgcttgtgctcacggtagaccagaggcccagattgtcccctttccactcagatggtcttctaactgaccgggcataagctgtatggtagttctcttccaagatcacacagcctggggtgacaaatcatgtcaagctctttctctgctgttccctgaagctcaacaccctgagggtcagcccccgagggccatcagcttccgtttcccaatgccaattttactttgtgtctctcacaacagggagaaaacttggtgttccttggagccttaatgggatgcagtgagcttaagtccttccaagagcttacccatcagtctgcccttagtcatcctcgagcggatgtatggtggtattgtggtggaccattactggacactgccgagtaactggagcagaacatgcactctaattccattggctatccctttcaccctggcatttcatcaaccagaaaagatagaaaccaaacactgtaaaacaagagaggcccctcatgggtctgttgactcccacatttatatagattccactggagtccaacgaggagtgccagatgaatttaaggcccaaaatcagacaactgcaggatttgaatccacgttcttctggtggttaactataaataaaaatgtgaattagataaactacatttattacaaccaatgatgatttttttttttttgacggagttttgctcttgttgcccaggctggaatgcaacggcatgatctcggctcaccacaacttctgcctcctgggtccaagcaattcttctgcctcggcctcccgagtagctgggattataaacatgtgccaccacgcctggctaattttgtatttttagtagagacagggtttctccatgatggtcaggctggtctcaaactcccgacctcaggttatctgcctgcctcggcctcccaaagtgctgggattacaggcatcagccactgtgcccagcctatgatttgttaattatactagagatgctcttaaagggacagctgaacaattaagacccaccagtcaaatggcctgggaaaataaaatagcattagacatgataccagcgaaagaaggtggagattgtgtcattatcggaacccgatgctatacttttatccctaataatactactccaggtgggatcacaccaaaagcactacaaggtcttaccgccttatcaaatgagctagccaaaaattctggactaaacgaccccttcacaaatttaatgggaaattctttggcaaatggaagggatttatgtcctcaatcctcatgtctcttgccatcagaatagggttgcttattcttgtaggatgctgtgtcataccctgtgccctaggattaatacaaaagctcattgaaacagctctccccaaaacctccctcaatcctcctcctcctcgttccagtgagctttttcttttagaagacccaagtagaataacagagccaaattatgttaaaagggtttaagaaaaaatactgtaaaatatttaacagggggattrtgttaaatacagtaagaaattcttcttcaaaggttcagcttgcttaagtttccttgttctttgctccctgctttcaaggccagacttccttactctctgtgttctccctgcgctggtaaacaaccttcccgccagtcgttatctatagagcccacattccacatctgctacccactctgtgaattacccctcccatcacaatggcttctcccggcaaaactgatcttcctgccttcccagcagtgtaatcacattcctgcacttttcaagttagccaaccgggttcagcttagattgtacggtccaactctagtcaatggagtcaggatacagtagcagggacaagctgcgttagacataaaaacctctgctttcctttgttcggggtgctctcgtggcaaccgaacctatgaggagcaaccttatgcaaaagtaaatttgccttgctgagagaccctttgtcctttgtctcagtgctagttcttctttgcagcaccaagcatttgttttcaacaattatatatttaaggaatctttgattatggaaaatttcaagaagtagagggaatagcatactaagccctctgttcccatctttgagttatctacattttattagaagtatctttggggtgactgagggaggtttctctttcctggagggcttctgaattattgccaatgtaccttcctgactgcagccagaaactgaggcccctatacctatgtacctttctatccctctctgaagcttctgcagaattcccagcaagacagagcttgctggaagcttggtatgctcagaagcagctaaagcgtgtatgtggggcggagggtgggggcaacttcttggtcctagcacttccatatattgattttcttttctggctgcttagtgggaggagtgtgtgtgtatgcatgtgtgtgtgtgcgtgtgtgtgtgtgtgtacatgcctgtgtgtgtggatgcatgcatgtgctgtgaagcagggagaccagctttccgttccttcttgttttctctctgcagtgacctgggagctgtcatttccctcctgctctggggacgacagctttttgcactgtactcaggcaatgatgtcacggatatttcaggtcagtctttgagttgggtaggagcatgcatccctggcactgccacatcccactctgactctctcgggtctgcattctttctttgagaacacacagttccccattcttatcctgacctctgggctttcaggactgccaagaacattggggatcctgccagaaatgaggggagcttgagctttcgttggcttctatttggggtagaaggagattgatatgcagagcagcttccactcatctgacttttcatgggtctctgggaacaatttccaaatggtaaactctctggcttctctctctttgcagatgaccgcttcccgaagccccctgagattgcaaatggctatgtggagcacttgtttcgctaccagtgtaagaactactacagactgcgcacagaaggagatggtaagacctggacaactatctcctgtgctctacctacaacccctgctctgacatttccatgatgggtggtgctgaggtgatttgccagaaagttcgttgctctccttggagccaggagatttagattctgataagcgttttgtcgccagtagccatggccctttgggcagactaacttttgtcagcctcaagttttctgttttgttaaggggaggtgatgccatgcagcctacctcatgtaaatctcagagtcagatttacatctccagcagatgtgggaaaagaaggaatgctgatgatgatgtcaccctcacctagtgagtcttgctgtcctggcactgctctaagggctttatatttatttgctcacttagtccttacagtttacctctgaacagaggttattattctcactttgccgataaggaaacagaggcaccgacaggttgagtatcttgcccaaattcaggcggcctgtaagaggcagagtcaggatttgaaccctgagccctccctgtactgcttggctgtgaccgccatcaccacagtgtgttctgctgggcttaactggtgtccaggcacttggcttccagcacagcactctttcccttcctccttctcgtattctctctcctttctcccttcctgtctgcctcctttcttcttcttctttttaattcttctccttaaatgccttctcactctgctctgggtgcagacttgacttttcctttggctcatttcttgccttttgtttcaggagtatacaccttaaatgataagaagcagtggataaataaggctgttggagataaacttcctgaatgtgaagcaggtgggtgctgagcactgagcacttaagagagcaggcaggcgtccagcggggaacgtcctagaggcacagccttccagtgcggcttcctctgagcacacaagagccaggaggagggatgtgggagaaccgcagctggccagggagagacttaagcagttaggtgatgactccctaaggatcaccagggtcttgttcattggggcctgaagggcactggctgaatccactgtcggcattgccacagatcaggagagcctgtgcatacagagagcctgctagagagccctgggtctaaggagaagcaagctccagggagaacaagtcaagggataacataaaatcttaatccatggaagcctagcaggaggctggacatgggctggaactcctgcttctcgttattaggaggaactgttgctctctcctttccatctcagaaccagaggcaaaggcccagcctcttctgctcttactggtgtggaaaggccagcctcttctgctgtgagtggtgtggaactaccaacctgcctcgtattaactgcaacatctataaagtatgagctccagccaatgctgctctagattcctctttcttcagagatgatgaattattgtagctcctagccctttcttttttctttctttctttttttttttttttgagacagagttttgctctcgtcgcctaggctggagtgcagtggtgctaattcctgacctcaggtgatccaactgcctcagcctcccaaagtgctgggattacaggcgtgagccaccgcatctggcccctagccctttcaatgaatttcagggaattgtgaaaattcctttgttgagataattgtttaaatataatatagttcaccagccaggcctcaaaaaactcagtatttctcatttccttatttagaaatagagctttttgtaatgtaaacaattaaaaaaattattttaaaactgcaactattggaaatgagatcagcaggtggtaaggacaaagcatttaaatctttccagtttatgcagcagtgacagccgccaatgctttcacccctttctcagatggaaaggctcttgcacatttccactcacgagtgtcttgctctccttgacagtatgtgggaagcccaagaatccggcaaacccagtgcagcggatcctgggtggacacctggatgccaaaggcagctttccctggcaggctaagatggtttcccaccataatctcaccacaggggccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggtggttctacaccctaactaccaccaggtagatattgggctcatcaaactcaaacagaaggtgcttgttaatgagagagtgatgcccatctgcctaccttcaaagaattatgcagaagtagggcgtgtgggttacgtgtctggctggggacaaagtgacaactttaaacttactgaccatctgaagtatgtcatgctgcctgtggctgaccaatacgattgcataacgcattatgaaggcagcacatgccccaaatggaaggcaccgaagagccctgtaggggtgcagcccatactgaacgaacacaccttctgtgtcggcatgtctaagtaccaggaagacacctgctatggcgatgcgggcagtgcctttgccgttcacgacctggaggaggacacctggtacgcggctgggatcctaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccatttctgtgttgtgttcagtcttgagtcttttttatttgctcctttatggtccagggtagtcagaaggtatagagtctactgggagtatggcagaaaacaccctaaacccactggaaatcccgaaggtgatacaaactcttccaccttagggaatcatgctcactgattgagtgcctattgaatgctaggtcccagaaagttaactgttgtccttgttttacagacaaggaaacagagactcagagatggtaagtgagttgcttaaggttacatagctatgaaacagggaagcagaactttgaacccaggtctgtttgatacaaactcagaggtcctttcactgcatgctgttgcctctttaaagcgaattaggagaaaaggcatgggccttggtgaggaagaggctagctcaaaatgggatggggaaaaagtgttttaacacagacagtacttcagagtttgggatctaacctaccagcactccagaaaacaaaagatgatgatgtaaaggcaggatctctggacttgctgagtccaaatcatggacttggcccaaacatttgctaagtaatttaggcttcctggccattagttcctggtctcttccatgaaggacttgagccagggtaacgtccctatcagcaataagcttctgggactctgtaaagagtggggaggtagagtccctgcctcagtagaagctgcttttcctacttggaacaaacccacccttcaccaggaatcaaaagcaagctgtctgagcctcctctcctgatggcaggtcatgctattaagggtctcccagaagaaagcttctggagctctgagaaacagaagctcttctaacagaactttttcttgctataagccttcaataccaccaatcaccaatgtgccttcttctcatgcgaaaatacggcccctcctttgcagggtgtaaaatgacactgatgtcactctctctcaccagggtctggccatctctgtgaagctctcctcacaggcaccctgcagattcatcaataaacttcaacaggttagcgctagtaggccaataagacctatattctagaacctgtgaaagagataatagccagaaactggcaaacaccagtcaaactctttaaaaaaatgcctcttagaacacagtcacttacacattttcctagatctcttcagcaccaagtttcttggaagaacaaggcactctgcagttttcttaaagaattgtctgattgtattaagtggcttctggtaaaacaacagtaagagaaaagctctggcattctcttgatcactcatcttctctgccttcctccccagcaccacctcctggcctcagcctgtgggcttctgcaaagtcaggtatcaggactaattagctagcatcaaattcctgttaattccagaagcagggacaaacctgttggggagggacttaatgacaaaattaggtataggtctatacgttaatcaaggaaaattctcaacttccttaaacttacttaccaccctagaggagagccaaatccactctgatgacgtttggtcataggaaggaaattggggaaaattacaagtttctccaatccatgtaaaattaagaaatcctggggaagttgtaaagagaaaacagtaccctattcccttgaaaggcagaataggtttataacttataatcgaaggtctccttcaggaaggacttcttgaaccccatatgtcctcttacaacactccaatattgcctgtaaaaaagtcggatgggtcatagcggctactgcgagaccttcgagccattaaccaaatagtccagaccacccaccctgtcattcctaacccttacaccattatcaagcaagatccctcatgactgccagtggttcacagtaatagacctcaatgatgacttctgggcttgccccttagctgtggatagctgggacatatttgcttttgaatgggaagaccctcattccagttggaagcaatagtagtgatggacagttctaccccaagggtttacagactctccaaacctattagtcaaagtttggaacaagtcctagagaatttccctctttcatcatccttatgtctactccaatatgtggatgacctgctcatttccagaggcaccaaagaccaagtaaccgcaattttaattagctttctaaatttcctaagggaacaatggttacgggtctcaaaaagtaaactccagcttgtagaacctgaggtaaaacacatggggcacttaataagcaaaggtaagcggaagatagggcccgaacaaatggaagggatcatatccctgccactgcctgagacaaaacaagaacttagaaaattcttcaggctagctggaaattattgcctgtggattgactcttatgccttaaaaacaaaacctctatatctaaaacttacccaagaagggcctgacccccttctttggaccccacaagaagtccagcaagttgaggaactaaaacatctacttataactgcccctgtcttagctctgccatccttagaacagccatttcacttttttgttaatataagcaatggggcagctataggagtactcactcaaaagcatggggccattgccagcccacagcctttctgtcaaacattcttgacgtggtaacctgtgggtggcccgaatgtatccaatctacagcagcaactgttttattgacagaggaaagtagaaaaataacctttgggggaagtctcatcctaagcacaccccatcaggttaaaaccattcttagccaaaaagcagggagatggcttacagattcaagaatcctaaaatatgaagctatcctattagaaaaaggtgatttaaccctaaccactgacaatgtgctcaaccccgccaccttccttttttttttttttttttgagatggagtttcactcttattgcccatgctagagtgcaatggcacaatctcggctcaccgcaacctttgcctcctgggttcaagcaattctcctgcctcagcctaccaagtagctgggattacaggcatgctaccatgcctggctaatttttttgtatttttagtagagatggggtttctccatgttggtcaggctggtcttgaactcctgtcctcaggtgatctgcctgccttggcctcccaaagtgttgggattacaggcgtgagccactgtgcccggcctaccccgccaccttcctgacaggaaatccaaacgctggagaccccaagcacaaatgtttagacttaatcatcaaactaaagtaaggcctgatctaagtgagacccctttaaaaacagggcaccatctctttgtagacagctcctctcgggtaattgaagagaaaaaacataatggctactcagtggttgatggagaaactctcacagaagtagaatcagggagacttcccaataactggtctaaccaaacatgtgagttgttcgcactaaatcaagccttaaaatagctgcagaatcaagaagggactatttacaccaattccaaatatgcttttggagtggcccatacctttgggaagatttggactgaacaaggccttattactagcaagggccaaaaccttgtccacaaagagttaatcatgcaagtattagaaaaccttcagctaccagaggacatagccattgttcatgtcccaggacaccataaaggtctctctttcaagagttacttcccaagaagcacccattttccactgaaccccttgtctcccttccccatctgcaacccccatcttctcccaagcagacaaagaaaaattaaagaaaataggggctaacaaaaactcagaagagaaacggatactgccagataacaaagaaatgttgtccaaacccctaatgacagaaatcttaacacaacttcaccaagggacccattggggtccccaagccatgtgtgatgcagtccttagaacctatgggtgtacagggatttataccctcaccaggcaagtcacagacagttgtatggtatgcagaaaaaccaataaacaaactttaagaaaactgcccttcggcggaagaaatcctgggctaaggcattccaaagtgtccaaattgacttataccgaaatgccccaaataggctgcctcaaatgtttgtcagttatagtggaccatctcactcactgggtggaagccattcacctgccaagtgcaactgccaataatgtagttaaagtactaatagaagataccatacccagattcagattaatagagaacattgactcagacaatggaacccattttactgcacaggtcattaaaggattaacccagacactagagataaaatgggaatatcatactccctggcacccaccctcatcaggaaaagcagaaagaatgaatcaaaataaataaatcacctaactaaactaatcttggaaacccgactgccatgaactaagtacctccctgtcgccttactaagaattcgtatcgccccttgaaaagatatcggcctgtccccttatgaaacgctctacagattgccatgcttaaatccaccactgacatccctacatttgaaacaaaacatcaatttcttaaaaactatatacgtggtctatcttccacccttttttccctcaggacccaaggcctcttagcacaaatgccgcctctagagtttccagtacacccacatcagcctggggattatgtgctcatcaaaagctggagggaggaaaactcgaaccatcgtgggaaggaccctatctaatactcctaacaactgaaacagcagcccgaacggctgaacaggggtggactcatcacacttgggtaaaaaggaccccaccccttatggaatcatggaccatcactccaggaccaactccctcaaaattaacattcaaaaaggtctaatttgtctcttcttccccctaatgcccaggggcatttcatttttttttttttttgagatggagtctcgctctgtgcccaggctggagtgcagtggcaagatctccactcactgcaagctctgcctcctgggttcacaccattctcctgcctcagcctcccgagtagctgggactacaggtgcctgccaccatgcccggctaattttttgtatttttagtagagacggggtttcacagtgttagccaggatggttgatctcctgaccttgtgaactgcccgcctcggcctcccaaagtcctgggattacaggcatgagtcaccgtgcccagcccgcatttcattgttaatgtaaccaggtcagcctcccctcaagctgttgtgtttgatgcttgcctagtcataccttgcagagacctacaaagtcaaaggcaactagcctcttcagaaaagtatctttgcccttggtactcaccccaccctcaatctgatccttgcaagattccctataattggctaccttgccattcctggtacgatattctctggaccactcagtcccaaggctggacttcttcagcaggctgcacctccctaaaaccttatatccattttactaaacaaaataactcctctaactgccaacacctccgatgtaacctggtgcatatttctattactacttcaacctccactaaccatattcccaccctaagtcgcttttatggtatggggcagatgttagcgggaaagaccccatagggtcctttgagattcgttttgtcgcccctccagcaccccttcctttccgaagatctcctccaaatgaaacaactatcttgccactacctaatgacaaaactaaagtgcacatagtggaagtcaaagacctaaaacaaaccctggccatcgagaccggacaccaagaaatgaatgcctggctgaaatggatcaaatattcagtttgcactttaaataaaagcgactgttatgcctgtgcaatgggcaggccagaaatccaaatagttcccttcctgcttggatggtctaaccagccgggcatggactgtacagtagctctcttccagcaccccacagcctggggtaataagtcatgcaccagtctctcactgcttttcccaaaagtcaaagacttccctgtgggtcagcccccaaggccatctggctcctggcctctgatgccaattttacctcttgtctctcatgacggggaaaacttacatctttcaggaatctaacgggatgcagtgaatccgagccttttcgagagctcaccaatcagactgcccttgtctatccacaagcagatgtttggtggtattgtggaggacctctactgggtatgctgccaaataattggagcagcacttgcactctaatccaactggccatctctttcaccctggcatttcatcagtccgacaaaaagtcaccctcttgggaagaaagagaagctcctcaagggtcttttgacccccatgtctatatagaggcaattggagtaccacgagtagccgccaatgaatttaaagctcgaaatcaaatagctgccaggtttgagtcagtcttattttggtggtctactataaacaaaaacgtagactggataaactatatatattacaatcaacaaggatttgttaattataccagagataccatttggggatagccgagcaatttggtcccaccagccaaatggcctgggaaaactggatagctcttgacatgacattagcagaaaacggcagagtttgtgtaatcatcagagcccaatgttgtactttgatcccaaataacacagctcccgatggaaccactaccaaggctctacaaggcctcaccaccctagcaaatgagctggctgaaaattctggaatagatgaccccttcacaggtttaaccaaaagatggtttggtaaatggaaaggacttatggcctctatcctcacttcccttgccattgttacaagtgcactcatccttgtaggctgttgcatccaccttgtgtttgtgggttagttcaaagactcatagaaatggcttgcactaaaaccttctctgtctctccccctccatattcagaccaacttttactcctagacaatcaagaagagcaacaaagccaaatcttactaaaataatttgaagaggaagaattataacaacaaaaagggggaaattgtcagaataaaagaaaggttcctcttcaaagatccaactttcttgtcataggctgtaaaccgccttgctccaaaccttaacctgtaacctttaccctctgccttatttgagaaagattcaagcgcatagccaattggagtcagcctagactgtgcggtccaacccagccaatggggaaagacaaagaagcaggaactgcgttagggttagaaacctgttctcctttattaggtgtgcttttgcgactgctataggcgcgggcagcacccttctgcagaagtaaactttgccttgctgagaaaacttttcttctgagtgctcctcttcctctgcggcactcagaaataagagaacttatttctaacaggaggaagaacatgataattttgcacacgcaggttctgagttttggctggtgcgctgagtgcttaccataccgcaaatactgtgtgatctgggctgaagaacagaaaacccaactaacagtgacttaggcaaaaagaaattttattctttcacagaagtctgtggcaggcaggtccagagttcatgcaggagctcaataacatcagagattgggctctttttgttcttcttagcatatcgatgttttacgtgatgctgggcacctcggggacctaagatggctgttgtagctccatgcatcatgattaaaccagagacaggaagtgtacgatggaagaggccagaggtgaatggcctcctcctctccttttgtcttattcctggagtgactccctcctcttatcagcagggatacaagcagtgcctggcagccttccctctctcctgtatgagccaaaagcaagactcatggcacccctccctgcaggaaaggggtggaatgggcccctggcaaagggggctggacttgcagtgacagtgctgctctgaacaggactaggcttctgttggagaggtggtcagcggctgctaggcaggtcacaagctgcctcacccatggctctccagctctgcacctaccctctccttctaataaatctgtttcaataaccaccacgaggtttaaagaggagcagtaacttgcccacggttgcgccaggcacccctagaagagcaagaattccttacaaagttattaaaacaggcaaatcccatataggtgacatttctatccaacaatgctgacatctcttttagttttgagccttagtaatccaattttctgcctcaggaagaggcagcagcttctgggaaaaatctgctcctgctaaggtggctcattcattgttttacctgaatagagtggtggatggtgaaggttcaaattctcactttccatattaaattttataacataaaaaataattaaatggggtgcgcgcggtggctcacgcctataatcccagcacctgggaggctgaggagggcagatcacgaggtcaggagatcgagaccatcttggctaacacggtgaaactgcgcctctataaaaatacaaaaaattagctgggcatggtggcgggcgcctgtagtcccagctactcaggaggctgaagcaggaaaatggcatgaacccaggaggtggagcttgcagtgagccgagatcgcgccactgcactccagcctgggtgacaagagtgactcggtctcaaaaaaaaaaaaaaaaattaaatgagccaaccaggacaatgtttaaaagccagagccaaaaaggctccaaaataaaacaaacttgtctgctcaattaattatttgaagagaacctacagtaatacagactggagatgggaaagcacgagggatgtatgagtcgaggaaccggcaaaggggtgtgggggtgtgtgtgtgtgtgtgtgtgcacacacatgcacccagctagggtagaaaataagtacttttgcttctatgggaagtgactttccagttcacttgtgtatcaaatgaacatgatgaaatcaagttgtactagccaatgtttgggagggttctgtattaaagggataaagaaaaacattagaaagagaagttacaaaaatcactgtggggaaaatgaacaccaatgacttggctgctctgaggcttgcagggagtagacagttaggcatcccaggtcatcagagaaccagtggggtcttttcctcaggggccgggttttctacacgcaagtcaaacctcttctcctctgggacacatgtttccaaaggactccagaaacacagcacagctggattcaggtactgtgtcaagatgtgccttcttcttcatctttgacagcaattaatgtactaaatgtcttgatagagaaggtcatatttgggaatattcttgggatcaataggactttggacaataagcttccccctcattgctcctcgatagattacttcaatcaaatctatgaagtcttgtttggttttgaagcttcccacaaacttagtgtgatctggagatctagacagcatagaagagaaacaactgctctaagaactttgagtaatcacttgttgaagactttcaggataaacaaaatgaaaaacagctttgttattttcttcttgtcaatgagacttcagaaataaagcccaacattaatttttcaatcatttttttccatttagtaaatgactcaacccagtgctgtgggccactagcatgaaagctctgtcatgtgcaaagctttagcttcaaaagctattgtaatcacaaaatctatttttatcacttctcaaatcatcaagcagctttcaccaagtgttggagtgcacattgtttcttcgtttgctgttgttgcttttgcctgggcattgtgcaaagcccattcaatattcatggccttttgatgttggtaggtattaaaaaattgattttttagaaaagtatctcaaacaccaggtatgttactgtaaaattttaaagaaataaataaaatcacggatgaggccagaggccagaggtcacttttcagtgtctatccttccaatcttgtgtgtgtgtgtgtgtgtgtgtgtgtgtgtgtgtgtgtgtgtatgtgcagatacaaacttcttttttcccaacattgggaattatgtcatacaaattattcagtaatatgcttttttctagctgacatatatttctttcatttgttaaatgtgcatctgcaacatcatattaaagtcctggaagtgtaattactgggttaaaaagtttaaaaactttaaactatttgatacataatgttgaactggcccccataaaaggttgtaccaaattaatacttacatcaaatgtgttccagtagaataaacagctttaaaatacttttgtgctttttttttgttgttgttgttgttgttttgcgatggagccttgctctgtcaccagactggagtgtagtggtgcgttctctgctcactgcaacctccacctcctgggctcaagcggaatctcctgcctcagcctcctgagtagttgggattacaggcacccaccatcatgcctggctaatcttttttttgagacggcagctctgttgcccaggatggagtgcagtggcgcgctctcggctcactgcaagctccgcctcctgggttcatgccatttttctgcctcagcctccagagtagccgggactacatgcgcccggccaccacgcccagctaattttttgtatttttagtagagacagggtttcactgtgttagccaggatggtctcgatctgctgacctcataatctgcctgcctcggcttcccaaagtgctgggattacacgcgtgagccactgcgcctgccatgcctggctaatttttgtatttttagtagagatggggtttcactatgttggccatgctggttttgaactcctgacctcaaatgatccgcctgccttggcctcccaaagtgctgggattacaggcgtgagccactatgcccagcctaaaacactttaaaacagataatatttcacatactttcaaataaagacaaaaagggtacacagatctagataagtggacacatcaaccacagtaagaaatgatcagaatcatggatgcacccaatcatcaatattaagtattgagatggtaattggatggagtggaaggcactaaagaggacatcatgttacttattcttgacaggctttgttgaagaatgatttaaaattcaatggcgaaaagcaggaaactttgcaagagagaagcacatcttagtttgctagaaggaatgtttctttcataaatgccacttaccagtccaaaagcttaagaaaaactacatgttttgtttcaaatgaaattcctgggaaaattttaagagttctttatttagctaatatggctattttatattttatagacttatggatggtagtgctttgccacctgtactatttcttcatacttaagtcggaaattaaaatttgacaaaccatgtgtatgcactattaaaaaaataaataaaatttgacaatcataccagaagcactctcattttgtagcaaggttttgcataaaaaattagagagtgcgtcaattggggaaacagtactgatataagactcacagagccaacatcacatggaaataggcaagctactgaaaatgcttaccaaggaaacttacaaggttgcaattaacttcagatcaactgcacttacccataatccactttcatatgctgcccattgaagaaaaagacagtagatggaatataactgatgtcaaaatactgtgtataaactgcagtttggtccacatctaccaggtatatagcagccattttacttaagtcagaagaggtcttagaaagctgcaaatgacgagagagacaaaggttacaatatgagaggcagcttaatgtttcttctgtgaaacttgttactacaacagagtgttttgtttgaaaaaactcctgggccaacaatgcagcccatgtggctcccacattagacaaagcataaccccatcctccagagtctcattcactgcaacggggttaggagggaatcttggggaaaactgtatgaggttaggaagggaccttggggaaaactgtgtgaggttagaaggggaccttggggaaaactgtgtgagtttaggagggtgacagcatgggagtattcctgtctattcattgtggagagagacctaacccagaactaagacctctacccgccatttctactgctgccatcccgtgttaagagctgagcatacaaatccttgggaaaactgtgtgaatctgtttcttcctttaatttctttctttgaatgcttttataaaagtagcccatgctcatttaaaacaacacaacttagaaatatgttatgtggatactgaaattccctaacgtctccttcctcccaaccctcccagaaatgaaaaccacgataattagtagcagacacttatataacattactatgtggtaactgtcctaagtgcttaacatttgttaactcatttaatccttacatcattcttataaggtatatgctatcagagtccccattttatagatagggaaactgaggcacaggaaggtaaagtaatttgccccaggtcacacagctcctgagcagtagggctggggtttgaactttggcagtgtggcttcagtataccactgacctctgtggtatttcctcccgcctatgactgagggtagagttctctggcccttttttccccaagttaacacactgcaccttcattgcaattatggaaacgaggatttgtacgctcagctcttacacgggatggtagcagtagaaatggcggtaagaggtcttagttctgggttaggtctctctctgcatcaggcacatactggaggcagcacactagaaaagattatttgcagctacagaaaactgagttcaaatctcaactctgcaatatagtggatctgcacctgtgggaaattacttgacctctctggttttttttttcctactgggaaatggggctactattatcccactattgcgagcactgaaaagatagtgttcacagatgcttgttaattagttttctgctcttccatcaccttgttggtgaaacagcattgacagcttctagtgggtaaggacatccaagacagattcagcacacacagatttgggggcttggttcagggcaggcacacaatcagctcttagtgaactaacaaatgaacagctatatacccaaccctgactactccctctcatgtactactcagatgctgaagtgagaatatacagattattaataaaaaccaattattttagacattcacttacaatatcatctagctgcagacagacaggatcttcatctctcccaaacctgagaaccaacaccttctcagcagtactttttatcgcctggtctacttcctttttgctagtcagcttgggcagtaggaagctcatcttgaaataacccaaatgtgaatcctcactgtcactcctgaaataaaggtgcaatgtttaaagacagtttagattaagtgcgtgagccctcattaaaaaaaattaattcacagagtagctaatgtattcacatggtatgaaagaaacatcatagaaaggtaacactgtggagtttccctctctttgtccctgctccttggaaccagttttcattgttccctcccccgggccagataactgctttcattagtttctcatgtatctttcagagttttgttatgcattcaccaaaaaatacaaatatgcgttccgatattttttttctctctctccccacccccaaaagatagcctattgtatcagtgttccagcaggaaagcgatgacactctcaactttggtaatttgaaggcagtttaattaaggcaggtgtagaaaaacaatacggtaaagtgtactactttggggctagtaacagcgggatgctgtacccatccctaggccatgggaggaagaagccacggtaggaagcagttataagaattgtgtggcagaaagaggtgagtggagaggacagtgtataggagggcagcctgctgttaggtgttgcagccactctcctccctccctctgacctttgctcatgcaaccaccggctgactccaactggaaccagagggcaggggcctgttgaggcaggcaaacctcccttgtcccagagcagtctaagaggaggttgaagaatggctgtggagggaccaatggcagatgttcagcacacacactttatctactcttcagcacttttcattatttcaaacctaacctatgaagattgccccatatctctatatagagaacatccatattctctgtcatggcagcatattatttcttgcatgtgttacactttttaaacagtcctttattgtatactttatattattacaagtcctggcttaaagattcttcgtgtgcaccttactaggtgcacctcattacacggcaaacagatcaattaagagtgtgtgtgaaaactacaataccataaacaaataataaaatactgaggtacaagggacctaatacaaccccgtcattttacaaactagcttgcttacagttgcactttctactacaatgacaaggactagaatacaggccaccaaatttctagtccaggggtccttctactacaataatctattttcatgattggtagaaatttcatttttgagaaactctttgacaaagaagcttagttttgtggtcatctataagagactccattgagatggtcggtattttggaattc

SEQ ID NO: 9 - DNA, 22250 bp, Homo sapiens
  misc_feature (11552)..(11651): n is a, c, g, t or u
aactgccatcatactgaaggtaatcttctgaaacttgcggttttctttcaaaattatgtttataagatgatccatgttcttgcagagtttattcattttatgctgtataatattccattatatccacatacaatgcagtattgacccttcctcctgttgatgggcatttgtcttgtttctagttactttgctattatatcagtgtcaccatgattatccaaaagtaattcttttgtacactctaatttaagaacaactaaccctttttaatgaataaatcaaccttgtattgagttgctactaagtttcagttgactagtacctgggatacacacaggtgcagacatttgactgagacatattgatttttctcatctgcctatttaggctaatcaccagactataaaaccatgagaaccactgccattgagtatagtctgtgtcagtctacactatagctttaactagttgtgtgatttcttgcaaagagcaatcagagaagacacaataaacacatttactgatttcaggctggagagcttttaagcaatagggagatggccacacacaaggtggagaaaattactgtgaaaaggatacgtacttttctttagagccccacctaagctaggctgcagaaatgtctacaatggctttgaaaaaactcaaaatgagcctttctgcagtgtgaaaatcctccaagataaagagacagattgatggttcctgccgccgccctgtcctgcccagttgctgatttcaggaaatactttggcaggtttgtgggcatagagttgccaggtttcttgggatttgtaatagaacatcacaagaaaatcaagtgtgaagcaagagctcaactcttaacaggggtattgtttgtggttttgttactggaaaagatagtgaccttaccagggccaaagtttgtagacacaggaattacgaaatggagaagggggagaagtgagctagtggcagcataaaaagaccagcagatgccccacagcactgctcttccagaggcaagaccaaccaagatgaggtgggtccacagctttccctcctgcctttcctctggttctttatttcagtcttttttgcatacatcggtagagatgcagaaatagaacaaagaaacgggcaaatgggctaaattatagtgaaccaaagggcttagtgtgttaaatcttctccttttctgcatccatagaagacagtgctgctgtctttcccaggagataagatttactctcaggagtgtctttttccttcaggttacatttttgactttatagggtatgtcatcagctcccgtggtaggcttcctggcatcctgagtatatttattagcagatattttcctctttaaaaatgtacaataaggaagactaatagtaacacatttgaatgacacaattaattgactagtacctgggatacacactaatacctgggatacatctaattaaggcacttagatcttataaaaataaacactttttgaaatgttgaaataataagactagaaactttttttttttttgagatggagtctcgctttgtcaccaggctgcagtgcagtggcatgatctcggctcactgcaacctccacctcccaggttcaagtgattctcctgcctcagcctcccaggtagctgggactacaggcgtgcgccactatgcccacctaatttttgtatttttagtagagacgggctttcaccatgttggccaggatgacctcgatctcttgacctcgtgatctgcctgccttggcctcccaaagtgctgggattacaggcatgagccaccgtgtctggcctagaaactattttaatagaagcaagtagtgcccgaatggttggcatttgttagtgagatggtgaactggcagacggcacctgtgggtcaatgcccatggccaccgtcctgcttttggacaccagttctcttccaggtaaccttctggcattttggggtttcagataccatttcctaaaggtgaattattataaaatactagaaatacccacgtgtttaaaattatatatttaaggaaccttttattacggaaaatatcaagaagtagaggaatagcacagtaagcccaaagttcccatctctgagttatctacattttattaccactatctttgcggtgtctgagggaggtttctctttcctggagggctcctgtattattgccaatgtactttcctgaatgcagccagaaactgagcccacccctccacctatgtgcctttctatccctctctgaagcttctgcagaattcccagcaggacagggcttgctggaagcttggtatgctcagaagctgctaaagtgtgtatgggcaggtgtgggggcaatttcttggtcctagcacttccatatatcgactttcttttctggctgctaagtgggaggagtgtgtgtgtatgcatgtgtgtgtgtgtgtgtgtacatgcatgtgtgtgtggatgcatgcatgtgctgtgaagcagggagactagctttccactcctccttgtcttctctctgcagtgccctgggagctgtcattgccctcctgctctggggacagctttttgcagtggactcaggcaatgatgtcacggatatcgcaggtcagtctttggttgggtaggagtgtgcatcccactctgaccctctcgggtctgcactctctctgagaacacccaattcccccttcttatctcgacctctgggctttcaggaccataaagaacattggggttcctgccagaaatgaggggagcttgcctttccattggcttctattcggggtggaaggagattgatgtgcagagcagctcccgctcatctgacttttcacggttcactgggaacaatttccaaatagcaaactctctggcttctctctctttgcagatgacggctgcccgaagccccccgagattgcacatggctatgtggagcactcggttcgctaccagtgtaagaactactacaaactgcgcacagaaggagatggtaagatgtggacaactgtctccatgccctacatacaacccccttctctgacatttccatgatgggtggtgctgaggtgattcgccagaaagttcgttgctctccttggagccaggagatttagattctaataagcgttttgtcgccagtagccatggccctttgggcagactaacttttgtcagcctcaagttttctgttttgttaaggggaggcgatgccatgcagcctacctcatgtaaatctcagagtcagatttacatctccagcagatgtgggaaaagaaggaatgctgatgatgatgtcaccctcacctagtgagtcttgctgtcctggcactgctctaagggctttatacttatttgctcacttagtcctcacagtatccctctgaacagagtttattgttttcactttgctgataaggaaactgaggcacagacaggttgagtatcttgcccaaattcaggcagcctgtaagaggcagagtcaggatttgaaccctgagccctccctgtactgcttggctgtgaccgccatgaccacagtgtgttctgctgggcttaactggtgtccaggcacttggcttccagcacagcactctttcccttcctccttctcatattctctctcctttctcccttcctgtctgcctcctttcttcttcttctttttaattcttctccttaaatgccttctcactctgctctgggtgcagacttgacttttcctttggctcatttcttgccttttgtttcaggagtatacaccttaaatgataagaagcagtggataaataaggctgttggagataaacttcctgaatgtgaagcaggtgggtgctgagcactgagcacttaagagagcaggcaggcgtccagcggggaacgtcctagaggcacagccttccagtgcggcttcctctgagcacacaagagccaggaggagggatgtgggagaaccgcagctggccagggagagacttaagcagttaggtgatgactccctaagggtcaccaagggtcttgttcattggggcctgaagggcactggctgaatccactgtcggcactgcccacagatcaggagagcctgtgcatacagagagcctgctagaaagccctgggtctaaggagaagcaagctccagggagaacaagtcaaggaatgacataaaatcttaatccatggaagcctagcaggaggctggacatgggctggaactcctgcttctcgttattaggaggagctgttgctctctcctttcattctcagaaccagaggcaaagacccagcctcttctgctcttactggtgtggaaatgccaacctgcctcgtattaactgcaccatctacaaaatctgagctccagccagtgctgctctagattcatctttctttagagagaatgaattattgtagcccctagccctttcaatgaatttcagggaattgtggaaattcctttattgggataattgtttaaatataatacagttcgcgagcttctattcggggtggaaggagattgatgtgcagagcagctcccgctcatctgacttttcacggttcactgggaacaatttccaaatagcaaactctctggcttctctctctttgcagatgacggctgcccgaagccccccgagattgcacatggctatgtggagcactcggttcgctaccagtgtaagaactactacaaactgcgcacagaaggagatggtaagatgtggacaactgtctccatgccctacatacaacccccttctctgacatttccatgatgggtggtgctgaggtgattcgccagaaagttcgttgctctccttggagccaggagatttagattctaataagcgttttgtcgccagtagccatggccctttgggcagactaacttttgtcagcctcaagttttctgttttgttaaggggaggcgatgccatgcagcctacctcatgtaaatctcagagtcagatttacatctccagcagatgtgggaaaagaaggaatgctgatgatgatgtcaccctcacctagtgagtcttgctgtcctggcactgctctaagggctttatacttatttgctcacttagtcctcacagtatccctctgaacagagtttattgttttcactttgctgataaggaaactgaggcacagacaggttgagtatcttgcccaaattcaggcagcctgtaagaggcagagtcaggatttgaaccctgagccctccctgtactgcttggctgtgaccgccatgaccacagtgtgttctgctgggcttaactggcatccaggcacttggcttccagcacagcactctttcccttcctccttctcatatactctctccttttccccttcctttttgtccccttttcctcttccttttagttcttctctttaaatgccttctcactctgcacggggtctagacttgacttctcctttggctcacttcttgccttttgtttcaggagtgtacaccttaaacaatgagaagcagtggataaataaggctgttggagataaacttcctgaatgtgaagcaggtgggtgctgagcacttaagagagcaggcaggcgtccagcggggaacgtcctagaggcacagccttccagtgcggcttcctctgagcacacaagagccaggaggagggatgtgggagaaccgcagctggccagggagagacttaagcagttaggtgatgactccctaagggtcaccaagggtcttgttcattagggcctgaagggcactggctgaatccattgtctacatcgcccacagattaggagagcctgtgcatacagagagcctgctagagagccctgggtctaaggagaagcaagctccagggagaacaagtcaaggaatgacataaaatcttaatccatggaagcctagcaggaggctggacatgggctggaactcctgcttctcgttattaggaggagctgttgctctctcctttcattctcagaacaagaggcaaaggcccagcctcttctgctcttactggtgtggaaatgccaacctgcctcgtattaactgcaccatctacaaaatctgagctccagccagtgctgctctagattcatctttctttagagagaatgaattattgtagcccctagccctttcaatgaatttcagggaattgtggaaattcctttattgggataattgtttaaatataatacagttcaccagccagggctcaaaaatctcagtatttcccacttcctttgttagaaaagtgggaaatagagctttttgtaatgtaaacaatttaaaaaacagaattattttaaaactgcaactattggaaatgagatcagcaggtggtaagggcaaagcatttaaatctttctactttacgcagcagtgacagccgcccatgctttcacccctttctcagatggaaaggctcttgcacatttccactcacgagtgtcttgctctccttgacagtatgtgggaagcccaagaatccggcaaacccagtgcagcggatcctgggtggacacctggatgccaaaggcagctttccctggcaggctaagatggtttcccaccataatctcaccacaggtgccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggttgttctacaccctaactactcccaggtagatattgggctcatcaaactcaaacagaaggtgtctgttaatgagagagtgatgcccatctgcctaccttcaaaggattatgcagaagtagggcgtgtgggttatgtttctggctgggggcgaaatgccaattttaaatttactgaccatctgaagtatgtcatgctgcctgtggctgaccaagaccaatgcataaggcattatgaaggcagcacagtccccgaaaagaagacaccgaagagccctgtaggggtgcagcccatactgaatgaacacaccttctgtgctggcatgtctaagtaccaagaagacacctgctatggcgatgcgggcagtgcctttgccgttcacgacctggaggaggacacctggtatgcgactgggatcttaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccatttctgtgttgtgttcagtcttgagtcttttttatttgctcctttatggtccagggtagtcagaaggtatagagtctactgggagtatggcagaaaacaccctaaacccactggaaatcccgaaggtgatacaaactcttccaccttagggaatcatgctcactgattgagtgcctattgaatgctaggtcccagaaagttaactgttgtccttgttttacagacaaggaaacagagactcagagatggtaagtgagttgcttaaggttacatagctatgaaacagggaagcagaactttgaacccaggtctgtttgatacaaactcagaggtcctttcactgcatgctgttgcctcctcaaagtgaattaggagaaaaggcatgggcctggttgaggaagaggctagctcaaaatgggatggggaaaaagtgttttaacacagacagtaattcagagtttgggatctaacctaccagcactccagaaaacaaaagacaatgatgtaaaggcaggatctctggacttactgagtccaaatcacagaatggcccataaatttgctaggtaatttaggtttcctggccatcagttcctggtctcttccatgaaggacttgagccaggcgatggctaatgtccctatcagcaataaccttctggtattccataaagagtggggaggtagagtccctgcatcagtagaagctgcttttcctactgggaacaaacccaccctgcaccaggaatcaaatgcaggctgtctgagcctcctctcctgatggcaggtcatgctattaagggtctcccagaagaaaggttctggaactctgagaaacagaagctcttctaacagaactttttcttgctataagctttcaataccaccaatcaccagtgcaccttcttctcatgccaaaatacggcccctcctttgtagggtgtaaaacgacactgatgtcactctctctcaccaaggtctggccatctctgtgaagctctcctcacaggcaccctgcagattcatcaataaacttcaacaggttagcgccagtaggccaataagatgcacacttatattctagaacctgtgaaagaagtaatagccagaaactggaaaataacagtcaaactcttaaaaaaatgcctctcagaacacagtcacttacaccttttcctctggtagttctcttcagcaccaagtttcctggaagaacaagctactccccagttttcttaaagaattgtctgattgtattaagtaagtggcttctggtaaaataatggtaagagaaaagctctggcattctcttgatcactcatcttctctgccttcctccctagcaacatctcctggcctcagcctgtgggcttcagcaacaccaaactccatgttgctctctgacaagcccggccgtctccagttcttgggctttatttcctcgggggccctctgctgaaattcccttccccttgctgtattagcacctcctacccgccttcaacatgcaggcactgcctcctctaaggagccatctgtacttacgtagctgtgagcataggatggggcatacagcaggcacttaacaaatacttgtggaatggaaagaaactaagataggatcaaaaatacaaatcaagatgtgagagaaagacgatcaaaagccctcacgagggggtcgggcgtgatggctcacacctgtaatctcaacactttgggaggctaaggcgggtggatcacttgagggtgggagtttgagatcagccagagcaacatggagaaacctcgtctctactaaaaatacaaaaaattagccaggcatggtgatatatgcctgtaatcccagctactcgggaggctgaggcaggagaattgcttgaacctcggaggtagaagttgtggtgagctgagatcatgccattgtactccagcctgggcaacaggagtgaaactctgtctcaaaaaaaacaaaaaacaaaaaagaacaccacgggaaaatcaagtgtgaagcaagagctcaactcttaacaggggcaatgtttggagttttgttactggaaaagatagagaccttaccaggtccaaagtttgtagacacaggaattacgaaatggagaagggggagaagtgagctagtggcagcataaaaagaccagcagatgccccacagcactgctcttccagaggcaagaccaaccaagatgaggtgggtccacagctttccctcctgcctttcctctggttctttatttcagtcttttttgcatacattggtacagatgcagaaatagaacaaagaaacagggcaaatgggctaaattatagtgaaccaaagggcttagtgtgttaaatcttctccttttctgcatccatagaagacagtgctgctgtctttcccaggagataagatttactctcaggagtgtctttttccttgaggttacgtttttgtctttgtagggtatgtcatcagctcccgtggtaggcttcctggcattcggaatatatttactagcagatattttcctctttaaaaatgtataataagaaagactaatagtaacacatttgaatgacacaattaattgactagtacttgggatacacactagtacctgggatacatctaattaagacacttagatcttaaaaaataaaaagactttttgaaatgttgaaataataagactagaaacttttttttttgtgagacagagtctcactctgtcaccaggatggagtgagtggcgcgatctcagctcatcgcaacctccccctccccggttcaagcaattctcctgcctcagcctcccaagtagctaggactacaggcgtgtgccaccacgcccagctaatttttgtatttttagtagcgatgtggttcaccatgttggcccggatgatctcgatctcttgacctcgtgatccacctgtctcggcctcccaaagtgttgggattacaggtgtgagccaccgcaccctgcctagaaactattttaatagaagcaaatagtgcctgaatggtgggcgtctgttagcgagatggtgaactggcagatggcgcctgtgtgtcaatgcccatggccaccgtcctgctttcagacaccagttctcttccaggtaaccttctggcattttggggtttgagataccatttcctaaaggtgaattatcacaaaatactagaaataaccacataagtgtttaaaattattgttaaatacagtaagaaattcttcttcaaaagtttagcctgcttaagtttccttgtcctttgtttcctgctttcaaggccagacttccttactctctgtgtttcccctaccctggtaaacaaccttcctgccagtccttacccatacagcccacattccacatctgctacccactctgtgatttacctctcccgtcgcaatagcctctcccaccaaaactgatcttcccgccttcccaccagtgcaaccacattcctgcactttgcaagttagccaaccgggttcggattgtgcagtccaactccagccaatggagtcaggacacagtagcagggacaagctgcgttagacataaaaacctctgctttcctttgtttagggtgctctcgtggcaaccagacttaccaggagctctattctgcaaaagtaaatttgccttgctgagagaccctttgtcctttggctcagtgttggttcttctttgcagcaccgagcatttgtttccaacaaatttggtggcccatacagggaaaacattgtcctccgggaaagggttctttgatcatcctcttgagaggagaacacatcccactgtccttgttgcggtggcctcatgagtaggaatcgagacccacctgtctgatgaataaccccagactctcaacaacgtggggagaaaaagacttgcaacactatggtggccaggtaactctgtgcgcagaccaaggtaagaaatgtcgcaggagtgacaaagtacttccttggtggtcactatattctggtggctgaaagttcatgaatggtaacaagtgctactgctgtgtggagtgaatgagtccaatctgtgggtctatggttacctcatacggcttagccttctctgaaggatccgggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagcgtcctcctcccatctccaaaacaacccaactaaagtaacggttgtaaaggtaaaaaatttaaagcagactatagccctagagacaggatatcaagatgcaaagtcttggctaaaatggattaaatattccatctgcactttaaacaaatatgaatgttatgcttgtgctcacggtagaccagaggcccagattgtcccctttccactcagatggtcttctaactgaccgggcataagctgtatggtagttctcttccaagatcacacagcctggggtgacaaatcatgtcaagctctttctctgctgttccctgaagctcaacaccctgagggtcagcccccgagggccatcagcttccgtttcccaatgccaattttactttgtgtctctcacaacagggagaaaacttggtgttccttggagccttaatgggatgcagtgagcttaagtccttccaagagcttacccatcagtctgcccttagtcatcctcgagcggatgtatggtggtattgtggtggaccattactggacactgccgagtaactggagcagaacatgcactctaattccattggctatccctttcaccctggcatttcatcaaccagaaaagatagaaaccaaacactgtaaaacaagagaggcccctcatgggtctgttgactcccacatttatatagattccactggagtccaacgaggagtgccagatgaatttaaggcccaaaatcagacaactgcaggatttgaatccacgttcttctggtggttaactataaataaaaatgtgaattagataaactacatttattacaaccaatgatgatttttttttttttgacggagttttgctcttgttgcccaggctggaatgcaacggcatgatctcggctcaccacaacttctgcctcctgggtccaagcaattcttctgcctcggcctcccgagtagctgggattataaacatgtgccaccacgcctggctaattttgtatttttagtagagacagggtttctccatgatggtcaggctggtctcaaactcccgacctcaggttatctgcctgcctcggcctcccaaagtgctgggattacaggcatcagccactgtgcccagcctatgatttgttaattatactagagatgctcttaaagggacagctgaacaattaagacccaccagtcaaatggcctgggaaaataaaatagcattagacatgataccagcgaaagaaggtggagattgtgtcattatcggaacccgatgctatacttttatccctaataatactactccaggtgggatcacaccaaaagcactacaaggtcttaccgccttatcaaatgagctagccaaaaattctggactaaacgaccccttcacaaatttaatgggaaattctttggcaaatggaagggatttatgtcctcaatcctcatgtctcttgccatcagaatagggttgcttattcttgtaggatgctgtgtcataccctgtgccctaggattaatacaaaagctcattgaaacagctctccccaaaacctccctcaatcctcctcctcctcgttccagtgagctttttcttttagaagacccaagtagaataacagagccaaattatgttaaaagggtttaagaaaaaatactgtaaaatatttaacagggggattgttaaatacagtaagaaattcttcttcaaaggttcagcttgcttaagtttccttgttctttgctccctgctttcaaggccagacttccttactctctgtgttctccctgcgctggtaaacaaccttcccgccagtcgttatctatagagcccacattccacatctgctacccactctgtgaattacccctcccatcacaatggcttctcccggcaaaactgatcttcctgccttcccagcagtgtaatcacattcctgcacttttcaagttagccaaccgggttcagcttagattgtacggtccaactctagtcaatggagtcaggatacagtagcagggacaagctgcgttagacataaaaacctctgctttcctttgttcggggtgctctcgtggcaaccgaacctatgaggagcaaccttatgcaaaagtaaatttgccttgctgagagaccctttgtcctttgtctcagtgctagttcttctttgcagcaccaagcatttgttttcaacaattatatatttaaggaatctttgattatggaaaatttcaagaagtagagggaatagcatactaagccctctgttcccatctttgagttatctacattttattagaagtatctttggggtgactgagggaggtttctctttcctggagggcttctgaattattgccaatgtaccttcctgactgcagccagaaactgaggcccctatacctatgtacctttctatccctctctgaagcttctgcagaattcccagcaagacagagcttgctggaagcttggtatgctcagaagcagctaaagcgtgtatgtggggcggagggtgggggcaacttcttggtcctagcacttccatatattgattttcttttctggctgcttagtgggaggagtgtgtgtgtatgcatgtgtgtgtgtgcgtgtgtgtgtgtgtgtacatgcctgtgtgtgtggatgcatgcatgtgctgtgaagcagggagaccagctttccgttccttcttgttttctctctgcagtgacctgggagctgtcatttccctcctgctctggggacgacagctttttgcactgtactcaggcaatgatgtcacggatatttcaggtcagtctttgagttgggtaggagcatgcatccctggcactgccacatcccactctgactctctcgggtctgcattctttctttgagaacacacagttccccattcttatcctgacctctgggctttcaggactgccaagaacattggggatcctgccagaaatgaggggagcttgagctttcgttggcttctatttggggtagaaggagattgatatgcagagcagcttccactcatctgacttttcatgggtctctgggaacaatttccaaatggtaaactctctggcttctctctctttgcagatgaccgcttcccgaagccccctgagattgcaaatggctatgtggagcacttgtttcgctaccagtgtaagaactactacagactgcgcacagaaggagatggtaagacctggacaactatctcctgtgctctacctacaacccctgctctgacatttccatgatgggtggtgctgaggtgatttgccagaaagttcgttgctctccttggagccaggagatttagattctgataagcgttttgtcgccagtagccatggccctttgggcagactaacttttgtcagcctcaagttttctgttttgttaaggggaggtgatgccatgcagcctacctcatgtaaatctcagagtcagatttacatctccagcagatgtgggaaaagaaggaatgctgatgatgatgtcaccctcacctagtgagtcttgctgtcctggcactgctctaagggctttatatttatttgctcacttagtccttacagtttacctctgaacagaggttattattctcactttgccgataaggaaacagaggcaccgacaggttgagtatcttgcccaaattcaggcggcctgtaagaggcagagtcaggatttgaaccctgagccctccctgtactgcttggctgtgaccgccatcaccacagtgtgttctgctgggcttaactggtgtccaggcacttggcttccagcacagcactctttcccttcctccttctcgtattctctctcctttctcccttcctgtctgcctcctttcttcttcttctttttaattcttctccttaaatgccttctcactctgctctgggtgcagacttgacttttcctttggctcatttcttgccttttgtttcaggagtatacaccttaaatgataagaagcagtggataaataaggctgttggagataaacttcctgaatgtgaagcaggtgggtgctgagcactgagcacttaagagagcaggcaggcgtccagcggggaacgtcctagaggcacagccttccagtgcggcttcctctgagcacacaagagccaggaggagggatgtgggagaaccgcagctggccagggagagacttaagcagttaggtgatgactccctaaggatcaccagggtcttgttcattggggcctgaagggcactggctgaatccactgtcggcattgccacagatcaggagagcctgtgcatacagagagcctgctagagagccctgggtctaaggagaagcaagctccagggagaacaagtcaagggataacataaaatcttaatccatggaagcctagcaggaggctggacatgggctggaactcctgcttctcgttattaggaggaactgttgctctctcctttccatctcagaaccagaggcaaaggcccagcctcttctgctcttactggtgtggaaaggccagcctcttctgctgtgagtggtgtggaactaccaacctgcctcgtattaactgcaacatctataaagtatgagctccagccaatgctgctctagattcctctttcttcagagatgatgaattattgtagctcctagccctttcttttttctttctttctttttttttttttttgagacagagttttgctctcgtcgcctaggctggagtgcagtggtgctaattcctgacctcaggtgatccaactgcctcagcctcccaaagtgctgggattacaggcgtgagccaccgcatctggcccctagccctttcaatgaatttcagggaattgtgaaaattcctttgttgagataattgtttaaatataatatagttcaccagccaggcctcaaaaaactcagtatttctcatttccttatttagaaatagagctttttgtaatgtaaacaattaaaaaaattattttaaaactgcaactattggaaatgagatcagcaggtggtaaggacaaagcatttaaatctttccagtttatgcagcagtgacagccgccaatgctttcacccctttctcagatggaaaggctcttgcacatttccactcacgagtgtcttgctctccttgacagtatgtgggaagcccaagaatccggcaaacccagtgcagcggatcctgggtggacacctggatgccaaaggcagctttccctggcaggctaagatggtttcccaccataatctcaccacaggggccacgctgatcaatgaacaatggctgctgaccacggctaaaaatctcttcctgaaccattcagaaaatgcaacagcgaaagacattgcccctactttaacactctatgtggggaaaaagcagcttgtagagattgagaaggtggttctacaccctaactaccaccaggtagatattgggctcatcaaactcaaacagaaggtgcttgttaatgagagagtgatgcccatctgcctaccttcaaagaattatgcagaagtagggcgtgtgggttacgtgtctggctggggacaaagtgacaactttaaacttactgaccatctgaagtatgtcatgctgcctgtggctgaccaatacgattgcataacgcattatgaaggcagcacatgccccaaatggaaggcaccgaagagccctgtaggggtgcagcccatactgaacgaacacaccttctgtgtcggcatgtctaagtaccaggaagacacctgctatggcgatgcgggcagtgcctttgccgttcacgacctggaggaggacacctggtacgcggctgggatcctaagctttgataagagctgtgctgtggctgagtatggtgtgtatgtgaaggtgacttccatccaggactgggttcagaagaccatagctgagaactaatgcaaggctggccggaagcccttgcctgaaagcaagatttcagcctggaagagggcaaagtggacgggagtggacaggagtggatgcgataagatgtggtttgaagctgatgggtgccagccctgcattgctgagtcaatcaataaagagctttcttttgacccatttctgtgttgtgttcagtcttgagtcttttttatttgctcctttatggtccagggtagtcagaaggtatagagtctactgggagtatggcagaaaacaccctaaacccactggaaatcccgaaggtgatacaaactcttccaccttagggaatcatgctcactgattgagtgcctattgaatgctaggtcccagaaagttaactgttgtccttgttttacagacaaggaaacagagactcagagatggtaagtgagttgcttaaggttacatagctatgaaacagggaagcagaactttgaacccaggtctgtttgatacaaactcagaggtcctttcactgcatgctgttgcctctttaaagcgaattaggagaaaaggcatgggccttggtgaggaagaggctagctcaaaatgggatggggaaaaagtgttttaacacagacagtacttcagagtttgggatctaacctaccagcactccagaaaacaaaagatgatgatgtaaaggcaggatctctggacttgctgagtccaaatcatggacttggcccaaacatttgctaagtaatttaggcttcctggccattagttcctggtctcttccatgaaggacttgagccagggtaacgtccctatcagcaataagcttctgggactctgtaaagagtggggaggtagagtccctgcctcagtagaagctgcttttcctacttggaacaaacccacccttcaccaggaatcaaaagcaagctgtctgagcctcctctcctgatggcaggtcatgctattaagggtctcccagaagaaagcttctggagctctgagaaacagaagctcttctaacagaactttttcttgctataagccttcaataccaccaatcaccaatgtgccttcttctcatgcgaaaatacggcccctcctttgcagggtgtaaaatgacactgatgtcactctctctcaccagggtctggccatctctgtgaagctctcctcacaggcaccctgcagattcatcaataaacttcaacaggttagcgctagtaggccaataagacctatattctagaacctgtgaaagagataatagccagaaactggcaaacaccagtcaaactctttaaaaaaatgcctcttagaacacagtcacttacacattttcctagatctcttcagcaccaagtttcttggaagaacaaggcactctgcagttttcttaaagaattgtctgattgtattaagtggcttctggtaaaacaacagtaagagaaaagctctggcattctcttgatcactcatcttctctgccttcctccccagcaccacctcctggcctcagcctgtgggcttctgcaaagtcaggtatcaggactaattagctagcatcaaattcctgttaattccagaagcagggacaaacctgttggggagggacttaatgacaaaattaggtataggtctatacgttaatcaaggaaaattctcaacttccttaaacttacttaccaccctagaggagagccaaatccactctgatgacgtttggtcataggaaggaaattggggaaaattacaagtttctccaatccatgtaaaattaagaaatcctggggaagttgtaaagagaaaacagtaccctattcccttgaaaggcagaataggtttataacttataatcgaaggtctccttcaggaaggacttcttgaaccccatatgtcctcttacaacactccaatattgcctgtaaaaaagtcggatgggtcatagcggctactgcgagaccttcgagccattaaccaaatagtccagaccacccaccctgtcattcctaacccttacaccattatcaagcaagatccctcatgactgccagtggttcacagtaatagacctcaatgatgacttctgggcttgccccttagctgtggatagctgggacatatttgcttttgaatgggaagaccctcattccagttggaagcaatagtagtgatggacagttctaccccaagggtttacagactctccaaacctattagtcaaagtttggaacaagtcctagagaatttccctctttcatcatccttatgtctactccaatatgtggatgacctgctcatttccagaggcaccaaagaccaagtaaccgcaattttaattagctttctaaatttcctaagggaacaatggttacgggtctcaaaaagtaaactccagcttgtagaacctgaggtaaaacacatggggcacttaataagcaaaggtaagcggaagatagggcccgaacaaatggaagggatcatatccctgccactgcctgagacaaaacaagaacttagaaaattcttcaggctagctggaaattattgcctgtggattgactcttattcttaaaaacaaaacctctatatctaaaacttacccaagaagggcctgacccccttctttggaccccacaagaagtccagcaagttgaggaactaaaacatctacttataactgcccctgtcttagctctgccatccttagaacagccatttcacttttttgttaatataagcaatggggcagctataggagtactcactcaaaagcatggggccattgccagcccacagcctttctgtcaaacattcttgacgtggtaacctgtgggtggcccgaatgtatccaatctacagcagcaactgttttattgacagaggaaagtagaaaaataacctttgggggaagtctcatcctaagcacaccccatcaggttaaaaccattcttagccaaaaagcagggagatggcttacagattcaagaatcctaaaatatgaagctatcctattagaaaaaggtgatttaaccctaaccactgacaatgtgctcaaccccgccaccttccttttttttttttttttttgagatggagtttcactcttattgcccatgctagagtgcaatggcacaatctcggcttatcgtaatctttgcctcctgggttcaagcaattctcctgcctcagcctattaagtagctgggattacaggcatgctaccatgcctggctaatttttttgtatttttagtagagatggggtttctccatgttggtcaggctggtcttgaactcctgtcctcaggtgatctgcctgccttggcctcccaaagtgttgggattacaggcgtgagccactgtgcccggcctaccccgccaccttcctgacggaaatccaaacgctgaagaccccaagcacaaatgtttgaacttaatcatcaaactaaagtaaggcctgatctaagtgagacccctttaaaaacagggcaccatctctttgtagacagctcctctcgggtaattgaagagaaaaaacataatggctactcagtggttgatggagaaactctcacagaagtagaatcagggagacttcccaataactggtctaaccaaacatgtgagttgttcgcactaaatcaagccttaaaatagctgcagaatcaagaagggactatttacaccaattccaaatatgcttttggagtggcccatacctttgggaagatttggactgaacaaggccttattactagcaagggccaaaaccttgtccacaaagagttaatcatgcaagtattagaaaaccttcagctaccagaggacatagccattgttcatgtcccaggacaccataaaggtctctctttcaagagttacttcccaagaagcacccattttccactgaaccccttgtctcccttccccatctgcaacccccatcttctcccaagcagacaaagaaaaattaaagaaaataggggctaacaaaaactcagaagagaaacggatactgccagataacaaagaaatgttgtccaaacccctaatgacagaaatcttaacacaacttcaccaagggacccattggggtccccaagccatgtgtgatgcagtccttagaacctatgggtgtacagggatttataccctcaccaggcaagtcacagacagttgtatggtatgcagaaaaaccaataaactttaagaaaactgcccttcggcggaagaaatcctgggctaaggccatttccaaagtgtccatattgacttataccgaaatgccccaaataggctgcctcaaatgtttgtcagttatagtggaccatctcactcactgggtggaagccattcacctgccaagtgcaactgccaataatgtagttaaagtactaatagaagactccatacccagattcagattaatagagaacattgactcagacaatggaacccattttactgcacaggtcattaaaggattaacccagacactagagataaaatgggaatatcatactccctggcacccaccctcatcaggaaaagcagaaagaatgaatcaaaataaataaatcacctaactaaactaatcttggaaacccgactgccatgaactaagtacctccctgtcgccttactaagaattctcatgtttgtccttatcatcgataag

SEQ ID NO: 10 - protein, 662 aa, Homo sapiens
MVVSEVDIAKADPAAASHPLLLNGDATVAQKNPGSVAENNLCSQYEEKVRPCIDLIDSLRALGVEQDLALPAIAVIGDQSSGKSSVLEALSGVALPRGSGIVTRCPLVLKLKKLVNEDKWRGKVSYQDYEIEISDASEVEKEINKAQNAIAGEGMGISHELITLEISSRDVPDLTLIDLPGITRVAVGNQPADIGYKIKTLIKKYIQRQETISLVVVPSNVDIATTEALSMAQEVDPEGDRTIGILTKPDLVDKGTEDKVVDVVRNLVFHLKKGYMIVKCRGQQEIQDQLSLSEALQREKIFFENHPYFRDLLEEGKATVPCLAEKLTSELITHICKSLPLLENQIKETHQRITEELQKYGVDIPEDENEKMFFLIDKVNAFNQDITALMQGEETVGEEDIRLFTRLRHEFHKWSTIIENNFQEGHKILSRKIQKFENQYRGRELPGFVNYRTFETIVKQQIKALEEPAVDMLHTVTDMVRLAFTDVSIKNFEEFFNLHRTAKSKIEDIRAEQEREGEKLIRLHFQMEQIVYCQDQVYRGALQKVREKELEEEKKKKSWDFGAFQSSSATDSSMEEIFQHLMAYHQEASKRISSHIPLIIQFFMLQTYGQQLQKAMLQLLQDKDTYSWLLKERSDTSDKRKFLKERLARLTQARRRLAQFPG

SEQ ID NO: 11 - DNA, 2828 bp, Homo sapiens
atctgattcagcaggcctgggttcgggcccgagaacctgcgtctcccgcgagttcccgcgaggcaagtgctgcaggtgcggggccaggagctaggtttcgtttctgcgcccggagccgccctcagcacagggtctgtgagtttcatttcttcgcggcgcggggcggggctgggcgcggggtgaaagaggcgaagcgagagcggaggccgcactccagcactgcgcagggaccggaattctgtggccacactgcgaggagatcggttctgggtcggaggctacaggaagactcccactccctgaaatctggagtgaagaacgccgccatccagccaccattccaagcttactttgcaaagaaggaagatggttgtttccgaagtggacatcgcaaaagctgatccagctgctgcatcccaccctctattactgaatggagatgctactgtggcccagaaaaatccaggctcggtggctgagaacaacctgtgcagccagtatgaggagaaggtgcgcccctgcatcgacctcattgactccctgcgggctctaggtgtggagcaggacctggccctgccagccatcgccgtcatcggggaccagagctcgggcaagagctccgtgttggaggcactgtcaggagttgcccttcccagaggcagcgggatcgtgaccagatgcccgctggtgctgaaactgaagaaacttgtgaacgaagataagtggagaggcaaggtcagttaccaggactacgagattgagatttcggatgcttcagaggtagaaaaggaaattaataaagcccagaatgccatcgccggggaaggaatgggaatcagtcatgagctaatcaccctggagatcagctcccgagatgtcccggatctgactctaatagaccttcctggcataaccagagtggctgtgggcaatcagcctgctgacattgggtataagatcaagacactcatcaagaagtacatccagaggcaggagacaatcagcctggtggtggtccccagtaatgtggacatcgccaccacagaggctctcagcatggcccaggaggtggaccccgagggagacaggaccatcggaatcttgacgaagcctgatctggtggacaaaggaactgaagacaaggttgtggacgtggtgcggaacctcgtgttccacctgaagaagggttacatgattgtcaagtgccggggccagcaggagatccaggaccagctgagcctgtccgaagccctgcagagagagaagatcttctttgagaaccacccatatttcagggatctgctggaggaaggaaaggccacggttccctgcctggcagaaaaacttaccagcgagctcatcacacatatctgtaaatctctgcccctgttagaaaatcaaatcaaggagactcaccagagaataacagaggagctacaaaagtatggtgtcgacataccggaagacgaaaatgaaaaaatgttcttcctgatagataaagttaatgcctttaatcaggacatcactgctctcatgcaaggagaggaaactgtaggggaggaagacattcggctgtttaccagactccgacacgagttccacaaatggagtacaataattgaaaacaattttcaagaaggccataaaattttgagtagaaaaatccagaaatttgaaaatcagtatcgtggtagagagctgccaggctttgtgaattacaggacatttgagacaatcgtgaaacagcaaatcaaggcactggaagagccggctgtggatatgctacacaccgtgacggatatggtccggcttgctttcacagatgtttcgataaaaaattttgaagagttttttaacctccacagaaccgccaagtccaaaattgaagacattagagcagaacaagagagagaaggtgagaagctgatccgcctccacttccagatggaacagattgtctactgccaggaccaggtatacaggggtgcattgcagaaggtcagagagaaggagctggaagaagaaaagaagaagaaatcctgggattttggggctttccagtccagctcggcaacagactcttccatggaggagatctttcagcacctgatggcctatcaccaggaggccagcaagcgcatctccagccacatccctttgatcatccagttcttcatgctccagacgtacggccagcagcttcagaaggccatgctgcagctcctgcaggacaaggacacctacagctggctcctgaaggagcggagcgacaccagcgacaagcggaagttcctgaaggagcggcttgcacggctgacgcaggctcggcgccggcttgcccagttccccggttaaccacactctgtccagccccgtagacgtgcacgcacactgtctgcccccgttcccgggtagccactggactgacgacttgagtgctcagtagtcagactggatagtccgtctctgcttatccgttagccgtggtgatttagcaggaagctgtgagagcagtttggtttctagcatgaagacagagccccaccctcagatgcacatgagctggcgggattgaaggatgctgtcttcgtactgggaaagggattttcagccctcagaatcgctccaccttgcagctctccccttctctgtattcctagaaactgacacatgctgaacatcacagcttatttcctcatttttataatgtcccttcacaaacccagtgttttaggagcatgagtgccgtgtgtgtgcgtcctgtcggagccctgtctcctctctctgtaataaactcatttctagcagacaaaaaaaaaaaaaaaaaa

SEQ ID NO: 12 - DNA, 45622 bp, Homo sapiens
atcattcttcacagaactagaaaaaacattcttaacattcatatggaaccaaaaaaggagcccgcatagccaaagcaaaactaagcaaaaagaataaacctggaggcatcatattacccaacttcaaactatactacaaggctatagtccccaaaacagcatgatactgatataaaaataggcacatagaccaatggaacagaatacataacccagaaataaagccaaatacttatagctgactgaactttgacaaagcaaacaaaagcataaagtggggaaaggacaccctattcaacacatggtgctgggataattggcaagctacatgtagaagaatgaaactggatcctcatatcttgccttatacaaaagtcaactcaagatggatcaaagacttaaatctaggacctaaaaccataaaaattctagaagataacattggaaaaacccttgtagacattggcttaggcaaagagttcatgaccaagaacccaaaagcaaatgcaacaaaaatgaagacaaatggatcggacttaattaagctgaaaagcttctacacagcaaaagaaataattagcagagtaaatagacaacatacagagtgggagaaaattttgcaaactatgcagccaacgaagggctaatatccagaatctacaaggaactcaaacaaatcagcaagaagaaaacaaatactccctcacaaagtgggctaaggatgtgaatagacaattctcaaaagaagatatacaaatggccaacatataaaaaaaaatcaacatcactaattattaggaaaatgcaaatcaaaaccacaatgggataccaccttactcctgcaagaagggccacaatcaagaaatcaaaaaataatagatgttggcattaatgtggtgaaagggatcacttttacactgctggcaggaatgtaaactagtacaaccaatggaaaacagtagagtttccttaaataactgaaagtagatctaccatttgatccaacaatcccactagtgggtatttagccagtggaaaataagtcattatatgaaaaagacacttgcacacacatgtttatagcagcacaattccccaagtgcccaccaatcaacaagtggacaaaatgttatatatatatgtgtatatatatgtatatatatataatgtgatatatatgtatatatatgtatatatatataatgtgatatatatgtatatatataatgtgatatatatgtatatataataatgtgatatatatgtgtatatataatgtgatatatatgtatatatatataatgtgatatatatgtatatatatataatgtgatatatatgtgtgtgtgtatatatataatgtgatatatatatgtgaatactactactactactactactactactaatactcagccataaaaaggaatgaaataatggcattcacagcaacctggatggagttggagaccattattctaagtgaagtaactcgggaataaaaaccaaacattgtatattctcactcataagtgagattatacaagctatgaggatgcaaagtcataagaatgatacaataaacattgggaactcagggggaagggtgagggtggggtgagggataaaagactagacatcgggtacagtatacactgcttgggtgatgggtgcaccaaaatcccagaaatcacgattaaagaacttgtcaatgtaaccaaacaccacctgttcctcaaaaactattgaaataaaaaataaatacataaaagaagttgaggttaaattaatctaagtagggagtgcatttgggccaagcttgaggattgcaacgttggagcacagatttaagttgccctgaatctacactctgataagtggcatttataagtggatttttaaaggcaaaagggggagacagggagagggctgatacaaagctgtttgtcaggaattcttattggtttacagaaataacattgataagtgcttattagtccattttcatgctgctgataaagacatacctcagactgggcaatttacaaaagaaagaggtttattggacctatagttccacatggctgaggaggcctcacaatcatggtggaaggcaaggaggagcaagtcacatcttacatggatgacagcaggaaaaaagagagagcttgtgcagagaaactcccttttttaaaaccatcagatcttgtgagacccgttcaggatcatgagaacagcacaggaaagacccacccccaagattcaatcatctcccaccgggtctctcccacaacatgagtgaattatgggagctacaggatgagatttgggtgaggacacagagccaaaccatatcagagtgattgactatacattgttaagctacagggtgtggattacagcgtccagtgtggcattattaggttaatccatagctattaatacctgtggcaatagtaagaggtttcaagagatgaacacacagctgaaagtggggagtagggagtggttgctgtcttattttaatgtctctctgggcctgataattttaaaagccttgcattcttcggatacaagttcctttctttttttcaagaacaacagagaaaggctgcctggctacctttacctgcagcacctctgatagtggctgcttggcagctggttaggtaaacccaatttctactggataaacttcatggacattcacccatgttagtactggggaatgctctgaaatagcaaacttgtccatacagctcccttgctgctcagagggtctggagaggaacagcagaggcccagccccaaatacttcttgtaccggcttctattatgagaggtgatggcacctttaggcacttatatttcaggtagaaggagcaggaataaagaagagagggccacaatctgaaatatcagttctggatctccctgggggcaagacactggcccttagctgagctattctgtcatcactgtgagccctcatgctcgacctcaccggagcacccctcgatcatgtgctgaatgcctagcacgcgcctggcactgtggggtacagaaatctagagcacagagctgagttttcagggcttagctgtcctcacatctgtgacacaaacaaggatcaaggaaagttttgtttttgttatttttatttttttttaagttggggccttactctgtagcccaagctagagtgcagtggtgcaaacatagctcattgcaccctcgaactcctgggctcaagtcatcctcccaccttggcctccagagtagctggaagcccagctcaaggaaagcattaacatgttctacttttcaccataactcctgtgtattgggaaatggaaaatgagcttttgaagtggcctattcgcttcctccatgagtacttcctgaacactttcctcttggccactgctcccaagaccccctcaaagtctctctcctggacttccagcttctggaatgagccccaagagtggcacttagctccagctcctagtagctcactccttccttgctcacctgtgccccccacctgtctccttaggggcagtggctttgtcttactcatttgcatatcacaaatgtccagcatagaagccagcatggggcccagtaagtgtttgtggagaaagaaagggaagaggtgaagaagggaggagacggagggagaaaaggagggcaagagaggggatgtcatgtcatgtcatgtcatgtcatgtcagttctcccttcctagatctggacaagggaaagaaaagcaagcaagcctgaccgcctcagtgcagaccctctaagatgcaagccttccccaggtaagacagttccgcagggtgcttctgtgggctggctttctgagcagccatctcaaagtatgcgaaagaaatagattttgggccgggcacggtggctcatgcctgtaatcccagcactttaggaggccgaggcgtgtggatcacctgaagtcaggagttcaagaccagcctggccaacatggtgagaccttgtatctactaaaaatacaaaaactagccgggcttggtggcgcatgcctgtagtcccagctactcaggaggctgaggcaggagaatcgcttgaacccaggaggtaaatgttgcagtaagccgagattgccccactgcactccagcctggatgacaagagtgagactccatctcaaaaaaaaaaaaaaaaaaagattttaggggtaaaatattttgctttccttcagtggctgggccacctgtacacaatgcctgaacccaggtcctgcagcccccagcttggctctccaaagctcaccagtatcaaaggatttcagcttctgctcctctccgcagggctctctggtgggccccagacagagatgaaggaaaagtctggccttgaccttgaggaccaaaagcgaccacgtcagacgccagcagtctcgtgcccagttgccaagggatcacctgaggaaggcccttaaaatacaggtggtaatgcaggggtcttgggatggccccggaccgttcatttctaataatttcccaggtgaagccaatgctgctgttccaggaaccaccctttgagaagcacgggtctataccacacgcacagaagaggaaacgaaacgaggcaaaggaaaccagtccctttactcgcgcccaggcaggacaaagcgggcaagggtgggggcgggggcgcgggcgcgggcgcgggcaggcggacggacttgcaggaaatgcagccgggaggatgggcaggttgtgggatgctccgccgttccgcgaccacccgcctccccggcccagcagaaccgggtccaggctgggggaggtgctagcggtgtatgtgagtaacactgccccctcgtcgtggcaccgcccccttcttccctgcccccttccccgccccctggattctgaagtctgaagtcatgcaatttgacttttcctttctggaaaccagcgagcagaaatgaaaccgaaactgaattgtccgggaaattcgcggtgggggcggagagcgcagggagaagtaagcccagtgcaggatcctgaggcccgtgtttgcaggaccagggccggccttccgattccccattcattccagaagcaccgaaccacgctgtgcccggatcccaagtgcagcggcacccagcgtgggcctggggttgccggttgacccggtcctcagcctggtagcagaggccaggccagtgccacaaggtcaggctccactgggccctgcccgcccaggagcctgtggtgtcaggcacctgaaggggtgggttgcccctccacacctgtgggtgtttctcgttaggtggaaggagagacttggaaaagaaaaagacacagagacaaagtatagagaaagaaataaggggacccagggaaccagcgttcagcatatggaggatcccgccagcctctgagttcccttagtatttattgatcatttgtgggtgtttctccgagagggggatgtgtcagggtcacaagacaatagtggggagagagtcagcagacaaacacgtgaacaaaggtctttgcatcatagacagggtaaagaatcaagtgctgtgcttttagatatgcatacacataaacgtctcaatgctttacaaagcagtattgctgcctgcatgtcccacctccagccttaaagtggtttttccctatctcagtagatggaacgtacaatcgggttttataccgagacattccattgcccagggacgggcaggagacagatgccttcctcttgtctcaactgcaagaggcatgccttcctcttatattaatcctcctcagcacagaccctttatgggtgtcggactgggggacggtcaggtctttcccttcccacgaggccgtatttcagactatcacatggggagaaaccttggacaatacctggctttcctaggcagaggtccctgcggccttccgcagtgtttgtgtccctgggtacttgagattagggcgtggtgatgactcttaaggagcatgctgccttcaagcatctgtttaacaaagcacatcttgcaccgcccttaatccatttaaccctgagtggacacagcacatgtttcagagagcatggggttgggggtaaggttatagattaacagcatcccaaggcagaagaatttttcttagtacagaacaaaatggagtctcctatgtctacttctttctacacagacacagtaacaatctgatctctcttgcttttccccacaggcacctaagtccacctgggcctggagcaggacaggttgcaaaagaaaatatctcgggacccccaaactccttatgctaagggaaacatcgagcctgggaactgagccatcaacgctgccattctttttcccaaacagaaccctgttgtcagaggtacacccagagcaactccacaccgggtgcatgccacagcaactccatcttaaataggagctggtaaaacgaggctgatacctactgggctgcattcccagacgggtaaggcattctaagtcacagggtgagataggaggtcagcacaaggtcataaaggtcataaagaccttgctcataaaacaggttgcaataaagaagctggttaaaactcaccacaaccaagatggccacgagagtgacctgtggttgtcctcatttttacactcccaccagcgcatgacagtttacaaatgccatggcaatgtcaggaagttaccctacatgtatgtctaaaacggggaggcatgaataatccaccccgtgtttagcatatcatcaagaataaccacaaaaatgggcaaccagcagccctcagggttgctctgtggagtagccattctcttactccttcacttcaggcaggggagatgaatttccctttgagcctgtggacagagggcagcctggcagagggacaggcatcaacaaagcctctgagtgtgacctgggtcctgagttcagggatggcaatggggtaactgagactggatcagaagttgcctggaagaagagagggccattatcagagtataagagtgtgaaacttatctggtaggaaaaaaaaaagagtaactgctgttcgtttaatagagcacacatcctctgcaattcaggtgggaggttgtgagcttcttaagtacagaagctgcctcttgatttcagcatagcgaggaggtgctgaagagcgcaggtttggagaatgatcacctggattggaaccatagctctaccaatatggaacccagctccttaggcctcggtcttctcatggagaacatggtgtgataatcctactcctctgggagggtggctgttaaggtgagtgattgcacacgagtccctctagctgatgttaccgagcggtccttgtttttagagctcccaagagggtggcgggccacttccaagatggcggcaagcctcttgttctctgacctggggttcttggcctcacagattccaaggaatggaatcttggggcatgtggtgagtgttctcattaggacgaacccaggcacttagacatgcgggaacaatggcgagcctttagcccgaatgggagcggcaatgggtgcctcgctggatcaggagcacagcggacacactgccggatccagaggggtggacatcagtggtgggtctgtgatggcggcaaacaacagcagacagcaaatggcagtggtggatggcaagcgaaagctcagctccagccataacaaacacggaccagaagagtgtgcagttgcaagatttaacagagtgaaaacagacgtcccatacaaagggagggaacccaaagggggttgccattgctgggtcgaatgcctgggtttctgtctcaatcactgtccctccccctgtgctctcaggcaatagatgattggctatttctttacttcctgtttttgcctaattagtattttagtgagctctctttactacctgattggtcgggtgtgagctaagttgcaagccctgtgtttaaaggtggatgtggtcaccttcccagctaggcttagggattcttagtcggcctgggacatccagctcgtcccgtctctcactgacacctaacacatcctacatgctcagtagacaggagctattattgtatgactgactgctgaattacaccccaaaacaaccacctatactccccaaggccctgccatatcatatgcatattagaggcaatgaagcaccaaaatctaaggccagggaaggactgcggtggacaagggaagtgcctagggcgttaaaacttaaggaggcattgcaccaactgggagtgagctgtgtgtgctgtgtttcctgggcagggcgcctcactgcccttccgagtcctgccctggctgaggttgtgcctctatctggttatcagttgcctgcagctctttctgattttgttcgtgaacaagggcccttagggaagcctcccgctgttggaaacagcagcagcatcacccagagtgaagtctctgttcacatgagtgccagctctgctcacacctaccctgcgttcccaaggcactgttcctgcttcagaaagcatcagcccagcctgctcacagtcccccaggatgtgctgttgcctgggttcttgactaggaagtaaaaggcccagaggggttgagtgtgtgtgtgtgtgtgtgtgtgtgtgtgtgtgtgttaaagtgaggtcattgctaattcctgggagaacttgtgttccccttggatgaaccccttggatgaagaaccccttggatggggttgcccagccctaggagtggatcctgcaaagcaaaatagatttcagcacagggagctggctggggggctcctccccctcttgtaggtgcagcagtattgaatttgattccatccaggtttcccctgaccccaggacctaaagctggggcaggttctgggccttgaggttggttgggggtgacccttcctaatcatggaagtctttagcgggggcttgtaagggactgtgagctgagctaaggagaatggaggtggggtgccaaccgctcccaaagggaaatgccatctcccactcacagccagttagccgagaatgggagctcccaaagggaggaccacccatgggtctgcttgactcagccctccccaacccctttcacctttgcagtaaaactctagccaaggaagacaaagagacctttggagaccaaaacagaacttttaattcgggccaacagcaggctcatgcccaaaatggctgccaaccccaacaaagaaagcagctagcttatatgtcgtttgagatgggaaaaacaaggtaggatacaggtttcagacaaagacagtaaattacttaacccgtgacaattctgaggaaactggcaattcagttatattgactagtcatcctccaagctggactagggttggaggctggggtcccgaggcaggtgataagcttttgagataagcttgcatctgcaacttgttacaatgctgggaggggctgcttaaaattttagcctatgtgttacttctaaatagcttatactaaatgttaactgttttcatgtgcgtccatgtgaagagaccaccaaacaggctttgtgtgagcaacatggctgtgtatttcacctgggtgcaggcgggctgagtccgaaaagagagtcagcgaagggagataggggtggggccgttttataggatttgggaaggtaatggaaaattacagtcaaagggggttgttctctggtgggcaggggtgaatctcacaaagtacattctcaagggtggggagaattacaaataaccttcttaagggtggggaagattacaaagtacattgatcagttagggtggggcaggaacaaatcacaatggtggaatgtcatcagttaaggctgtttttacttcttttgtggatcttcagttactttaggccatctggatgtatacctgcaagtcacaggggatgcgatggcctggcctgggatgcgatggcctggcctgacaactattacctatgttatgtttattattttaagctttattattactattttatttattttattttattttccttccacacacccgtttccaccctggagaggccagatgagccagactccagggaggcctagaagtgggcaaggggaaacgggaaaggaggaagatggtatgggtgtgcctggttaggggtgggagtgctggacggagttcgggacaagaggggctctgcagccattggcacacaatgcctgggagtccctgctggtgctgggatcatcccagtgagccctgggagggaactgaagacccccaattaccaatgcatctgttttcaaaaccgacggggggaaggacatgcctaggttcaaggatacgtgcaggcttggatgactccgggccattagggagcctccggagcaccttgatcctcagacgggcctgatgaaacgagcatctgattcagcaggcctgggttcgggcccgagaacctgcgtctcccgcgagttcccgcgaggcaagtgctgcaggtgcggggccaggagctaggtttcgtttctgcgcccggagccgccctcagcacagggtctgtgagtttcatttcttcgcggcgcggggcggggctgggcgcggggtgaaagaggcgaagcgagagcggaggccgcactccagcactgcgcagggaccggtgagtgtcgcttctgggggcagcgcccagtaaccgcgctaggagcgcggagaagggcattgggagagcggcgttcgtggcggagactagcgctccggagcacgggcacgacgggggcaccttctcggctgctagtaactaacaataataataatcataatcatagcaagggcgctgatgggcgggctcggagcacgcctgattctggttcccaccaggctgcccaggctcctgatgacgcatcagaaacatccccctaacccgcggccttcctgcaggagaggttgggaaggggtgggggacggggctcgggggaggtctccgagggactctagtaagcggggaagggcgccgggaaagtttcagatccacggctgcgcgggccacgagcccacccgaacgccgaccactgctttccgtcgacttctatttcctgggaacgcgcgaaagcaaacccaagtcagactgcggaggtcgctggggagggaaggttcaaggagttctcgccgatcctgctgaataaagggggttccgagctgggccgagatggggcatgcgcgggaagacccctgcccgctgttcccccccaccgccccagtggatgccatgcctggggcctccccggcgcgtggggctgacgcaccctcggggtccatcgtagttggccgggatcgtggagtgggtgcggtggacgaagggaggcaggacagtcccgggggtggcagaaggagcccgggcacagctgagacctgcgctcccatcccaccaacactcacagcaggtgctgccgagctgggcaattgggatggcccaagttatttggttaaattttaaatcacgtttgttactgggaagtagagtccagtgatgctaaccgcgcctctacctccaccaccggtgtcagtcccaaagggctcctaaaatggctgtgtcatctttcagccttggaccgcagttgccggccaggaatcccagtgtcacggtggacacgcctccctcgcgcccttgccgcccacctgctcacccagctcaggggctttggtaggtagcagtgcatttggtctaaagggcaagatgttctctcttttattcataacaaatttaaataccagcagggtttggggggaaaaacgctttcagaagaaaaggtgaatgtcagtcctgcaagagttagttttaaaactagactgaattggcacatgtatacctatgtaacaaacctgcacgttctgcacatgtaccccagaacttaaaagcttataaaaaaagaaaaaactagactggattatgttgggaaagtgtagcctcttccatcttaggcatttcctagaacgtaggcagtaggtggtccttattaggagttttgggagaggaagggggctgaatcctacctcccatccctgctcctctatggggtctgagctgaggaagcttcaccacaaggagagaaccccctgacaaccctggatgccacctttaccctcactgcaggaattctgtggccacactgcgaggagatcggttctgggtcggaggctacaggaagactcccactccctgaaatctggagtgaagaacgccgccatccagccaccattccaaggtaaggcagaaatgaagtgggccgttgggttctttcttttctttctttcttttttttgagacaaggtctcactctgtcgcccaggctggagtgcagtggcgccatctcagctcactgcaacccccgcctcccaggttcaagcgattctggtgcctcagtctcctgagtagctgggattacaggcacacatcaccacacctggctaatttgtatgtgtttagtagagacagcatttcaccatattggccaggctggtttcaaactcctggcctcaaatgatccacccgccttggcttcccaaagtactgggattacaggcacgagccactgcacccagtcaggttcattttagttgttatgttaaccaggtttcctgcacctgtgcgctaactttcactttcccaaaaggtttcagggtgacccagcaggcaatgagtgattctcaaattcaggatttattgtgagagattcacacacacaattgagcagacattcacagtacaatgattaaagggagtgatagggtaaggacccacagtggaggctctggaggccagcccactgacagccactccagggagtccagaagtcccgctctagtgctgggtggtggagggaaatctgttcctccagggacctcgtcctcggctgcccagctgccaaagtcaggaataagctttcagaaatctcactgccaagattccgaaaacgcttcagacattgctagtcccttgtcgcttttgcgatcctccacaggtgtgcgtgccactgggtccttattcactggggtctctggtggcattgggccacagcaagtgttccctcatccccttagtctaccacacacatgcttaccactttgaagaaaaacccctttactatgagcgaaagtgagaaacacgtatgtttattgtttctaaagaaagaaacttaatatgggcttaatgctacctagtgagtgcctccattttgagacattagggtcacaagtcattattatatatcatgggcacaaacctgccctgggcagggacggaaggaagcccctgcacaggggcagttgctcaggatgtgagaagagcctggtgcataaccccatccatgcccacctaacatctcaggctctgaccagtggggctgtgcagtagcgagtggatggagggctggaaccctgcagcctcctctccaaacacagggtgcagccaagacattttaggagcaatttgggatggagagctaggagtcgccacctcttggctcttccaaggccggaactggtgcctgcactcagttcagtttgaagactgcagctggatgccaagttccatggaggagtaagaaaccggtttgaactcccgagattgccctgcccctgaaatccaaactgatgttccgaatgatcagggaaaggtacaaacgtttatggtttacagacaaaacccataaggtttagctttcagagaatctcattttatgaagcaaattagggaagggaatctactcaccaagtcctgtttcagctgattgagtggaacctgtggtcatgtggtacaagtcctggtctcaatgatgctccttatctggctgcagaaaggccaactgaggcaaccatagcccagaagactggtactcctgagaggcagatgaagtggtggtctttgatatcgagcctgggatgccctgggcacatgaggtatttccaaaggcatgggagttttagggaataaattcccagattgtcagactccataagtaccgtttacaatggattaccttttataaccatcccaatcctacctgacaaaagaggtgggcagattacgaggtcaggaaattgagaccatcctggctaacactgtgagaccccatctctactaaataaatacaaaaaattagccgggtctggtggcgtgcacctgtagtcccagctacttgggagcctgaggcaggataatcgcttgaacccgggaggtggaggttgcagtgagccaagattgcgccactgctctccagcctggtgacaaagcgagactgtctcaaaaaaaaaaaaaaaaagaaagaaatacatccctttcttcccttccaaatcgagcaaggatgcctgccctggaagtgtataaacccggggaagggagacagaaaaggatagttttaagtattggtgttggggacgtgttctttagccaaggcagcatgaacccatggcagcacttcccaaccttcctgacatgggcgtttctgtgaactccagtgtgatggagaaatggcattggctcaggtgtgcagctagatatgttacagagcagggtgacaggcaggggtgatgagttttgttttaacaacctgtcccttcaaccctcatggtactgacaaagatcacatggctctcgggggagattcctgcgaggggaagcaaggagagcatccttacatattattgatccaggcagcagatttgcagcaaagctctgtgctttattcatctgttaaaatagttaaaatagtcaaaacataggaaaaggattctgggaagtcagaatcggcttcagagcacacccctcctgcacttgcccggttctcagacttgggaatgggactgggtgggtgggtactctcgggtgttccgcgggtttgggtcttacttgtacactttgcttgatttcaaggaggtgcaggagaacagctctgtgataccatttaacttgttgacattacttttatttgaaggaacgtatattagaggtaagttggtgcatgctattttctgtaacatttattttgagtcataggagaaagattttcagttacttttatccaagattattagacactgtaaaatttcatatttaggcacttgtcctacaacattttaaaaatgaatttcaaatacatacgtgtgtatttgtaatgcagacaagtataaggcagtcagttacatgctttcaagagtaaaatgaatgacatttcatttcccccatttgtgggagtaaaagaatgacaatatgaaattgatgatcaaaagaaagagcataaaagatttagagctcacgtgttttttaaactaaaggtttgggtatcaaattaccgtaatatttggattctcttggctacattggaaacagttctataacaattttatttttaaatgtaaagtttttgtttgttgttgtttttaagacggggtctcgctctgtcgcccaggctggagtgcagtggtgcgatctcggctcactgcaaccttcacctcccaggttcaagtgattctcctgcctcagcctcccaagtagctgggactacaaacacacaccaccacttccagctaatttttgtatatttagtagagatggggtttcactatgttggccaagctcgcttcgaactcctgacctcaggtgatccactcaccttggtctcccaaagtgctgggtgggatacaggcgtgagctactgtgcccagcctttaaatgtaaactttttaattgtattacaactgcatcagaagttgtatactttgcaactattcaaatttatactgaaaacgtttttgaagttcaacctaaaattatgacaggagatagttttagaaaatattttggggaacagaggcatattctattttttttttttgagacagagtcttgctctgttgcccagactggggtgcagtggcgtgatctcagctgactgcatcctctgcctgccaggttcaagcaattctttgcctcagcctcctgagtagttgggattacaggtgcccgccaccatgcctggctaatttttttgtatttttagtagagacaagttttaccatcttggccagggtggtattgaactcctgacctcatgatctacctgcctcagcctcccaaagtgctgagattacaggcgtgagccccggggcccggcctggagggatattcttgaagcgccttgagcagggaggcagcgtttgtctttatctgaccttggcttctttgggtcactctgtttctctttccgtgaataaaaagccagtgagcacacactgtgtcccaggcactcttctacgctctggggacatcaccatgaacaactagtcagagtccccacctccaggggccttccgttctggtggtgggtttggttccatggttgaatgcacccagctgcttatctgttcaataggcatctgctttattttaagcttactttgcaaagaaggaagatggttgtttccgaagtggacatcgcaaaagctgatccagctgctgcatcccaccctctattactgaatggagatgctactgtggcccagaaaaatccaggctcggtaagttgctctctgaaagtcgctatccatgtgacatgagccatgcccattcgaggctgcccttttctacagcggtgctactgctgcccagatatgcctgctctctcgcctctcctgtgccaggacgcagatcctgaccctctacttgccagctgacaaccatgtatagactcgttgccttagctcaccgtgggtggaggtgctgctggggttggggacactggctgagctgtcgatgggttcagctcatctatactagaaggaactggcccaggccctgggttcaaggagcactaggtttgtttttcctgcccacatcatgattcagtctcaagctacaaaccctggggcatcattaagatattatctttgtagggaagccacgtggtgaattttttgcccagaatcaagacatatttttggtgggaaccaatcatggcccctggaatcagtccaacctcagttggtcccgacgctgacactctcagctgtgtgattgtgggtagttattcctcctgttataagcctggttttccatatctaaaatgagaataatggtctttgctttatatggctgagagcaaagtgcagggagtgtccaggtactcagagtgctcagtttcttattaccgtggatcactggtctgtttcaggctggctctgttttcctaggcaatgttaaacaatttttcaaacaatattcaaaaaacattgaagcgtttagaaaaatacagaggaccaccacctttccaccaccctgataccaccgtttacatttttctgcatttccgtccctggtgcgtctcttgtctggctgtaagagatgtaattatgtcagggtatggggcaggaggaactctcacccactcatacttgcgtgctttggaagcagcttggccttttccagtcaaggagaacagagtgtgtctcgtgatctggcagttccacttctggggatagactgtggttctcaaagtgtggcccccagcccagcccattagcatcacctgggaagttgactgaaatgcaaatgaccagcccctcctcccactcttagacctggtgaattggacactctggggcagggcccaccccctgtgctttaccaggctctccaggtgattctgctgtgtgctggagtttgaggaccatgtgtaaacccttgcacatccccctgggagccacatgcatagcagcactgtttataacagcaaaactcagaccctgtctacgtgcctgtcatggtggaatggatactgggagtgtggtatgttcatgtagcagaattctatacagtagtaaagaggaatgaactagagttccaggtatcaaaatggctgcatcaaatgaacaacgttgaaccaaagaatcaagttacaggaaatatacaaaatgattccacttacgtaaaattccccaacaggcagcaataggcaatataccattgtgggaaaaacatataggtggcaaaactataaagaaaagcaaggatgtgatgattgcaagcctcaggatagagggaccctctggaggtgaaggagttgacccagggaggtgtgtgcaatgtcctaagtactgggagtgtttgtgttcttaacccaagtggttggtacatgcatatttgctttattatgtttgacacacttttgtaaatagatagtataaataaatgaaaacaaacaaaaagttataaggtgaactaagaccgaggctaccaactgtattcatgcatttggtaaggctgtggttctttctcagtcagggcccattttgctccctggaacttgtggccaggtctagagacatcttggttgtcacaactcagggtgaatagtgaatagaggccaggtatttggctaaacctcctacaatgtgtagggtagcccctgcaacaaacaatcttctaaccccaaatatgaatagcttcttgtcctgttataaagaagctattctagtaaaaacgtctgtctatgatgaagcatgcacaaaaatagtcattagaaagaggtaaaagacaaaatgattttctcatattttcttcctgaacctcaatcagcccactttaggaaaattgcacccagctgctggtaggtaggcaggaccgagtgtgaagtctgctgctttctctgtttttatgcaagtacttcactattttgattactttagttttatagtaattatggaaattaggtaatgctagtcctctgactttgttcttctttttcaaagtcattttgactaagaatatttagatcatttctatttaatgtgactggtaatataattagatttgagaatatcatcttgctatttgttttctatttgtcccgtctgttcttcgttccccctttcgtctttttctgccttctcttggattattttttatgattccatcatgtctcctttgttgtcttattaagtataactcttggtgtttttagtatatatctttaacttaataagtcaaccttcaagtgatagtctgtcacttcatgtatagtgcgagaaccttagaatagtgtatttccatctctctgctctgagccttcatactatttctatcatgcatcttttacatacatcataaaccccacaatacattgttattattgatgttcaaacattcaattatcttttaaataaagatagcaaaggaatacaaaaaagtgtagtagttaccccttctagtatagactcctttgtatagatccagatttccattcagtatcattttccttctacctaaagaacttctttaacatttcctgtagtgcaggtctgctggtaatgaattagttaagcttttgaatggctaaaaaagtctttgttttgccttcatttttaaaagttatttttgctgggtatagaattctagattgatggtgtttttcagtactttaaaaatactgcttcactgtcttctcgcttgttattgcttctgataagattgacagcagatttctcatttgtgtccctctgctcacactgtatcattctctggctgctcctaacattttctctttattactggttttgagcaatttgaccctcttatggcttgatgatgtttatgtttgctgtgcttaatgtctgttgagtttctgggatctttgggtttatggttttcattaagtttgagggaattgtatgtattatttcttcaaatatttttttctgtctctcttccattctcttttggggattccagtaacctgtgtattagacttattgaagttggccgtctttaatggagtgtattggttcattctcacactgctataaagaactgcctgaaactaggtaatttataaagaaaagaggtttaattgactcacagtccacatggctggggaggcctcaggaaacttacaatcatggaggaaggcatctcttcacaaggtggcaggagagagaatgactgaaggaggaacttgccaaacacttataaaaccatcagacctcatgaaaactcactatcatgagaacagcatgggggaaacctcccccacaatccaattacctccacctggtctctcccttgacacgtagggattatggggattacaattcgagatgagatttgggtagggacacagaaccaaaccatatcatgagcatgatttgcaggccatgaagaattctccatttttgtttcctccaggtggctgagaacaacctgtgcagccagtatgaggagaaggtgcgcccctgcatcgacctcattgactccctgcgggctctaggtgtggagcaggacctggccctgccagccatcgccgtcatcggggaccagagctcgggcaagagctccgtgttggaggcactgtcaggagttgcccttcccagaggcagcggtaagaacttacattctgtgttagtctgctcaggctgccataacaaaataccacagacagggtggcttatacaacaaaagtttattttctcacagttctggagactggaagtccaaaatcagggtttagcttctcctgaggcctttctccatggcttgcagatggccacctcctcaccgctcccccatgcggccttccttccacacacaagcatctctgctgtctcttccccttcttcataagggcaccagtcatattggattggggcctaccctaatgacctcatttaaccttaattgcctctttaaaggccttatcttcaaatacagtcccattaggggttagggcttcaacataggaatttggagggaacaccatttcataacgctatctcattgcacatttttttcacataagtcatatgtaatctacagtttgaggagaatctgaataaacacatttgggtcccccagttcagaactatatcagtgaggtctcaaagagttcaggcctgggacgggtcttgcaatacagatcaggtgtggtaggaagaatatggaaggagtttacagtaggaggactgttgtaaggtggcctggtagcagcaggatgctttcctaatgggggtagagtgtgtatgctggggggataatggaagatgttcggatgagtttcgggggattcccaatgtggtctgccacctgagctgatggcagaacactgggatgaggcaggaagccaaaagtggtggctttcaagcgttagtaagcaaaaactcacctgggttacatactcagaatgcatgttcttgaggtcacccagacacagtgcgatgtccccgcatatcagagggtaagaccagaaagtttccagttttaaatgtctccccatatgattgtataaaagtttgagaaccatgggcctaaggcgctatgtaggtcttttaagagcaaagtggagcactgatgtgggcgtggcctcctagggatcgtgaccagatgcccgctggtgctgaaactgaagaaacttgtgaacgaagataagtggagaggcaaggtcagttaccaggactacgagattgagatttcggatgcttcagaggtagaaaaggaaattaataaaggtgagtaccccctgtttggatgcctggtcaagccttctgacatgcatggggtctgtttgtaactgttcatactcccacctccctgggcctgtgctgtcaggacacctttctcctgcacatcaggccacggttccttctacttcttttacctcattatgaccagcacgcttggatatcagcatctgatagcaatcatttatttctggccaggcacagtggcttgtgcctgtaatcccagctctttgggaggctgacacggggggattgcttgagcctaggagttcaagaccagccagggcaacatagtgagaccccgtctcttaaaaaaaaaaaattaaaatagctgggcagggtggcatgcacctgtggtcccagctattcaggaggctgaggtgggagagttgcttgagcctgggaagtcaaggctgcagtgagccgtgatcttgctacggcactctagcctgacaacagagtgagaccctgtctcaaaaacacatgtattgcttattatgtaagtatctagaataatgtgaattttaaaatgtccccacatatggatgatctgtcccttattcaagggcttccctacttagatctggcaggaagaggagccagatatgggggtgagggagctcctccccctgttcctttgtacaaggaactccacattgtggacaggatcgtcactgaaccccactcagaaccagcacccttttctaagaaagaggagtgactgtgtttgcataatcccagcttaggctaattcatggcaggcctccataaatgcaaaccacaaggatcaatttgaagtgtctgcaaggggaaatgacaccagcagtgtagacagggtagagtagctgacaaagaacagcctctgtgagatgcatggataacatcttcctatcgaccttcatgtttttctggcatgtcacatgtttaagtttcattcacactgggaaggtactgaagagacatgaactaatgcccagcagtaggaagggacgggtttagcatttgtaaagatggagcattaatcacatttgttgactgtttaaagaaagataaataatgttattgacaaaccgtgattttgaattagttgggattaggttggctgcttgcagcagaaaactcaaaataactgtgtgccttagcccttgctgtataacaaacccatcttaaaactaatggcttgaccacttttatttctcatgatttgatggaccagctgggcagttcttctctgggctagctgggctggggctgatggtccaggatggcccttggctggagtgaccgggccttctgtctgtgtggtctctcaccctccagaaggctaagctggacttatcaatgtgggtggaggggttcccagcagcaagagagggcaagacccgacatctaacacctttcggtctctcctgacgtggcactgtgtaacatccccttagccagagaaagtcacgtggccaagcccaatttcaagggaccgattttctctctctccatggaagtgacgaaggcaccctgtaaagtagcgtgcatacagggatggaagggaatgtggatgccattgtgccagcaagttgctgagacagcggtctaaacaatgtagaggctttctgtccctctcatataagtctgaaggcgggcagaccagagctgattggggattccaatgtcaggaaccaagatttattcttctcccatttcttcattgggtgtggcctctgtttccaaggcccccccgtgacttagtcagcaagtccgctttgaagccagctggaccatggcaaggggcatatctctgcctttgtaagcacactttctggaagttgcacataacattttcacatggcccattggccagaacccgatctcatgaccacatggcaggtacatggatattgggggaacaattagtggaccataaccactgatatttcctaagttctaaattgatatcaaacatcccaaaaaggcattctagatttagaaaagagtaaagtggtgttagccaacaatttgatgaaacaaattcatatcctaaaattcattaaggaggaaggagcaaaataaaatctcttaatgggatgttaacagccagtgcttatcttagctaaaataagcacatttccccatataattttccagtttatattttaggcatttccatatatttttatttgtttttattttgcttggttgctaatttcctactgacatcaatgagaaggatttaggaatgctaccaggaagaacttcttgcctccgcccagctttggactggtctaagtgggtgtcactcatggtgacgttctcacaaggtctctctacacacagtgctggccaacagcagggaaaatactgagttatcctttgagatctcttttatcccaatcacagaaaattgaatctgctccaaatatgcttttatccatgactcgcagagaggagaagatgctttcagagtattcaccatcatgagatccgtttatcctaagctctgtttgggtttgattttccctgtctcttttctagcccagaatgccatcgccggggaaggaatgggaatcagtcatgagctaatcaccctggagatcagctcccgagatgtcccggatctgactctaatagaccttcctggcataaccagagtggctgtgggcaatcagcctgctgacattgggtataaggtcagacttcagacccattctgaccttggccgtggcgtggggatgggggagtggaggggtgggaggagaaagagggtactgtattagagtaaccgtgagtccagagctgagttttggagttagtatttggaggtgtgagtggggaatttagagagcccgttggtcacagtctgttctgtcaagttgaatggaagcttctttggagaaagtgaggccagtgggcacagttggaaatgtgttctgtgtatttgttttatgttttatgcaatgacttgtttttggttatatacattttgcagcatatctaaagtgctgtgtattaggaaggggtcttatgtgggaagagagcattaaaaataagtataatgggccacacacagtggctcagtcctataatcccagcactttgggaggctgaggcaggaggattccttgagcccaagagtttgacagaaacctgagcaacatagtgagacccccttctctataaaagaaaggttaaaaaattagccaggtatggtggcgtgcacctgtcagctactaggaggattgcttgaaccagggaggctgtgatgagccgtgattgtgccactgcactccagcctgggcaacagagcaagaatctgtctcaaaacaaaaaacaaaacaaaacaagcaagaaagaaataggtataatgatattttagtatcagtgaatctcactttacagattaaagatttaggggtgaagtggggttttttggccaccatttttcattgtgaccatcagatctgaggtcttaggggttaattatctgaaacttcatggttttccctgagcctatagctctgcttctgccacagataatttattttctcataattccagcttggtacctccagggttgtgtttgtgggttcatttctccaaagttacttcttttgggggaaatacccctgggactcttagggcctaaagcaagtgcaaggtcaggacttgtctcacctctcacttgcctttgccatactcacgagtcacctcctctcatttccttacagatcaagacactcatcaagaagtacatccagaggcaggagacaatcagcctggtggtggtccccagtaatgtggacatcgccaccacagaggctctcagcatggcccaggaggtggaccccgagggagacaggaccatcggtgagagtgggggagccccactgtgctcagtgagaatgggggagcccgcctgtgctcggtgagaatgggggagcccacctgtgctcggtgagaatgggggagcccgcctgtgctcggtgagaatgggggagcccgcctgtgctcggtggtctgccagtgggcaagcgtccctccagtctccatgggctttgctcagtggggacctgcctccactaagacctgctaagggagcaggtttggtgcccaccaaggccaagtgaaatgagctgcttttgactctcactggctaggttgccttgtaagccttatctacttgctcagaaaggcacagtgggctcggaagcaggtcaaactcaggaggcacatggtactcattaagaatgcatttgagatgggatgtccataactcaagggataaacaaaacgtggcgtgttctacagtggacccgggtgaaggagcttggggagagccacatgctgttctgggaggcatccctgccttcacgcggcttgtcgtggagttcttttctggagcggggctccactgcccccatggttctgcaggggctatggcctgtcctcaagcaaggatgggaggaaaccctgggaggccgggggcgtgagcagttgttcgttcacctctgcctcgtgactgagcacgttctctccccaaatacatctggctcgcaggaatcttgacgaagcctgatctggtggacaaaggaactgaagacaaggttgtggacgtggtgcggaacctcgtgttccacctgaagaagggttacatgattgtcaagtgccggggccagcaggagatccaggaccagctgagcctgtccgaagccctgcagagagagaagatcttctttgagaaccacccatatttcaggtgcgcttgcctgggtttcatcatggatcagtccaagcccaggatgtcaggccttccaggggacagtggcagccgtcccacagatgtgtggagtgtgtgtgtgtgtgtgtgcgtgtgtgtgtgtgcgcgtgtgtgtgtgactatgcttgttccccaacaaggactatggaattcacctagaagaataggaaggggattacaaaatactgccaagaaaaaaaaaactaaaaaccaatcaaaatagggagagaacaatgtacaataatttacgtagcatggtgctggaaccatattttataaaaacataaatagaagagaataggaaaaaagtagaaagcccagaaatagacctagatatatatatttgacacatgataaatgcagcatttcaaatcaaatagtggactatatcagcttgagtatctattagtggtgttaggataattaatatttgggaaaaaactaaaatactgcccttacttatctcattataccaaaatagttaaagtttaaagttaaatattaagaggaagcaataagcatctcagaagaaaggtaggtgaatagtttataagatttttctatccctcctaccaaaagtgacattttttaaaaggaaaagactgacaaattggtaagatttaaaatgatgagactatgtagagttgtaaacattcttacattcagttctcccagaagcctacagagagccattactcagaattccaggaatatcaaatggaaacttacatcctgttctgcacattcacaattgccagaagatgagatgattcagtgtccattgatggatggatgcagaaagcaatgtggtctgtacaaaaacatggaatactttcagccttagaaaggaaagacattctgacacatgctacaacatggatgaagcttgggaacattctactaagtgaaagaacccagtcgtaagaggacggatactgtctgattccacttagctgaggtccctggagtagtcagattcatagagacagaaagaatgatgggcaccaggggctgggagagagagaatgggcaggtagtgtttaatggggccattgtttcagtttgggaatatgaaaagttctgaagacagacagtggtgatggttgcataatagtgtgaatgtacttaatgccactcaagtgtactcttaaagatggttaaatggtcaattttatatgtagtttaccacaatttagaaaaattgacagagaaactgaagcttaggtatgagtatactcacaaaaaggcacagaaactcatgcttcactgctgcctttatcctaaaatgtcctataaaatgtgggaaaccctgtaataactcactctgtgagcacaaatttggatcgagtgagaagatacttgacttccttcctccaggcagcccatggtttaagtttttatcttggacaagatatcttgtgtctcttctcctcagtgttctgctacccatttatctcaatatgcttcaatgtatttgtatgaagatatgtctgtatccattatgatcacctacacatattacacatagaagggggtatgtgttataaaaacatatctatacatgtctgtgtatttttgtgatgaccaagtctatagtcagacaccatgatacacatttattatatcagctggaagagctcattccatatcattgtggaaatatcctagattgctaaaattcagtcataatcctattcaatccagttctgagtatttgttgggtaccaactgcaagacattccatccagttgtaagcaactgaaatttgccttgactttccccaacagcaaaaaggcagacatgcgtgttctggctacatcaaggtggaaatcggtcctgtgttctcttctagggatctgctggaggaaggaaaggccacggttccctgcctggcagaaaaacttaccagcgagctcatcacacatatctgtgtaagcacgggcagagctgtgggttctctaaaaagaatactacgaccgcagagctgaaccttgctggcttcttaaacatcactgtacacacagatcttctgaggatcttgttaagatgcagtttcagattctgtgggtctggagtggggcctagaattctgcatttccagcaagcccccagacaatgtggatattccttttcaggggaccacagtcagggggaatgctgatagactatatctactgggccaaaataaaaattaaaatcttatgcacaagctactaactcttcctttctcattgacaaccactattataatgtcttagtcattctaatgaacatattttttaacttctaaaagctttgtaaaagctctctgtggttctttttaaaagtctgcctgaatatagtgtcttcctttttcaaattttcttttcttttcttttctttcttttttttttttttttttttttttttgagacagagtctcactctgttgcccaggctggagtgcagtggtgtgatctcagctcactgcaacctctgcctcctgggttcaagcaattctcctacctcagcctcctgagtagctgggattacaggtgcccaccaccatgcctggctaatttttgtatttttagtagagacagggtttcaccatgttgaccagactggcctttttcaaattttcaactcagcaccagagtgcaaggtcttccacgtggtccccaggaatgcgggtgcataacagggttgtttccagccgaccatgatgagtgcagagctctctggggtcccactgtatgcagaaagaggatgcttccttattagattccccacctcgagcaagcccatggggattgattttttgcctctgcaccaagtcaggttcataagttcccgttcgaattttcttacctagacagatgcccttgtggctgagccgggcttcattgctgccttctccttgagcccctgcctggccactgttactggggctggcctctgactacccctcactaacttgtgagtccaccgatacatttaaaggtgcagctttcacatgtcagctggcatttttagatgtttgccgtggaagggtgagccagcatatggcgtcaaccgtattgttaaaaacataagtctctgatcactttttattgattgcaagcaacataaaagttgttgaatctcaaattgctccaaatgccactttttcagaacctactagacaagtggatctctccagtctccctccagagagtttacctaatatgaccacagaggaactgctcccgggtcactctgccggggcctaggacccatgcacagtgggtgccacagtgctgctcatgaggctgctgtcgcaggagtggggaagggggaagacctgggcagaaaacagtgcccccagtgtgtgcccccctgcacctcccccgggtctggaaaagcttccttttagaggaagccaggaagtcaaatggcccacacaactcctctgcagagggaggcccgggacctccttttcattctctgttcatctttacacatttccattattttctctccattttcctcagaaatctctgcccctgttagaaaatcaaatcaaggagactcaccagagaataacagaggagctacaaaagtatggtgtcgacataccggaagacgaaaatgaaaaaatgttcttcctgatagatgtgagtgttgccagctgcatggagctggagaagcacatgtcatggtcaaaaaagggaccctgggccttatgcacttccttcttcactcccccaaggctgatccaaagacatctggcccgtagcactcaaagggtggacagggctgagggaggcagggcagggagtgcagatgtgggggtggagtcagcagcgagggatgctcaggctgcgttgtgcctactctgtcacgagcatcacccagatccctaaggcagtaaggggtgggataggattttctagtgccaaaacctcttctttcccctgatccacagtgtcccataagaaagcaaagaatgactccccaccctccacataggcacggcctccaaatgaccttgacacttggatttgaagtctatccacttatactgatgtttttcttcttgacagaaagttaatgcctttaatcaggacatcactgctctcatgcaaggagaggaaactgtaggggaggaagacattcggctgtttaccagactccgacacgagttccacaaatggagtacaataattgaaaacaattttcaagaaggtgagtgtcttagtcccttcttttgggctgctacaaccgaatacctgagactgggtcatttataaacagtagaaacttattgctcattgttctggaggtgagaaatctattcttaaggaatcaggaaatttggtgtctggtgagagcttgttctctgcttcaaagatggcaccttctagctgtgtcctctcatgggataagggacgaacaagcttcctcggacctcttttttacaggggtaccacaggcatacctcagagatattgtgggttcagttccagacaaaaagaatattgcaataatgcaagtcatataaacttcttggttcctggtgcataaacagttcatttatgccctactgcagtctattaagtatgtaatagcattaggcctaaaaaatatgtatgtaccttagtttaaaacaccttattgctaaaaaattgctggtacagaaacaaaaagtgagcatgtgctactggaaaaaaatggtgctgatttgcttgacatgggattgccacagactttcaatttgtaaaaaatacggtatcagtgaagtgcaataaaacaagatatgcctggaatgccattatgcgggcagagtgctcataacccaatccttcctaaaggtctcctctgttgataccatcacactggggattaagtttcaacataggaatttttaggggacaccaacatgtagaccatagcaatgagtcaataccgtggtaaacctgatacgttggcttaagacagagaagagtggggcagttggggaggatggtcaggataaggagctagtgacaactaaagccatgtttgctctcttctatatcactgaacccaaatgaccatccactgatgaattgataaaccaactggcttgtgtctgtgtgtagcggttggctttggctgtcataacaaagtaccacagacttgggggggcttaaacagtagaaatttattttcttacagttctggaggctggaagtccaagatcaagatgttggtggagctggttccttctaaggcctctctccttgccttgcagatggcctcttctcattgggtcctcatgtggttgtccctcagtgtgtgtgtcctcatctcctcccacaaggacactaggcagatgggatgagggcccaccctagtgacctgatttcaatttaattacctctttgcctgtctccaaacacagtcagattctgagtttctgagggttaggacttcaacattggagtttgaagaggtcacaactcaagctcagcccgtaacaccaagtcctggaatatttccagccacagacaggcacagagtgctggtccacagcaccccatggatgaaccttgaaaatgtcatgctgagtgaaagaagccagccacaaaggccacacggtctatgattccattgatagaaaatggctagaacaggcaaacccaggcaggcagaaagcagaatagtggctgccaggggctggggagggaaaagtgggaagttatcactgatgggtgatggatgtggggtttgggagttatgtctggggatggtcgcacaactttgtgaatatactaaaattcactcacccatacacttttttttttctttttctttggagacagggtctcactctgttgcccaggctggggtgcagtggctcagtctcggctcactgcaccctctgcctcccagattcaagcgattctcctgcttcagcctccacctccctagtagctgggattataggcacctgtctaatttttgtatttttagtagagatggggtttcaccatgttggccaggctggtctcgacctcctgacctcaggtgatcactggcctcagcctcccagagtgttgggattacgggcgtgagccactgtgcctggcctgaaccatatatttttaacagagtgaatgttatactatgtaaatgacatctcaattagaaaaatccttatgggaaaatatttcctgactaaaaaaagtgttctagattaccactcaaaaaggaactcaaaccctctgaacttctgatggggctaactctctctagtgtggattgttgggagtacaaatcattccaaaagtttaaagaaaaatgcagcatcttacacagtgaacagtgctactgtatcacattcatacaagttgatgtgcctggtttactctgttatcccatttgacttgtaaacactttctacacatggcaatactttcgacacatgaatatgtgatgtattgttaattccagaaagtgttcatgctcatttctaatgggcatgcagttgagggcaaggagtgtattatggtacaatttctttggtaaaactaaaattggattcacaaaacttcatactcgagtacgtttttaagaaggggtcttggccgggcacggtggctcacgcctgtaatcccagcactttgggaggccgaggcaggtggatcatgaggtcaggaaattgagaccatcctggctaacacggtgaaaccccgtctctactaaaaatacaaaaaaattagccgggcgtggtggcgggtacctgtagtcccagctacttgggaggctgaggcagaagaatggcgtgaacccgggaggcagagcttgcagtgagctgagatcacgccactgcactccagcctgggtgacagagcgagactctgtcacaaacaaacaaatgaaaaaaaagggtcttactcgaagtttctgcgtatgtgggttcctggcatcgtacctggctctgcactccccttcctgagatgactaaggaaaattatcttcagatctggttttgtgtgtgtgtgtgtgtgtgtgtgtgtgtgtgtgtgtgtgtgtgtgtgtaatccctggatatttttagtttaccagttagatttgatttgataccactttttcttgccatttatattttcagaaaatttagaatggtattgtgtttagaaaaatgtgcaagattatttttgtaaaataatttagagggttttttttcctgctataggccataaaattttgagtagaaaaatccagaaatttgaaaatcagtatcgtggtagagagctgccaggctttgtgaattacaggacatttgagacaatcgtgaaacagcaaatcaaggcactggaagagccggctgtggatatgctacacaccgtgacgggtgagtgctcagtttcacctctgagcattgatttctaaagaaaggaaaggttcgaaccaaagccagcaccaaacttcagcactttcctcctggggtgcatcccacaccaacgagcaaacctctcattctccagatgccaagttggtattcaacaattcaattcaattctgacactaactaccctcagtcagtgtggaccccatagcttaagggctcagttccacaacactggccccaactacaaatgccggtcacaagtcccagacctcctattcttctgatggactgtttataaatcaaggttcttgcgacccattcctcaggtcaaccaagaactctggaacacacttcactgacatttactggtctattagaaaggatttgataaggggcacaaatgaagctgttggagaggcacatagtaggggcctgaacacagaagcttctgtccccacggggttgggggcaccatcctcatggcacagagatgtggtcatcaaccagggagctcttggaacctcaccgcggagaaggttttatggaggcctcatcatgaaggcatgatggaggattgactcaatctccaggccctccctcctctgtggagctggaagttctaagtttctagccaaggcttggtctttctagtgcccggccccaatcctgaagctatgtaggggcccaccaggcatcatctcttcaaaacacgagatactcctaaggctggacattccaagagatgtaggggctctatgttaggaaatggggacaaagacaaaatatttatatttttcctatcacaccacacctcccccctgctccactgctatgctggcttcacactcaaaatcggctgtttatttgaaatctccgaggagtaaagccaatggttccataactgcacgtgtagatgtgtttggaaccttttggagtgctgtaggaatctaggtgtgtcacggataggtaggaaactagatcctactgtggatccactcccttcttgaaatgctttgctttcttggttttccaggtattaaatctctattcttcatcctctccttgactgacagtatccttactcacacttcagctgcctcatcttagcagtaattaataatcactcatggatccatgaactaaggagctggagatagcctcagaacagctcattcagaggtgtatttccagtaaaattgaccttttagccctgataatcatataccaaaacctgcaatcatgttgttttggtccattgtagactcttaactcattccagaggaaagtttataatacttagagccttatagtcataaaaatcaacatagatatacctatttctttttcagaaatgtatgacatggagatcaataagaggttttcaatcataaagatactataccttgtattacaataaaattctgtgaggaagtagaatagaaatgagtttcaaaaataaaagataaataatataaattttttaatctaagagcttgttcttgtatttttttcaaatggataatgtagacactcaaattccattgatatatttaagagtgatttgacttatattaagagttgtattataaaatattaatatttataatttaaaagaaattacattctttgcagctatttaggataaaaagtttaaatatcaaataaatgtatgccaggggtcatttgcttttaagattcttccagcaaattattaagcaaaaagagcatgccttgctttttcatggtaaagagaagaagggagcggggagaggggaaactttacttcataccatttgatcctcatatttttttgcatcttaagaagagaacaaatgatcctaccaatattgaactatttttctctctttgattagatatggtccggcttgctttcacagatgtttcgataaaaaattttgaagagttttttaacctccacagaaccgccaaggtaaaaccaaccatgtgttgtttaaaaaaaaaaaagaaaagaaattaagcttgacactagaaaatagatttcttggatgaggattatttcaactttattgtatacttttagaacagcaaataacatcactcactagtgcttcttctgatgttaccggtgatgtctggttaaaagcaataaaggagggagtgcttaaacgcacagaacaagagatccacagttagcggagaagattatcacatctaagggcaatggctccaaatccagaaactcactgaggaaactacatataaaaatagaatatttctggcccgagtgggcatgatgagcctgtaatcccagcactttgggaggctgaggccggtagatgacttaaagccaggagtttgagaccagcctggcccacatggcaaaaccccatctctactaaaaatacaaaaaagtagctggacgtggtggtgcatgcctgtaatcccagctacttgggaggctgacactttagaattgattgagcccaggaggtggaagttgcagtgagccaacattgcatcactgcactctagcctaggcgatggagcgagaccccgtctcaaaaaaaaaaaaaaaaacaaacaaaaaaactttccatccagagtgaggaaagagcctacaggaaatgagcctgggggacagactgggccaagagaccagacttagccactcttagaaataggtgtccccggcacagatgaggagcctggccccatgattcaccagctggaggccttgggatgtgccacttccagcctgtgcccctgactcctcattcataaaagaagactgataaggccttcctcagaaggttgagatggacgtggagtaagatgtttaggatgcacctgccactgtgcactgtgcctctcctcaaggcctggagggtccaggggtgaagtttctcctcctcaggttttggcaaccagtttctctaaaccccgggaacataaaacataattttctgacttaaacatggctttcctgctcatccctgtggattatctgatggatatgacaatcctcgccatcagatatagaagcccctaaaagagaaaggaaagaagctgagttacggggcctgaaagcaagcctgtgcaggtccccaggccccgggatgggggtccggcccatctgtggctcaagcctcctgggaagctctgaccctcagccagggctagaaacctgccttagatacaccagggcgcggcccagagggctgttccaggaaacgtgctgtttcactcacgttgggtaacctggtatttacggacttcttacctactttcctgtgactcaggaatttgtgtcttgagggaaactgtatttatttattttttactgtagtccaaaattgaagacattagagcagaacaagagagagaaggtgagaagctgatccgcctccacttccagatggaacagattgtctactgccaggaccaggtatacaggggtgcattgcagaaggtcagagagaaggagctggaagaagaaaagaagaagaaatcctgggattttggggctttccagtccagctcggcaacagactcttccatggaggagatctttcagcacctgatggcctatcaccaggtacgtcttcgcgtggttcaggatgccagcttccattctttccttttcttctgaacgcctctctctttagtcttgctctctctgtaggtgacgttggtcagctctgtcgtttacctccttgttagcctcctgtattagtccattttcatgctgctgataaagacatacctgagactgggcaatttacaaaagaaagaggtttaacggacttacagttccacatggctggggaggccttctaccatcacggcagaaggcaatgggcacttcttacctggcggcggtggcaagagagagaatgagagccaagtgaaaggggtttccccttatcaaaccatcagatctcatgagagttactcactaccatgagaacagtattggagaaactgcccccatgattcagttatctccccctgagtccctcccacaacaggtgggaattatgggagtacaattcaagatgagatttgggtggggacacagagccaaaccatatcgccttcgtagaagcagctcaacctcagacagagagatggtggcttagagccagtgacatctggttttgatggctgtctagctctggccaagttacttaacctctctgagcctcagctttctttgtaaaatggtgtctcctcatagattctagtgcatattccaggagacgagtgtggatgatgataatggattgctaatggaaaaaccaaactctgttaaaatatttgaaagaggtttattctgagccaaatatgagggaccatggctctgggaacagtctcaggaggtcctgaggaagtgtgcctgaggctgtcaggatgcagtttgattttatacatttcagagaggcaggaattgtaggtaaaatcataaatcaatacatgggaggtgtacttgccctccctaaagaggcaggacaccttgaaggagggggagcttaccggtcataggtgggttcagagattttctggttgacgattgcttgaaagagttaaactttgtctacaaacttgacatcaatagaaagaaatgcctgagttaaggcagtgttagaggccaaaggtatgtagatgaagactctgggtagcagccttcagagagaataaatggtaaatgtttcttttcaggccttagaggcagcaggctctcagttaatctctcctagattcagggaaggcctagaaggggagaggtctgactgcattaatggagattctctacaggtgcaaattccccccccacaaaacatggcagggccatttcaatctgttggtcctgttacagccgtttcaaaatatgtccacaaaatatatttttaggtaaaatatttgtatttcctttagggtctgcaatctgtcttgtgatgctataccagagtcgggttggaaagtaagccattttatactgagttcatggaaactcatccaaggagatttcatggtttgtggggtgtgtgtgacttaacccctgcctcacatgactttataatatggtatcttactactccagagtctttttggccaaccttatgatctcaatttcaacctaaactccaaaagggcctggcttctcttcctgttacggccaggaattcagattttcaggtttctctggggtccacttggccaagagggggtctgttgagttggctggaaggcataggattttatttctggtttacaacaatttccttagtgcagcattggaatgcaatggtagcagactaaatggaagctatcgcgtagacacatgctttggttgatactgcacgattcagttaacctgaagtacaatctaattcatcctagggaaggaggcagtgaacacagacacaactcaggtagagcccttgggatgtgtaaacacctgaggaggtaaagcaaattgtaatctctcggtttatcagatgtccccattgccttactatttggatgctttaaagcagggcctctcaaactccacccagcacagaggcctcctgggatcttgtggaaatgcagatgctgattgcaggtcaggatgaagctgagattctgcctttcttttttttttttttttgaaaccgagtctcactccattacccaggctggagtgcagtggcacaatctcagctcactgcaacctctacatcctgggttcaagcaattcacctgcctcagcctcccaagtagctgggattacaggcttaccttgccaccatgcctagctattttttctatttttagtagagatggacttttaccatgttggccaggctggtcttgaactcctgacctcaagtgatctgcctgcctcggcctcccaaagtgctgggattacaggcgtgagccaccatgcctggcctctgcatttctaacgggctctcaggggtcaccatactactggatagaggccacacttggaggagcaaggctctaaaccgagggtcaacatccattcctccagacactgggagctgcatgcacgtgagtgaagccagttaaggggaagacaggcatgcacatcagcttctcctgcagccaagctcacacctgtccgctgcttccactgcctcctagaatgaacagttaccttgagagtaggtgaggcatatacatgcacagaatccaaacaataggatgagtgacaatggcagaggagtctccgagccaagcagctccctggacagaagcagcccttctccgggttcatttctgtcctccgaggctgactcatgcactcaaaagctcccatgcatatacattttataatggtttttacacaaaggttagcagaggagtggacgtgctgctctgtaccctgcctcttttgctgtacctgggagattgttctgcctcagttctgatggggctgccttgttcttttcaatggctgctgagtatcccattttatggatgtggtatattgagccagctccctttaagcgaacagtttgtttgcagtcttttgctaatgcaggtgtgttgctgtgaataggtttgtttgtatatcatgtatctggaagcatcaattcctagaaatgagattcctggtatattaggattgtgcagggaaacagaaccacagatatatgtatgtaaagaagtatatttcagccaggcatggtggctcatgcctgtaatcccggcactctggaaggctgaggtgggtggatctcttgaggccaggagtttgagaccagcctggccaacatggcgaaacccggtctctactaaaaatacaaaaaaattagctgtgcatggtggcccatgcctatagtcccagctacttgggaagctgagatatgagaattgctcgaacctgggaggcagaggttgcagtgagccaagatcacaccactgcactccagcctgggtgacagagcgagactccatctcaaaacaaacaaacaaacaaaaaacaaaacaaaacaaaaaacagaaggaaagaaagaaatatatgtatatttcaaggaattggcttctgccattttgggagctggcaagtccaaaatcccagggcaggccagcaggaagagcaggccagaaattgcagcaggagctaaggctgagtccacaggcggaatttcttctttttagggaaacctcatttttcttcttaagaccctcaactgattggatgaggcccatccacatcattgagaatggtctccttcacttaaagtcagtgggttacacatgttacccacatctacagaatacctccgcagcaatacctagattcgtgtttgatggaatcactggggactcgagcctagccaagctaacacatgaaacacaccatcacagctggggaaaggatggcttattttagactgataaagatgacccagagaaggcctgctccatccacactggccgctttagtctgcactaaagttgttggttttttttgtttgtttggtttttttttggtgacagagtctcactctgtcgcccaggctggagtgcagtggcgcagtctcagctcactgcaacctctgcatcctgagatcaagcgattctcctgcctcagcctcctgagtagctgggactacaggcacgtgccaccacactcggctaatttttgttttttcagtagagacggggtttcaccatattggccaggctggtcttgaacgcctgaccatgtgatccatccgcctcagtctaccaaagtgctgggtttacaggcgtgagccaccacgcccggccttgttggggttttttgacagcctaataggtgaaaatgacatctcattacaatcttaattggcattctcttatgacaacaagctggtacatctttttgtgtgttgagggttatttctatttcttgctcagcaaacagttcatccaggaagagcttcttggtgagatagtagacctctgcgatttctgttgcagacgatctacattttgtcatttgctttgtcatttttgtctatggtggttttagactatgcgtaagttttctagagcagaaactcaagttggatttgggcctcagtggttattgccatactttaaaaggactttgtctccctgagatgataaatgaggtggacaatattttctttaagtaatttcttattttaactgttacatgatacctttggcccatttggagttctttgatgtcaagaatgaggcaggatccagatggcagcagaggtcccagtcccatcctggaagggtcgtctagttcccactggtactccacacgcccactcaggcactcacttcccctctgcgttgggtcttgtctgcaagactctcttatgttttaccatctagtgcagccagcacccccacatcaccctcactttttctttctttaaattgtgcagaaatattcatcatgtctattttgccatcttaaccatttgggggtacatagttcagtggcattaagtacattcatattgtgccagcatcaccagcagccatctccaggaccctatcaccttcccacactgaaactctgtccccattaaacacattccccattccccgcccctgaatccctgacagctaccatcctactgtctgtctctgtgaattcaactaacctaagtacctcataggagttgtgactggcttgtttcatgcagtatgatgtcctcatccaggtggtagcaagtgtcagagtttcacgcctatttatttattattatgagacagagtcttgctctgtcgcccagcctggagtacaatggcgcgatcccagctcactgcagcctccccctgcctgggttcaaacaattctcctgcctcagcctcccatggtgtgccgccacacctggctattttttgtatttttagtagagacgcggtttcaccacgttgaccaggctggtctggaaatgcagtttttgcactgtctgcctgcttacctttatagagcatattttgccctcttccatcagaattacccatttaatggtcaggaaaagctgctgggaatatgactcatagctgggacattctctgcactgtgcatagttcctctctgccaccaccatggaggagattgatgggtttgaaacccaggggaaggtcattgccctgcgagggtctccctcattgagaatctggatcccctcatgtgcacatggtgaggtcagagtcccctcctcacagtgtcccctccaccctcccgtgaactgttctttccttccaggaggccagcaagcgcatctccagccacatccctttgatcatccagttcttcatgctccagacgtacggccagcagcttcagaaggccatgctgcagctcctgcaggacaaggacacctacagctggctcctgaaggagcggagcgacaccagcgacaagcggaagttcctgaaggagcggcttgcacggctgacgcaggctcggcgccggcttgcccagttccccggttaaccacactctgtccagccccgtagacgtgcacgcacactgtctgcccccgttcccgggtagccactggactgacgacttgagtgctcagtagtcagactggatagtccgtctctgcttatccgttagccgtggtgatttagcaggaagctgtgagagcagtttggtttctagcatgaagacagagccccaccctcagatgcacatgagctggcgggattgaaggatgctgtcttcgtactgggaaagggattttcagccctcagaatcgctccaccttgcagctctccccttctctgtattcctagaaactgacacatgctgaacatcacagcttatttcctcatttttataatgtcccttcacaaacccagtgttttaggagcatgagtgccgtgtgtgtgcgtcctgtcggagccctgtctcctctctctgtaataaactcatttctagcagacactgctctgccatgttttgtattttggcgagaagcctgaaactagcaggtagggtgcagtggagcagtggacgtaaagctgccctctgtggcggggccaggtaggagcaagcaaaagaacagggtctgatgatttcctagagactggagggaaatgggggaacacactttaaaaaatgaaacaatatgaagccaacggtgctaacttaataatgtgcattgatcaaatgtccaatttgttgagtcactgggcacctggcatggagttgaaaagcacaaggcccagtcaggaagcggttgccaggccaggtgcgatggctcacgctgtaatcccttcactttgggaggccgaggcgggtggatcacctgaggtcaggagttcgggaccagcctgaccaatatggtgaaaccctgtctctactaaaaatacaaaaattagctgggtgtggtggcgtgtgcctatagtcccagctactcaggaggctgagacatgagaattgcttaaacccgggaggtggaggttgcagtgagcccagatcgtgccactgcactccagcctgggctacagagcaagactccatctcagggaaaaaaaaaggtttcccagtgggactttacctgcagaggctgtggctgggggagcggtctgggaagcagagaaggcggaacactaaccttggaggagaacctgactgaagttctctggtctagcctggttgggggagactcgagccacactcctacgccctgactctgagaaccatggctcccaatcagtgctgctgctgggcctcctcaccatgaggccagggcaggctcaagggcaggaaatcgaacagtggactagatggatggggcaggagaggaccggacactctgtcccttaaggggagagttgaaggatcaggattgccccagcctaggaaagagaaggccaaaaggcaacaagagagatgaagtgtgagcaccaaggtgtgacatcctctggggaggtatattctagatgatcagtgtggggaatgggggtactttctacagaggggcccccggatggcatggcttcctcaagagctgtgaggtcccaggcagtgggggcatcaggccaagctgcacagctgcttgtcaggttgggggtgggggctggaccagcaacaccatcctctcccctctggaccacgcccccccatcccagatctcttctgactcttgggactgtgtaattttcaattcttcgggaagtttgagaatacatttataagagaggatgtccggagagcagtggacatttcacaagcccaggtcagcagctgccgctgggcagcaactttatggttcccccaggtgctgaagtgcacctgcaggttgacttgatctactgttaacagagggtgatcagtttgactcatgcttaagggagagacgacaccaacacacacacagaggtttgcaagagtaaggcagggtggatccaaacattcttaattcaatactaggcacgcgttctcgcagtggacatggcccaggtcctctggctgtgttggtgcatcagacagctcggcccttccatgaggcaggtgccctctcgtggcaggcctgggaaacttctccactacaagacgtcccttccttccaccaccacccaaatcagtcttccacttgccttaagtttctgctgcacgggaaacagagtcagaggtgtcaaagtgttgcacaggattctattttgctgttgttagaattttgctagtgataaccagagaggatggaggaagaaagttgacagtatgtgtcccagagaacagcaatttcaatttccccagcagagtgaacctgctggggatgccccaaggtgcccgcaaggatgttcctttcagccaaaatgaacatgtgtggtgatcccccaggcctgtcctaactagtcatccagagccgagaggggagtggccccagcggccagaactgggagagagccctggagaccactttgtgtgactccttcctg

(19) United States
(12) Patent Application Publication
(10) Pub. No.: US 2023/0258660 A1
(43) Pub. Date: Aug. 17, 2023

(54) A METHOD FOR DISTINGUISHING HEALTHY INDIVIDUALS FROM INDIVIDUALS HAVING INFECTIOUS OR INFLAMMATORY CONDITIONS

(71) Applicant: ABACUSLABS LTD., Sligo (IE)

(72) Inventors: Heinrich ANHOLD, Sligo (IE); Ruth CANDON, Sligo (IE)

(73) Assignee: ABACUSLABS LTD., Sligo (IE)

(21) Appl. No.: 17/921,459

(22) PCT Filed: Apr. 27, 2021
(86) PCT No.: PCT/EP2021/061033

§ 371 Date: Oct. 26, 2022

(30) Foreign Application Priority Data
Apr. 27, 2020 (EP) 20171474.8

(51) Int. Cl. G01N 33/68; G01N 33/92

(52) U.S. Cl. CPC G01N 33/6893; G01N 33/92; G01N 2800/7095; G01N 2800/26

(57) ABSTRACT

A method of determining a health status of an individual, the method comprising the steps of assaying a biological sample from the individual for the expression of at least genes, or proteins encoded by said genes, wherein the at least three genes are selected from SAA, CRP, Hp and MxA, wherein the expression of the at least three genes, or the proteins encoded by said genes, above or below a specified threshold level correlates with the health status of the individual.

### CROSS-REFERENCE TO RELATED APPLICATIONS

This application is a 35 U.S.C. § 371 National Phase Entry Application of International Application No. PCT/EP2021/061033 filed Apr. 27, 2021, which claims benefit under 35 U.S.C. § 119(a) of EP Application No. 20171474.8 filed Apr. 27, 2020, the contents of both of which are incorporated herein by reference in their entireties.

### FIELD OF THE INVENTION

The invention relates to measuring levels of biomarkers in a biological sample to determine the health status of an individual by distinguishing whether the individual is healthy, has an infectious condition or has an inflammatory condition.

### BACKGROUND TO THE INVENTION

Infectious disease in mammals is typically characterized by the invasion of host tissues by infectious agents, including bacteria and viruses, and encompasses the host response to these agents and the resulting inflammatory response. Other conditions lacking an infectious element, such as autoimmune and degenerative diseases are also driven by inflammatory processes initiated by less characterised means. Mechanisms of the host immune system, led by an initial innate response frequently associated with inflammation, are activated when the body encounters infectious agents. Soluble and cellular defences including the complement cascade, collectin, and phagocytic cells are activated to destroy and dispose of the invading stimulus. This non-specific innate response is vital to control the early stages of an infection and is responsible for many of the familiar hallmarks of inflammation (pain, swelling and fever). This is followed by the adaptive immune response wherein specific antibodies, cell mediated immunity and immunological memory are developed.

Successful clearance of the infectious stimulus leads to a resolution of the inflammatory response and typically a return to the normal physiological state.

The host immune response is often complimented by the administration of selected antibiotics or anti-virals in cases of bacterial and viral infections, respectively. However, bacteria and viruses have proved adaptable and many strains have overcome the mechanisms of inhibition of antimicrobials and have become resistant to treatment. This has serious implications for the healthcare of individuals and healthcare systems, particularly in a clinical setting. Several organizations now recommend that antimicrobials are not administered unless there is clear evidence of infection.

Despite advances in diagnostic methods, medical professionals are often required to employ antimicrobial interventions before the presence of an infection can be determined with accuracy in order to reduce risk of negative patient outcomes. A useful clinical tool would provide means of early and sensitive differentiation between healthy and inflammatory or infectious conditions and would more accurately dictate the requirement for antimicrobial intervention by providing clear evidence of infection.

Such a clinical tool may offer additional insight when infection is detected by further differentiating infectious conditions into viral or bacterial categories. Bacterial and viral symptoms are often similar and difficult to differentiate without engaging pathogen-specific detection techniques, which cannot be performed rapidly, and/or at the patients/users' side. A need therefore remains for a point-of-care tool which can rapidly differentiate between healthy individuals, and inflammatory, viral, and/or bacterial infection in a patient.

When considering judicious use of antimicrobials, it is important to identify the causative agent, as viral infections do not respond to antibiotic treatment. Viral infections reportedly frequently affect more than one bodily system at a time and are less frequently associated with inflammation and pain when compared to bacterial infection. However, these observations are the limit of differentiation that can be made without further in vitro assessment.

A clinical tool that leads to a reduction in the number of viral conditions treated unnecessarily, and/or an increase in the number of bacterial infections identified correctly immediately, provides a valuable solution to over-prescription of anti-microbial treatments.

In certain settings, for example in a hospital, when the presence of infection is confirmed in a patient, prudent antimicrobial treatment is an important part of patient management and overall outcome. Multiple studies indicate that health care associated infection is among the most common types of adverse events affecting hospitalized patients. The US centre for Disease Control and Prevention has determined that almost 1.7 million hospitalised patients annually acquire healthcare associate infections while being treated for other issues. More than 98,000 patients die due to these infections.

Access to biomarkers that accurately monitor a patient's response to antimicrobial therapy allows the clinician to withdraw treatment at an earlier stage thereby restricting the unnecessary use of antibiotics further. This also allows the clinician to change or augment the therapy or monitoring regime if the patient's physiological response is not deemed satisfactory, i.e., if a decrease in the concentration or ratio of the biomarker(s) is not observed within a certain period of time or observation of a sudden elevation which might indicate an escalation in disease severity. Biomarkers with the ability to differentiate between healthy, infectious, and inflammatory conditions have the potential to be employed in any scenario where antibiotics are used to treat known or suspected bacterial infections, as well as in conjunction with post-operative antimicrobial therapy. Such biomarkers would have the potential create positive behavioural change by supporting the clinician in choice of treatment and improve treatment outcomes by decreasing unnecessary exposure to antibiotics and the side effects associated with overuse whilst ensuring condition appropriate therapies are engaged for healthy patients and those with non-infectious inflammatory illness.

Inflammation is an important part of the host response to infectious agents and is triggered by immune cell recognition of highly conserved pathogen associated molecular patterns (PAMPs). However, chronic inflammation triggered in the absence of infectious agents causes significant harm to tissues and organs. This chronic inflammation is often associated with the release of damage associated molecular patterns (DAMPs) which activate specific immune pathways. For example, the endogenous protein β-amyloid is associated with Alzheimer's disease by acting as an inflammatory stimulus. DAMPs have also been associated with disease severity and prognosis in conditions such as infection, asthma, ischemic heart disease, and cancer and they may prove to be useful tools in distinguishing between infectious versus non-infectious inflammation.

Although there is an array of acute phase biomarkers and test methods now available in the field to identify the onset of the innate immune response, these tests lack the sensitivity and specificity to contribute in a meaningful way to differentiation between healthy, inflammatory and infectious disease and thus, do not provide information at the point of care about what therapy should be pursued. Consequently, they are not compelling enough to create behaviour change and promote better patient care through more judicious use of antimicrobials and administration of condition appropriate therapies.

People of skill in the field are routinely interested in identifying and improving features such as the clinical utility and performance. Single biomarker tests that differentiate between infectious and inflammatory conditions have been challenging to establish to date. Furthermore, there does not appear to be a method available that allows differentiation between these infectious and inflammatory groups and healthy conditions. Such a method would be of great benefit in a home setting, in a clinical setting, as screening protocols including screening during disease outbreak or within immunocompromised and vulnerable populations.

It is an object of the present invention to overcome at least one of the above-mentioned problems.

### SUMMARY OF THE INVENTION

The main objective of this invention is to determine and monitor an individual's health status by distinguishing between healthy, infectious and inflammatory conditions, together with monitoring the efficacy of any treatment being administered to the individual.

In a home testing setting, particularly where testing is engaged as a screen or as a monitoring tool, such a test may be employed as a self-test to guide a home users decision making as to whether or when to present to a clinician for medical intervention. Users who suspect the onset of illness, e.g., after exposure to infection, may use the test to differentiate between healthy and non-healthy status and furthermore to differentiate between infectious and non-infectious causes. The user may further differentiate between viral and bacterial infection, as well as the identification of non-infectious inflammation (for example, sterile inflammatory diseases such as atherosclerosis and lung diseases; and autoimmune inflammatory conditions such as Alzheimer's disease and multiple sclerosis).

Infectious and inflammatory disease often cause the same symptoms and are difficult to distinguish. From the point of view of the clinician, it is important to identify the causative agent, as inflammatory conditions do not respond to antibiotic treatment and would benefit from other types of therapy such as NSAIDS, corticosteroids or a monitoring regime.

A clinical or prognostic method of assessing and monitoring inflammatory status would be extremely beneficial in non-infectious inflammatory conditions. Significantly, particularly as these groups are often considered as immunocompromised and vulnerable groups, early detection of the onset of pathogen associated infection in such groups enables rapid clinical decision making, assessment of disease severity and prediction of clinical outcome.

The inventors have devised a method with much improved clinical utility and performance indicators (sensitivity, specificity, positive and negative predictive values) for determining an individual's health status by measuring SAA, CRP, Hp and MxA concentrations in a sample and using those values to determine the health status of the individual by distinguishing between: healthy and unhealthy (non-infectious inflammatory and infectious conditions); healthy and non-infectious inflammatory status; non-infectious inflammatory and Infectious inflammatory status; bacterial Infection and viral infection; and further differentiating between non-infectious inflammation and bacterial infection, or further differentiating between non-infectious inflammation and viral infection in the individual.

There is provided, as set out below, a method of determining a health status of an individual, the method comprising the steps of assaying a biological sample from the individual for the positive expression of a gene, or protein encoded by said genes, selected from SAA, CRP, Hp and MxA, and wherein the positive expression of the at least one gene, or a protein encoded by said gene, above or below a specified threshold level correlates with the health status of the individual.

A method of monitoring the effectiveness of treatment of an infectious disease in an individual with an infectious disease, the method comprising the step of assaying a biological sample from the individual with an infectious disease for the positive expression of at least one gene, or a protein encoded by said gene, selected from SAA, CRP, Hp, and MxA, wherein the positive expression of the at least one gene, or a protein encoded by said gene, above or below a specified threshold level correlates with the effectiveness of treatment of the infectious disease in the individual.

A method of determining the progression of treatment of an individual with an inflammatory disease, the method comprising the step of assaying a biological sample from the individual with the inflammatory disease for the positive expression of at least one gene, or a protein encoded by said gene, selected from CRP, Hp and MxA, and negative expression of SAA, wherein the positive expression of the at least gene, or a protein encoded by said gene, above or below a specified threshold level, and negative expression of SAA, correlates with the effectiveness of treatment of the inflammatory disease or disorder in the individual.

A method of identifying an individual with an infection or an infectious disease that is suitable for treatment with a therapy for treating the infectious disease, the method comprising the steps of assaying a biological sample from the individual with the infection or infectious disease for the positive expression of at least one gene, or a protein encoded by said gene, selected from SAA, CRP, Hp and MxA, wherein the positive expression of the at least one gene, or protein encoded by said gene, above or below a specified threshold level correlates with the effectiveness of treatment of the infection or infectious disease in the individual.

A method of treating an infection, an infectious disease, an inflammatory condition, or an autoinflammatory condition in a subject, the method comprising: receiving the results of an assay that indicates that the level of at least one gene, or a protein encoded by said gene, in a sample obtained from the subject is above or below a threshold level; and administering a treatment for the disease or condition.

It should be understood that where there is positive expression of one of said two genes, for example SAA, and the previously positively expressing second gene but which is now negative for expression, the user should then retest (for example, after 6 to 12 hours) to confirm the presence of infection, as other biomarkers are expected to also be elevated by then. For example, Hp may be elevated alone in the case of non-infectious inflammation but the elevated levels of SAA may have decreased or is not being detected. Thus, there is a way to monitor the progress of treatment or the progress of the condition by determining whether the expression levels of the already detected genes (or proteins encoded by said genes) decrease below the threshold levels over time.

A method of determining a health status of an individual, the method comprising the steps of assaying a biological sample from the individual for the positive expression of at least two genes, or proteins encoded by said genes, selected from SAA, CRP, Hp and MxA, and wherein the positive expression of the at least two genes, or proteins encoded by said genes, above or below a specified threshold level correlates with the health status of the individual.

In one aspect, the at least two genes, or proteins encoded by said genes, are SAA and one selected from CRP, Hp and MxA. In one aspect, the at least two genes, or proteins encoded by said genes, are SAA and CRP. In one aspect, the at least two genes, or proteins encoded by said genes, are SAA and Hp. In one aspect, the at least two genes, or proteins encoded by said genes, are SAA and MxA. In one aspect, the at least two genes, or proteins encoded by said genes, are CRP and Hp.

A method of determining a health status of an individual, the method comprising the steps of assaying a biological sample from the individual for the positive expression of at least three genes, or proteins encoded by said genes, selected from SAA, CRP, Hp and MxA, and wherein the positive expression of the at least three genes, or proteins encoded by said genes, above or below a specified threshold level correlates with the health status of the individual.

A method of monitoring the effectiveness of treatment of an infectious disease in an individual with an infectious disease, the method comprising the step of assaying a biological sample from the individual with an infectious disease for the positive expression of at least three genes, or proteins encoded by said genes, selected from SAA, CRP, Hp and MxA, and wherein the positive expression of the at least three genes, or proteins encoded by said genes, above or below a specified threshold level correlates with the effectiveness of treatment of the infectious disease in the individual.

A method of determining the progression of treatment of an individual with an inflammatory disease, the method comprising the step of assaying a biological sample from the individual with the inflammatory disease for the positive expression of at least three genes, or proteins encoded by said genes, selected from SAA, CRP, Hp and MxA, and wherein the positive expression of the at least three genes, or proteins encoded by said genes, above or below a specified threshold level correlates with the effectiveness of treatment of the inflammatory disease or disorder in the individual.

A method of identifying an individual with an infection or an infectious disease that is suitable for treatment with a therapy for treating the infectious disease, the method comprising the steps of assaying a biological sample from the individual with the infection or infectious disease for the positive expression of at least three genes, or proteins encoded by said genes, selected from SAA, CRP, Hp and MxA, and wherein the positive expression of the at least three genes, or proteins encoded by said genes, above or below a specified threshold level correlates with the effectiveness of treatment of the infection or infectious disease in the individual.

A method of treating an infection, an infectious disease, an inflammatory condition, or an autoinflammatory condition in a subject, the method comprising: receiving the results of an assay that indicates that the level of at least three genes, or proteins encoded by said genes, selected from SAA, CRP, Hp and MxA, are above or below a specified threshold level; and administering a treatment for the disease or condition.

In one aspect, and for all embodiments described herein, the at least three genes, or proteins encoded by said genes, are SAA and two selected from CRP, Hp and MxA. In one aspect, the at least three genes, or proteins encoded by said genes, are SAA, CRP and Hp. In one aspect, the at least three genes, or proteins encoded by said genes, are SAA, CRP and MxA. In one aspect, the at least three genes, or proteins encoded by said genes, are SAA, Hp and MxA. In one aspect, the at least three genes, or proteins encoded by said genes, are CRP, Hp and MxA.

In one aspect, the at least three genes, or proteins encoded by said genes, are SAA and two selected from CRP, Hp and MxA.

In one aspect, and for all embodiments described herein, all four genes, or proteins encoded by said genes, that is SAA, CRP, Hp and MxA, are assayed.

In one aspect and for all embodiments described herein, the threshold levels for SAA protein expression levels are between about 10 ug/ml to about 30 ug/ml; preferably between about 15 ug/ml to about 25 ug/ml; and ideally about 20 ug/ml. In one aspect, the threshold levels for Hp protein expression levels are between about 100 mg/dl and 550 mg/dl; preferably between about 100 mg/dl and about 525 mg/dl; more preferably between about 100 mg/dl and about 500 mg/dl, and ideally about 114 mg/dl or 160 mg/dl. In one aspect, the threshold levels for CRP protein expression levels are between about 3 ug/ml and 29 ug/ml; preferably between about 3.5 ug/ml and 5.75 ug/ml; more preferably between about 4 ug/ml and 5.25 ug/ml; even more preferably between about 4.25 ug/ml and 4.8 ug/ml; preferably about 4.7 ug/ml; and ideally about 5 ug/ml.

In one aspect, the threshold levels for SAA protein expression levels are 20 ug/ml, the threshold levels for Hp protein expression levels are 114 mg/dl and the threshold levels for CRP protein expression levels are 5 ug/ml. In one aspect, where MxA is assayed, the MxA threshold levels are 0.174 ug/ml.

In one aspect, when the protein expression levels of SAA are less than 6 ug/ml (between 0 ug/ml and 5.99 ug/ml) and wherein when the protein expression levels of Hp are less than 114 mg/dl, the individual is healthy. In one aspect, where MxA is assayed, when the MxA levels are below 0.174 ug/ml, the individual is healthy.

In one aspect, when the protein expression levels of SAA are less than 6 ug/ml (between 0 ug/ml and 5.99 ug/ml), the protein expression levels of Hp are less than 114 mg/dl, and the protein expression levels of CRP are less than 10 ug/ml, and optionally where MxA is assayed and the MxA levels are below 0.174 ug/ml, the individual is healthy.

In one aspect, wherein when the protein expression levels of SAA are less than 20 ug/ml and wherein when the protein expression levels of Hp are greater than 114 mg/dl, the individual is experiencing an inflammatory event or is suffering from an inflammatory condition.

In one aspect, wherein when the protein expression levels of SAA are less than 20 ug/ml, when the protein expression levels of Hp are greater than 114 mg/dl, and when the protein expression levels of CRP are less than 5 ug/ml, the individual is experiencing an inflammatory event or is suffering from an inflammatory condition. In one aspect, where MxA is assayed, and the MxA levels are above 0.174 ug/ml, the individual is experiencing an inflammatory event or is suffering from an inflammatory condition.

In one aspect, wherein when the protein expression levels of SAA are greater than 20 ug/ml and wherein when the protein expression levels of Hp are greater than 114 mg/dl, the individual is experiencing an infection.

In one aspect, wherein when the protein expression levels of SAA are greater than 20 ug/ml, when the protein expression levels of Hp are greater than 114 mg/dl, and when the protein expression levels of CRP are greater than 5 ug/ml, the individual is experiencing an infection. In one aspect, where MxA is assayed, and the MxA levels are above 0.174 ug/ml, the individual is experiencing an infection.

In one aspect, when the protein expression levels of SAA are about 18 ug/ml to about 21 ug/ml, and wherein when the protein expression levels of Hp are about 50 mg/dl to 220 mg/dl, the individual is at risk of onset of an autoimmune and/or an autoinflammatory condition.

In one aspect, wherein when the protein expression levels of SAA are about 19 ug/ml to about 21 ug/ml, when the protein expression levels of Hp are about 50-220 mg/dl, and when the protein expression levels of CRP are about 3 ug/ml to 29 ug/ml, the individual is at risk of onset of an autoimmune and/or an autoinflammatory condition.

In one aspect, there is provided a method of monitoring the effectiveness of treatment of an infectious disease in an individual with an infectious disease, the method comprising the step of assaying a biological sample from the individual with an infectious disease for the positive expression of at least two genes, or proteins encoded by said genes, selected from SAA, CRP, Hp, and MxA, wherein the positive expression of the at least two genes, or proteins encoded by said genes, above or below a specified threshold level correlates with the effectiveness of treatment of the infectious disease in the individual.

In one aspect, the at least two genes, or proteins encoded by said genes, are SAA and one selected from CRP, and Hp, and MxA. In one aspect, the at least two genes, or proteins encoded by said genes, are SAA and CRP. In one aspect, the at least two genes, or proteins encoded by said genes, are SAA and Hp. In one aspect, the at least two genes, or proteins encoded by said genes, are SAA and MxA. In one aspect, the at least genes, or proteins encoded by said genes, are CRP and Hp.

In one aspect, there is provided a method of monitoring the effectiveness of treatment of an infectious disease in an individual with an infectious disease, the method comprising the step of assaying a biological sample from the individual with an infectious disease for the positive expression of at least three genes, or proteins encoded by said genes, selected from SAA, CRP, Hp, and MxA, wherein the positive expression of the at least three genes, or proteins encoded by said genes, above or below a specified threshold level correlates with the effectiveness of treatment of the infectious disease in the individual.

In one aspect, and for all embodiments described herein, the at least three genes, or proteins encoded by said genes, are SAA and two selected from CRP, Hp and MxA. In one aspect, the at least three genes, or proteins encoded by said genes, are SAA, CRP, and Hp. In one aspect, the at least three genes, or proteins encoded by said genes, are SAA, CRP and MxA. In one aspect, the at least three genes, or proteins encoded by said genes, are SAA, Hp and MxA. In one aspect, the at least three genes, or proteins encoded by said genes, are CRP, Hp and MxA.

In one aspect, and for all embodiments described herein, all four genes, or proteins encoded by said genes, that is SAA, CRP, Hp and MxA, are assayed.

In one aspect, wherein the threshold levels for SAA protein expression levels are 20 ug/ml, the threshold levels for Hp protein expression levels are 114 mg/dl and the threshold levels for CRP protein expression levels are 5 ug/ml. In one aspect, where MxA is assayed, the MxA threshold levels are 0.174 ug/ml.

In one aspect, wherein when the protein expression levels of SAA are less than 20 ug/ml and wherein when the protein expression levels of Hp are less than 114 mg/dl, the individual is responding to treatment.

In one aspect, when the protein expression levels of SAA are less than 20 ug/ml (between 0 ug/ml and 19.99 ug/ml), the protein expression levels of Hp are less than 114 mg/dl, and the protein expression levels of CRP are less than 5 ug/ml, the individual is responding to treatment. In one aspect, where MxA is assayed and the MxA levels are below 0.174 ug/ml, the individual is responding to treatment.

In one aspect, wherein when the protein expression levels of SAA are greater than 20 ug/ml and wherein when the protein expression levels of Hp are greater than 114 mg/dl, the individual still has the infection and is not responding to treatment.

In one aspect, when the protein expression levels of SAA are greater than 20 ug/ml, the protein expression levels of Hp are greater than 114 mg/dl, and the protein expression levels of CRP are greater than 5 ug/ml, and optionally where MxA is assayed and the MxA levels are greater than 0.174 ug/ml. the individual still has an infection and is not responding to treatment.

In one aspect, there is provided a method of determining the progression of treatment of an individual with an inflammatory disease, the method comprising the step of assaying a biological sample from the individual with the inflammatory disease for the positive expression of at least two genes, or proteins encoded by said genes, selected from CRP, Hp and MxA, and negative expression of SAA, wherein the positive expression of the at least two genes, or proteins encoded by said genes, above or below a specified threshold level, and negative expression of SAA, correlates with the effectiveness of treatment of the inflammatory disease or disorder in the individual.

In one aspect, the at least two genes, or proteins encoded by said genes, are SAA CRP and one selected from Hp and MxA. In one aspect, the at least two genes, or proteins encoded by said genes, are CRP and Hp. In one aspect, the at least two genes, or proteins encoded by said genes, are MxA and Hp. In one aspect, the at least two genes, or proteins encoded by said genes, are CRP and MxA.

In one aspect, there is provided a method of determining the progression of treatment of an individual with an inflammatory disease, the method comprising the step of assaying a biological sample from the individual with the inflammatory disease for the positive expression of at least three genes, or proteins encoded by said genes, selected from CRP, Hp and MxA, and negative expression of SAA, wherein the positive expression of the at least two genes, or proteins encoded by said genes, above or below a specified threshold level, and negative expression of SAA, correlates with the effectiveness of treatment of the inflammatory disease or disorder in the individual.

In one aspect, wherein the threshold levels for SAA protein expression levels are 20 ug/ml, the threshold levels for Hp protein expression levels are 114 mg/dl and the threshold levels for CRP protein expression levels are 5 ug/ml. In one aspect, where MxA is assayed, the threshold levels for MxA protein expression are 0.174 ug/ml.

In one aspect, wherein when the protein expression levels of SAA are less than 20 ug/ml and wherein when the protein expression levels of Hp are less than 114 mg/dl, the individual is responding to treatment.

In one aspect, when the protein expression levels of SAA are less than 20 ug/ml (between 0 ug/ml and 19.99 ug/ml), the protein expression levels of Hp are less than 114 mg/dl, and the protein expression levels of CRP are less than 5 ug/ml, the individual is responding to treatment. In one aspect, where MxA is assayed and the MxA protein expression levels are below 0.174 ug/ml, the individual is responding to treatment.

In one aspect, wherein when the protein expression levels of SAA are less than 20 ug/ml and wherein when the protein expression levels of Hp are greater than 114 mg/dl, the individual is experiencing an inflammatory response and is not responding to treatment.

In one aspect, when the protein expression levels of SAA are less than 20 ug/ml, the protein expression levels of Hp are greater than 114 mg/dl, and the protein expression levels of CRP are greater than 5 ug/ml, the individual is experiencing an inflammatory response and is not responding to treatment. In one aspect, where MxA is assayed and the MxA protein expression levels are greater 0.174 ug/ml, the individual is experiencing an inflammatory response and is not responding to treatment.

In one aspect, there is provided a method of identifying an individual with an infection or an infectious disease that is suitable for treatment with a therapy for treating the infectious disease, the method comprising the steps of assaying a biological sample from the individual with the infection or infectious disease for the positive expression of at least two genes, or proteins encoded by said genes, selected from SAA, CRP, Hp and MxA, wherein the positive expression of the at least two genes, or proteins encoded by said genes, above or below a specified threshold level correlates with the effectiveness of treatment of the infection or infectious disease in the individual. In one aspect, at least three genes or proteins encoded by said genes, selected from SAA, CRP, Hp and MxA are assayed.

In one aspect, the at least two genes, or proteins encoded by said genes, are SAA and one selected from CRP, Hp, and MxA. In one aspect, the at least two genes, or proteins encoded by said genes, are SAA and CRP. In one aspect, the at least two genes, or proteins encoded by said genes, are SAA and Hp. In one aspect, the at least two genes, or proteins encoded by said genes, are SAA and MxA. In one aspect, the at least genes, or proteins encoded by said genes, are CRP and Hp.

In one aspect, and for all embodiments described herein, the at least three genes, or proteins encoded by said genes, are SAA and two selected from CRP, Hp and MxA. In one aspect, the at least three genes, or proteins encoded by said genes, are SAA, CRP, and Hp. In one aspect, the at least three genes, or proteins encoded by said genes, are SAA, CRP and MxA. In one aspect, the at least three genes, or proteins encoded by said genes, are SAA, Hp and MxA. In one aspect, the at least three genes, or proteins encoded by said genes, are CRP, Hp and MxA.

In one aspect, and for all embodiments described herein, all four genes, or proteins encoded by said genes, that is SAA, CRP, Hp and MxA, are assayed.

In one aspect, the threshold levels for SAA protein expression are 20 ug/ml, the threshold levels for Hp protein expression are 114 mg/dl and the threshold levels for CRP protein expression are 5 ug/ml. In one aspect, where MxA is assayed the MxA protein expression threshold levels are 0.174 ug/ml.

In one aspect, wherein when the protein expression levels of SAA are less than 20 ug/ml and wherein when the protein expression levels of Hp are less than 114 mg/dl, the individual is responding to treatment.

In one aspect, wherein when the protein expression levels of SAA are less than 20 ug/ml, when the protein expression levels of Hp are less than 114 mg/dl, and when the protein expression levels of CRP are less than 10 ug/ml, the individual is responding to treatment. In one aspect, where MxA is assayed and the MxA protein expression levels are below 0.174 ug/ml, the individual is responding to treatment.

In one aspect, wherein when the protein expression levels of SAA are greater than 20 ug/ml and wherein the protein expression levels of Hp are greater than 114 mg/dl, the individual is not responding to treatment.

In one aspect, wherein when the protein expression levels of SAA are greater than 20 ug/ml, when the protein expression levels of Hp are greater than 114 mg/dl, and when the protein expression levels of CRP are greater than 5 ug/ml, the individual is not responding to treatment. In one aspect, where MxA is assayed and the MxA protein expression levels are greater than 0.174 ug/ml, the individual is not responding to treatment.

In one aspect, the individual is a mammal. Preferably, the individual is a human.

In one aspect, there is provided a kit for determining the health status of a subject, the kit comprising a control oligonucleotide and a set of oligonucleotides for detecting SEQ ID NO: 2, 3, 5, 6, 8, 9, 11 and 12, wherein detection of one or more of SEQ ID NO: 2, 3, 5, 6, 8, 9, 11 or 12 above a threshold in the sample is indicative of the health status of the subject, and wherein the sensitivity of the assay for detecting the at least one sequence from SEQ ID NO: 2, 3, 5, 6, 8, 9, 11 or 12 is at least 80%.

In one aspect, the kit further comprises a support having at least one oligonucleotide selected from group SEQ ID NO: 2, 3, 5, 6, 8, 9, 11 and 12 anchored thereon.

In one aspect, the kit comprises a support having two, three, or four oligonucleotides anchored thereon selected from SEQ ID NO: 2, 3, 5, 6, 8, 9, 11 and 12.

In one aspect, there is a kit for determining the health status of a subject, the kit comprising a control oligonucleotide and a set of oligonucleotides for detecting SEQ ID NO: 2, 3, 5, 6, 8, 9, 11 and 12, wherein detection of at least three of SEQ ID NO: 2, 3, 5, 6, 8, 9, 11 or 12 above a threshold in the sample is indicative of the health status of the subject, and wherein the sensitivity of the assay for detecting the at least three sequences from SEQ ID NO: 2, 3, 5, 6, 8, 9, 11 or 12 is at least 80%, wherein the at least three oligonucleotides are selected from one of SEQ ID NO: 2 or 3, one of SEQ ID NO: 5 or 6, and one of SEQ ID NO: 8 or 9.

In one aspect, the support further comprises having at least four or more oligonucleotides anchored thereon selected from one or both of SEQ ID NO: 2 or 3, one or both of SEQ ID NO: 5 or 6, and one or both of SEQ ID NO: 8 or 9.

In one aspect, the support further comprises at least one or both oligonucleotides anchored thereon selected from SEQ ID NO: 11 and 12.

In one aspect, there is a method of treating an infection, an infectious disease, an inflammatory condition, or an autoinflammatory condition in a subject, the method comprising: receiving the results of an assay that indicates that the level of at least two genes, or proteins encoded by said genes, in a sample obtained from the subject is above or below a threshold level; and administering a treatment for the disease or condition.

In one aspect, the method comprises: receiving the results of an assay that indicates that the level of at least three genes, or proteins encoded by said genes, in a sample obtained from the subject is above or below a threshold level; and administering a treatment for the disease or condition. The three genes, or proteins encoded by said genes, are SAA, CRP and Hp.

When the method, assays, kits or tests show negative expression of all of the genes, or proteins encoded by said genes, below the threshold level, the result indicates a healthy status in the subject, and the treatment, if being administered, can be stopped.

In one aspect, the method is carried out on individuals with known sterile inflammatory conditions to monitor health status, disease severity and/or early onset of pathogen-related infection for which the individual may be more at risk for.

In one aspect, the method is carried out on individuals with a suspected infection.

In one aspect, the method is used to predict the worsening of a condition by monitoring the individual over time. For example, every hour, every 2 hours, every 6 hours, every 12 hours, every 18 hours, every 24 hours, every 2 days, every 3 days, every 4 days, every 5 days, every 6 days, once a week, one a fortnight, once a month, and the like.

In one aspect, individuals with progressively decreasing expression levels of SAA are likely to have a better prognosis.

In one aspect, individuals with progressively decreasing protein expression levels for SAA and Hp are likely to have a better prognosis from disease state.

In one aspect, the method is carried out by a home user to determine their or an individual's health status. Measurements may be taken at additional/multiple time points.

In one aspect, threshold levels for biomarkers described herein are engaged to distinguish between healthy, inflammation and infectious conditions.

In one aspect, an individual diagnosed with a disease or condition may be selected for therapy associated with the disease or condition.

In one aspect, the method is used to detect onset of a condition before the onset of symptoms is detected.

In one aspect, there is provided a method for distinguishing if a pathogen-associated infection has been eradicated following a treatment step, the method comprising the step of assaying a biological sample from the individual for the positive expression of at least two genes, or proteins encoded by said genes, selected from SAA, CRP, Hp and MxA, and wherein the positive expression of the at least two genes, or proteins encoded by said genes, above or below a specified threshold level correlates with whether the pathogen-associated infection has been eradicated following the treatment step. In one aspect, at least three genes or proteins encoded by said genes, selected from SAA, CRP, Hp and MxA are assayed. Preferably, the at least three genes are SAA, CRP and Hp.

In one aspect, there is provided a method of monitoring an individual diagnosed with a sterile inflammatory disease, the method comprising the step of providing the individual with a treatment, assaying a biological sample from the individual for the positive expression of at least two genes, or proteins encoded by said genes, selected from SAA, CRP, Hp and MxA, and wherein the positive expression of at least two genes, or proteins encoded by said genes, above or below a specified threshold level correlates with whether the patient diagnosed with the sterile inflammatory disease is responding to the treatment step. In one aspect, at least three genes or proteins encoded by said genes, selected from SAA, CRP, Hp and MxA are assayed. Preferably, the at least three genes are SAA, CRP and Hp.

In one aspect, there is provided a method for differentiating whether an individual is healthy, has an inflammatory condition or has a viral or bacterial infection, the method comprising the steps of assaying a biological sample from the individual for the positive expression of at least three genes, or proteins encoded by said genes, selected from SAA, CRP, Hp and MxA, and wherein the positive expression of the at least three genes, or proteins encoded by said genes, above or below a specified threshold level correlates with the health status of the individual, wherein if the concentration of SAA is >20 ug/ml, and the concentration of CRP is >10 ug/ml, the individual is determined to have an infection; wherein if the concentration of CRP>97 ug/ml or the concentration of Hp>182 mg/dl and the concentration of MxA>0.174 ug/ml, the individual has a bacterial infection or a viral infection; and wherein if the concentration of SAA<6 ug/ml or the concentration of Hp<114 mg/dl, the individual is determined to be healthy and wherein if the conditions of the above are not met the individual is determined to have inflammation.

As described herein, levels of at least two genes, or proteins encoded by said genes, selected from SAA, CRP, Hp and MxA, can be increased in infectious diseases, inflammatory diseases, autoinflammatory diseases, cancer and/or in subjects with heart disease. In some embodiments of any of the aspects, the level of at least two genes, or proteins encoded by said genes, selected from SAA, CRP, Hp and MxA, can be increased in infectious diseases and/or in subjects with inflammatory diseases. Accordingly, in one aspect of any of the embodiments, described herein is a method of treating an infectious diseases in a subject in need thereof, the method comprising administering a suitable antibiotic to a subject determined to have an expression level of at least two genes, or proteins encoded by said genes, selected from SAA, CRP, Hp and MxA, above a specified threshold level. In one aspect of any of the embodiments, described herein is a method of treating an inflammatory disease in a subject in need thereof, the method comprising: a) determining the expression level of at least two genes, or proteins encoded by said genes, selected from SAA, CRP, Hp and MxA, in a sample obtained from a subject; and b) administering an anti-inflammatory drug, such as non-steroidal anti-inflammatory drugs (NSAIDs), to the subject if the expression level of at least two genes, or proteins encoded by said genes, selected from SAA, CRP, Hp and MxA, is above or below a specified threshold level. In one aspect of any of the above embodiments, at least three genes or proteins encoded by said genes, selected from SAA, CRP, Hp and MxA are assayed. Preferably, the at least three genes are SAA, CRP and Hp.

In some embodiments of any of the aspects, the method comprises administering a suitable treatment as set out in Table 1 to a subject previously determined to have an expression level of at least two genes, or proteins encoded by said genes, selected from SAA, CRP, Hp and MxA, that is above or below a specified threshold level. In some embodiments of any of the aspects, described herein is a method of treating a diseases or condition set out in Table 1 in a subject in need thereof, the method comprising: a) first determining the expression level of at least two genes, or proteins encoded by said genes, selected from SAA, CRP, Hp and MxA, in a sample obtained from a subject; and b) then administering a suitable treatment as set out in Table 1 to the subject if the expression level of at least two genes, or proteins encoded by said genes, selected from SAA, CRP, Hp and MxA, is above or below a specified threshold level. In one aspect of any of the above embodiments, at least three genes or proteins encoded by said genes, selected from SAA, CRP, Hp and MxA are assayed. Preferably, the at least three genes are SAA, CRP and Hp.

In one aspect of any of the embodiments, described herein is a method of treating a disease or condition as set out in Table 1 in a subject in need thereof, the method comprising: a) determining if the subject has an expression level of at least two genes, or proteins encoded by said genes, selected from SAA, CRP, Hp and MxA, above or below a threshold level; and b) administering a suitable treatment as set out in Table 1 to the subject if the expression level of at least two genes, or proteins encoded by said genes, selected from SAA, CRP, Hp and MxA, is above or below a threshold level. In some embodiments of any of the aspects, the step of determining if the subject has an expression level of at least two genes, or proteins encoded by said genes, selected from SAA, CRP, Hp and MxA, above or below a threshold level can comprise i) obtaining or having obtained a sample from the subject and ii) performing or having performed an assay on the sample obtained from the subject to determine/measure the expression level of at least two genes, or proteins encoded by said genes, selected from SAA, CRP, Hp and MxA, in the subject. In some embodiments of any of the aspects, the step of determining if the subject has a specified threshold expression level of at least two genes, or proteins encoded by said genes, selected from SAA, CRP, Hp and MxA, can comprise performing or having performed an assay on a sample obtained from the subject to determine/measure the expression level of at least two genes, or proteins encoded by said genes, selected from SAA, CRP, Hp and MxA, in the subject. In some embodiments of any of the aspects, the step of determining if the subject has an expression level of at least two genes, or proteins encoded by said genes, selected from SAA, CRP, Hp and MxA, above or below a threshold level can comprise ordering or requesting an assay on a sample obtained from the subject to determine/measure the expression level of at least two genes, or proteins encoded by said genes, selected from SAA, CRP, Hp and MxA, in the subject. In some embodiments of any of the aspects, the step of determining if the subject has an expression level of at least two genes, or proteins encoded by said genes, selected from SAA, CRP, Hp and MxA, above or below a threshold level can comprise receiving the results of an assay on a sample obtained from the subject to determine/measure the expression level of at least two genes, or proteins encoded by said genes, selected from SAA, CRP, Hp and MxA, in the subject. In some embodiments of any of the aspects, the step of determining if the subject has positive expression of at least two genes, or proteins encoded by said genes, selected from SAA, CRP, Hp and MxA can comprise receiving a report, results, or other means of identifying the subject as a subject with an expression level of SAA, CRP, Hp and/or MxA above or below a threshold level. In one aspect of any of the above embodiments, at least three genes or proteins encoded by said genes, selected from SAA, CRP, Hp and MxA are assayed. Preferably, the at least three genes are SAA, CRP and Hp.

In one aspect of any of the embodiments, described herein is a method of treating a disease or condition as set out in Table 1 in a subject in need thereof, the method comprising: a) determining if the subject has a positive expression of at least two genes, or proteins encoded by said genes, selected from SAA, CRP, Hp and MxA is above or below a threshold level; and b) instructing or directing that the subject be administered a suitable treatment as set out in Table 1 if the level of at least two or more genes, or proteins encoded by said genes, selected from SAA, CRP, Hp and MxA is above or below a specified level. In some embodiments of any of the aspects, the step of determining if the subject has a specified expression level above or below a specified threshold of at least two or more genes, or proteins encoded by said genes, selected from SAA, CRP, Hp and MxA can comprise i) obtaining or having obtained a sample from the subject and ii) performing or having performed an assay on the sample obtained from the subject to determine/measure the level of at least two genes, or proteins encoded by said genes, in the subject. In some embodiments of any of the aspects, the step of determining if the subject has positive expression of at least two genes, or proteins encoded by said genes, selected from SAA, CRP, Hp and MxA, can comprise performing or having performed an assay on a sample obtained from the subject to determine/measure the expression level of at least two genes, or proteins encoded by said genes, selected from SAA, CRP, Hp and MxA, in the subject. In some embodiments of any of the aspects, the step of determining if the subject has a specified threshold level of expression level of at least two genes, or proteins encoded by said genes, selected from SAA, CRP, Hp and MxA, can comprise ordering or requesting an assay on a sample obtained from the subject to determine/measure the expression level of at least two genes, or proteins encoded by said genes, selected from SAA, CRP, Hp and MxA, in the subject. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results. In some embodiments of any of the aspects, the step of instructing or directing that the subject undergoing a particular treatment as set out in Table 1 for a particular disease or condition as set out in Table 1 is to cease receiving that particular treatment can comprise providing a report of the assay results. In some embodiments of any of the aspects, the step of instructing or directing that the subject stop being administered a particular treatment can comprise providing a report of the assay results and/or treatment recommendations in view of the assay results. In one aspect of any of the above embodiments, at least three genes or proteins encoded by said genes, selected from SAA, CRP, Hp and MxA are assayed. Preferably, the at least three genes are SAA, CRP and Hp.

TABLE 1
Diseases and Conditions, and their prescribed treatments*

| Infectious Disease | Common Treatment |
|---|---|
| Sepsis | Intravenous broad-spectrum antibiotics (selected from |
| | ceftriaxone, azithromycin, ciprofloxacin, vancomycin, |
| | piperacillin-tazobactam); Intravenous fluids; |
| | and/or vasopressors |
| Meningitis | Intravenous antibiotics (selected from cephalosporins, various |
| | penicillin-type antibiotics, aminoglycoside drugs such as |
| | gentamicin); and/or corticosteroids. |
| Eye Infection | Antibiotic eye drops and/or aminoglycosides and |
| | cephalosporins. |
| Bacterial Pneumonia | Antibiotics (macrolides, fluoroquinolones, tetracyclines), cough |
| | medicine, fever reducer/pain reliever, acetaminophen/NSAIDS, |
| | or combinations thereof. |
| Tuberculosis | Antibiotics such as isoniazid, rifampin (Rifadin ®, Rimactane ®), |
| | ethambutol (Myambutol ®), pyrazinamide. |
| Viral Pneumonia | Anti-virals (such as oseltamivir, zanamivit, peramivir, Ribavirin), |
| | cough medicine, fever reducer/pain reliever, |
| | acetaminophen/NSAIDs. |
| Gastritis | Antibiotics (such as clarithromycin, amoxicillin, metronidazole), |
| | proton pump inhibitors (PPIs), histamine blockers, |
| | and/or antacids. |
| Nail Infection | Antibiotics such as amoxicillin, clavulanic acid, clindamycin, or |
| | combinations thereof. |
| Skin Infection | Antibiotics such as dicloxacillin, erythromycin, tetracycline; or |
| | combinations thereof. |
| Influenza Pneumonia | Antivirals (such as oseltamivir, zanamivit, peramivir), cough |
| | medicine, fever reducer/pain reliever, acetaminophen/NSAIDS, |
| | or combinations thereof. |
| Rhinovirus Pneumonia | Antivirals (such as ribavirin), cough medicine, fever |
| | reducer/pain reliever, acetaminophen/NSAIDs, or |
| | combinations thereof. |
| Whooping cough | Antibiotics (such as erythromycin), cough medicine, fever |
| | reducer/pain reliever, acetaminophen/NSAIDs, or |
| | combinations thereof. |
| Gastrointestinal | Fever reducer/pain reliever, acetaminophen/NSAIDs, or |
| Rotavirus | combinations thereof. |
| Norovirus | |
| Urinary Tract Infection | Antibiotics (such as Levaquin ® (levofloxacin), ciprofloxacin, |
| | Keflex ® (cefalexin), Zotrim ® (sulfamethoxazole, trimethoprim, |
| | phenazopyridine), Bactrim ® (rimethoprim/sulfamethoxazole)), |
| | fever reducer/pain reliever, acetaminophen/NSAIDs, or |
| | combinations thereof. |
| Inflammatory Disease | |
| Acne | Topical ointment (benzoyl peroxide), hormonal treatment, or |
| | bomcinations thereof |
| Autoimmune disorders | |
| Inflammatory | Anti-inflammatoryes (corticosteroids, aminosalicylates), |
| bowel disease | immunosuppressors (azathioprine, mercaptopurine, |
| | cyclosporine, methotrexate, TNF-alpha inhibitors (infliximab, |
| | adalimumab, golimumab)), anti-diarrheal medication, pain |
| | relievers (acetaminophen, NSAIDs), iron supplements, calcium |
| | & Vitamin D supplements, or combinations thereof. |
| Lupus | Non-steroidal anti-inflammatory drugs (NSAIDS) (e.g. |
| | Ibuprofen, naproxen), anti-malarial drugs, BLyS-specific |
| | inhibitors, immunosuppressive agents, chemotherapy, or |
| | combinations thereof. |
| Rheumatoid Arthritis | Anti-inflammatoryes (NSAIDs), corticosteroids, disease- |
| | modifying antirheumatic drugs (DMARDs), biologic agents |
| | (abatacept, adalimumab, anakinra, baricitinib, certolizumab, |
| | etanercept, golimumab, infliximab, rituximab, sarilumab, |
| | tocilizumab, tofacitinib), or combinations thereof. |
| Multiple Sclerosis | Corticosteroids, plasma exchange, ocrelizumab, beta |
| | interferons, glatiramer acetate, fingolimod, dimethyl fumarate, |
| | teriflunomide, siponimod, ocrelizumab, natalizumab, |
| | alemtuzumab, mitoxantrone, or combinations thereof. |
| Type 1 diabetes | Diet management, insulin, or combinations thereof. |
| Chronic Inflammatory | Corticosteroids, immunosuppressant drugs, plasmapheresis, |
| demyelinating | intravenous immunoglobulin therapy, or combinations thereof. |
| polyneuro phathy | |
| Guillian-Barre syndrome | Plasmapheresis, intravenous immunoglobulin therapy, pain |
| | relievers, laxatives, or combinations thereof. |
| Psoriasis | Topical treatments (corticosteroids, Vitamin D and analogues |
| | thereof, anthralin, topical retinoids, calcineurin inhibitors, |
| | salicylic acid, coal tar), light therapy, oral/intravenous |
| | medication (retinoids, methotrexate, cyclosporin), or |
| | combinations thereof. |
| Vasculitis | Corticosteroids, steroid-sparing medication (methotrexate, |
| | azathioprine, mycophenolate, cyclophosphamide), surgery, o |
| | combinations thereof. |
| Grave’s disease | Radioactive iodine therapy, anti-thyroid medication, beta- |
| | blockers (propranolol, atenolol, metoprolol, nadolol), surgery, |
| | corticosteroids, or combinations thereof. |
| Myasthenia gravis | Cholinesterase inhibitors, corticosteroids, |
| | immunosuppressants, plasmapheresis, intravenous |
| | immunoglobulin therapy, monoclonal antibodies, surgery, or |
| | combinations thereof. |
| Hashimoto’s thyroiditis | Synthetic hormones (such as levothyroxine) |
| Autoinflammatory | |
| conditions | |
| Cystic Fibrosis | Medication targeting gene mutation (cystic fibrosis |
| | transmembrane conductance regulator (CTFR) modulators); |
| | antibiotics (to treat and prevent infection); anti-inflammatoryes; |
| | mucus-thinning drugs (for example, hypertonic saline); |
| | bronchodilators; oral pancreatic enzymes; stool softeners; acid- |
| | reducing medication; vest therapy; pulmonary rehabilitation; |
| | surgery; lung/liver transplant; or combinations thereof. |
| Chronic Obstruction | Smoking cessation; short-acting bronchodilators (albuterol, |
| Pulmonay Disease (COPD) | Ipratropium, Levalbuterol); long-acting bronchodilators |
| | (aclidinium, arformoterol, formoterol, indacaterol, tiotropium, |
| | salmeterol, umeclidinium); inhaled steroids (fluticasone, |
| | budesonide); combination inhalers; oral steroids |
| | (corticosteroids); phosphodiesterase-4 inhibitors; theophylline; |
| | Antibiotics (if infection present); lung therapies (oxygen therapy, |
| | pulmonary rehabilitation program); surgery; lung transplant, or |
| | combinations thereof. |
| Alllergy | Antihistamines, corticosteroids, decongestants, NSAIDs, or |
| | combinations thereof. |
| Asthma | Corticosteroids, albuterol, or combinations thereof. |
| Celiac disease | Diet management, anti-inflammatorys, or combinations thereof. |
| Glomerulonephritis | Dialysis |
| Hepatitis | Anti-virals (entecavir, tenofovir, lamivudine, adefovir, |
| | telbivudine), interferon injections, surgery (liver transplant), or |
| | combinations thereof. |
| Chronic Prostatitis | NSAIDs, alpha blockers, or combinations thereof. |
| Diverticulitis | Pain relievers (acetaminophens), antibiotics (if infectious), |
| | surgery, or combinations thereof. |
| Cancer | Surgery, £ radiotherapy, chemotherapy, immunotherapy, |
| | hormone therapy, targeted drug therapy, cryoablation, |
| | radiofrequency ablation, or combinations thereof. |
| Heart disease | ACE inhibitors, aldosterone inhibitors, angiotension II receptor |
| | blockers, beta-blockers, vasodilators, or combinations thereof. |

*other diseases which are associated with those listed above are also described below.

### Definitions

In the specification, the term “healthy” should be understood to mean where the individual or patient has no underlying medical condition, or does not have or is not suffering from an infection, an inflammatory response, event, disorder, condition or otherwise.

In the specification, the term “health status” should be understood to mean whether a subject is healthy (see above), or is found to be, or is, suffering from one or more from an infection, an infectious disease, an infectious condition, an inflammatory disease, an inflammatory condition, an autoinflammatory condition, an autoimmune condition, inflammatory bowel disease, heart disease, cancer, or combinations thereof.

In the specification, the term “infection” or “infectious condition” should be understood to mean infections or diseases caused by microorganisms or microbes such as viruses, bacteria, fungi, protozoa and helminths. Examples of infections include septicaemia, meningitis, eye infections, tuberculosis, upper respiratory tract infections, pneumonia, gastritis, nail and skin infections and the like. Common bacteria that cause disease and infections include Staphylococcus, Streptococcus, Pseudomonas, Clostridium difficile, Escherichia coli and Listeria monocytogenes.

In the specification, the term “inflammatory condition” should be understood to mean immune-related conditions resulting in allergic reactions, myopathies and abnormal inflammation and non-immune related conditions having causal origins in inflammatory processes. Examples include as acne, autoimmune conditions, autoinflammatory condition, chronic prostatitis, diverticulitis, cancer, heart disease, and the like.

In the specification, the term “autoinflammatory condition” should be understood to mean a group of diseases characterised by seemingly unprovoked episodes of fever and inflammation of skin, joints, serosal surfaces and other organ involvement including the nervous system. Examples of autoinflammatory conditions include allergy, asthma, autoimmune conditions, celiac disease, glomerulonephritis, hepatitis, cryopyrinopathies or cryopyrin-associated periodic syndromes (CAPS) (a group of three rare autoinflammatory diseases that includes familial cold autoinflammatory syndrome (FCAS), Muckle-Wells syndrome (MWS) and chronic infantile neurologic cutaneous articular syndrome (CINCA)), and the like.

In the specification, the term “autoimmune condition” should be understood to mean a condition in which your immune system mistakenly attacks your body. Examples of autoimmune conditions include asthma, inflammatory bowel diseases, lupus, rheumatoid arthritis, multiple sclerosis, Type 1 diabetes, Chronic inflammatory demyelinating polyneuropathy, Guillian-Barre syndrome, psoriasis, vasculitis, Grave's disease, myasthenia gravis, Hashimoto's thyroiditis, and the like.

In the specification, the term “inflammatory bowel disease” should be understood to mean disorders that involve chronic inflammation of the digestive tract. Examples of inflammatory bowel disease include colitis, ulcerative colitis, and Crohn's disease.

In the specification, the term “cancer” should be understood to mean a cancer selected from the group comprising node-negative, ER-positive breast cancer; early stage, node positive breast cancer; multiple myeloma, prostate cancer, glioblastoma, lymphoma, fibrosarcoma; myxosarcoma; liposarcoma; chondrosarcoma; osteogenic sarcoma; chordoma; angiosarcoma; endotheliosarcoma; lymphangiosarcoma; lymphangioendotheliosarcoma; synovioma; mesothelioma; Ewing's tumour; leiomyosarcoma; rhabdomyosarcoma; colon carcinoma; pancreatic cancer; breast cancer; ovarian cancer; squamous cell carcinoma; basal cell carcinoma; adenocarcinoma; sweat gland carcinoma; sebaceous gland carcinoma; papillary carcinoma; papillary adenocarcinomas; cystadenocarcinoma; medullary carcinoma; bronchogenic carcinoma; renal cell carcinoma; hepatoma; bile duct carcinoma; choriocarcinoma; seminoma; embryonal carcinoma; Wilms' tumour; cervical cancer; uterine cancer; testicular tumour; lung carcinoma; small cell lung carcinoma; bladder carcinoma; epithelial carcinoma; glioma; astrocytoma; medulloblastoma; craniopharyngioma; ependymoma; pinealoma; hemangioblastoma; acoustic neuroma; oligodendroglioma; meningioma; melanoma; retinoblastoma; and leukemias. Also included are metastases selected from the group comprising: bone metastases; lung metastases; liver metastases; bone marrow metastases; breast metastases; and brain metastases.

In the specification, the term “heart disease” should be understood to mean cardiovascular disease selected from the group comprising: ischemic heart disease, cardiac hypertrophy; myocardial infarction; stroke; arteriosclerosis; and heart failure.

In the specification, the term “subject”, “individual” or “patient” should be understood to mean all mammals, for example, a human, primates, non-human primates, farm animals (such as pigs, horses, goats, sheep, cows (including bulls, bullocks, heifers etc.), donkey, reindeer, etc.), veterinary mammals (such as dogs, cats, rabbits, hamsters, guinea pigs, mice, rats, ferrets, etc.), and mammals kept in captivity (such as lions, tigers, elephants, zebras, giraffes, pandas, rhino, hippopotamus, etc.), and other mammals and higher mammals for which the use of the invention is practicable.

In this specification, the term “biological sample” should be understood to mean blood or blood derivatives (serum, plasma etc.), urine, saliva or cerebrospinal fluid.

In the specification, the term “treatment” should be understood to mean prohibiting, preventing, restraining, and slowing, stopping or reversing progression or severity of a disease or condition associated with inflammatory or non-inflammatory diseases, conditions or infections.

Serum Amyloid A (SAA) is small protein primarily associated with the innate response and is predominantly secreted by hepatocytes. The protein responds very quickly to the onset of a challenge with levels increasing 1000-fold or more within 24 hours. The SAA sequence is remarkably conserved across many species indicating an important role. The literature widely reports SAA as being associated with non-specific inflammatory responses.

Haptoglobin (Hp) is a glycoprotein composed of 2α and 2β subunits. It is primarily engaged in haemolysis where it limits haemoglobins oxidative activity but additionally levels are seen to elevate during infectious and inflammatory episodes. The protein is produced by hepatocytes with some reports of production at other sites such as the lung and kidney.

C Reactive Protein (CRP) is another acute phase protein whose levels are seen to increase in response to acute and chronic challenges. CRP levels are widely reported as non-specific but clinically useful markers for many types of inflammation. CRP is a member of the pentraxin family of plasma protein and are described as stably conserved in vertebrate evolution.

Myxovirus Resistance Proteins (Mx) are innate intracellular GTPases activated by the hosts interferon system (IFN) in response to viral challenge. They are understood to play an important role in the host immune response. Myxovirus Resistance Protein A (MxA), the best understood of the Mx family, accumulates in the cytoplasm of cells and in human peripheral blood mononuclear cells (PBMC), and macrophages are the principle producers. MxA is evolutionarily conserved and present in almost all vertebrates.

In the specification, the term “test platform” in relation to determining the levels of the biomarkers should be understood to mean determining biomarker concentrations by immunoassay-based methods such as lateral flow immunoassays (LFAs), enzyme-linked immunosorbent assay (ELISA), immunoturbidimetric assays, quantitative polymerase chain reaction (PCR), reverse transcriptase PCR (RT-PCR), quantitative real time RT-PCR (qRT-PCR), Western Blot, protein determination on polyacrylamide gels, and the like.

In this specification, the term “at least two” should be understood to mean and encompass that at least two, three or all of the proteins can be selected from the group consisting of SAA, Hp, CRP and MxA. The combinations of the genes are as follows: SAA, CRP; SAA, Hp; SAA, MxA; CRP, Hp; CRP, MxA; Hp, MxA; SAA, CRP, Hp; SAA, CRP, MxA; SAA, Hp, MxA; CRP, Hp, MxA; and SAA, CRP, Hp, MxA.

In the specification, the term “variant” should be understood to mean proteins having amino acid sequences which are substantially identical to wild-type SAA, CRP, Hp and MxA proteins, respectively. Thus, for example, the term should be taken to include proteins or polypeptides that are altered in respect of one or more amino acid residues. Preferably such alterations involve the insertion, addition, deletion and/or substitution of 5 or fewer amino acids, more preferably of 4 or fewer, even more preferably of 3 or fewer, most preferably of 1 or 2 amino acids only. Insertion, addition and substitution with natural and modified amino acids is envisaged. The variant may have conservative amino acid changes, wherein the amino acid being introduced is similar structurally, chemically, or functionally to that being substituted. Generally, the variant will have at least 70% amino acid sequence identity, preferably at least 80% sequence identity, more preferably at least 90% sequence identity, and ideally at least 95%, 96%, 97%, 98% or 99% sequence identity with wild-type human proteins described herein. In this context, sequence identity is a polypeptide sequence that shares 70% amino acid identity with wild-type human protein is one in which any 70% of aligned residues are either identical to, or conservative substitutions of, the corresponding residues in the wild-type human proteins. It should be noted that this is the amount of characters which match exactly between two different sequences. Hereby, gaps are not counted, and the measurement is relational to the shorter of the two sequences.

The term “variant” is also intended to include chemical derivatives of the wild-type protein, i.e. where one or more residues of Cf5 is chemically derivatized by reaction of a functional side group. Also included within the term variant are Cf5 molecules in which naturally occurring amino acid residues are replaced with amino acid analogues.

The protein sequences encoded by the human genes of SAA, CRP, Hp and MxA are provided in SEQ ID NO: 1, 4, 7 and 10, respectively. The Accession Numbers for the human proteins are also provided. The nucleotide sequences of the human genes of SAA, CRP, Hp and MxA are provided in SEQ ID NO: 2, 3, 5, 6, 8, 9, 11 and 12. The Accession Numbers for the human genes are also provided.

In the specification, the term “Youden Index (J)” should be understood to mean the sum of sensitivity and specificity minus one and reflects the overall capacity of an early warning model to detect outbreaks and non-outbreaks.

In some embodiments of any of the aspects, measurement of the level of a target and/or detection of the level or presence of a target, e.g. of an expression product (nucleic acid or polypeptide of one of the genes described herein) or a mutation can comprise a transformation. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample, e.g., from whole blood to blood serum by differential centrifugation. A biological/chemical transformation can involve the action of at least one enzyme and/or a chemical reagent in a reaction. For example, a DNA sample can be digested into fragments by one or more restriction enzymes, or an exogenous molecule can be attached to a fragmented DNA sample with a ligase. In some embodiments of any of the aspects, a DNA sample can undergo enzymatic replication, e.g., by polymerase chain reaction (PCR).

Transformation, measurement, and/or detection of a target molecule, e.g. an SAA mRNA or polypeptide, can comprise contacting a sample obtained from a subject with a reagent (e.g. a detection reagent) which is specific for the target, e.g., a target-specific reagent. In some embodiments of any of the aspects, the target-specific reagent is detectably labeled. In some embodiments of any of the aspects, the target-specific reagent can generate a detectable signal. In some embodiments of any of the aspects, the target-specific reagent generates a detectable signal when the target molecule is present.

Methods to measure gene expression products are known to a skilled artisan. Such methods to measure gene expression products, e.g., protein level, include ELISA (enzyme linked immunosorbent assay), western blot, immunoprecipitation, and immunofluorescence using detection reagents such as an antibody or protein binding agents. Alternatively, a peptide can be detected in a subject by introducing into a subject a labeled anti-peptide antibody and other types of detection agent. For example, the antibody can be labeled with a detectable marker whose presence and location in the subject is detected by standard imaging techniques.

For example, antibodies for the various targets described herein are commercially available and can be used for the purposes of the invention to measure protein expression levels, e.g. anti-SAA (Cat. No. ab687; Abcam, Cambridge Mass.). Other antibodies for each of SAA, CRP, Hp and MA are available from Abcam, Lifespan Biosciencs, Novus Biological, Bio-Rad, Thermofisher, Abcepta, and the like. Examples of the available antibodies can be found in Table 2 below.

TABLE 2
Commercially available antibodies

| Biomarker | Supplier | Immunogen |
|---|---|---|
| SAA | Abcam | aa 1-100 |
| SAA | Lifespan Bioscience | Highly purified recombinant human SAA, 12 kD |
| SAA | Novus Biological | E. coli-derived recombinant human SAA1. |
| | | Arg19-Tyr122 |
| SAA | Bio-Rad | Human Serum Amyloid A |
| CRP | Abcam | Synthetic peptide within Human C Reactive Protein |
| | | aa 200 to the C-terminus (C terminal). The exact |
| | | sequence is proprietary. |
| CRP | Lifespan Bioscience | Human CRP |
| CRP | Novus Biological | Human plasma-derived CRP |
| CRP | Bio-Rad | N/A |
| Haptoglobin | Abcam | Full length native protein (Purified haptoglobin types |
| | | 1-1, 2-1, and 2-2 from pooled human plasma). |
| Haptoglobin | Lifespan Bioscience | aa 19-406 |
| Haptoglobin | Novus Biological | C terminal human Haptoglobin |
| Haptoglobin | Bio-Rad | Purified protein from Human plasma |
| MxA | Abcam | Synthetic peptide within Human MX1 aa 100-200. |
| | | The exact sequence is proprietary. |
| MxA | Novus Biological | Full length human recombinant protein of human |
| | | MX1 (NP_002453) produced in HEK293T cell. |
| MxA | Lifespan Bioscience | aa 617-646 |
| MxA | ThermoFisher | Full length human recombinant protein of MX1 |
| | | produced in HEK293T cell |

Alternatively, since the amino acid sequences for the targets described herein are known and publicly available at the NCBI website, one of skill in the art can raise their own antibodies against these polypeptides of interest for the purpose of the methods described herein.

The amino acid and nucleotide sequences of the polypeptides described herein have been assigned NCBI accession numbers for different species such as human, mouse and rat. In particular, the NCBI accession numbers for the amino acid sequence of human SAA, human CRP, human Hp and human MxA is included herein, e.g. SEQ ID NO: 1, 4, 7, and 10, respectively. The NCBI accession numbers for the nucleotide sequence of human SAA, human CRP, human Hp and human MxA is included herein, e.g. SEQ ID NO: 2, 3, 5, 6, 8, 9, 11 and 12.

In some embodiments of any of the aspects, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change of color, upon encountering the targeted molecules. In some instances, signal amplification can be integrated into the particular protocol, wherein a secondary antibody, that includes the marker stain or marker signal, follows the application of a primary specific antibody.

In some embodiments of any of the aspects, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiments of any of the aspects, the immunoassay can be a quantitative or a semi-quantitative immunoassay.

An immunoassay is a biochemical test that measures the concentration of a substance in a biological sample, typically a fluid sample such as blood or serum, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (e.g., any of the targets as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., a blood sample) from a subject is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3,3′,5,5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce significant color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests used for medical diagnostics, either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tissue samples etc. Strip tests are also known as dip stick tests, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments of any of the aspects, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. Nos. 10/278,676; 09/579,673 and 10/717,082, which are incorporated herein by reference in their entirety, are non-limiting examples of such lateral flow test devices. Examples of patents that describe the use of “dip stick” technology to detect soluble antigens via immunochemical assays include but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, an adaptation of Western blotting (Towbin et al., Proc. Nat. Acad. Sci. 76:4350 (1979)). In a Western blot, the polypeptide or fragment thereof can be dissociated with detergents and heat and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. A dot blot immobilizes a protein sample on a defined region of a support, which is then probed with antibody and labelled secondary antibody as in Western blotting. The intensity of the signal from the detectable label in either format corresponds to the amount of enzyme present, and therefore the amount of polypeptide. Levels can be quantified, for example by densitometry.

In some embodiments of any of the aspects, the level of a target can be measured, by way of non-limiting example, by Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy and/or immunoelectrophoresis assay.

In certain embodiments, the gene expression products as described herein can be instead determined by determining the level of messenger RNA (mRNA) expression of the genes described herein. Such molecules can be isolated, derived, or amplified from a biological sample, such as a blood sample. Techniques for the detection of mRNA expression is known by persons skilled in the art, and can include but not limited to, PCR procedures, RT-PCR, quantitative RT-PCR Northern blot analysis, differential gene expression, RNAse protection assay, microarray based analysis, next-generation sequencing; hybridization methods, etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments of any of the aspects, the level of an mRNA can be measured by a quantitative sequencing technology, e.g. a quantitative next-generation sequence technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

The nucleic acid sequences of the genes described herein have been assigned NCO accession numbers for different species such as human, mouse and rat. For example, the human SAA nucleotide sequences (e.g. SEQ ID NO: 2 and 3) is known. Accordingly, a skilled artisan can design an appropriate primer based on the known sequence for determining the mRNA level of the respective gene.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In some embodiments of any of the aspects, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments of any of the aspects, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled reagent is exposed to light of the proper wavelength, its presence can then be detected due to fluorescence. In some embodiments of any of the aspects, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc.; BODIPY dyes and quinoline dyes. In some embodiments of any of the aspects, a detectable label can be a radiolabel including, but not limited to ³H, ¹²⁵I, ³⁵S, ¹⁴C, ³²P, and ³³P. In some embodiments of any of the aspects, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments of any of the aspects, a detectable label is a chemiluminescent label, including, but not limited to lucigenin, luminol, luciferin, isoluminol, theromatic acridinium ester, imidazole, acridinium salt and oxalate ester. In some embodiments of any of the aspects, a detectable label can be a spectral colorimetric label including, but not limited to colloidal gold or colored glass or plastic (e.g., polystyrene, polypropylene, and latex) beads.

In some embodiments of any of the aspects, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i.e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e.g. from DAKO; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

A level which is less than a reference level can be a level which is less by at least about 10%, at least about 20%, at least about 50%, at least about 60%, at least about 80%, at least about 90%, or less relative to the reference level. In some embodiments of any of the aspects, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments of any of the aspects, a level which is more than a reference level can be a level which is statistically significantly greater than the reference level.

In some embodiments of any of the aspects, the reference can be a level of the target molecule in a population of subjects who do not have or are not diagnosed as having, and/or do not exhibit signs or symptoms of infection, inflammation or diseases associated with these symptoms. In some embodiments of any of the aspects, the reference can also be a level of expression of the target molecule in a control sample, a pooled sample of control individuals or a numeric value or range of values based on the same. In some embodiments of any of the aspects, the reference can be the level of a target molecule in a sample obtained from the same subject at an earlier point in time, e.g., the methods described herein can be used to determine if a subject's sensitivity or response to a given therapy is changing over time.

In some embodiments of any of the aspects, the level of expression products of no more than 200 other genes is determined. In some embodiments of any of the aspects, the level of expression products of no more than 100 other genes is determined. In some embodiments of any of the aspects, the level of expression products of no more than 20 other genes is determined. In some embodiments of any of the aspects, the level of expression products of no more than 10 other genes is determined.

In some embodiments of the foregoing aspects, the expression level of a given gene can be normalized relative to the expression level of one or more reference genes or reference proteins.

In some embodiments, the reference level can be the level in a sample of similar cell type, sample type, sample processing, and/or obtained from a subject of similar age, sex and other demographic parameters as the sample/subject for which the level of SAA, CRP, Hp and/or MxA is to be determined. In some embodiments, the test sample and control reference sample are of the same type, that is, obtained from the same biological source, and comprising the same composition, e.g. the same number and type of cells.

The term “sample” or “test sample” as used herein denotes a sample taken or isolated from a biological organism, e.g., a blood or plasma sample from a subject. In some embodiments of any of the aspects, the present invention encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine (and urine derivatives); sperm; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject. In some embodiments of any of the aspects, the test sample can be whole blood, venous blood, arterial blood, capillary blood, serum, buffy coat (white blood cells and platelets), plasma from processed blood tubes (EDTA, K2 EDTA, sodium or lithium heparin, sodium citrate, potassium oxalate).

The test sample can be obtained by removing a sample from a subject but can also be accomplished by using a previously isolated sample (e.g. isolated at a prior timepoint and isolated by the same or another person).

In some embodiments of any of the aspects, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments of any of the aspects, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments of any of the aspects, the test sample is a clarified test sample, for example, by centrifugation and collection of a supernatant comprising the clarified test sample. In some embodiments of any of the aspects, a test sample can be a pre-processed test sample, for example, supernatant or filtrate resulting from a treatment selected from the group consisting of centrifugation, filtration, thawing, purification, and any combinations thereof. In some embodiments of any of the aspects, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of an expression product as described herein.

In some embodiments of any of the aspects, the methods, assays, and systems described herein can further comprise a step of obtaining or having obtained a test sample from a subject. In some embodiments of any of the aspects, the subject can be a human subject. In some embodiments of any of the aspects, the subject can be a subject in need of treatment for (e.g., having or diagnosed as having an infection, a non-infectious disease, an inflammatory disease) an infection, a non-infectious disease, an inflammatory disease or a subject at risk of or at increased risk of developing an infection, a non-infectious disease, an inflammatory disease as described elsewhere herein.

In some embodiments of any of the aspects, the sample obtained from a subject can be a biopsy sample. In some embodiments of any of the aspects, the sample obtained from a subject can be a blood or serum sample.

Sequences
(hSAA) (Accession No. AAA60297)
SEQ ID NO: 1
MKLLTGLVFCSLVLGVSSRSFFSFLGEAFDGARDMWRAYSDMREANYIGSDKYFHARGNYDAAKRGPGGV
WAAEAISDARENIQRFFGHGAEDSLADQAANEWGRSGKDPNHFRPAGLSEKY
(hSAA) (Accession No. M1906.1)
SEQ ID NO: 2
ATGAAGCTTCTCACGGGCCTGGTTTTCTGCTCCTTGGTCCTGGGTGTCAGCAGCCGAAGCTTCTTTTCGT
TCCTTGGCGAGGCTTTTGATGGGGCTCGGGACATGTGGAGAGCCTACTCTGACATGAGAGAAGCCAATTA
CATCGGCTCAGACAAATACTTCCATGCTCGGGGGAACTATGATGCTGCCAAAAGGGGACCTGGGGGTGTC
TGGGCTGCAGAAGCGATCAGCGATGCCAGAGAGAATATCCAGAGATTCTTTGGCCATGGTGCGGAGGACT
CGCTGGCTGATCAGGCTGCCAATGAATGGGGCAGGAGTGGCAAAGACCCCAATCACTTCCGACCTGCTGG
CCTGTCTGAGAAATACTGA
(hSAA) (Accession No. NG_021330)
SEQ ID NO: 3
TTTTCACAGTTCATTTACGGCCCTCTTTCCAACACATTCACTGCCAATACTCTTATTGACAATAACTGTA
TTGTTGAACCTTCCAGTATCCTGCATTCCCGGATCAAGGCCCCCTCAAAGCCCTGATATGCAAATATCTG
GGAAAAGAATGTTCCAGAGGAAAGGAACAGCTAATCCGAGGCCCCTAGGGTAAGATGTGCCTGGGGGTTT
GGAGACCAGTGTGGCCAGAGCAAAATGAGCAGGAGGAGAGAATTGGATGATGAGGTACGAGAGGAAGGAG
TTAGGACAGTTTGAGTAAAGTTTGAAAACCATTATAAGGGCTTTGACTTCAACTATGAGTGGAAGTGGAA
TCCTCCGGAGAGTTTTGAATGGAGAGTGATAGAAGTTGTCTTGTGTTGTAACAGTCTGGCTGCTATACTG
AAAAGAGACTAGTTGGCGGCAAAGGGGGAAATGTGGAAGCCAGTTAAGAAGCCATCATAACCCAGAAGGT
GATGCCTAATAACATCTCTCTGGGAGCAGCGGAGAGATGATAAGGGTTTGCCTTCTGAATATGTTTTTTG
ACAATTAATGTAAACATTTCAAGTAGGCTGAGATTTTATTGCATATTAACAATGTCCATGTTCACTCGCG
GCAGCCGCCCCCTTCTGCGCGGTCATGCCGAGCCAGCACCTGGGCCTGGAACTGGGCCGCAGCCCCCAGC
TTCACCCACCACCTCCCTACCATGGACCCCTGCAAAGTGAACGAGCTTCGGGCCTTTGTGAAAATGTGTA
AGCAGGATCCGAGCGTTCTGCACACCGAGGAAATGCGCTTCCTGAGAGAGTGGGTGGAGAGCATGGGAGG
TAAAGTACCACCTGCTACTCAGAAGGCTAAATCAGAAGAAAATACCAAGGAAGAAAAACCTGATAGTAAG
AAGGTGGAGGAAGACTTAAAGGCAGACGAACCATCAACTGAGGAAAGTGATCTAGAAATTGATAAAGAAG
GTGTGATTGAACCAGACACTGATGCTCCTCAAGAAATGGGAGATGAAAATGTGGAGATAACGGAGGAGAT
GATGGATCAGGCAAATGATAAAAAAGTGGCTGCTATTGAAGTCCTAAATGATGGTGAACTCCAGAAAGCC
ATTGACTTATTCACAGATGCCATCAAGCTGAATCCTCGCTTGGCCATTTTGTATGCAAAGAGGGCCAGTG
TCTTCGTCAAATTACAGAAGCCAAATGCTGCCATCCAAGACTGTGACAGAGCCATTGAAATAAATCCTGA
TTCAGCTCAGCCTTACAAGTGGCGGGGGAAAGCACACAGACTTCTAGGCCACTGGGAAGAAGCAGCCCAT
GATCTTGCCTTTGCCTGTAAATTGGATTATGATGAAGATGCTAGTGCAATGCTGAAAGAAGTTCAACCTA
GGGCACAGAAAATTGCAGAACATTGGAGAAAGTATGAGCGAAAACATGAAGAGCGAGAGATCAAAGAAAG
AATAGAACGAGTTAAGAAGGCTCAAGAAGAGCAGGAGAGAGCCCAGAGGGAGGAAGAAGCCAGACGACAG
TCAGGAGCTCACTATGGCCCTTTTCCAGGTGGCTTTCCTGGTGGAATGCCTGGTAATTTTCCCGGAGGAA
TGCCTGGAATGGGAGGGGACATGCCTGGAATGGCCGGAATGCCTGGACTCAATGAAATTCTTAGTGATCC
AGAGGCTCTTGCAGCCATGCAGGATCCAGAAGTTATGGTGGCCTTCCAGGATGTGGCTCAGAACCCAGCA
AATATGTCAAAATACCAGAGCAACCCAAAGGTTATGAATCTCATCAGTAAATTGTCAGCCAAATTTGGAG
GTCAAGCATAATGCCCTTCTGATAAATAAAGCCCTGCTGAAGGAAAAGCAACCTAGATCACCTTATGGAT
GTCGCAATAATACAAACCAACGTACCTCTGACCTTCTCATCAAGAGAGCTGGGGTGCTTTGAAGATAATC
CCTACCCCTCTCCCCCAAATGCAGCTGAAGCATTTTACAGTGGTTTGCCATTAGGGTATTCATTCAGATA
ATGTTTTCCTACTAGGAATTACAAACTTTAAACACTTTTTAAATCTTCAAATATTTAAAACAAATTTAAA
GGGTCTGTTAATTCTTATATTTTTCTTTACTAATCATTGTGGATTTTTCCTTAAATTATTGGGCAGGGAA
TATACTTATTTATGGAAGATTACTGCTCTAATTTGAGTGAAATAAAAGTTATTAGTGCGAGGCAAACATA
AAAAAAAAAAGTCCATGTTCATCTCTAAATGACATCATTGTTCCAAAGCTTTTCCATTCTTCTTAACCTT
CCACCTGTCAATCTATAGGAGATGACTTCTCCTACTTCACTCATGCATTGACTCCTTCAATCAATAAAAG
TGACTAAGAACCTGCTACAGGTGAGGTGCTGTGTTTGGTGTTAAAGTGACAACAGTTATCTGTCAATAAG
CCTGACAAGGTTCCTATCCCTGTGTTTTGTGCACTCTGGGTCAAACTCAGAAATGCAAACAGGTGGAGAG
CGATGAGTTCTATGACTGGTAAAGAAAAGGGCCTGCTGGTTTCCCTCAGGATCTCTGTCCTTCATCTCAA
AATGCATCTTCCTTGTTATCGTTCCTCTCCTTCCTGTCTCAGAGGAAGACCTGCTCCTGCTACACTCTGG
GCAACCTTGTCCCCGTGGCCCTGTGGCCCCTTGGTTGTTGAAGTCTATGTTATGCCCTATCTTTTACCCT
CAGTCACTCTCTCTGTTAACATTCTCCCTGTGCCCTGTAACCCTCCCTCATCTTTAAATAAATCCTCCTC
CTTTGACCTTCGCATGTATTCAGTCATGCAACTCAACAAGCATTTATTGCACAGTGATATTCAATTTGCC
ACTTGCTAAAAGTCTGAACCTTGGCAGCTGAATGTGATCAGAAAAAAAGCACGACTGCTATGACTAGTCT
CACTTTAAATTCATGGTCGTTGACCAAGAGCTACCATACAATCCACTACCTTTCTCAAGTTCAGTCACAT
TCTTCCTTTCCTAGATGTCTGCTTTCTACTTCTCTTCTCTTCTGAAACTTCCCACAACTCCTCGTTCATT
CTCTTCTCAGTTGACAACTTTGCTTCCTATTTCACTGAAAAATAGAAGCAATCAGATATGAACTTCTGGC
TGGGCATGGTAGCTCATGCCTATAATCTCAGCACTTTGGGAGGCCAAGGCAGGAGGACTGCAGGTTAGGA
ATTTGAGACCAGCCTGGGCAACATGGTGAAACTCCCACTGTACTAAAAATTTTAAAAATTACTCAAACAT
ATTGGCAAACAACTGCAGTCCCAGCTACTTGGGAGGTTGAGATGCAAGGATCACTTAAACCTGGGAGGCT
GAGGCTGCAGTGAGCCATGATTGCACCACTGCACTCCAGCTCAGGCAACAGAGCAAGACCCTGTCTTGAG
AGGAGAGGAGAAGAGAGGAGGGGAGGGGAGGGCAGGGGAGGGGAGGGGAGGGGAAGGGAGAGGGGAGGGG
AGAGGGGAGGAGAGAGGGGAGGGGAGGGGAGGGGAGGGGAGGGGAGGAGAGGAGGATCAGGTGAGGAGTA
TGCCAAGGAGTGTTTTTAAGACTTACTGTTTTCTCTTTCCCAACAAGATTGTCATTTCCTTTAAAAAGTA
GTTATCCTGAGGCCTATATTCATAGCATTCTGAAAGAAAGAAAAGAAAAGAGGAAAGAAAGAGAGAGGAA
GGAAGGAAGGAGAAAGAGAGAGGAAGGAAGGAGAAAGAGAGAGGAAGGAAGGGAGGAAGAGAAGAAGGGA
GGAAGAAAAGAAGGAAGGAAGGAGGGAGGGAGGGAAGGGAGGGAGGGAAAGAGGAAGAAAGGAGGGAAAG
AAGGAAGGAAGAGAGAGAGGAAGGAAGGAGGAAGAGAGAAGAAGGAAGGAGGAAGACAGAGAGGGAGTAA
GGAAGGAAGGAAGGAGAAAGAGAGAGGAAGGAAGAAATGAAGGAAGGAAGGAAAGAAAGAAAAAATAAAA
GAGTGAAAACGGACTGGAGAAGAAGAAACCACAGTTGCTGCTATATCCACCAGCCTCTCTGCATGTCCTG
GCCTCAGCCCTGCTGGGCTCTGGTACTGACCACTTCCTTCCTTCCTAATTTCCTAATTGACTAGGCCAGC
TGAGCAGGGCTTTTCTGTGCTGAGGAGGTAAATCTCTGGATATCTAGACTGAGGGGTGGAAGGAGCCTTC
CAGGGCACACATGAGACATGGCAGGGGTAGGCTGCTAGTTTTATTTTGTTTTCTTTTAGACACAGGGTCT
TGCTCTGTTAACCAGGCTGGAGTGCAGTGGCGTGATTATAGCTCACTGCAGCCTTGACCTCCTGGGTCTC
CCACAATCCTTCCGCTTCAGCCTCTTGAGTAGCTGGGACTGCAGGTGCACACTACCACACCCGGTCCATT
TATTTTTATATTTCGTAGAGACAAGATCTTACAGTTTTGCACAGAGTGATCTTAAACTCTTGACCCCAAG
TGATCCTCCTGCCTTGGCCTCCAAAAGCATTGGGATTATAGGAGTGAGCCACTGTGCTGGACCTAGTCTG
TCAGCTTTGAAGCTTTAGATATGAACTCAGAGGGACTTCATTTCAGAGGCATCTGCCATGTGGCCCAGCA
GAGCCCATCCTGAGGAAATGACTGGTAGAGTCAGGAGCTGGCTTCAAAGCTGCCCTCACTTCACACCTTC
CAGCAGCCCAGGTGCCGCCATCACGGGGCTCCCACTCTCAACTCCGCAGCCTCAGCCCCCTCAATGCTGA
GGAGCAGAGCTGGTCTCCTGCCCTGACAGCTGCCAGGCACATCTTGTTCCCTCAGGTTGCACAACTGGGA
TAAATGACCCGGGATGAAGAAACCACTGGCATCCAGGAACTTGTCTTAGACCGTTTTGTAGGGGAAATGA
CCTGCAGGGACTTTCCCCAGGGACCACATCCAGCTTTTCTTCGCTCCCAAGAAACCAGCAGGGAAGGCTC
AGTATAAATAGCAGCCACCGCTCCCTGGCAGGCAGGGACCCGCAGCTCAGCTACAGCACAGATCAGGTGA
GGAGCACACCAAGGAGTGATTTTTAAAACTTACTCTGTTTTCTCTTTCCCAACAAGATTATCATTTCCTT
TAAAAAAAATAGTTATCCTGGGGCATACAGCCATACCATTCTGAAGGTGTCTTATCTCCTCTGATCTAGA
GAGGTAAGCAGGGTCGGGCCTGGTAGTACTTGGATGGGAGAACACCTGGGAATACCAGGTGCTAAAGGCT
TTAAGAATAAAAAATAATGATCCTGCTTTGTGTTTATCCCATGTTGAGTTCTGTGCGGGGCAGAGGGAAC
ACACGGTAAATGCGTTATGGGGAATTATAGGCTACTTGAGGGAGTGACAGTCTGGTGGTAACTCCTGCCT
TCCTCCATCAGTGCCACGTTGGCATCCTCTTATGCAGTCAGGCTTCAGGGCTGATGGGTTCAGAACCGAG
GGCTTCTGGCTCTGAGTGAGGTCCTGCTGCAAGGTTTCCTAGATGAGCCACTGAGACTCTAATAAGATCC
AGTGGAAATAACCAGGCTCTCGTCGGAATATAAGTCCCAAGGGAAGCTGTGCCAGTCTTGTGGGCGACTG
CCTGACTTCTCCTTTCATTTCAGCACCATGAAGCTTCTCACGGGCCTGGTTTTCTGCTCCTTGGTCCTGG
GTGTCAGCAGCCGAAGCTTCTTTTCGTTCCTTGGCGAGGCTTTTGATGGTAAGGCTTCAGAAGGTTTGCA
GGATTTCTGAAGAGAAACATCACCCTGGACCTGATAAACTGGGGAAAATGATGCTTTCGGAAGGCTGCTT
TTGAACCACAGAGTTGCTAGTGTCTGCGTTGCTGAGGCCTGCCAGGAACTAGGGTTTGCTGGGTTGCCTG
TCTCGAGTCTTTCAGAGCTGCTGGGAATATCCCCTTTCCCCGTAGTGCAGCTTCTCAGGATGTGTTAAGT
GGATGGATCACATTTCAGAAGCCGCTGCAAGGTGTATCAAAAACACATCTCCTGAGCCGTAAGGGACGGG
GCATCCAGTAACAACGCACACGGGGTATTTTTGGGCTTCCTTAAGATTTGAGCCGCTGCCTTAGGTTGTG
CTGCCCAATGTGCCTGGGGAGCTGCTAAACAGATTAGAGAGTCGAGGATTGTTGTCAGTTACTCAGAGAA
AGAACAATCATCCTTTCCAGGAGCACCTGAGCTGTTTGTTTTGCGTAGAAGATGCAAAATAAGGCCTGCA
ATGGGTATAAAATGTCCCTCAGCATAAATCGCATAGGAGTATGACTAAGGCTGTTGACTCTTCTGTCTTC
TTTCTCCTTCCTCCTTCGATTTCCTAGTTGGATAATGTACAGGGCTCTTTAGCCTCGCTCTGTCAGGGGC
TCCCTTCCTGGTTTGTTCTGTTTCCATTCTTCCTTCTCCAGCCTTCTTGACAAGAGCTGGGAACTAACGT
GCCTCAAGCCCCCACAAGGACCACAGCATTTTCTCATTTAGTTTCAGAATGACTCTGTGACGCAATCTTC
CTCTCTTGGAAGGTGAGAAAGCTGATCTTGGAAGGTGAGAAAGCTGAGACTTAGAGCAGCTGAAGCCAAT
GCCCAGGGACTTACTGCCAGTCAGCAGGTGGCAGGGCAGAGGTTTGAGCCCGGCTGTGCTTGAGGTCAGG
GCTCTTGCCAGGTAGACGCATCACTGACCACCTCCTAGAGGTTGATGGTTATGAATCTCAGGCACACCTT
GGCATCACCTGAAATACCCATGCCTTCAACTCCCCAGCAGAGTCTGCAGAAACTGGCCTGGGGTGTGGCC
TGGGCACTGGGACTTTCAGTTTCTCTCTGGGTGATTAGAAAGTGCAGCCAAGGCTCACGCCTGTAATTCC
AGCACTTTGGGAGGCCAAGGTGGATGAATCACTTGAGGTCATGAGTTCCGGAGCAGCCTGGCCAACATGG
TGAAACCCCGTCTCTACTAAAAATACTAAAATGTAGCCAGGCGTGGTGGCAGGCACCTGTAATCCCAGCT
ACTCAGGAGGCTGAAGCACGAGAATCACTTGAACCCGAGAAGCAGAGGTTGCAGTGACTAGAGATCGCAC
CAGTGTCCTCCAACCTGGGTGACAGAGCGAGACTCCATCTAAAAAAAATGAAAAAGAAAGTGCAGCCAAG
GCAGAGCACCACTGCCCTATTGCTTCCTCAAGCAACCCACAGCATCAGTACAGCCTACTAAGAAAGTATT
TAGGGACTTTTATGCTCCTAACAGTCACTGGAACTCACGTCACAATGACGTGTATTCCATTTGCAAGAAT
ATATACTTTAGGTCGGGGTGCGGTGGCTCACGCCTGTAATCCCAGCACTTTGGGAGGCCAAGGCAGGGGG
ATCACGAGGTCAGGAGTTCGAGACCAGCCTGACCAACATGGTGAAATCCCCGTCTCTACTAAAAATACAA
AAATTAGCCAGGCGTGATGGCGCATGCCTGTAATCTCAGCTACTCAGGAGGCTGAGGCAGAAGAATCTCT
TGAACCTGGGAGGTGGAGGTTGCGATGAGCTGAGATAGCACCACTGCACTCCAGCCTGGGCGACAGAGCA
AGACTCTGTCTAAAAAAAAAAAAAAAAAAAAAAAAAAAAAAAAGAATATAAACTTTAGTAGTCAGGGCAG
AAGTACTCTGTGTCTGCCACCTTTCTCAGCATCAGTATTCCATGTCACTACCTCATTCATACACACTCCT
GGATCTTATCATAGGCAGCTTCATTCTATAGCAGTGGCTCTTCACCAGGGCACTTGAAGAAGCCAACTAG
GATAAAGGAATGTGCTTCTCAACCCATGGTATCCAAGGCTGCTATGATCACAGGCTGAAAGCTTGAAGTC
AGTGGAAGATTTGTCCTTCCTCATTCCCCTCTAAGGTGTTGTTGGAGTCTTTATGTTCTCCTGATGTCCC
TTCTGCCTTTCCTTTCCTTTCCAGGGGCTCGGGACATGTGGAGAGCCTACTCTGACATGAGAGAAGCCAA
TTACATCGGCTCAGACAAATACTTCCATGCTCGGGGGAACTATGATGCTGCCAAAAGGGGACCTGGGGGT
GCCTGGGCTGCAGAAGTGATCAGGTAACTGGAGCTCCTGGGACGTTAGGGCTGGGTGAGCAGAGCTTGCC
TGCCTTGGACAGTCAGGAGGGAGACGAGCTCCTTGTGGAGAAGTTAGAGGCTGCGGCCCCTCCTCCTCTT
GCCCTCTCTCTGCCTCTGTGCTCAGTGTGAGGTCTGAGTGGATGGTAGGAGTGAGTGATTCCTCATCCTC
CCTCTCTGGGTGCTGTTCATCCAGCCTAGGGGTGCCCAGCCTGGCTGAATGGGGTGGTGCCCAGTGTTTT
CATCCCTCCTTCCTTGGCCTTTCTGGGCTCCTCTCTGAGCCCTCCCTTGGAACAGGGAGAATGGGAGGGT
GGGCTATTGCTCACTGGCCTGATTATTAATCTCCTTCTTGCCTGCCTTGATTACAGCGATGCCAGAGAGA
ATATCCAGAGATTCTTTGGCCATGGTGCGGAGGACTCGCTGGCTGATCAGGCTGCCAATGAATGGGGCAG
GAGTGGCAAAGACCCCAATCACTTCCGACCTGCTGGCCTGCCTGAGAAATACTGAGCTTCCTCTTCACTC
TGCTCTCAGGAGATCTGGCTGTGAGGCCCTCAGGGCAGGGATACAAAGCGGGGAGAGGGTACACAATGGG
TATCTAATAAATACTTAAGAGGTGGAATTTGTGGAAACTGGGTGTTATACTTTGTGGTATAGACTGCCTG
TTTAGTATGAAGGGGCGATCCATGCACATCTAAGTGAACGTGGAGGCTGGGTGGGTGGGAGACGACTCCT
GGGCACACAGGGCATCCTGGGCATCCCTGAGGCAAGGACATGATGAGTTCAGTGGCCACCCCCACAGGAT
CCCAGGGGCTTCAGCAGATCCCACCCCTTACCCCATGTGAGCAGCTGCCCAGTGAGTCTGTAGGAACCCG
AGCCACATTCCCAGTGAGTTCAACTGCACCCCGGCACGTTTTGCTAGCACCTCAATGGAGAGCTCCTTGC
TTGCAGCTTTGGCTTGTGGCACCCAGCAAAAGCTTCCTGCCACCCAGTGGCTACAGCCACACACTCTCCA
GCAAGATTTAATCTCAGCCTTGTGAGGAGCCCTTTCCCAAATTTATTTCTTTCTGTGTTTTTTATCCCTT
AGTAGCTAATCTCATGTTAGCCATTAATAACTCTCTATGTTAAACCCTTCCTTTTGTATCTGCGGCTACA
TTGATCAATTGTCTCACACCGCTCACCCACCCCCTCTCCCTGGTCATGCAGAGGCCTCACCAGTCATTTT
ATTGCTATTCCCAGGCCTCTGTGTGCCCAGATCTCTTCACTGCCCTGTCAGTTGTGTCCTGTCCCCTTCT
CGACCTCCTGGCCTTGTCCTCAATGATGTTTCTATGAGGCTTTGGAAAGCCTCATCCCAAGAGTCCTGGC
AACTGATACATTAGTCTCACCAACACTAGCCCCTACTCCTGATCTCATTTTAAAATTTTATTTTCTTATT
TTATTATTATTATTTTTAGGGACATCACCCTGCTTCTGACTGACCCAATTTTTAAAGTTTCTCTTTATTC
TCCTTAAAGAATGGCTCTCCCATTTGTTTCCCTCATCTTTCTTTTCACTGACTTAGAATTCAGGTCCAGA
CAAAAATTCCACTTCCTTGAAGAGCCTTCCCAGTAGCCATGAACCACACTCTGGGGCAGAGTTTGTGGCT
CCCAAGCACTTTGTTCACACCTGCTCGTTCATCTTTACCCCCTCCTCTCAGAATTGGTTTTGTATGTCAG
TTCCCCTGCTGGACTGGAAGCCCCTGTAACATAACTAGCATTTGAACAGTTCATAAAAACACTTTCATTT
CTATTGTCCTGTGTAACTGTTCACAAGATACATTAGTCTCACTCATTGTCGTTTGACAATGTTTATTGTT
CTAAAGAGAAAGCGAGATATGTAGGTAGACACAACACAATAAGAGCCTCAATACAGGCACACACTGGTGG
CAGTCCAAACTCTGCTGGAATATGGAAGGCCAGTGAATAGTAACTAGAATCGCTGCCATCTTTTACACTG
CAATTGCAAGACTGTGTCCTATACTTTACATGCTTTATCGGATTGACACTGCAGAGAAGCCTTTGATGTT
CTCGGTTGGATAAACAGGTTTAGAGGTATGTGTTCAGGCCTTCTCCAAGGTCACACATGTAACAGGTGCA
GAGCCAAGACTGCAGGCTGGTTTGTCTGACTCCAGAACCTGTGATACACTGAACAAGGCCAAGAGATTCA
GCAGTGGAGACCTCCTCTCTTCTTCAGGAAGATAGGGAACATGGTAAGAGCAGTGAATTCCATAAGCATG
GGACCCCGTTATACTTCATTTTTTGTAAAATTAGTTCCTTGATCAGAAGCAACGCCTTTGGGTCTCTCGC
TATTGAGTTAAGTGTGGATGTGCCACTCTCCCTAGTACCCCAGAGTGTAGTTGGAGGCTTGCCTAGAGAT
TAGAGGTTGAGGATCCTTCAGTAGTTCCTATACCAAGCCATAAGTTGTGCTGCATCTGCAGGTAGGGTCA
GGGAGCTCCGCTGAACAGATGTGGAGGACATCAGAGTGGGAGGAAAAGGAAGCAAGTCGTGTGGGGAGAG
AAGACCCAGCCTGCAATGATGACTGGTTAACTACTGTTTCTTACCTAATACACACTCAGATAACAGACTA
AGTCTAG
(hCRP) (Accession No. AAA52075)
SEQ ID NO: 4
MEKLLCFLVLTSLSHAFGQTDMSRKAFVFPKESDTSYVSLKAPLTKPLKAFTVCLHFYTELSSTRGYSIF
SYATKRQDNEILIFWSKDIGYSFTVGGSEILFEVPEVTVAPVHICTSWESASGIVEFWVDGKPRVRKSLK
KGYTVGAEASIILGQEQDSFGGNFEGSQSLVGDIGNVNMWDFVLSPDEINTIYLGGPFSPNVLNWRALKY
EVQGEVFTKPQLWP
(hCRP) (Accession No. NM_001329057)
SEQ ID NO: 5
AAGGCAAGAGATCTAGGACTTCTAGCCCCTGAACTTTCAGCCGAATACATCTTTTCCAAAGGAGTGAATT
CAGGCCCTTGTATCACTGGCAGCAGGACGTGACCATGGAGAAGCTGTTGTGTTTCTTGGTCTTGACCAGC
CTCTCTCATGCTTTTGGCCAGACAGACATGTCGAGGAAGGCTTTTGTGTTTCCCAAAGAGTCGGATACTT
CCTATGTATCCCTCAAAGCACCGTTAACGAAGCCTCTCAAAGCCTTCACTGTGTGCCTCCACTTCTACAC
GGAACTGTCCTCGACCCGTGGGTACAGTATTTTCTCGTATGCCACCAAGAGACAAGACAATGAGATTCTC
ATATTTTGGTCTAAGGATATAGGATACAGTTTTACAGTGGGTGGGTCTGAAATATTATTCGAGGTTCCTG
AAGTCACAGTAGCTCCAGTACACATTTGTACAAGCTGGGAGTCCGCCTCAGGGATCGTGGAGTTCTGGGT
AGATGGGAAGCCCAGGGTGAGGAAGAGTCTGAAGAAGGGATACACTGTGGGGGCAGAAGCAAGCATCATC
TTGGGGCAGGAGCAGGATTCCTTCGGTGGGAACTTTGAAGGAAGCCAGTCCCTGGTGGGAGACATTGGAA
ATGTGAACATGTGGGACTTTGTGCTGTCACCAGATGAGATTAACACCATCTATCTTGGCGGGCCCTTCAG
TCCTAATGTCCTGAACTGGCGGGCACTGAAGTATGAAGTGCAAGGCGAAGTGTTCACCAAACCCCAGCTG
TGGCCCTGAGGCCCAGCTGTGGGTCCTGAAGTGCTTTCTTAATTTTATGGCTCTTCTGGGAAACTCCTCC
CCTTTTCCACACGAACCTTGTGGGGCTGTGAATTCTTTCTTCATCCCCGCATTCCCAATATACCCAGGCC
ACAAGAGTGGACGTGAACCACAGGGTGTCCTGTCAGAGGAGCCCATCTCCCATCTCCCCAGCTCCCTATC
TGGAGGATAGTTGGATAGTTACGTGTTCCTAGCAGGACCAACTACAGTCTTCCCAAGGATTGAGTTATGG
ACTTTGGGAGTGAGACATCTTCTTGCTGCTGGATTTCCAAGCTGAGAGGACGTGAACCTGGGACCACCAG
TAGCCATCTTGTTTGCCACATGGAGAGAGACTGTGAGGACAGAAGCCAAACTGGAAGTGGAGGAGCCAAG
GGATTGACAAACAACAGAGCCTTGACCACGTGGAGTCTCTGAATCAGCCTTGTCTGGAACCAGATCTACA
CCTGGACTGCCCAGGTCTATAAGCCAATAAAGCCCCTGTTTACTTGA
(hCRP) (Accession No. M11725)
SEQ ID NO: 6
TTTGCTTCCCCTCTTCCCGAAGCTCTGACACCTGCCCCAACAAGCAATGTTGGAAAATTATTTACATAGT
GGCGCAAACTCCCTTACTGCTTTGGATATAAATCCAGGCAGGAGGAGGTAGCTCTAAGGCAAGAGATCTG
GGACTTCTAGCCCCTGAACTTTCAGCCGAATACATCTTTTCCAAAGGAGTGAATTCAGGCCCTTGTATCA
CTGGCAGCAGGACGTGACCATGGAGAAGCTGTTGTGTTTCTTGGTCTTGACCAGCCTCTCTCATGCTTTT
GGCCAGACAGGTAAGGGCCACCCCAGGCTATGGGAGAGTTTTGATCTGAGGTATGGGGGTGGGGTCTAAG
ACTGCATGAACAGTCTCAAAAAAAAAAAAAAAAGACTGTATGAACAGAACAGTGGAGCATCCTTCATGGT
GTGTGTGTGTGTGTGTGTGTGTGTGTGTGGTGTGTAACTGGAGAAGGGGTCAGTCTGTTTCTCAATCTTA
AATTCTATACGTAAGTGAGGGGATAGATCTGTGTGATCTGAGAAACCTCTCACATTTGCTTGTTTTTCTG
GCTCACAGACATGTCGAGGAAGGCTTTTGTGTTTCCCAAAGAGTCGGATACTTCCTATGTATCCCTCAAA
GCACCGTTAACGAAGCCTCTCAAAGCCTTCACTGTGTGCCTCCACTTCTACACGGAACTGTCCTCGACCC
GTGGGTACAGTATTTTCTCGTATGCCACCAAGAGACAAGACAATGAGATTCTCATATTTTGGTCTAAGGA
TATAGGATACAGTTTTACAGTGGGTGGGTCTGAAATATTATTCGAGGTTCCTGAAGTCACAGTAGCTCCA
GTACACATTTGTACAAGCTGGGAGTCCGCCTCAGGGATCGTGGAGTTCTGGGTAGATGGGAAGCCCAGGG
TGAGGAAGAGTCTGAAGAAGGGATACACTGTGGGGGCAGAAGCAAGCATCATCTTGGGGCAGGAGCAGGA
TTCCTTCGGTGGGAACTTTGAAGGAAGCCAGTCCCTGGTGGGAGACATTGGAAATGTGAACATGTGGGAC
TTTGTGCTGTCACCAGATGAGATTAACACCATCTATCTTGGCGGGCCCTTCAGTCCTAATGTCCTGAACT
GGCGGGCACTGAAGTATGAAGTGCAAGGCGAAGTGTTCACCAAACCCCAGCTGTGGCCCTGAGGCCAGCT
GTGGGTCCTGAAGGTACCTCCCGGTTTTTTACACCGCATGGGCCCCACGTCTCTGTCTCTGGTACCTCCC
GCTTTTTTACACTGCATGGTTCCCACGTCTCTGTCTCTGGGCCTTTGTTCCCCTATATGCATTGAGGCCT
GCTCCACCCTCCTCAGCGCCTGAGAATGGAGGTAAAGTGTCTGGTCTGGGAGCTCGTTAACTATGCTGGG
AAATGGTCCAAAAGAATCAGAATTTGAGGTGTTTTGTTTTCATTTTTATTTCAAGTTGGACAGATCTTGG
AGATAATTTCTTACCTCACATAGATGAGAAAACTAACACCCAGAAAGGAGAAATGATGTTATAAAAAACT
CATAAGGCAAGAGCTGAGAAGGAAGCGCTGATCTTCTATTTAATTCCCCACCCATGACCCCCAGAAAGCA
GGAGCATTGCCCACATTCACAGGGCTCTTCAGTCTCAGAATCAGGACACTGGCCAGGTGTCTGGTTTGGG
TCCAGAGTGCTCATCATCATGTCATAGAACTGCTGGGCCCAGGTCTCCTGAAATGGGAAGCCCAGCAATA
CCACGCAGTCCCTCCACTTTCTCAAAGCACACTGGAAAGGCCATTAGAATTGCCCCAGCAGAGCAGATCT
GCTTTTTTTCCAGAGCAAAATGAAGCACTAGGTATAAATATGTTGTTACTGCCAAGAACTTAAATGACTG
GTTTTTGTTTGCTTGCAGTGCTTTCTTAATTTTATGGCTCTTCTGGGAAACTCCTCCCCTTTTCCACACG
AACCTTGTGGGGCTGTGAATTCTTTCTTCATCCCCGCATTCCCAATATACCCAGGCCACAAGAGTGGACG
TGAACCACAGGGTGTCCTGTCAGAGGAGCCCATCTCCCATCTCCCCAGCTCCCTATCTGGAGGATAGTTG
GATAGTTACGTGTTCCTAGCAGGACCAACTACAGTCTTCCCAAGGATTGAGTTATGGACTTTGGGAGTGA
GACATCTTCTTGCTGCTGGATTTCCAAGCTGAGAGGACGTGAACCTGGGACCACCAGTAGCCATCTTGTT
TGCCACATGGAGAGAGACTGTGAGGACAGAAGCCAAACTGGAAGTGGAGGAGCCAAGGGATTGACAAACA
ACAGAGCCTTGACCACGTGGAGTCTCTGAATCAGCCTTGTCTGGAACCAGATCTACACCTGGACTGCCCA
GGTCTATAAGCCAATAAAGCCCCTGTTTACTTGAGTGAGTCCAAGCTGTTTTCTGATAGTTGCTTTAGAA
GTTGTGACTAACTTCTCTATGACCTTTGAA
(hHp) (Accession No. AAA8808)
SEQ ID NO: 7
MSALGAVIALLLWGQLFAVDSGNDVTDIADDGCPKPPEIAHGYVEHSVRYQCKNYYKLRTEGDGVYTLND
KKQWINKAVGDKLPECEADDGCPKPPEIAHGYVEHSVRYQCKNYYKLRTEGDGVYTLNNEKQWINKAVGD
KLPECEAVCGKPKNPANPVQRILGGHLDAKGSFPWQAKMVSHHNLTTGATLINEQWLLTTAKNLFLNHSE
NATAKDIAPTLTLYVGKKQLVEIEKVVLHPNYSQVDIGLIKLKQKVSVNERVMPICLPSKDYAEVGRVGY
VSGWGRNANFKFTDHLKYVMLPVADQDQCIRHYEGSTVPEKKTPKSPVGVQPILNEHTFCAGMSKYQEDT
CYGDAGSAFAVHDLEEDTWYATGILSFDKSCAVAEYGVYVKVTSIQDWVQKTIAEN
(hHp) (Accession No. M69197)
SEQ ID NO: 8
TCTAGAAGCTGCCATCATACTGAAGGTAATCTTCTGAAACTTGCGGTTTTCTTTCAAAAATTATGTTTAT
AAGATGATCCATGTTCTTGCAGAGTTTATTCATTTTATGCTGTATAATATTCCATTATATCCACATACAA
TGCAGTATTGACCCTTCCTCCTGTTGATGGGCATTTGTCTTGTTTCTAGTTACTTTGCTATTATATCAGT
GTCACCATGATTATCCAAAAGTAATTCTTTTGTACACTCTAATTTAAGAACAACTAACCCTTTTTAATGA
ATAAATCAACCTTGTATTGAGTTGCTACTAAGTTTCAGTTGACTAGTACCTGGGATACACACAGGTGCAG
ACATTTGACTGAGACATATTGATTTTTCTCATCTGCCTATTTAGGCTAATCACCAGACTATAAAACCATG
AGAACCACTGCCATTGAGTATAGTCTGTGTCAGTCTACACTATAGCTTTAACTAGTTGTGTGATTTCTTG
CAAAGAGCAATCAGAGAAGACACAATAAACACATTTACTGATTTCAGGCTGGAGAGCTTTTAAGCAATAG
GGAGATGGCCACACACAAGGTGGAGAAAATTACTGTGAAAAGGAAGTACTTTCTTTAGAGCCCCACCTAA
GCTAGGCTGCAGAAATGTCTACAATGGGTTTGAAAAAACTCAAAATGAGCCTTTCTGCAGTGTGAAAATC
CTCCAAGATAAAGAGACAGATTGATGGTTCCTGCCGCCGCCCTGTCCTGCCCAGTTGCTGATTTCAGGAA
ATACTTTGGCAGGTTTGTGGGTCATAGAGTTGCCAGGTTTCTTGGGATTTGTAATAGAACATCACAAGAA
AATCAAGTGTGAAGCAAGAGCTCAACTCTTAACAGGGGTATTGTTTGTGGTTTTGTTACTGGAAAAGATA
GTGACCTTACCAGGGCCAAAGTTTGTAGACACAGGAATTACGAAATGGAGAAGGGGGAGAAGTGAGCTAG
TGGCAGCATAAAAAGACCAGCAGATGCCCCACAGCACTGCTCTTCCAGAGGCAAGACCAACCAAGATGAG
GTGGGTCCACAGCTTTCCCTCCTGCCTTTCCTCTGGTTCTTTATTTCAGTCTTTTTTGCATACATCGGTA
GAGATGCAGAAATAGAACAAAGAAACGGGCAAATGGGCTAAATTATAGTGAACCAAAGGGCTTAGTGTGT
TAAATCTTCTCCTTTTCTGCATCCATAGAAGACAGTGCTGCTGTCTTTCCCAGGAGATAAGATTTACTCT
CAGGAGTGTCTTTTTCCTTCAGGTTACATTTTTGACTTTATAGGGTATGTCATCAGCTCCCGTGGTAGGC
TTCCTGGCATCCTGAGTATATTTATTAGCAGATATTTTCCTCTTTAAAAATGTACAATAAGGAAGACTAA
TAGTAACACATTTGAATGACACAATTAATTGACTAGTACCTGGGATACACACTAATACCTGGGATACATC
TAATTAAGGCACTTAGATCTTATAAAAATAAACACTTTTTGAAATGTTGAAATAATAAGACTAGAAACTT
TTTTTTTTTTTGAGATGGAGTCTCGCTTTGTCACCAGGCTGCAGTGCAGTGGCATGATCTCGGCTCACTG
CAACCTCCACCTCCCAGGTTCAAGTGATTCTCCTGCCTCAGCCTCCCAGGTAGCTGGGACTACAGGCGTG
CGCCACTATGCCCACCTAATTTTTGTATTTTTAGTAGAGACGGGCTTTCACCATGTTGGCCAGGATGACC
TCGATCTCTTGACCTCGTGATCTGCCTGCCTTGGCCTCCCAAAGTGCTGGGATTACAGGCATGAGCCACC
GTGTCTGGCCTAGAAACTATTTTAATAGAAGCAAGTAGTGCCCGAATGGTTGGCATTTGTTAGTGAGATG
GTGAACTGGCAGACGGCACCTGTGGGTCAATGCCCATGGCCACCGTCCTGCTTTTGGACACCAGTTCTCT
TCCAGGTAACCTTCTGGCATTTTGGGGTTTCAGATACCATTTCCTAAAGGTGAATTATTATAAAATACTA
GAAATACCCACGTGTTTAAAATTATATATTTAAGGAACCTTTTATTACGGAAAATATCAAGAAGTAGAGG
AATAGCACAGTAAGCCCAAAGTTCCCATCTCTGAGTTATCTACATTTTATTACCACTATCTTTGCGGTGT
CTGAGGGAGGTTTCTCTTTCCTGGAGGGCTCCTGTATTATTGCCAATGTACTTTCCTGAATGCAGCCAGA
AACTGAGCCCACCCCTCCACCTATGTGCCTTTCTATCCCTCTCTGAAGCTTCTGCAGAATTCCCAGCAGG
ACAGGGCTTGCTGGAAGCTTGGTATGCTCAGAAGCTGCTAAAGTGTGTATGGGCAGGTGTGGGGGCAATT
TCTTGGTCCTAGCACTTCCATATATCGACTTTCTTTTCTGGCTGCTAAGTGGGAGGAGTGTGTGTGTATG
CATGTGTGTGTGTGTGTGTGTACATGCATGTGTGTGTGGATGCATGCATGTGCTGTGAAGCAGGGAGACT
AGCTTTCCACTCCTCCTTGTCTTCTCTCTGCAGTGCCCTGGGAGCTGTCATTGCCCTCCTGCTCTGGGGA
CAGCTTTTTGCAGTGGACTCAGGCAATGATGTCACGGATATCGCAGGTCAGTCTTTGGTTGGGTAGGAGT
GTGCATCCCACTCTGACCCTCTCGGGTCTGCACTCTCTCTGAGAACACCCAATTCCCCCTTCTTATCTCG
ACCTCTGGGCTTTCAGGACCATAAAGAACATTGGGGTTCCTGCCAGAAATGAGGGGAGCTTGCCTTTCCA
TTGGCTTCTATTCGGGGTGGAAGGAGATTGATGTGCAGAGCAGCTCCCGCTCATCTGACTTTTCACGGTT
CACTGGGAACAATTTCCAAATAGCAAACTCTCTGGCTTCTCTCTCTTTGCAGATGACGGCTGCCCGAAGC
CCCCCGAGATTGCACATGGCTATGTGGAGCACTCGGTTCGCTACCAGTGTAAGAACTACTACAAACTGCG
CACAGAAGGAGATGGTAAGATGTGGACAACTGTCTCCATGCCCTACATACAACCCCCTTCTCTGACATTT
CCATGATGGGTGGTGCTGAGGTGATTCGCCAGAAAGTTCGTTGCTCTCCTTGGAGCCAGGAGATTTAGAT
TCTAATAAGCGTTTTGTCGCCAGTAGCCATGGCCCTTTGGGCAGACTAACTTTTGTCAGCCTCAAGTTTT
CTGTTTTGTTAAGGGGAGGCGATGCCATGCAGCCTACCTCATGTAAATCTCAGAGTCAGATTTACATCTC
CAGCAGATGTGGGAAAAGAAGGAATGCTGATGATGATGTCACCCTCACCTAGTGAGTCTTGCTGTCCTGG
CACTGCTCTAAGGGCTTTATACTTATTTGCTCACTTAGTCCTCACAGTATCCCTCTGAACAGAGTTTATT
GTTTTCACTTTGCTGATAAGGAAACTGAGGCACAGACAGGTTGAGTATCTTGCCCAAATTCAGGCAGCCT
GTAAGAGGCAGAGTCAGGATTTGAACCCTGAGCCCTCCCTGTACTGCTTGGCTGTGACCGCCATGACCAC
AGTGTGTTCTGCTGGGCTTAACTGGTGTCCAGGCACTTGGCTTCCAGCACAGCACTCTTTCCCTTCCTCC
TTCTCATATTCTCTCTCCTTTCTCCCTTCCTGTCTGCCTCCTTTCTTCTTCTTCTTTTTAATTCTTCTCC
TTAAATGCCTTCTCACTCTGCTCTGGGTGCAGACTTGACTTTTCCTTTGGCTCATTTCTTGCCTTTTGTT
TCAGGAGTATACACCTTAAATGATAAGAAGCAGTGGATAAATAAGGCTGTTGGAGATAAACTTCCTGAAT
GTGAAGCAGGTGGGTGCTGAGCACTGAGCACTTAAGAGAGCAGGCAGGCGTCCAGCGGGGAACGTCCTAG
AGGCACAGCCTTCCAGTGCGGCTTCCTCTGAGCACACAAGAGCCAGGAGGAGGGATGTGGGAGAACCGCA
GCTGGCCAGGGAGAGACTTAAGCAGTTAGGTGATGACTCCCTAAGGGTCACCAAGGGTCTTGTTCATTGG
GGCCTGAAGGGCACTGGCTGAATCCACTGTCGGCACTGCCCACAGATCAGGAGAGCCTGTGCATACAGAG
AGCCTGCTAGAAAGCCCTGGGTCTAAGGAGAAGCAAGCTCCAGGGAGAACAAGTCAAGGAATGACATAAA
ATCTTAATCCATGGAAGCCTAGCAGGAGGCTGGACATGGGCTGGAACTCCTGCTTCTCGTTATTAGGAGG
AGCTGTTGCTCTCTCCTTTCATTCTCAGAACCAGAGGCAAAGACCCAGCCTCTTCTGCTCTTACTGGTGT
GGAAATGCCAACCTGCCTCGTATTAACTGCACCATCTACAAAATCTGAGCTCCAGCCAGTGCTGCTCTAG
ATTCATCTTTCTTTAGAGAGAATGAATTATTGTAGCCCCTAGCCCTTTCAATGAATTTCAGGGAATTGTG
GAAATTCCTTTATTGGGATAATTGTTTAAATATAATACAGTTCGCGAGCTTCTATTCGGGGTGGAAGGAG
ATTGATGTGCAGAGCAGCTCCCGCTCATCTGACTTTTCACGGTTCACTGGGAACAATTTCCAAATAGCAA
ACTCTCTGGCTTCTCTCTCTTTGCAGATGACGGCTGCCCGAAGCCCCCCGAGATTGCACATGGCTATGTG
GAGCACTCGGTTCGCTACCAGTGTAAGAACTACTACAAACTGCGCACAGAAGGAGATGGTAAGATGTGGA
CAACTGTCTCCATGCCCTACATACAACCCCCTTCTCTGACATTTCCATGATGGGTGGTGCTGAGGTGATT
CGCCAGAAAGTTCGTTGCTCTCCTTGGAGCCAGGAGATTTAGATTCTAATAAGCGTTTTGTCGCCAGTAG
CCATGGCCCTTTGGGCAGACTAACTTTTGTCAGCCTCAAGTTTTCTGTTTTGTTAAGGGGAGGCGATGCC
ATGCAGCCTACCTCATGTAAATCTCAGAGTCAGATTTACATCTCCAGCAGATGTGGGAAAAGAAGGAATG
CTGATGATGATGTCACCCTCACCTAGTGAGTCTTGCTGTCCTGGCACTGCTCTAAGGGCTTTATACTTAT
TTGCTCACTTAGTCCTCACAGTATCCCTCTGAACAGAGTTTATTGTTTTCACTTTGCTGATAAGGAAACT
GAGGCACAGACAGGTTGAGTATCTTGCCCAAATTCAGGCAGCCTGTAAGAGGCAGAGTCAGGATTTGAAC
CCTGAGCCCTCCCTGTACTGCTTGGCTGTGACCGCCATGACCACAGTGTGTTCTGCTGGGCTTAACTGGC
ATCCAGGCACTTGGCTTCCAGCACAGCACTCTTTCCCTTCCTCCTTCTCATATACTCTCTCCTTTTCCCC
TTCCTTTTTGTCCCCTTTTCCTCTTCCTTTTAGTTCTTCTCTTTAAATGCCTTCTCACTCTGCACGGGGT
CTAGACTTGACTTCTCCTTTGGCTCACTTCTTGCCTTTTGTTTCAGGAGTGTACACCTTAAACAATGAGA
AGCAGTGGATAAATAAGGCTGTTGGAGATAAACTTCCTGAATGTGAAGCAGGTGGGTGCTGAGCACTTAA
GAGAGCAGGCAGGCGTCCAGCGGGGAACGTCCTAGAGGCACAGCCTTCCAGTGCGGCTTCCTCTGAGCAC
ACAAGAGCCAGGAGGAGGGATGTGGGAGAACCGCAGCTGGCCAGGGAGAGACTTAAGCAGTTAGGTGATG
ACTCCCTAAGGGTCACCAAGGGTCTTGTTCATTAGGGCCTGAAGGGCACTGGCTGAATCCATTGTCTACA
TCGCCCACAGATTAGGAGAGCCTGTGCATACAGAGAGCCTGCTAGAGAGCCCTGGGTCTAAGGAGAAGCA
AGCTCCAGGGAGAACAAGTCAAGGAATGACATAAAATCTTAATCCATGGAAGCCTAGCAGGAGGCTGGAC
ATGGGCTGGAACTCCTGCTTCTCGTTATTAGGAGGAGCTGTTGCTCTCTCCTTTCATTCTCAGAACAAGA
GGCAAAGGCCCAGCCTCTTCTGCTCTTACTGGTGTGGAAATGCCAACCTGCCTCGTATTAACTGCACCAT
CTACAAAATCTGAGCTCCAGCCAGTGCTGCTCTAGATTCATCTTTCTTTAGAGAGAATGAATTATTGTAG
CCCCTAGCCCTTTCAATGAATTTCAGGGAATTGTGGAAATTCCTTTATTGGGATAATTGTTTAAATATAA
TACAGTTCACCAGCCAGGGCTCAAAAATCTCAGTATTTCCCACTTCCTTTGTTAGAAAAGTGGGAAATAG
AGCTTTTTGTAATGTAAACAATTTAAAAAACAGAATTATTTTAAAACTGCAACTATTGGAAATGAGATCA
GCAGGTGGTAAGGGCAAAGCATTTAAATCTTTCTACTTTACGCAGCAGTGACAGCCGCCCATGCTTTCAC
CCCTTTCTCAGATGGAAAGGCTCTTGCACATTTCCACTCACGAGTGTCTTGCTCTCCTTGACAGTATGTG
GGAAGCCCAAGAATCCGGCAAACCCAGTGCAGCGGATCCTGGGTGGACACCTGGATGCCAAAGGCAGCTT
TCCCTGGCAGGCTAAGATGGTTTCCCACCATAATCTCACCACAGGTGCCACGCTGATCAATGAACAATGG
CTGCTGACCACGGCTAAAAATCTCTTCCTGAACCATTCAGAAAATGCAACAGCGAAAGACATTGCCCCTA
CTTTAACACTCTATGTGGGGAAAAAGCAGCTTGTAGAGATTGAGAAGGTTGTTCTACACCCTAACTACTC
CCAGGTAGATATTGGGCTCATCAAACTCAAACAGAAGGTGTCTGTTAATGAGAGAGTGATGCCCATCTGC
CTACCTTCAAAGGATTATGCAGAAGTAGGGCGTGTGGGTTATGTTTCTGGCTGGGGGCGAAATGCCAATT
TTAAATTTACTGACCATCTGAAGTATGTCATGCTGCCTGTGGCTGACCAAGACCAATGCATAAGGCATTA
TGAAGGCAGCACAGTCCCCGAAAAGAAGACACCGAAGAGCCCTGTAGGGGTGCAGCCCATACTGAATGAA
CACACCTTCTGTGCTGGCATGTCTAAGTACCAAGAAGACACCTGCTATGGCGATGCGGGCAGTGCCTTTG
CCGTTCACGACCTGGAGGAGGACACCTGGTATGCGACTGGGATCTTAAGCTTTGATAAGAGCTGTGCTGT
GGCTGAGTATGGTGTGTATGTGAAGGTGACTTCCATCCAGGACTGGGTTCAGAAGACCATAGCTGAGAAC
TAATGCAAGGCTGGCCGGAAGCCCTTGCCTGAAAGCAAGATTTCAGCCTGGAAGAGGGCAAAGTGGACGG
GAGTGGACAGGAGTGGATGCGATAAGATGTGGTTTGAAGCTGATGGGTGCCAGCCCTGCATTGCTGAGTC
AATCAATAAAGAGCTTTCTTTTGACCCATTTCTGTGTTGTGTTCAGTCTTGAGTCTTTTTTATTTGCTCC
TTTATGGTCCAGGGTAGTCAGAAGGTATAGAGTCTACTGGGAGTATGGCAGAAAACACCCTAAACCCACT
GGAAATCCCGAAGGTGATACAAACTCTTCCACCTTAGGGAATCATGCTCACTGATTGAGTGCCTATTGAA
TGCTAGGTCCCAGAAAGTTAACTGTTGTCCTTGTTTTACAGACAAGGAAACAGAGACTCAGAGATGGTAA
GTGAGTTGCTTAAGGTTACATAGCTATGAAACAGGGAAGCAGAACTTTGAACCCAGGTCTGTTTGATACA
AACTCAGAGGTCCTTTCACTGCATGCTGTTGCCTCCTCAAAGTGAATTAGGAGAAAAGGCATGGGCCTGG
TTGAGGAAGAGGCTAGCTCAAAATGGGATGGGGAAAAAGTGTTTTAACACAGACAGTAATTCAGAGTTTG
GGATCTAACCTACCAGCACTCCAGAAAACAAAAGACAATGATGTAAAGGCAGGATCTCTGGACTTACTGA
GTCCAAATCACAGAATGGCCCATAAATTTGCTAGGTAATTTAGGTTTCCTGGCCATCAGTTCCTGGTCTC
TTCCATGAAGGACTTGAGCCAGGCGATGGCTAATGTCCCTATCAGCAATAACCTTCTGGTATTCCATAAA
GAGTGGGGAGGTAGAGTCCCTGCATCAGTAGAAGCTGCTTTTCCTACTGGGAACAAACCCACCCTGCACC
AGGAATCAAATGCAGGCTGTCTGAGCCTCCTCTCCTGATGGCAGGTCATGCTATTAAGGGTCTCCCAGAA
GAAAGGTTCTGGAACTCTGAGAAACAGAAGCTCTTCTAACAGAACTTTTTCTTGCTATAAGCTTTCAATA
CCACCAATCACCAGTGCACCTTCTTCTCATGCCAAAATACGGCCCCTCCTTTGTAGGGTGTAAAACGACA
CTGATGTCACTCTCTCTCACCAAGGTCTGGCCATCTCTGTGAAGCTCTCCTCACAGGCACCCTGCAGATT
CATCAATAAACTTCAACAGGTTAGCGCCAGTAGGCCAATAAGATGCACACTTATATTCTAGAACCTGTGA
AAGAAGTAATAGCCAGAAACTGGAAAATAACAGTCAAACTCTTAAAAAAATGCCTCTCAGAACACAGTCA
CTTACACCTTTTCCTCTGGTAGTTCTCTTCAGCACCAAGTTTCCTGGAAGAACAAGCTACTCCCCAGTTT
TCTTAAAGAATTGTCTGATTGTATTAAGTAAGTGGCTTCTGGTAAAATAATGGTAAGAGAAAAGCTCTGG
CATTCTCTTGATCACTCATCTTCTCTGCCTTCCTCCCTAGCAACATCTCCTGGCCTCAGCCTGTGGGCTT
CAGCAACACCAAACTCCATGTTGCTCTCTGACAAGCCCGGCCGTCTCCAGTTCTTGGGCTTTATTTCCTC
GGGGGCCCTCTGCTGAAATTCCCTTCCCCTTGCTGTATTAGCACCTCCTACCCGCCTTCAACATGCAGGC
ACTGCCTCCTCTAAGGAGCCATCTGTACTTACGTAGCTGTGAGCATAGGATGGGGCATACAGCAGGCACT
TAACAAATACTTGTGGAATGGAAAGAAACTAAGATAGGATCAAAAATACAAATCAAGATGTGAGAGAAAG
ACGATCAAAAGCCCTCACGAGGGGGTCGGGCGTGATGGCTCACACCTGTAATCTCAACACTTTGGGAGGC
TAAGGCGGGTGGATCACTTGAGGGTGGGAGTTTGAGATCAGCCAGAGCAACATGGAGAAACCTCGTCTCT
ACTAAAAATACAAAAAATTAGCCAGGCATGGTGATATATGCCTGTAATCCCAGCTACTCGGGAGGCTGAG
GCAGGAGAATTGCTTGAACCTCGGAGGTAGAAGTTGTGGTGAGCTGAGATCATGCCATTGTACTCCAGCC
TGGGCAACAGGAGTGAAACTCTGTCTCAAAAAAAACAAAAAACAAAAAAGAACACCACGGGAAAATCAAG
TGTGAAGCAAGAGCTCAACTCTTAACAGGGGCAATGTTTGGAGTTTTGTTACTGGAAAAGATAGAGACCT
TACCAGGTCCAAAGTTTGTAGACACAGGAATTACGAAATGGAGAAGGGGGAGAAGTGAGCTAGTGGCAGC
ATAAAAAGACCAGCAGATGCCCCACAGCACTGCTCTTCCAGAGGCAAGACCAACCAAGATGAGGTGGGTC
CACAGCTTTCCCTCCTGCCTTTCCTCTGGTTCTTTATTTCAGTCTTTTTTGCATACATTGGTACAGATGC
AGAAATAGAACAAAGAAACAGGGCAAATGGGCTAAATTATAGTGAACCAAAGGGCTTAGTGTGTTAAATC
TTCTCCTTTTCTGCATCCATAGAAGACAGTGCTGCTGTCTTTCCCAGGAGATAAGATTTACTCTCAGGAG
TGTCTTTTTCCTTGAGGTTACGTTTTTGTCTTTGTAGGGTATGTCATCAGCTCCCGTGGTAGGCTTCCTG
GCATTCGGAATATATTTACTAGCAGATATTTTCCTCTTTAAAAATGTATAATAAGAAAGACTAATAGTAA
CACATTTGAATGACACAATTAATTGACTAGTACTTGGGATACACACTAGTACCTGGGATACATCTAATTA
AGACACTTAGATCTTAAAAAATAAAAAGACTTTTTGAAATGTTGAAATAATAAGACTAGAAACTTTTTTT
TTTGTGAGACAGAGTCTCACTCTGTCACCAGGATGGAGTGAGTGGCGCGATCTCAGCTCATCGCAACCTC
CCCCTCCCCGGTTCAAGCAATTCTCCTGCCTCAGCCTCCCAAGTAGCTAGGACTACAGGCGTGTGCCACC
ACGCCCAGCTAATTTTTGTATTTTTAGTAGCGATGTGGTTCACCATGTTGGCCCGGATGATCTCGATCTC
TTGACCTCGTGATCCACCTGTCTCGGCCTCCCAAAGTGTTGGGATTACAGGTGTGAGCCACCGCACCCTG
CCTAGAAACTATTTTAATAGAAGCAAATAGTGCCTGAATGGTGGGCGTCTGTTAGCGAGATGGTGAACTG
GCAGATGGCGCCTGTGTGTCAATGCCCATGGCCACCGTCCTGCTTTCAGACACCAGTTCTCTTCCAGGTA
ACCTTCTGGCATTTTGGGGTTTGAGATACCATTTCCTAAAGGTGAATTATCACAAAATACTAGAAATAAC
CACATAAGTGTTTAAAATTATTGTTAAATACAGTAAGAAATTCTTCTTCAAAAGTTTAGCCTGCTTAAGT
TTCCTTGTCCTTTGTTTCCTGCTTTCAAGGCCAGACTTCCTTACTCTCTGTGTTTCCCCTACCCTGGTAA
ACAACCTTCCTGCCAGTCCTTACCCATACAGCCCACATTCCACATCTGCTACCCACTCTGTGATTTACCT
CTCCCGTCGCAATAGCCTCTCCCACCAAAACTGATCTTCCCGCCTTCCCACCAGTGCAACCACATTCCTG
CACTTTGCAAGTTAGCCAACCGGGTTCGGATTGTGCAGTCCAACTCCAGCCAATGGAGTCAGGACACAGT
AGCAGGGACAAGCTGCGTTAGACATAAAAACCTCTGCTTTCCTTTGTTTAGGGTGCTCTCGTGGCAACCA
GACTTACCAGGAGCTCTATTCTGCAAAAGTAAATTTGCCTTGCTGAGAGACCCTTTGTCCTTTGGCTCAG
TGTTGGTTCTTCTTTGCAGCACCGAGCATTTGTTTCCAACAAATTTGGTGGCCCATACAGGGAAAACATT
GTCCTCCGGGAAAGGGTTCTTTGATCATCCTCTTGAGAGGAGAACACATCCCACTGTCCTTGTTGCGGTG
GCCTCATGAGTAGGAATCGAGACCCACCTGTCTGATGAATAACCCCAGACTCTCAACAACGTGGGGAGAA
AAAGACTTGCAACACTATGGTGGCCAGGTAACTCTGTGCGCAGACCAAGGTAAGAAATGTCGCAGGAGTG
ACAAAGTACTTCCTTGGTGGTCACTATATTCTGGTGGCTGAAAGTTCATGAATGGTAACAAGTGCTACTG
CTGTGTGGAGTGAATGAGTCCAATCTGTGGGTCTATGGTTACCTCATACGGCTTAGCCTTCTCTGAAGGA
TCCTGATGTTGGGGTTTATATAGTCCTCCCAATGCTAAGCGGGACTTAAAATATTCCTAGGAGGAAAGTG
GCCAGAGTGGATGAAGCAAAAGGAGAAGAGTGTGAAGAACCTCCAGGAGGTGGGGCTAAAAGATAGGCAA
GAAATCTCTAATACGAGGGATTGAGCCACAGAAAGCTCCAGACAGATAAAAAAGAAATTCCTAATATGAG
GAACTGAGCCACAGCAAGCCTCCAGCAGGCAACAAATCCCTAATATGAGGGATTGAGCCTAGCTAAGACC
CAATATGGGAAATACCCCAAGAAAGACAGAGAATAAGAAGGATGAAAATAGTAACAAGGATATACCCCTT
GATAGTCCCCTAGGTTTCATGTTAAAATACTGGAAAGATAATGAGAGGACTAAGCATAAGAAGAAGGAGC
AAATGATAAAATATTGCTGTTTCATTTGGACCCAAGGTCCCATCCTCAAACCCTCAATCTTCTGGCCAAA
GTATGGGTCGAATGAGGGTGTAATGAGTCAACTCCTAATCCAATATGTTAATGATAAAAGTCTGGTTTCT
CAAGAAGAACTAGACTATGCTCTTTGTTGGAAGCAGGGACCTGTCCTCCTCTTTCCCTTAAAGACAACTA
GGGAAGAATCCGATCCAGCATCTCAAATTGAGAAGTCAGACAAGCCGACTCCCACACCTAAAGTCAGCAC
ATGGGATCCCCTAGACTGTCTTCCTTTGCTTACTGCCCCAATCCTAACCCCCCTACTCCTCAGGCAGCTG
CTGCTGCCCCAGATCCCATCCCAGATTCTTCCCCAACTCATGATGTTCCTCCCGCTTACAACCCTGACTC
TCAGGGGCAGTCGTCCCAAGAGCCTGTTCATTGCCAACCTAAATATCCTTCCTTAAAAGGGCTCCAACAT
GAAATAGAGCAGTGTAAAAAGGACATTCAGAACTCCCCTTTTCTTTCCACACCTAAGGAGTCAGCCATAA
CTTTCTTCCCTTTAAAAGGGGTATCACAAGGAGGGGAAGTCATTGGTTTTGTAAATGCTCTCTTGACTAT
TTCAGAAGTCTGAGGTCTGAAGAAAGAACTTAAGCCACTGCTAGATGACCCCTATGGAGTGGCAGATCAA
GTTGATCAATTCTTAGGGACCTCAGTTATACACTTGGGTCGAGCAAATGTCTATCCTAGGCTCTTCTTTT
CAGGGGAGGAAAGAAAGCATGATCCGTAGGGCTGCTATGGCAATTTGGGAACATGAACACCCTCCTGGTC
AAAACGTTCCTACTGTGGACCAAACATTTGCTGCCCAAGCAAGACTCCTGGTGGGACAACAGCAATGCAG
CCCACTGAGAAAACATGCAGGACCTAAGGGAAATAATAATAAAAGGAATCAGTGAATCCATACCCAGAAC
TCAAAAGCTCTCTAAAGCATTTGATATACAACAGGAGAAAGATGAGGGACCTATAAGATTCCTAGACAGA
CTAAAGGAGCAAATGAAGCAATATACAGGTCTGAATTTAGAAGATCCCCTTGGGCAAAGGATGTTAAAGA
TCCATTTTGTCACTAAAGGCTGGCCAGATGTTTCAAAAAAGTTACAAAAATTGGAGGACTGAGAAAACCG
ACCTCTAAGAGAACTTCTCAGAGAAGCTCAAAAGGTGTATGTGAGGAGGGACAGGGAAAAACAAAAACAG
AAGTCAAAACCAATGTTATCTACTTTCCAGCAGGTGGCTCCAAACCCATATGCTACTAAATGAGGCTTCC
AGGGAGCCAGAAACTATAAAAGGTCCCAAGCCTCCCAAACCCAGTTTAGAGAAACCAAACCTTCAGCTAG
AGGACCCAAGTCTACATTTCCCAGGCCCCCTAAAGAGCATAGAAAAGCAAGACCAAAAAATCCCAAAACT
GAGAGAGGGGAAGGACAAGATAAGTGTTACAAATGTGGAAGGACAGCCCCCTTCAAAAGAGAATGTCCCA
AATTAGAAAAGGAGAGAGAAGCCCTTCCACTCACGACCTTTGAAGAGGAACAGGGAAGTCAGGGGCTCTG
TCTATATTATCTTGAGTCCCACCAGGAGCCCTTGATAAATTTGGAGGTGGGACCTACACATGAGCTTATC
ACATTTTTGGTTGATTCAGGAGCGGCCTGTTCCTCTGTTTGTTTCCCCTCATCTAATGTTGCCTGCTCTT
CAGAAGAACTTATAGTCTCTGAGATAAAAGGGGAAGGATTTACGGTGAGAATCTTAGAAAATACAGAAGT
CAAGTACCAAGACTAAACAACCCAGGTTCAATTTTTGTTAATCCCTGAAGCAGCAACTAGTTTGTTAGGA
AGAGACTTAATGTTAAAGTTAGGCATAGGCCTACAAGTCAGCCCAAAGGGATTCCTTACTTCATTAAACT
TACTCACCACGGCGGATGAGAAATACATTCATCCTGATGTTTGGTCAAGGGAAGAAAACTGAGGAAAGCT
TCGAATTCTCCCAATCCACATCAAGCTAAACACCCCGCACTGGGAAGTAGTGAGGAGGAAGCAATTCCCC
ATTCCCTTAGAGGGCATGCTAGGGCTAAAACCTATAATTGAAAGTCTCATTAATGATGGGCTTCTTGAAC
CCTGTATGTCTCCTTATAACACCCCAATACTGCCTGTCAAAAAATCAGATGGGTCATACCGGCTGGTGAA
AGACCTCAGAGCCATTAACCAAACAGTCCAGACCACTAACCCTGTTGTCCCCAACCCTTACACCATTCTC
AGCAAAATTCCATATAATCATCAATGGTTTACTGTAATAGATTTAAAGGATGCTTTTTGGGCATGTCCCT
GGCTGAAGAGAGCCGAGACACATTTGCCTTTGAGTGGGAAGATCCCCAGTTAGGGTGAAAACAATGGTAT
CAATGGACAGTCTTGCCTCAGGGGTTCATGGATTCACCCAACCTTTTTGGTCAAATTTTAGAACAAGTGC
TAGACAAAGTTTCTGTTCCAAAACAATTATGCCTGCTTCAATATGTCGATGATATTCTCATATCTGGTGA
GGATATAGAGAAAGAAGCTGGCTTCTCTACACATATTTTTGACCATCTACAGTTCGAGGGGTTACGGGTC
TCAAAGGGAAAGCTTCAGTGTATGGAACCTAAAGTTAAATATTTAGGCCGCTTAATAAGTGCGGGCAAGC
GAAGGATAGGGCCTGAATGGGTCGAAGGAATCGTGTCCTTACCCTTGCCTCAGACTAAACAGGAACTCAG
GAAATTTTTAGGGTTAGTTGGATACTGCCGCTTAAGGATTAACTCACATGCCCTAAACAGTAAACTTTTA
TACCAAAAACTTGCCCAGGGAAAACCTGAGCATCTCCTGTGGACTTCTAAAGAGGTCGATCAGGTCAAAG
AGCTAAAAGGAATAGCTTATAACTGCTCTTGCCCTAGCCTTACCTTCCCTAGAAAATACACTTCACCTTT
TCGTCAGCGTGAAAAATGGGGTGGCTTTAGGGGTGCTTATCCAAGAGCACAGAGGCTGCTGGCAGCCCAT
GGCCTTCCTGTCAAAAATTTTGGACCTGGTCACCTGTGGATGGCCTCAGTGCATCCAATCCATTGCAGCT
ACAGCAGTATTAGTTGAAGAGAGTAGAAAATTAACCTTTGGGTGGAGATTAACAGTAAGCACACCCCACC
AAGTTAGAGAGCTATTTTAAATAAAAAAGCAGGAAGGCGACTAACTGACTCCAGAATCTTAAAATATGGG
GCTATTCTACTAAAAAAAGATGAGAGAACACCTATGTCTAGATTTAATTGACTACCAAACAAAACTCAGG
CCAGATCTAGGAAAGATCCCTTTCAAAACAGGACGGCACTTATTTATAGATGGTTCCTCCCAGCTGATTG
AGGGAAAAAGACACAACGGGTATCCAGTAATCGATGGAGAAATTCTCTATGTATAAGAACAGAGTCAAGA
AAATTGCCTAATAATTGGTCTGCCCAAACTTGTGAACTGTTTGCACTCAGCCAAGCCTTAAAGCACTTGC
AAAACCAGGAAGGAACCATCTATACTGATTCTAAGTATGCCTTTGGAGTGGCTCATACATTTGGAAAAAG
TTGGACTGAATGTGGCCTCACTAATAGTAAAGGTCATGACCTTGTTCATAAGGAGTTAATCATCCAAGTA
CTGGATAACCTTCAGTTGCCAGAAGAAATAGCTATTGTCCGTGTACCCGGGCACCAGAAAAGCCTTTCTT
TTGAAAGTCGAGGAAATAACCTAACAAACCAGATAGCCAAACAAGCTGCTGTTTCCTCCGAAACACCTAT
GTTTCACTTAACTCCTTGTCTTCCTTCCCCTACTGCAATTTCCTTTTTCTCTTCCATTGAAAAAGAAGAA
TAAAGATAGGAGCCAAAGGAGAAGACCAGAATGAAAACGGCTGTTACTAGACCAAAAGGAAATGTTATCC
AAGCCCGTTATGTGGAAGATCTAGTCTCAACTATATCTGAGGACACACTGGGGATCCCAAGCCATGTGCG
ATGCAGTTCTCAGGGTCTATGGATGTATAAGAATTTACACCCTAGCCAAACAAGTTACAGATAGTTGCTT
AATATGTAAAAAGACTAGTAAGCAGATTCTAAGAAAACCGCCCCTTGGAGAAAGAGATTCAGGGCTAAGA
CCATTTCCAAGTGTTCAATTAATTATACTGAAATGCCCCCAATTGGTCATTTAAAATACTTATTAGTAAT
AATAGACCACTTTACCTACTGGGTAGAGACTATCCCACACTCAAATGCAACCACCAGTAACGTAGTTAAG
GCATTAATTGAAAACATTGTACCCAGATTTGGACTAATACAAAGCACTGATTCAGACAATGGAACCCATT
TCACTGCATATGTCATTAAAAAGTTAGCCCAGGTACTAGACATAAAATGGAAAAACCATATCCCTTGGCA
TCTCTCCTCCTCAAGAAGAGTAGAAAGGATGAATCAGACTCTAAAAAGCCACTTAACTAAATTAGTTCTA
GAAACTTGATTGCCATGGACTAAATGTCTTCCTATTGCCTTGTTAAGAATCCGAACTGCTCCTCAGAGAG
ATACTGGCCTTTCCCCTTATGAGATGCTCTATGGATTGCCCTATTTATACTCCACTGCTAACATTCCTAC
ATTCAAAATAAAAGATCAGTTCCTCAAAAATTATATACTTGGTCCATACTCTACTTTCTCTTCCCTTAAG
ACTAAAGGTCTCCTAGCACAGGCGCCACCGCTGGAGTTTCCAGCACATTAGCGTCAGCCTGGAGACCATG
TCCTCATAAAAGGGTGGAAGGAAGGCAAACTCAAACCAGCTTGGGAAGGACCCTACTTGGTGCTCCTAAA
TACTAAGACTGCAGTCCGAACAGCAGAATGAGGATGGACTGATCACACCCGCGTCAAAAAGGCGCTGCCA
CCTCCAGGATCATAAACCGTCACTCCAGGGCTCACCCCAACCAAATTAACTCTAAAAAGGGCTTAATAAT
CACTTGTTTATTTTTTCTTTTCTTTCCAACAGAAGGTCATCTTGTCATCAATGTAACTTGGGCTAACCAT
CCTTTAATCCTTCAGTTTGATGCTTGTTCAGTCATCCTGTGTGGAGACAAGCAAGCTCAAAGGAAGCTGT
CTCATGTAGATAAGTACCTATGTCCATACCATAAAAAGTCAACCAAGTATAAGTATAGAACCTTAAAAAG
TCCCTGTGGTGACTGGACAGATGTTTGGAAACCACTCAGTATGGAGGGTGGACAGCCAGGCCCCCTTTTC
AAATAAGTTATGGGGACTAAAACAGAAACTCCAACTAATTCATGGTCCCACCCCACCAAACTGTAAGCCA
CTGCAGTGTAACCCCTCATTGCTAATTATAGCTAACCCCCAAACAATGGCCCAAGAACCCACTATATTCA
AACAGTATGGATTAGGAGCAAATGTTACAAAACAAAATCCCATAAAAATCTTCAACCACTGGTTAACAAT
GATAATTAAAAAAAAATCCACACCCAGAGTCTGGGAAACGAGTGGAATCTAACACTCTCTCCAAGTATAG
CGAGCTCAGCGTCCTCCTCCCATCTCCAAAACAACCCAACTAAAGTAACGGTTGTAAAGGTAAAAAATTT
AAAGCAGACTATAGCCCAACTAAAGTAACGGTTGTAAAGGTAAAAAATTTAAAGCAGACTATAGCCCTAG
AGACAGGATATCAAGATGCAAAGTCTTGGCTAAAATGGATTAAATATTCCATCTGCACTTTAAACAAATA
TGAATGTTATGCTTGTGCTCACGGTAGACCAGAGGCCCAGATTGTCCCCTTTCCACTCAGATGGTCTTCT
AACTGACCGGGCATAAGCTGTATGGTAGTTCTCTTCCAAGATCACACAGCCTGGGGTGACAAATCATGTC
AAGCTCTTTCTCTGCTGTTCCCTGAAGCTCAACACCCTGAGGGTCAGCCCCCGAGGGCCATCAGCTTCCG
TTTCCCAATGCCAATTTTACTTTGTGTCTCTCACAACAGGGAGAAAACTTGGTGTTCCTTGGAGCCTTAA
TGGGATGCAGTGAGCTTAAGTCCTTCCAAGAGCTTACCCATCAGTCTGCCCTTAGTCATCCTCGAGCGGA
TGTATGGTGGTATTGTGGTGGACCATTACTGGACACTGCCGAGTAACTGGAGCAGAACATGCACTCTAAT
TCCATTGGCTATCCCTTTCACCCTGGCATTTCATCAACCAGAAAAGATAGAAACCAAACACTGTAAAACA
AGAGAGGCCCCTCATGGGTCTGTTGACTCCCACATTTATATAGATTCCACTGGAGTCCAACGAGGAGTGC
CAGATGAATTTAAGGCCCAAAATCAGACAACTGCAGGATTTGAATCCACGTTCTTCTGGTGGTTAACTAT
AAATAAAAATGTGAATTAGATAAACTACATTTATTACAACCAATGATGATTTTTTTTTTTTTGACGGAGT
TTTGCTCTTGTTGCCCAGGCTGGAATGCAACGGCATGATCTCGGCTCACCACAACTTCTGCCTCCTGGGT
CCAAGCAATTCTTCTGCCTCGGCCTCCCGAGTAGCTGGGATTATAAACATGTGCCACCACGCCTGGCTAA
TTTTGTATTTTTAGTAGAGACAGGGTTTCTCCATGATGGTCAGGCTGGTCTCAAACTCCCGACCTCAGGT
TATCTGCCTGCCTCGGCCTCCCAAAGTGCTGGGATTACAGGCATCAGCCACTGTGCCCAGCCTATGATTT
GTTAATTATACTAGAGATGCTCTTAAAGGGACAGCTGAACAATTAAGACCCACCAGTCAAATGGCCTGGG
AAAATAAAATAGCATTAGACATGATACCAGCGAAAGAAGGTGGAGATTGTGTCATTATCGGAACCCGATG
CTATACTTTTATCCCTAATAATACTACTCCAGGTGGGATCACACCAAAAGCACTACAAGGTCTTACCGCC
TTATCAAATGAGCTAGCCAAAAATTCTGGACTAAACGACCCCTTCACAAATTTAATGGGAAATTCTTTGG
CAAATGGAAGGGATTTATGTCCTCAATCCTCATGTCTCTTGCCATCAGAATAGGGTTGCTTATTCTTGTA
GGATGCTGTGTCATACCCTGTGCCCTAGGATTAATACAAAAGCTCATTGAAACAGCTCTCCCCAAAACCT
CCCTCAATCCTCCTCCTCCTCGTTCCAGTGAGCTTTTTCTTTTAGAAGACCCAAGTAGAATAACAGAGCC
AAATTATGTTAAAAGGGTTTAAGAAAAAATACTGTAAAATATTTAACAGGGGGATTRTGTTAAATACAGT
AAGAAATTCTTCTTCAAAGGTTCAGCTTGCTTAAGTTTCCTTGTTCTTTGCTCCCTGCTTTCAAGGCCAG
ACTTCCTTACTCTCTGTGTTCTCCCTGCGCTGGTAAACAACCTTCCCGCCAGTCGTTATCTATAGAGCCC
ACATTCCACATCTGCTACCCACTCTGTGAATTACCCCTCCCATCACAATGGCTTCTCCCGGCAAAACTGA
TCTTCCTGCCTTCCCAGCAGTGTAATCACATTCCTGCACTTTTCAAGTTAGCCAACCGGGTTCAGCTTAG
ATTGTACGGTCCAACTCTAGTCAATGGAGTCAGGATACAGTAGCAGGGACAAGCTGCGTTAGACATAAAA
ACCTCTGCTTTCCTTTGTTCGGGGTGCTCTCGTGGCAACCGAACCTATGAGGAGCAACCTTATGCAAAAG
TAAATTTGCCTTGCTGAGAGACCCTTTGTCCTTTGTCTCAGTGCTAGTTCTTCTTTGCAGCACCAAGCAT
TTGTTTTCAACAATTATATATTTAAGGAATCTTTGATTATGGAAAATTTCAAGAAGTAGAGGGAATAGCA
TACTAAGCCCTCTGTTCCCATCTTTGAGTTATCTACATTTTATTAGAAGTATCTTTGGGGTGACTGAGGG
AGGTTTCTCTTTCCTGGAGGGCTTCTGAATTATTGCCAATGTACCTTCCTGACTGCAGCCAGAAACTGAG
GCCCCTATACCTATGTACCTTTCTATCCCTCTCTGAAGCTTCTGCAGAATTCCCAGCAAGACAGAGCTTG
CTGGAAGCTTGGTATGCTCAGAAGCAGCTAAAGCGTGTATGTGGGGCGGAGGGTGGGGGCAACTTCTTGG
TCCTAGCACTTCCATATATTGATTTTCTTTTCTGGCTGCTTAGTGGGAGGAGTGTGTGTGTATGCATGTG
TGTGTGTGCGTGTGTGTGTGTGTGTACATGCCTGTGTGTGTGGATGCATGCATGTGCTGTGAAGCAGGGA
GACCAGCTTTCCGTTCCTTCTTGTTTTCTCTCTGCAGTGACCTGGGAGCTGTCATTTCCCTCCTGCTCTG
GGGACGACAGCTTTTTGCACTGTACTCAGGCAATGATGTCACGGATATTTCAGGTCAGTCTTTGAGTTGG
GTAGGAGCATGCATCCCTGGCACTGCCACATCCCACTCTGACTCTCTCGGGTCTGCATTCTTTCTTTGAG
AACACACAGTTCCCCATTCTTATCCTGACCTCTGGGCTTTCAGGACTGCCAAGAACATTGGGGATCCTGC
CAGAAATGAGGGGAGCTTGAGCTTTCGTTGGCTTCTATTTGGGGTAGAAGGAGATTGATATGCAGAGCAG
CTTCCACTCATCTGACTTTTCATGGGTCTCTGGGAACAATTTCCAAATGGTAAACTCTCTGGCTTCTCTC
TCTTTGCAGATGACCGCTTCCCGAAGCCCCCTGAGATTGCAAATGGCTATGTGGAGCACTTGTTTCGCTA
CCAGTGTAAGAACTACTACAGACTGCGCACAGAAGGAGATGGTAAGACCTGGACAACTATCTCCTGTGCT
CTACCTACAACCCCTGCTCTGACATTTCCATGATGGGTGGTGCTGAGGTGATTTGCCAGAAAGTTCGTTG
CTCTCCTTGGAGCCAGGAGATTTAGATTCTGATAAGCGTTTTGTCGCCAGTAGCCATGGCCCTTTGGGCA
GACTAACTTTTGTCAGCCTCAAGTTTTCTGTTTTGTTAAGGGGAGGTGATGCCATGCAGCCTACCTCATG
TAAATCTCAGAGTCAGATTTACATCTCCAGCAGATGTGGGAAAAGAAGGAATGCTGATGATGATGTCACC
CTCACCTAGTGAGTCTTGCTGTCCTGGCACTGCTCTAAGGGCTTTATATTTATTTGCTCACTTAGTCCTT
ACAGTTTACCTCTGAACAGAGGTTATTATTCTCACTTTGCCGATAAGGAAACAGAGGCACCGACAGGTTG
AGTATCTTGCCCAAATTCAGGCGGCCTGTAAGAGGCAGAGTCAGGATTTGAACCCTGAGCCCTCCCTGTA
CTGCTTGGCTGTGACCGCCATCACCACAGTGTGTTCTGCTGGGCTTAACTGGTGTCCAGGCACTTGGCTT
CCAGCACAGCACTCTTTCCCTTCCTCCTTCTCGTATTCTCTCTCCTTTCTCCCTTCCTGTCTGCCTCCTT
TCTTCTTCTTCTTTTTAATTCTTCTCCTTAAATGCCTTCTCACTCTGCTCTGGGTGCAGACTTGACTTTT
CCTTTGGCTCATTTCTTGCCTTTTGTTTCAGGAGTATACACCTTAAATGATAAGAAGCAGTGGATAAATA
AGGCTGTTGGAGATAAACTTCCTGAATGTGAAGCAGGTGGGTGCTGAGCACTGAGCACTTAAGAGAGCAG
GCAGGCGTCCAGCGGGGAACGTCCTAGAGGCACAGCCTTCCAGTGCGGCTTCCTCTGAGCACACAAGAGC
CAGGAGGAGGGATGTGGGAGAACCGCAGCTGGCCAGGGAGAGACTTAAGCAGTTAGGTGATGACTCCCTA
AGGATCACCAGGGTCTTGTTCATTGGGGCCTGAAGGGCACTGGCTGAATCCACTGTCGGCATTGCCACAG
ATCAGGAGAGCCTGTGCATACAGAGAGCCTGCTAGAGAGCCCTGGGTCTAAGGAGAAGCAAGCTCCAGGG
AGAACAAGTCAAGGGATAACATAAAATCTTAATCCATGGAAGCCTAGCAGGAGGCTGGACATGGGCTGGA
ACTCCTGCTTCTCGTTATTAGGAGGAACTGTTGCTCTCTCCTTTCCATCTCAGAACCAGAGGCAAAGGCC
CAGCCTCTTCTGCTCTTACTGGTGTGGAAAGGCCAGCCTCTTCTGCTGTGAGTGGTGTGGAACTACCAAC
CTGCCTCGTATTAACTGCAACATCTATAAAGTATGAGCTCCAGCCAATGCTGCTCTAGATTCCTCTTTCT
TCAGAGATGATGAATTATTGTAGCTCCTAGCCCTTTCTTTTTTCTTTCTTTCTTTTTTTTTTTTTTTGAG
ACAGAGTTTTGCTCTCGTCGCCTAGGCTGGAGTGCAGTGGTGCTAATTCCTGACCTCAGGTGATCCAACT
GCCTCAGCCTCCCAAAGTGCTGGGATTACAGGCGTGAGCCACCGCATCTGGCCCCTAGCCCTTTCAATGA
ATTTCAGGGAATTGTGAAAATTCCTTTGTTGAGATAATTGTTTAAATATAATATAGTTCACCAGCCAGGC
CTCAAAAAACTCAGTATTTCTCATTTCCTTATTTAGAAATAGAGCTTTTTGTAATGTAAACAATTAAAAA
AATTATTTTAAAACTGCAACTATTGGAAATGAGATCAGCAGGTGGTAAGGACAAAGCATTTAAATCTTTC
CAGTTTATGCAGCAGTGACAGCCGCCAATGCTTTCACCCCTTTCTCAGATGGAAAGGCTCTTGCACATTT
CCACTCACGAGTGTCTTGCTCTCCTTGACAGTATGTGGGAAGCCCAAGAATCCGGCAAACCCAGTGCAGC
GGATCCTGGGTGGACACCTGGATGCCAAAGGCAGCTTTCCCTGGCAGGCTAAGATGGTTTCCCACCATAA
TCTCACCACAGGGGCCACGCTGATCAATGAACAATGGCTGCTGACCACGGCTAAAAATCTCTTCCTGAAC
CATTCAGAAAATGCAACAGCGAAAGACATTGCCCCTACTTTAACACTCTATGTGGGGAAAAAGCAGCTTG
TAGAGATTGAGAAGGTGGTTCTACACCCTAACTACCACCAGGTAGATATTGGGCTCATCAAACTCAAACA
GAAGGTGCTTGTTAATGAGAGAGTGATGCCCATCTGCCTACCTTCAAAGAATTATGCAGAAGTAGGGCGT
GTGGGTTACGTGTCTGGCTGGGGACAAAGTGACAACTTTAAACTTACTGACCATCTGAAGTATGTCATGC
TGCCTGTGGCTGACCAATACGATTGCATAACGCATTATGAAGGCAGCACATGCCCCAAATGGAAGGCACC
GAAGAGCCCTGTAGGGGTGCAGCCCATACTGAACGAACACACCTTCTGTGTCGGCATGTCTAAGTACCAG
GAAGACACCTGCTATGGCGATGCGGGCAGTGCCTTTGCCGTTCACGACCTGGAGGAGGACACCTGGTACG
CGGCTGGGATCCTAAGCTTTGATAAGAGCTGTGCTGTGGCTGAGTATGGTGTGTATGTGAAGGTGACTTC
CATCCAGGACTGGGTTCAGAAGACCATAGCTGAGAACTAATGCAAGGCTGGCCGGAAGCCCTTGCCTGAA
AGCAAGATTTCAGCCTGGAAGAGGGCAAAGTGGACGGGAGTGGACAGGAGTGGATGCGATAAGATGTGGT
TTGAAGCTGATGGGTGCCAGCCCTGCATTGCTGAGTCAATCAATAAAGAGCTTTCTTTTGACCCATTTCT
GTGTTGTGTTCAGTCTTGAGTCTTTTTTATTTGCTCCTTTATGGTCCAGGGTAGTCAGAAGGTATAGAGT
CTACTGGGAGTATGGCAGAAAACACCCTAAACCCACTGGAAATCCCGAAGGTGATACAAACTCTTCCACC
TTAGGGAATCATGCTCACTGATTGAGTGCCTATTGAATGCTAGGTCCCAGAAAGTTAACTGTTGTCCTTG
TTTTACAGACAAGGAAACAGAGACTCAGAGATGGTAAGTGAGTTGCTTAAGGTTACATAGCTATGAAACA
GGGAAGCAGAACTTTGAACCCAGGTCTGTTTGATACAAACTCAGAGGTCCTTTCACTGCATGCTGTTGCC
TCTTTAAAGCGAATTAGGAGAAAAGGCATGGGCCTTGGTGAGGAAGAGGCTAGCTCAAAATGGGATGGGG
AAAAAGTGTTTTAACACAGACAGTACTTCAGAGTTTGGGATCTAACCTACCAGCACTCCAGAAAACAAAA
GATGATGATGTAAAGGCAGGATCTCTGGACTTGCTGAGTCCAAATCATGGACTTGGCCCAAACATTTGCT
AAGTAATTTAGGCTTCCTGGCCATTAGTTCCTGGTCTCTTCCATGAAGGACTTGAGCCAGGGTAACGTCC
CTATCAGCAATAAGCTTCTGGGACTCTGTAAAGAGTGGGGAGGTAGAGTCCCTGCCTCAGTAGAAGCTGC
TTTTCCTACTTGGAACAAACCCACCCTTCACCAGGAATCAAAAGCAAGCTGTCTGAGCCTCCTCTCCTGA
TGGCAGGTCATGCTATTAAGGGTCTCCCAGAAGAAAGCTTCTGGAGCTCTGAGAAACAGAAGCTCTTCTA
ACAGAACTTTTTCTTGCTATAAGCCTTCAATACCACCAATCACCAATGTGCCTTCTTCTCATGCGAAAAT
ACGGCCCCTCCTTTGCAGGGTGTAAAATGACACTGATGTCACTCTCTCTCACCAGGGTCTGGCCATCTCT
GTGAAGCTCTCCTCACAGGCACCCTGCAGATTCATCAATAAACTTCAACAGGTTAGCGCTAGTAGGCCAA
TAAGACCTATATTCTAGAACCTGTGAAAGAGATAATAGCCAGAAACTGGCAAACACCAGTCAAACTCTTT
AAAAAAATGCCTCTTAGAACACAGTCACTTACACATTTTCCTAGATCTCTTCAGCACCAAGTTTCTTGGA
AGAACAAGGCACTCTGCAGTTTTCTTAAAGAATTGTCTGATTGTATTAAGTGGCTTCTGGTAAAACAACA
GTAAGAGAAAAGCTCTGGCATTCTCTTGATCACTCATCTTCTCTGCCTTCCTCCCCAGCACCACCTCCTG
GCCTCAGCCTGTGGGCTTCTGCAAAGTCAGGTATCAGGACTAATTAGCTAGCATCAAATTCCTGTTAATT
CCAGAAGCAGGGACAAACCTGTTGGGGAGGGACTTAATGACAAAATTAGGTATAGGTCTATACGTTAATC
AAGGAAAATTCTCAACTTCCTTAAACTTACTTACCACCCTAGAGGAGAGCCAAATCCACTCTGATGACGT
GAAGTTGTAAAGAGAAAACAGTACCCTATTCCCTTGAAAGGCAGAATAGGTTTATAACTTATAATCGAAG
GTCTCCTTCAGGAAGGACTTCTTGAACCCCATATGTCCTCTTACAACACTCCAATATTGCCTGTAAAAAA
GTCGGATGGGTCATAGCGGCTACTGCGAGACCTTCGAGCCATTAACCAAATAGTCCAGACCACCCACCCT
GTCATTCCTAACCCTTACACCATTATCAAGCAAGATCCCTCATGACTGCCAGTGGTTCACAGTAATAGAC
CTCAATGATGACTTCTGGGCTTGCCCCTTAGCTGTGGATAGCTGGGACATATTTGCTTTTGAATGGGAAG
ACCCTCATTCCAGTTGGAAGCAATAGTAGTGATGGACAGTTCTACCCCAAGGGTTTACAGACTCTCCAAA
CCTATTAGTCAAAGTTTGGAACAAGTCCTAGAGAATTTCCCTCTTTCATCATCCTTATGTCTACTCCAAT
ATGTGGATGACCTGCTCATTTCCAGAGGCACCAAAGACCAAGTAACCGCAATTTTAATTAGCTTTCTAAA
TTTCCTAAGGGAACAATGGTTACGGGTCTCAAAAAGTAAACTCCAGCTTGTAGAACCTGAGGTAAAACAC
ATGGGGCACTTAATAAGCAAAGGTAAGCGGAAGATAGGGCCCGAACAAATGGAAGGGATCATATCCCTGC
CACTGCCTGAGACAAAACAAGAACTTAGAAAATTCTTCAGGCTAGCTGGAAATTATTGCCTGTGGATTGA
CTCTTATGCCTTAAAAACAAAACCTCTATATCTAAAACTTACCCAAGAAGGGCCTGACCCCCTTCTTTGG
ACCCCACAAGAAGTCCAGCAAGTTGAGGAACTAAAACATCTACTTATAACTGCCCCTGTCTTAGCTCTGC
CATCCTTAGAACAGCCATTTCACTTTTTTGTTAATATAAGCAATGGGGCAGCTATAGGAGTACTCACTCA
AAAGCATGGGGCCATTGCCAGCCCACAGCCTTTCTGTCAAACATTCTTGACGTGGTAACCTGTGGGTGGC
CCGAATGTATCCAATCTACAGCAGCAACTGTTTTATTGACAGAGGAAAGTAGAAAAATAACCTTTGGGGG
AAGTCTCATCCTAAGCACACCCCATCAGGTTAAAACCATTCTTAGCCAAAAAGCAGGGAGATGGCTTACA
GATTCAAGAATCCTAAAATATGAAGCTATCCTATTAGAAAAAGGTGATTTAACCCTAACCACTGACAATG
TGCTCAACCCCGCCACCTTCCTTTTTTTTTTTTTTTTTTGAGATGGAGTTTCACTCTTATTGCCCATGCT
AGAGTGCAATGGCACAATCTCGGCTCACCGCAACCTTTGCCTCCTGGGTTCAAGCAATTCTCCTGCCTCA
GCCTACCAAGTAGCTGGGATTACAGGCATGCTACCATGCCTGGCTAATTTTTTTGTATTTTTAGTAGAGA
TGGGGTTTCTCCATGTTGGTCAGGCTGGTCTTGAACTCCTGTCCTCAGGTGATCTGCCTGCCTTGGCCTC
CCAAAGTGTTGGGATTACAGGCGTGAGCCACTGTGCCCGGCCTACCCCGCCACCTTCCTGACAGGAAATC
CAAACGCTGGAGACCCCAAGCACAAATGTTTAGACTTAATCATCAAACTAAAGTAAGGCCTGATCTAAGT
GAGACCCCTTTAAAAACAGGGCACCATCTCTTTGTAGACAGCTCCTCTCGGGTAATTGAAGAGAAAAAAC
ATAATGGCTACTCAGTGGTTGATGGAGAAACTCTCACAGAAGTAGAATCAGGGAGACTTCCCAATAACTG
GTCTAACCAAACATGTGAGTTGTTCGCACTAAATCAAGCCTTAAAATAGCTGCAGAATCAAGAAGGGACT
ATTTACACCAATTCCAAATATGCTTTTGGAGTGGCCCATACCTTTGGGAAGATTTGGACTGAACAAGGCC
TTATTACTAGCAAGGGCCAAAACCTTGTCCACAAAGAGTTAATCATGCAAGTATTAGAAAACCTTCAGCT
ACCAGAGGACATAGCCATTGTTCATGTCCCAGGACACCATAAAGGTCTCTCTTTCAAGAGTTACTTCCCA
AGAAGCACCCATTTTCCACTGAACCCCTTGTCTCCCTTCCCCATCTGCAACCCCCATCTTCTCCCAAGCA
GACAAAGAAAAATTAAAGAAAATAGGGGCTAACAAAAACTCAGAAGAGAAACGGATACTGCCAGATAACA
AAGAAATGTTGTCCAAACCCCTAATGACAGAAATCTTAACACAACTTCACCAAGGGACCCATTGGGGTCC
CCAAGCCATGTGTGATGCAGTCCTTAGAACCTATGGGTGTACAGGGATTTATACCCTCACCAGGCAAGTC
ACAGACAGTTGTATGGTATGCAGAAAAACCAATAAACAAACTTTAAGAAAACTGCCCTTCGGCGGAAGAA
ATCCTGGGCTAAGGCATTCCAAAGTGTCCAAATTGACTTATACCGAAATGCCCCAAATAGGCTGCCTCAA
ATGTTTGTCAGTTATAGTGGACCATCTCACTCACTGGGTGGAAGCCATTCACCTGCCAAGTGCAACTGCC
AATAATGTAGTTAAAGTACTAATAGAAGATACCATACCCAGATTCAGATTAATAGAGAACATTGACTCAG
ACAATGGAACCCATTTTACTGCACAGGTCATTAAAGGATTAACCCAGACACTAGAGATAAAATGGGAATA
TCATACTCCCTGGCACCCACCCTCATCAGGAAAAGCAGAAAGAATGAATCAAAATAAATAAATCACCTAA
CTAAACTAATCTTGGAAACCCGACTGCCATGAACTAAGTACCTCCCTGTCGCCTTACTAAGAATTCGTAT
CGCCCCTTGAAAAGATATCGGCCTGTCCCCTTATGAAACGCTCTACAGATTGCCATGCTTAAATCCACCA
CTGACATCCCTACATTTGAAACAAAACATCAATTTCTTAAAAACTATATACGTGGTCTATCTTCCACCCT
TTTTTCCCTCAGGACCCAAGGCCTCTTAGCACAAATGCCGCCTCTAGAGTTTCCAGTACACCCACATCAG
CCTGGGGATTATGTGCTCATCAAAAGCTGGAGGGAGGAAAACTCGAACCATCGTGGGAAGGACCCTATCT
AATACTCCTAACAACTGAAACAGCAGCCCGAACGGCTGAACAGGGGTGGACTCATCACACTTGGGTAAAA
AGGACCCCACCCCTTATGGAATCATGGACCATCACTCCAGGACCAACTCCCTCAAAATTAACATTCAAAA
AGGTCTAATTTGTCTCTTCTTCCCCCTAATGCCCAGGGGCATTTCATTTTTTTTTTTTTTTGAGATGGAG
TCTCGCTCTGTGCCCAGGCTGGAGTGCAGTGGCAAGATCTCCACTCACTGCAAGCTCTGCCTCCTGGGTT
CACACCATTCTCCTGCCTCAGCCTCCCGAGTAGCTGGGACTACAGGTGCCTGCCACCATGCCCGGCTAAT
TTTTTGTATTTTTAGTAGAGACGGGGTTTCACAGTGTTAGCCAGGATGGTTGATCTCCTGACCTTGTGAA
CTGCCCGCCTCGGCCTCCCAAAGTCCTGGGATTACAGGCATGAGTCACCGTGCCCAGCCCGCATTTCATT
GTTAATGTAACCAGGTCAGCCTCCCCTCAAGCTGTTGTGTTTGATGCTTGCCTAGTCATACCTTGCAGAG
ACCTACAAAGTCAAAGGCAACTAGCCTCTTCAGAAAAGTATCTTTGCCCTTGGTACTCACCCCACCCTCA
ATCTGATCCTTGCAAGATTCCCTATAATTGGCTACCTTGCCATTCCTGGTACGATATTCTCTGGACCACT
CAGTCCCAAGGCTGGACTTCTTCAGCAGGCTGCACCTCCCTAAAACCTTATATCCATTTTACTAAACAAA
ATAACTCCTCTAACTGCCAACACCTCCGATGTAACCTGGTGCATATTTCTATTACTACTTCAACCTCCAC
TAACCATATTCCCACCCTAAGTCGCTTTTATGGTATGGGGCAGATGTTAGCGGGAAAGACCCCATAGGGT
CCTTTGAGATTCGTTTTGTCGCCCCTCCAGCACCCCTTCCTTTCCGAAGATCTCCTCCAAATGAAACAAC
TATCTTGCCACTACCTAATGACAAAACTAAAGTGCACATAGTGGAAGTCAAAGACCTAAAACAAACCCTG
GCCATCGAGACCGGACACCAAGAAATGAATGCCTGGCTGAAATGGATCAAATATTCAGTTTGCACTTTAA
ATAAAAGCGACTGTTATGCCTGTGCAATGGGCAGGCCAGAAATCCAAATAGTTCCCTTCCTGCTTGGATG
GTCTAACCAGCCGGGCATGGACTGTACAGTAGCTCTCTTCCAGCACCCCACAGCCTGGGGTAATAAGTCA
TGCACCAGTCTCTCACTGCTTTTCCCAAAAGTCAAAGACTTCCCTGTGGGTCAGCCCCCAAGGCCATCTG
GCTCCTGGCCTCTGATGCCAATTTTACCTCTTGTCTCTCATGACGGGGAAAACTTACATCTTTCAGGAAT
CTAACGGGATGCAGTGAATCCGAGCCTTTTCGAGAGCTCACCAATCAGACTGCCCTTGTCTATCCACAAG
CAGATGTTTGGTGGTATTGTGGAGGACCTCTACTGGGTATGCTGCCAAATAATTGGAGCAGCACTTGCAC
TCTAATCCAACTGGCCATCTCTTTCACCCTGGCATTTCATCAGTCCGACAAAAAGTCACCCTCTTGGGAA
GAAAGAGAAGCTCCTCAAGGGTCTTTTGACCCCCATGTCTATATAGAGGCAATTGGAGTACCACGAGTAG
CCGCCAATGAATTTAAAGCTCGAAATCAAATAGCTGCCAGGTTTGAGTCAGTCTTATTTTGGTGGTCTAC
TATAAACAAAAACGTAGACTGGATAAACTATATATATTACAATCAACAAGGATTTGTTAATTATACCAGA
GATACCATTTGGGGATAGCCGAGCAATTTGGTCCCACCAGCCAAATGGCCTGGGAAAACTGGATAGCTCT
TGACATGACATTAGCAGAAAACGGCAGAGTTTGTGTAATCATCAGAGCCCAATGTTGTACTTTGATCCCA
AATAACACAGCTCCCGATGGAACCACTACCAAGGCTCTACAAGGCCTCACCACCCTAGCAAATGAGCTGG
CTGAAAATTCTGGAATAGATGACCCCTTCACAGGTTTAACCAAAAGATGGTTTGGTAAATGGAAAGGACT
TATGGCCTCTATCCTCACTTCCCTTGCCATTGTTACAAGTGCACTCATCCTTGTAGGCTGTTGCATCCAC
CTTGTGTTTGTGGGTTAGTTCAAAGACTCATAGAAATGGCTTGCACTAAAACCTTCTCTGTCTCTCCCCC
TCCATATTCAGACCAACTTTTACTCCTAGACAATCAAGAAGAGCAACAAAGCCAAATCTTACTAAAATAA
TTTGAAGAGGAAGAATTATAACAACAAAAAGGGGGAAATTGTCAGAATAAAAGAAAGGTTCCTCTTCAAA
GATCCAACTTTCTTGTCATAGGCTGTAAACCGCCTTGCTCCAAACCTTAACCTGTAACCTTTACCCTCTG
CCTTATTTGAGAAAGATTCAAGCGCATAGCCAATTGGAGTCAGCCTAGACTGTGCGGTCCAACCCAGCCA
ATGGGGAAAGACAAAGAAGCAGGAACTGCGTTAGGGTTAGAAACCTGTTCTCCTTTATTAGGTGTGCTTT
TGCGACTGCTATAGGCGCGGGCAGCACCCTTCTGCAGAAGTAAACTTTGCCTTGCTGAGAAAACTTTTCT
TCTGAGTGCTCCTCTTCCTCTGCGGCACTCAGAAATAAGAGAACTTATTTCTAACAGGAGGAAGAACATG
ATAATTTTGCACACGCAGGTTCTGAGTTTTGGCTGGTGCGCTGAGTGCTTACCATACCGCAAATACTGTG
TGATCTGGGCTGAAGAACAGAAAACCCAACTAACAGTGACTTAGGCAAAAAGAAATTTTATTCTTTCACA
GAAGTCTGTGGCAGGCAGGTCCAGAGTTCATGCAGGAGCTCAATAACATCAGAGATTGGGCTCTTTTTGT
TCTTCTTAGCATATCGATGTTTTACGTGATGCTGGGCACCTCGGGGACCTAAGATGGCTGTTGTAGCTCC
ATGCATCATGATTAAACCAGAGACAGGAAGTGTACGATGGAAGAGGCCAGAGGTGAATGGCCTCCTCCTC
TCCTTTTGTCTTATTCCTGGAGTGACTCCCTCCTCTTATCAGCAGGGATACAAGCAGTGCCTGGCAGCCT
TCCCTCTCTCCTGTATGAGCCAAAAGCAAGACTCATGGCACCCCTCCCTGCAGGAAAGGGGTGGAATGGG
CCCCTGGCAAAGGGGGCTGGACTTGCAGTGACAGTGCTGCTCTGAACAGGACTAGGCTTCTGTTGGAGAG
GTGGTCAGCGGCTGCTAGGCAGGTCACAAGCTGCCTCACCCATGGCTCTCCAGCTCTGCACCTACCCTCT
CCTTCTAATAAATCTGTTTCAATAACCACCACGAGGTTTAAAGAGGAGCAGTAACTTGCCCACGGTTGCG
CCAGGCACCCCTAGAAGAGCAAGAATTCCTTACAAAGTTATTAAAACAGGCAAATCCCATATAGGTGACA
TTTCTATCCAACAATGCTGACATCTCTTTTAGTTTTGAGCCTTAGTAATCCAATTTTCTGCCTCAGGAAG
AGGCAGCAGCTTCTGGGAAAAATCTGCTCCTGCTAAGGTGGCTCATTCATTGTTTTACCTGAATAGAGTG
GTGGATGGTGAAGGTTCAAATTCTCACTTTCCATATTAAATTTTATAACATAAAAAATAATTAAATGGGG
TGCGCGCGGTGGCTCACGCCTATAATCCCAGCACCTGGGAGGCTGAGGAGGGCAGATCACGAGGTCAGGA
GATCGAGACCATCTTGGCTAACACGGTGAAACTGCGCCTCTATAAAAATACAAAAAATTAGCTGGGCATG
GTGGCGGGCGCCTGTAGTCCCAGCTACTCAGGAGGCTGAAGCAGGAAAATGGCATGAACCCAGGAGGTGG
AGCTTGCAGTGAGCCGAGATCGCGCCACTGCACTCCAGCCTGGGTGACAAGAGTGACTCGGTCTCAAAAA
AAAAAAAAAAAATTAAATGAGCCAACCAGGACAATGTTTAAAAGCCAGAGCCAAAAAGGCTCCAAAATAA
AACAAACTTGTCTGCTCAATTAATTATTTGAAGAGAACCTACAGTAATACAGACTGGAGATGGGAAAGCA
CGAGGGATGTATGAGTCGAGGAACCGGCAAAGGGGTGTGGGGGTGTGTGTGTGTGTGTGTGTGCACACAC
ATGCACCCAGCTAGGGTAGAAAATAAGTACTTTTGCTTCTATGGGAAGTGACTTTCCAGTTCACTTGTGT
ATCAAATGAACATGATGAAATCAAGTTGTACTAGCCAATGTTTGGGAGGGTTCTGTATTAAAGGGATAAA
GAAAAACATTAGAAAGAGAAGTTACAAAAATCACTGTGGGGAAAATGAACACCAATGACTTGGCTGCTCT
GAGGCTTGCAGGGAGTAGACAGTTAGGCATCCCAGGTCATCAGAGAACCAGTGGGGTCTTTTCCTCAGGG
GCCGGGTTTTCTACACGCAAGTCAAACCTCTTCTCCTCTGGGACACATGTTTCCAAAGGACTCCAGAAAC
ACAGCACAGCTGGATTCAGGTACTGTGTCAAGATGTGCCTTCTTCTTCATCTTTGACAGCAATTAATGTA
CTAAATGTCTTGATAGAGAAGGTCATATTTGGGAATATTCTTGGGATCAATAGGACTTTGGACAATAAGC
TTCCCCCTCATTGCTCCTCGATAGATTACTTCAATCAAATCTATGAAGTCTTGTTTGGTTTTGAAGCTTC
CCACAAACTTAGTGTGATCTGGAGATCTAGACAGCATAGAAGAGAAACAACTGCTCTAAGAACTTTGAGT
AATCACTTGTTGAAGACTTTCAGGATAAACAAAATGAAAAACAGCTTTGTTATTTTCTTCTTGTCAATGA
GACTTCAGAAATAAAGCCCAACATTAATTTTTCAATCATTTTTTTCCATTTAGTAAATGACTCAACCCAG
TGCTGTGGGCCACTAGCATGAAAGCTCTGTCATGTGCAAAGCTTTAGCTTCAAAAGCTATTGTAATCACA
AAATCTATTTTTATCACTTCTCAAATCATCAAGCAGCTTTCACCAAGTGTTGGAGTGCACATTGTTTCTT
CGTTTGCTGTTGTTGCTTTTGCCTGGGCATTGTGCAAAGCCCATTCAATATTCATGGCCTTTTGATGTTG
GTAGGTATTAAAAAATTGATTTTTTAGAAAAGTATCTCAAACACCAGGTATGTTACTGTAAAATTTTAAA
GAAATAAATAAAATCACGGATGAGGCCAGAGGCCAGAGGTCACTTTTCAGTGTCTATCCTTCCAATCTTG
TGTGTGTGTGTGTGTGTGTGTGTGTGTGTGTGTGTGTGTGTATGTGCAGATACAAACTTCTTTTTTCCCA
ACATTGGGAATTATGTCATACAAATTATTCAGTAATATGCTTTTTTCTAGCTGACATATATTTCTTTCAT
TTGTTAAATGTGCATCTGCAACATCATATTAAAGTCCTGGAAGTGTAATTACTGGGTTAAAAAGTTTAAA
AACTTTAAACTATTTGATACATAATGTTGAACTGGCCCCCATAAAAGGTTGTACCAAATTAATACTTACA
TCAAATGTGTTCCAGTAGAATAAACAGCTTTAAAATACTTTTGTGCTTTTTTTTTGTTGTTGTTGTTGTT
GTTTTGCGATGGAGCCTTGCTCTGTCACCAGACTGGAGTGTAGTGGTGCGTTCTCTGCTCACTGCAACCT
CCACCTCCTGGGCTCAAGCGGAATCTCCTGCCTCAGCCTCCTGAGTAGTTGGGATTACAGGCACCCACCA
TCATGCCTGGCTAATCTTTTTTTTGAGACGGCAGCTCTGTTGCCCAGGATGGAGTGCAGTGGCGCGCTCT
CGGCTCACTGCAAGCTCCGCCTCCTGGGTTCATGCCATTTTTCTGCCTCAGCCTCCAGAGTAGCCGGGAC
TACATGCGCCCGGCCACCACGCCCAGCTAATTTTTTGTATTTTTAGTAGAGACAGGGTTTCACTGTGTTA
GCCAGGATGGTCTCGATCTGCTGACCTCATAATCTGCCTGCCTCGGCTTCCCAAAGTGCTGGGATTACAC
GCGTGAGCCACTGCGCCTGCCATGCCTGGCTAATTTTTGTATTTTTAGTAGAGATGGGGTTTCACTATGT
TGGCCATGCTGGTTTTGAACTCCTGACCTCAAATGATCCGCCTGCCTTGGCCTCCCAAAGTGCTGGGATT
ACAGGCGTGAGCCACTATGCCCAGCCTAAAACACTTTAAAACAGATAATATTTCACATACTTTCAAATAA
AGACAAAAAGGGTACACAGATCTAGATAAGTGGACACATCAACCACAGTAAGAAATGATCAGAATCATGG
ATGCACCCAATCATCAATATTAAGTATTGAGATGGTAATTGGATGGAGTGGAAGGCACTAAAGAGGACAT
CATGTTACTTATTCTTGACAGGCTTTGTTGAAGAATGATTTAAAATTCAATGGCGAAAAGCAGGAAACTT
TGCAAGAGAGAAGCACATCTTAGTTTGCTAGAAGGAATGTTTCTTTCATAAATGCCACTTACCAGTCCAA
AAGCTTAAGAAAAACTACATGTTTTGTTTCAAATGAAATTCCTGGGAAAATTTTAAGAGTTCTTTATTTA
GCTAATATGGCTATTTTATATTTTATAGACTTATGGATGGTAGTGCTTTGCCACCTGTACTATTTCTTCA
TACTTAAGTCGGAAATTAAAATTTGACAAACCATGTGTATGCACTATTAAAAAAATAAATAAAATTTGAC
AATCATACCAGAAGCACTCTCATTTTGTAGCAAGGTTTTGCATAAAAAATTAGAGAGTGCGTCAATTGGG
GAAACAGTACTGATATAAGACTCACAGAGCCAACATCACATGGAAATAGGCAAGCTACTGAAAATGCTTA
CCAAGGAAACTTACAAGGTTGCAATTAACTTCAGATCAACTGCACTTACCCATAATCCACTTTCATATGC
TGCCCATTGAAGAAAAAGACAGTAGATGGAATATAACTGATGTCAAAATACTGTGTATAAACTGCAGTTT
GGTCCACATCTACCAGGTATATAGCAGCCATTTTACTTAAGTCAGAAGAGGTCTTAGAAAGCTGCAAATG
ACGAGAGAGACAAAGGTTACAATATGAGAGGCAGCTTAATGTTTCTTCTGTGAAACTTGTTACTACAACA
GAGTGTTTTGTTTGAAAAAACTCCTGGGCCAACAATGCAGCCCATGTGGCTCCCACATTAGACAAAGCAT
AACCCCATCCTCCAGAGTCTCATTCACTGCAACGGGGTTAGGAGGGAATCTTGGGGAAAACTGTATGAGG
TTAGGAAGGGACCTTGGGGAAAACTGTGTGAGGTTAGAAGGGGACCTTGGGGAAAACTGTGTGAGTTTAG
GAGGGTGACAGCATGGGAGTATTCCTGTCTATTCATTGTGGAGAGAGACCTAACCCAGAACTAAGACCTC
TACCCGCCATTTCTACTGCTGCCATCCCGTGTTAAGAGCTGAGCATACAAATCCTTGGGAAAACTGTGTG
AATCTGTTTCTTCCTTTAATTTCTTTCTTTGAATGCTTTTATAAAAGTAGCCCATGCTCATTTAAAACAA
CACAACTTAGAAATATGTTATGTGGATACTGAAATTCCCTAACGTCTCCTTCCTCCCAACCCTCCCAGAA
ATGAAAACCACGATAATTAGTAGCAGACACTTATATAACATTACTATGTGGTAACTGTCCTAAGTGCTTA
ACATTTGTTAACTCATTTAATCCTTACATCATTCTTATAAGGTATATGCTATCAGAGTCCCCATTTTATA
GATAGGGAAACTGAGGCACAGGAAGGTAAAGTAATTTGCCCCAGGTCACACAGCTCCTGAGCAGTAGGGC
TGGGGTTTGAACTTTGGCAGTGTGGCTTCAGTATACCACTGACCTCTGTGGTATTTCCTCCCGCCTATGA
CTGAGGGTAGAGTTCTCTGGCCCTTTTTTCCCCAAGTTAACACACTGCACCTTCATTGCAATTATGGAAA
CGAGGATTTGTACGCTCAGCTCTTACACGGGATGGTAGCAGTAGAAATGGCGGTAAGAGGTCTTAGTTCT
GGGTTAGGTCTCTCTCTGCATCAGGCACATACTGGAGGCAGCACACTAGAAAAGATTATTTGCAGCTACA
GAAAACTGAGTTCAAATCTCAACTCTGCAATATAGTGGATCTGCACCTGTGGGAAATTACTTGACCTCTC
TGGTTTTTTTTTTCCTACTGGGAAATGGGGCTACTATTATCCCACTATTGCGAGCACTGAAAAGATAGTG
TTCACAGATGCTTGTTAATTAGTTTTCTGCTCTTCCATCACCTTGTTGGTGAAACAGCATTGACAGCTTC
TAGTGGGTAAGGACATCCAAGACAGATTCAGCACACACAGATTTGGGGGCTTGGTTCAGGGCAGGCACAC
AATCAGCTCTTAGTGAACTAACAAATGAACAGCTATATACCCAACCCTGACTACTCCCTCTCATGTACTA
CTCAGATGCTGAAGTGAGAATATACAGATTATTAATAAAAACCAATTATTTTAGACATTCACTTACAATA
TCATCTAGCTGCAGACAGACAGGATCTTCATCTCTCCCAAACCTGAGAACCAACACCTTCTCAGCAGTAC
TTTTTATCGCCTGGTCTACTTCCTTTTTGCTAGTCAGCTTGGGCAGTAGGAAGCTCATCTTGAAATAACC
CAAATGTGAATCCTCACTGTCACTCCTGAAATAAAGGTGCAATGTTTAAAGACAGTTTAGATTAAGTGCG
TGAGCCCTCATTAAAAAAAATTAATTCACAGAGTAGCTAATGTATTCACATGGTATGAAAGAAACATCAT
AGAAAGGTAACACTGTGGAGTTTCCCTCTCTTTGTCCCTGCTCCTTGGAACCAGTTTTCATTGTTCCCTC
CCCCGGGCCAGATAACTGCTTTCATTAGTTTCTCATGTATCTTTCAGAGTTTTGTTATGCATTCACCAAA
AAATACAAATATGCGTTCCGATATTTTTTTTCTCTCTCTCCCCACCCCCAAAAGATAGCCTATTGTATCA
GTGTTCCAGCAGGAAAGCGATGACACTCTCAACTTTGGTAATTTGAAGGCAGTTTAATTAAGGCAGGTGT
AGAAAAACAATACGGTAAAGTGTACTACTTTGGGGCTAGTAACAGCGGGATGCTGTACCCATCCCTAGGC
CATGGGAGGAAGAAGCCACGGTAGGAAGCAGTTATAAGAATTGTGTGGCAGAAAGAGGTGAGTGGAGAGG
ACAGTGTATAGGAGGGCAGCCTGCTGTTAGGTGTTGCAGCCACTCTCCTCCCTCCCTCTGACCTTTGCTC
ATGCAACCACCGGCTGACTCCAACTGGAACCAGAGGGCAGGGGCCTGTTGAGGCAGGCAAACCTCCCTTG
TCCCAGAGCAGTCTAAGAGGAGGTTGAAGAATGGCTGTGGAGGGACCAATGGCAGATGTTCAGCACACAC
ACTTTATCTACTCTTCAGCACTTTTCATTATTTCAAACCTAACCTATGAAGATTGCCCCATATCTCTATA
TAGAGAACATCCATATTCTCTGTCATGGCAGCATATTATTTCTTGCATGTGTTACACTTTTTAAACAGTC
CTTTATTGTATACTTTATATTATTACAAGTCCTGGCTTAAAGATTCTTCGTGTGCACCTTACTAGGTGCA
CCTCATTACACGGCAAACAGATCAATTAAGAGTGTGTGTGAAAACTACAATACCATAAACAAATAATAAA
ATACTGAGGTACAAGGGACCTAATACAACCCCGTCATTTTACAAACTAGCTTGCTTACAGTTGCACTTTC
TACTACAATGACAAGGACTAGAATACAGGCCACCAAATTTCTAGTCCAGGGGTCCTTCTACTACAATAAT
CTATTTTCATGATTGGTAGAAATTTCATTTTTGAGAAACTCTTTGACAAAGAAGCTTAGTTTTGTGGTCA
TCTATAAGAGACTCCATTGAGATGGTCGGTATTTTGGAATTC
(hHp) (Accession No. AH003344)
SEQ ID NO: 9
AACTGCCATCATACTGAAGGTAATCTTCTGAAACTTGCGGTTTTCTTTCAAAATTATGTTTATAAGATGA
TCCATGTTCTTGCAGAGTTTATTCATTTTATGCTGTATAATATTCCATTATATCCACATACAATGCAGTA
TTGACCCTTCCTCCTGTTGATGGGCATTTGTCTTGTTTCTAGTTACTTTGCTATTATATCAGTGTCACCA
TGATTATCCAAAAGTAATTCTTTTGTACACTCTAATTTAAGAACAACTAACCCTTTTTAATGAATAAATC
AACCTTGTATTGAGTTGCTACTAAGTTTCAGTTGACTAGTACCTGGGATACACACAGGTGCAGACATTTG
ACTGAGACATATTGATTTTTCTCATCTGCCTATTTAGGCTAATCACCAGACTATAAAACCATGAGAACCA
CTGCCATTGAGTATAGTCTGTGTCAGTCTACACTATAGCTTTAACTAGTTGTGTGATTTCTTGCAAAGAG
CAATCAGAGAAGACACAATAAACACATTTACTGATTTCAGGCTGGAGAGCTTTTAAGCAATAGGGAGATG
GCCACACACAAGGTGGAGAAAATTACTGTGAAAAGGATACGTACTTTTCTTTAGAGCCCCACCTAAGCTA
GGCTGCAGAAATGTCTACAATGGCTTTGAAAAAACTCAAAATGAGCCTTTCTGCAGTGTGAAAATCCTCC
AAGATAAAGAGACAGATTGATGGTTCCTGCCGCCGCCCTGTCCTGCCCAGTTGCTGATTTCAGGAAATAC
TTTGGCAGGTTTGTGGGCATAGAGTTGCCAGGTTTCTTGGGATTTGTAATAGAACATCACAAGAAAATCA
AGTGTGAAGCAAGAGCTCAACTCTTAACAGGGGTATTGTTTGTGGTTTTGTTACTGGAAAAGATAGTGAC
CTTACCAGGGCCAAAGTTTGTAGACACAGGAATTACGAAATGGAGAAGGGGGAGAAGTGAGCTAGTGGCA
GCATAAAAAGACCAGCAGATGCCCCACAGCACTGCTCTTCCAGAGGCAAGACCAACCAAGATGAGGTGGG
TCCACAGCTTTCCCTCCTGCCTTTCCTCTGGTTCTTTATTTCAGTCTTTTTTGCATACATCGGTAGAGAT
GCAGAAATAGAACAAAGAAACGGGCAAATGGGCTAAATTATAGTGAACCAAAGGGCTTAGTGTGTTAAAT
CTTCTCCTTTTCTGCATCCATAGAAGACAGTGCTGCTGTCTTTCCCAGGAGATAAGATTTACTCTCAGGA
GTGTCTTTTTCCTTCAGGTTACATTTTTGACTTTATAGGGTATGTCATCAGCTCCCGTGGTAGGCTTCCT
GGCATCCTGAGTATATTTATTAGCAGATATTTTCCTCTTTAAAAATGTACAATAAGGAAGACTAATAGTA
ACACATTTGAATGACACAATTAATTGACTAGTACCTGGGATACACACTAATACCTGGGATACATCTAATT
AAGGCACTTAGATCTTATAAAAATAAACACTTTTTGAAATGTTGAAATAATAAGACTAGAAACTTTTTTT
TTTTTTGAGATGGAGTCTCGCTTTGTCACCAGGCTGCAGTGCAGTGGCATGATCTCGGCTCACTGCAACC
TCCACCTCCCAGGTTCAAGTGATTCTCCTGCCTCAGCCTCCCAGGTAGCTGGGACTACAGGCGTGCGCCA
CTATGCCCACCTAATTTTTGTATTTTTAGTAGAGACGGGCTTTCACCATGTTGGCCAGGATGACCTCGAT
CTCTTGACCTCGTGATCTGCCTGCCTTGGCCTCCCAAAGTGCTGGGATTACAGGCATGAGCCACCGTGTC
TGGCCTAGAAACTATTTTAATAGAAGCAAGTAGTGCCCGAATGGTTGGCATTTGTTAGTGAGATGGTGAA
CTGGCAGACGGCACCTGTGGGTCAATGCCCATGGCCACCGTCCTGCTTTTGGACACCAGTTCTCTTCCAG
GTAACCTTCTGGCATTTTGGGGTTTCAGATACCATTTCCTAAAGGTGAATTATTATAAAATACTAGAAAT
ACCCACGTGTTTAAAATTATATATTTAAGGAACCTTTTATTACGGAAAATATCAAGAAGTAGAGGAATAG
CACAGTAAGCCCAAAGTTCCCATCTCTGAGTTATCTACATTTTATTACCACTATCTTTGCGGTGTCTGAG
GGAGGTTTCTCTTTCCTGGAGGGCTCCTGTATTATTGCCAATGTACTTTCCTGAATGCAGCCAGAAACTG
AGCCCACCCCTCCACCTATGTGCCTTTCTATCCCTCTCTGAAGCTTCTGCAGAATTCCCAGCAGGACAGG
GCTTGCTGGAAGCTTGGTATGCTCAGAAGCTGCTAAAGTGTGTATGGGCAGGTGTGGGGGCAATTTCTTG
GTCCTAGCACTTCCATATATCGACTTTCTTTTCTGGCTGCTAAGTGGGAGGAGTGTGTGTGTATGCATGT
GTGTGTGTGTGTGTGTACATGCATGTGTGTGTGGATGCATGCATGTGCTGTGAAGCAGGGAGACTAGCTT
TCCACTCCTCCTTGTCTTCTCTCTGCAGTGCCCTGGGAGCTGTCATTGCCCTCCTGCTCTGGGGACAGCT
TTTTGCAGTGGACTCAGGCAATGATGTCACGGATATCGCAGGTCAGTCTTTGGTTGGGTAGGAGTGTGCA
TCCCACTCTGACCCTCTCGGGTCTGCACTCTCTCTGAGAACACCCAATTCCCCCTTCTTATCTCGACCTC
TGGGCTTTCAGGACCATAAAGAACATTGGGGTTCCTGCCAGAAATGAGGGGAGCTTGCCTTTCCATTGGC
TTCTATTCGGGGTGGAAGGAGATTGATGTGCAGAGCAGCTCCCGCTCATCTGACTTTTCACGGTTCACTG
GGAACAATTTCCAAATAGCAAACTCTCTGGCTTCTCTCTCTTTGCAGATGACGGCTGCCCGAAGCCCCCC
GAGATTGCACATGGCTATGTGGAGCACTCGGTTCGCTACCAGTGTAAGAACTACTACAAACTGCGCACAG
AAGGAGATGGTAAGATGTGGACAACTGTCTCCATGCCCTACATACAACCCCCTTCTCTGACATTTCCATG
ATGGGTGGTGCTGAGGTGATTCGCCAGAAAGTTCGTTGCTCTCCTTGGAGCCAGGAGATTTAGATTCTAA
TAAGCGTTTTGTCGCCAGTAGCCATGGCCCTTTGGGCAGACTAACTTTTGTCAGCCTCAAGTTTTCTGTT
TTGTTAAGGGGAGGCGATGCCATGCAGCCTACCTCATGTAAATCTCAGAGTCAGATTTACATCTCCAGCA
GATGTGGGAAAAGAAGGAATGCTGATGATGATGTCACCCTCACCTAGTGAGTCTTGCTGTCCTGGCACTG
CTCTAAGGGCTTTATACTTATTTGCTCACTTAGTCCTCACAGTATCCCTCTGAACAGAGTTTATTGTTTT
CACTTTGCTGATAAGGAAACTGAGGCACAGACAGGTTGAGTATCTTGCCCAAATTCAGGCAGCCTGTAAG
AGGCAGAGTCAGGATTTGAACCCTGAGCCCTCCCTGTACTGCTTGGCTGTGACCGCCATGACCACAGTGT
GTTCTGCTGGGCTTAACTGGTGTCCAGGCACTTGGCTTCCAGCACAGCACTCTTTCCCTTCCTCCTTCTC
ATATTCTCTCTCCTTTCTCCCTTCCTGTCTGCCTCCTTTCTTCTTCTTCTTTTTAATTCTTCTCCTTAAA
TGCCTTCTCACTCTGCTCTGGGTGCAGACTTGACTTTTCCTTTGGCTCATTTCTTGCCTTTTGTTTCAGG
AGTATACACCTTAAATGATAAGAAGCAGTGGATAAATAAGGCTGTTGGAGATAAACTTCCTGAATGTGAA
GCAGGTGGGTGCTGAGCACTGAGCACTTAAGAGAGCAGGCAGGCGTCCAGCGGGGAACGTCCTAGAGGCA
CAGCCTTCCAGTGCGGCTTCCTCTGAGCACACAAGAGCCAGGAGGAGGGATGTGGGAGAACCGCAGCTGG
CCAGGGAGAGACTTAAGCAGTTAGGTGATGACTCCCTAAGGGTCACCAAGGGTCTTGTTCATTGGGGCCT
GAAGGGCACTGGCTGAATCCACTGTCGGCACTGCCCACAGATCAGGAGAGCCTGTGCATACAGAGAGCCT
GCTAGAAAGCCCTGGGTCTAAGGAGAAGCAAGCTCCAGGGAGAACAAGTCAAGGAATGACATAAAATCTT
AATCCATGGAAGCCTAGCAGGAGGCTGGACATGGGCTGGAACTCCTGCTTCTCGTTATTAGGAGGAGCTG
TTGCTCTCTCCTTTCATTCTCAGAACCAGAGGCAAAGACCCAGCCTCTTCTGCTCTTACTGGTGTGGAAA
TGCCAACCTGCCTCGTATTAACTGCACCATCTACAAAATCTGAGCTCCAGCCAGTGCTGCTCTAGATTCA
TCTTTCTTTAGAGAGAATGAATTATTGTAGCCCCTAGCCCTTTCAATGAATTTCAGGGAATTGTGGAAAT
TCCTTTATTGGGATAATTGTTTAAATATAATACAGTTCGCGAGCTTCTATTCGGGGTGGAAGGAGATTGA
TGTGCAGAGCAGCTCCCGCTCATCTGACTTTTCACGGTTCACTGGGAACAATTTCCAAATAGCAAACTCT
CTGGCTTCTCTCTCTTTGCAGATGACGGCTGCCCGAAGCCCCCCGAGATTGCACATGGCTATGTGGAGCA
CTCGGTTCGCTACCAGTGTAAGAACTACTACAAACTGCGCACAGAAGGAGATGGTAAGATGTGGACAACT
GTCTCCATGCCCTACATACAACCCCCTTCTCTGACATTTCCATGATGGGTGGTGCTGAGGTGATTCGCCA
GAAAGTTCGTTGCTCTCCTTGGAGCCAGGAGATTTAGATTCTAATAAGCGTTTTGTCGCCAGTAGCCATG
GCCCTTTGGGCAGACTAACTTTTGTCAGCCTCAAGTTTTCTGTTTTGTTAAGGGGAGGCGATGCCATGCA
GCCTACCTCATGTAAATCTCAGAGTCAGATTTACATCTCCAGCAGATGTGGGAAAAGAAGGAATGCTGAT
GATGATGTCACCCTCACCTAGTGAGTCTTGCTGTCCTGGCACTGCTCTAAGGGCTTTATACTTATTTGCT
CACTTAGTCCTCACAGTATCCCTCTGAACAGAGTTTATTGTTTTCACTTTGCTGATAAGGAAACTGAGGC
ACAGACAGGTTGAGTATCTTGCCCAAATTCAGGCAGCCTGTAAGAGGCAGAGTCAGGATTTGAACCCTGA
GCCCTCCCTGTACTGCTTGGCTGTGACCGCCATGACCACAGTGTGTTCTGCTGGGCTTAACTGGCATCCA
GGCACTTGGCTTCCAGCACAGCACTCTTTCCCTTCCTCCTTCTCATATACTCTCTCCTTTTCCCCTTCCT
TTTTGTCCCCTTTTCCTCTTCCTTTTAGTTCTTCTCTTTAAATGCCTTCTCACTCTGCACGGGGTCTAGA
CTTGACTTCTCCTTTGGCTCACTTCTTGCCTTTTGTTTCAGGAGTGTACACCTTAAACAATGAGAAGCAG
TGGATAAATAAGGCTGTTGGAGATAAACTTCCTGAATGTGAAGCAGGTGGGTGCTGAGCACTTAAGAGAG
CAGGCAGGCGTCCAGCGGGGAACGTCCTAGAGGCACAGCCTTCCAGTGCGGCTTCCTCTGAGCACACAAG
AGCCAGGAGGAGGGATGTGGGAGAACCGCAGCTGGCCAGGGAGAGACTTAAGCAGTTAGGTGATGACTCC
CTAAGGGTCACCAAGGGTCTTGTTCATTAGGGCCTGAAGGGCACTGGCTGAATCCATTGTCTACATCGCC
CACAGATTAGGAGAGCCTGTGCATACAGAGAGCCTGCTAGAGAGCCCTGGGTCTAAGGAGAAGCAAGCTC
CAGGGAGAACAAGTCAAGGAATGACATAAAATCTTAATCCATGGAAGCCTAGCAGGAGGCTGGACATGGG
CTGGAACTCCTGCTTCTCGTTATTAGGAGGAGCTGTTGCTCTCTCCTTTCATTCTCAGAACAAGAGGCAA
AGGCCCAGCCTCTTCTGCTCTTACTGGTGTGGAAATGCCAACCTGCCTCGTATTAACTGCACCATCTACA
AAATCTGAGCTCCAGCCAGTGCTGCTCTAGATTCATCTTTCTTTAGAGAGAATGAATTATTGTAGCCCCT
AGCCCTTTCAATGAATTTCAGGGAATTGTGGAAATTCCTTTATTGGGATAATTGTTTAAATATAATACAG
TTCACCAGCCAGGGCTCAAAAATCTCAGTATTTCCCACTTCCTTTGTTAGAAAAGTGGGAAATAGAGCTT
TTTGTAATGTAAACAATTTAAAAAACAGAATTATTTTAAAACTGCAACTATTGGAAATGAGATCAGCAGG
TGGTAAGGGCAAAGCATTTAAATCTTTCTACTTTACGCAGCAGTGACAGCCGCCCATGCTTTCACCCCTT
TCTCAGATGGAAAGGCTCTTGCACATTTCCACTCACGAGTGTCTTGCTCTCCTTGACAGTATGTGGGAAG
CCCAAGAATCCGGCAAACCCAGTGCAGCGGATCCTGGGTGGACACCTGGATGCCAAAGGCAGCTTTCCCT
GGCAGGCTAAGATGGTTTCCCACCATAATCTCACCACAGGTGCCACGCTGATCAATGAACAATGGCTGCT
GACCACGGCTAAAAATCTCTTCCTGAACCATTCAGAAAATGCAACAGCGAAAGACATTGCCCCTACTTTA
ACACTCTATGTGGGGAAAAAGCAGCTTGTAGAGATTGAGAAGGTTGTTCTACACCCTAACTACTCCCAGG
TAGATATTGGGCTCATCAAACTCAAACAGAAGGTGTCTGTTAATGAGAGAGTGATGCCCATCTGCCTACC
TTCAAAGGATTATGCAGAAGTAGGGCGTGTGGGTTATGTTTCTGGCTGGGGGCGAAATGCCAATTTTAAA
TTTACTGACCATCTGAAGTATGTCATGCTGCCTGTGGCTGACCAAGACCAATGCATAAGGCATTATGAAG
GCAGCACAGTCCCCGAAAAGAAGACACCGAAGAGCCCTGTAGGGGTGCAGCCCATACTGAATGAACACAC
CTTCTGTGCTGGCATGTCTAAGTACCAAGAAGACACCTGCTATGGCGATGCGGGCAGTGCCTTTGCCGTT
CACGACCTGGAGGAGGACACCTGGTATGCGACTGGGATCTTAAGCTTTGATAAGAGCTGTGCTGTGGCTG
AGTATGGTGTGTATGTGAAGGTGACTTCCATCCAGGACTGGGTTCAGAAGACCATAGCTGAGAACTAATG
CAAGGCTGGCCGGAAGCCCTTGCCTGAAAGCAAGATTTCAGCCTGGAAGAGGGCAAAGTGGACGGGAGTG
GACAGGAGTGGATGCGATAAGATGTGGTTTGAAGCTGATGGGTGCCAGCCCTGCATTGCTGAGTCAATCA
ATAAAGAGCTTTCTTTTGACCCATTTCTGTGTTGTGTTCAGTCTTGAGTCTTTTTTATTTGCTCCTTTAT
GGTCCAGGGTAGTCAGAAGGTATAGAGTCTACTGGGAGTATGGCAGAAAACACCCTAAACCCACTGGAAA
TCCCGAAGGTGATACAAACTCTTCCACCTTAGGGAATCATGCTCACTGATTGAGTGCCTATTGAATGCTA
GGTCCCAGAAAGTTAACTGTTGTCCTTGTTTTACAGACAAGGAAACAGAGACTCAGAGATGGTAAGTGAG
TTGCTTAAGGTTACATAGCTATGAAACAGGGAAGCAGAACTTTGAACCCAGGTCTGTTTGATACAAACTC
AGAGGTCCTTTCACTGCATGCTGTTGCCTCCTCAAAGTGAATTAGGAGAAAAGGCATGGGCCTGGTTGAG
GAAGAGGCTAGCTCAAAATGGGATGGGGAAAAAGTGTTTTAACACAGACAGTAATTCAGAGTTTGGGATC
TAACCTACCAGCACTCCAGAAAACAAAAGACAATGATGTAAAGGCAGGATCTCTGGACTTACTGAGTCCA
AATCACAGAATGGCCCATAAATTTGCTAGGTAATTTAGGTTTCCTGGCCATCAGTTCCTGGTCTCTTCCA
TGAAGGACTTGAGCCAGGCGATGGCTAATGTCCCTATCAGCAATAACCTTCTGGTATTCCATAAAGAGTG
GGGAGGTAGAGTCCCTGCATCAGTAGAAGCTGCTTTTCCTACTGGGAACAAACCCACCCTGCACCAGGAA
TCAAATGCAGGCTGTCTGAGCCTCCTCTCCTGATGGCAGGTCATGCTATTAAGGGTCTCCCAGAAGAAAG
GTTCTGGAACTCTGAGAAACAGAAGCTCTTCTAACAGAACTTTTTCTTGCTATAAGCTTTCAATACCACC
AATCACCAGTGCACCTTCTTCTCATGCCAAAATACGGCCCCTCCTTTGTAGGGTGTAAAACGACACTGAT
GTCACTCTCTCTCACCAAGGTCTGGCCATCTCTGTGAAGCTCTCCTCACAGGCACCCTGCAGATTCATCA
ATAAACTTCAACAGGTTAGCGCCAGTAGGCCAATAAGATGCACACTTATATTCTAGAACCTGTGAAAGAA
GTAATAGCCAGAAACTGGAAAATAACAGTCAAACTCTTAAAAAAATGCCTCTCAGAACACAGTCACTTAC
ACCTTTTCCTCTGGTAGTTCTCTTCAGCACCAAGTTTCCTGGAAGAACAAGCTACTCCCCAGTTTTCTTA
AAGAATTGTCTGATTGTATTAAGTAAGTGGCTTCTGGTAAAATAATGGTAAGAGAAAAGCTCTGGCATTC
TCTTGATCACTCATCTTCTCTGCCTTCCTCCCTAGCAACATCTCCTGGCCTCAGCCTGTGGGCTTCAGCA
ACACCAAACTCCATGTTGCTCTCTGACAAGCCCGGCCGTCTCCAGTTCTTGGGCTTTATTTCCTCGGGGG
CCCTCTGCTGAAATTCCCTTCCCCTTGCTGTATTAGCACCTCCTACCCGCCTTCAACATGCAGGCACTGC
CTCCTCTAAGGAGCCATCTGTACTTACGTAGCTGTGAGCATAGGATGGGGCATACAGCAGGCACTTAACA
AATACTTGTGGAATGGAAAGAAACTAAGATAGGATCAAAAATACAAATCAAGATGTGAGAGAAAGACGAT
CAAAAGCCCTCACGAGGGGGTCGGGCGTGATGGCTCACACCTGTAATCTCAACACTTTGGGAGGCTAAGG
CGGGTGGATCACTTGAGGGTGGGAGTTTGAGATCAGCCAGAGCAACATGGAGAAACCTCGTCTCTACTAA
AAATACAAAAAATTAGCCAGGCATGGTGATATATGCCTGTAATCCCAGCTACTCGGGAGGCTGAGGCAGG
AGAATTGCTTGAACCTCGGAGGTAGAAGTTGTGGTGAGCTGAGATCATGCCATTGTACTCCAGCCTGGGC
AACAGGAGTGAAACTCTGTCTCAAAAAAAACAAAAAACAAAAAAGAACACCACGGGAAAATCAAGTGTGA
AGCAAGAGCTCAACTCTTAACAGGGGCAATGTTTGGAGTTTTGTTACTGGAAAAGATAGAGACCTTACCA
GGTCCAAAGTTTGTAGACACAGGAATTACGAAATGGAGAAGGGGGAGAAGTGAGCTAGTGGCAGCATAAA
AAGACCAGCAGATGCCCCACAGCACTGCTCTTCCAGAGGCAAGACCAACCAAGATGAGGTGGGTCCACAG
CTTTCCCTCCTGCCTTTCCTCTGGTTCTTTATTTCAGTCTTTTTTGCATACATTGGTACAGATGCAGAAA
TAGAACAAAGAAACAGGGCAAATGGGCTAAATTATAGTGAACCAAAGGGCTTAGTGTGTTAAATCTTCTC
CTTTTCTGCATCCATAGAAGACAGTGCTGCTGTCTTTCCCAGGAGATAAGATTTACTCTCAGGAGTGTCT
TTTTCCTTGAGGTTACGTTTTTGTCTTTGTAGGGTATGTCATCAGCTCCCGTGGTAGGCTTCCTGGCATT
CGGAATATATTTACTAGCAGATATTTTCCTCTTTAAAAATGTATAATAAGAAAGACTAATAGTAACACAT
TTGAATGACACAATTAATTGACTAGTACTTGGGATACACACTAGTACCTGGGATACATCTAATTAAGACA
CTTAGATCTTAAAAAATAAAAAGACTTTTTGAAATGTTGAAATAATAAGACTAGAAACTTTTTTTTTTGT
GAGACAGAGTCTCACTCTGTCACCAGGATGGAGTGAGTGGCGCGATCTCAGCTCATCGCAACCTCCCCCT
CCCCGGTTCAAGCAATTCTCCTGCCTCAGCCTCCCAAGTAGCTAGGACTACAGGCGTGTGCCACCACGCC
CAGCTAATTTTTGTATTTTTAGTAGCGATGTGGTTCACCATGTTGGCCCGGATGATCTCGATCTCTTGAC
CTCGTGATCCACCTGTCTCGGCCTCCCAAAGTGTTGGGATTACAGGTGTGAGCCACCGCACCCTGCCTAG
AAACTATTTTAATAGAAGCAAATAGTGCCTGAATGGTGGGCGTCTGTTAGCGAGATGGTGAACTGGCAGA
TGGCGCCTGTGTGTCAATGCCCATGGCCACCGTCCTGCTTTCAGACACCAGTTCTCTTCCAGGTAACCTT
CTGGCATTTTGGGGTTTGAGATACCATTTCCTAAAGGTGAATTATCACAAAATACTAGAAATAACCACAT
AAGTGTTTAAAATTATTGTTAAATACAGTAAGAAATTCTTCTTCAAAAGTTTAGCCTGCTTAAGTTTCCT
TGTCCTTTGTTTCCTGCTTTCAAGGCCAGACTTCCTTACTCTCTGTGTTTCCCCTACCCTGGTAAACAAC
CTTCCTGCCAGTCCTTACCCATACAGCCCACATTCCACATCTGCTACCCACTCTGTGATTTACCTCTCCC
GTCGCAATAGCCTCTCCCACCAAAACTGATCTTCCCGCCTTCCCACCAGTGCAACCACATTCCTGCACTT
TGCAAGTTAGCCAACCGGGTTCGGATTGTGCAGTCCAACTCCAGCCAATGGAGTCAGGACACAGTAGCAG
GGACAAGCTGCGTTAGACATAAAAACCTCTGCTTTCCTTTGTTTAGGGTGCTCTCGTGGCAACCAGACTT
ACCAGGAGCTCTATTCTGCAAAAGTAAATTTGCCTTGCTGAGAGACCCTTTGTCCTTTGGCTCAGTGTTG
GTTCTTCTTTGCAGCACCGAGCATTTGTTTCCAACAAATTTGGTGGCCCATACAGGGAAAACATTGTCCT
CCGGGAAAGGGTTCTTTGATCATCCTCTTGAGAGGAGAACACATCCCACTGTCCTTGTTGCGGTGGCCTC
ATGAGTAGGAATCGAGACCCACCTGTCTGATGAATAACCCCAGACTCTCAACAACGTGGGGAGAAAAAGA
CTTGCAACACTATGGTGGCCAGGTAACTCTGTGCGCAGACCAAGGTAAGAAATGTCGCAGGAGTGACAAA
GTACTTCCTTGGTGGTCACTATATTCTGGTGGCTGAAAGTTCATGAATGGTAACAAGTGCTACTGCTGTG
TGGAGTGAATGAGTCCAATCTGTGGGTCTATGGTTACCTCATACGGCTTAGCCTTCTCTGAAGGATCCGG
GNNNNNNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNAGCGTCCTCCTCCCATCTCCAAAACAACCCAACTAAAGT
AACGGTTGTAAAGGTAAAAAATTTAAAGCAGACTATAGCCCTAGAGACAGGATATCAAGATGCAAAGTCT
TGGCTAAAATGGATTAAATATTCCATCTGCACTTTAAACAAATATGAATGTTATGCTTGTGCTCACGGTA
GACCAGAGGCCCAGATTGTCCCCTTTCCACTCAGATGGTCTTCTAACTGACCGGGCATAAGCTGTATGGT
AGTTCTCTTCCAAGATCACACAGCCTGGGGTGACAAATCATGTCAAGCTCTTTCTCTGCTGTTCCCTGAA
GCTCAACACCCTGAGGGTCAGCCCCCGAGGGCCATCAGCTTCCGTTTCCCAATGCCAATTTTACTTTGTG
TCTCTCACAACAGGGAGAAAACTTGGTGTTCCTTGGAGCCTTAATGGGATGCAGTGAGCTTAAGTCCTTC
CAAGAGCTTACCCATCAGTCTGCCCTTAGTCATCCTCGAGCGGATGTATGGTGGTATTGTGGTGGACCAT
TACTGGACACTGCCGAGTAACTGGAGCAGAACATGCACTCTAATTCCATTGGCTATCCCTTTCACCCTGG
CATTTCATCAACCAGAAAAGATAGAAACCAAACACTGTAAAACAAGAGAGGCCCCTCATGGGTCTGTTGA
CTCCCACATTTATATAGATTCCACTGGAGTCCAACGAGGAGTGCCAGATGAATTTAAGGCCCAAAATCAG
ACAACTGCAGGATTTGAATCCACGTTCTTCTGGTGGTTAACTATAAATAAAAATGTGAATTAGATAAACT
ACATTTATTACAACCAATGATGATTTTTTTTTTTTTGACGGAGTTTTGCTCTTGTTGCCCAGGCTGGAAT
GCAACGGCATGATCTCGGCTCACCACAACTTCTGCCTCCTGGGTCCAAGCAATTCTTCTGCCTCGGCCTC
CCGAGTAGCTGGGATTATAAACATGTGCCACCACGCCTGGCTAATTTTGTATTTTTAGTAGAGACAGGGT
TTCTCCATGATGGTCAGGCTGGTCTCAAACTCCCGACCTCAGGTTATCTGCCTGCCTCGGCCTCCCAAAG
TGCTGGGATTACAGGCATCAGCCACTGTGCCCAGCCTATGATTTGTTAATTATACTAGAGATGCTCTTAA
AGGGACAGCTGAACAATTAAGACCCACCAGTCAAATGGCCTGGGAAAATAAAATAGCATTAGACATGATA
CCAGCGAAAGAAGGTGGAGATTGTGTCATTATCGGAACCCGATGCTATACTTTTATCCCTAATAATACTA
CTCCAGGTGGGATCACACCAAAAGCACTACAAGGTCTTACCGCCTTATCAAATGAGCTAGCCAAAAATTC
TGGACTAAACGACCCCTTCACAAATTTAATGGGAAATTCTTTGGCAAATGGAAGGGATTTATGTCCTCAA
TCCTCATGTCTCTTGCCATCAGAATAGGGTTGCTTATTCTTGTAGGATGCTGTGTCATACCCTGTGCCCT
AGGATTAATACAAAAGCTCATTGAAACAGCTCTCCCCAAAACCTCCCTCAATCCTCCTCCTCCTCGTTCC
AGTGAGCTTTTTCTTTTAGAAGACCCAAGTAGAATAACAGAGCCAAATTATGTTAAAAGGGTTTAAGAAA
AAATACTGTAAAATATTTAACAGGGGGATTGTTAAATACAGTAAGAAATTCTTCTTCAAAGGTTCAGCTT
GCTTAAGTTTCCTTGTTCTTTGCTCCCTGCTTTCAAGGCCAGACTTCCTTACTCTCTGTGTTCTCCCTGC
GCTGGTAAACAACCTTCCCGCCAGTCGTTATCTATAGAGCCCACATTCCACATCTGCTACCCACTCTGTG
AATTACCCCTCCCATCACAATGGCTTCTCCCGGCAAAACTGATCTTCCTGCCTTCCCAGCAGTGTAATCA
CATTCCTGCACTTTTCAAGTTAGCCAACCGGGTTCAGCTTAGATTGTACGGTCCAACTCTAGTCAATGGA
GTCAGGATACAGTAGCAGGGACAAGCTGCGTTAGACATAAAAACCTCTGCTTTCCTTTGTTCGGGGTGCT
CTCGTGGCAACCGAACCTATGAGGAGCAACCTTATGCAAAAGTAAATTTGCCTTGCTGAGAGACCCTTTG
TCCTTTGTCTCAGTGCTAGTTCTTCTTTGCAGCACCAAGCATTTGTTTTCAACAATTATATATTTAAGGA
ATCTTTGATTATGGAAAATTTCAAGAAGTAGAGGGAATAGCATACTAAGCCCTCTGTTCCCATCTTTGAG
TTATCTACATTTTATTAGAAGTATCTTTGGGGTGACTGAGGGAGGTTTCTCTTTCCTGGAGGGCTTCTGA
ATTATTGCCAATGTACCTTCCTGACTGCAGCCAGAAACTGAGGCCCCTATACCTATGTACCTTTCTATCC
CTCTCTGAAGCTTCTGCAGAATTCCCAGCAAGACAGAGCTTGCTGGAAGCTTGGTATGCTCAGAAGCAGC
TAAAGCGTGTATGTGGGGCGGAGGGTGGGGGCAACTTCTTGGTCCTAGCACTTCCATATATTGATTTTCT
TTTCTGGCTGCTTAGTGGGAGGAGTGTGTGTGTATGCATGTGTGTGTGTGCGTGTGTGTGTGTGTGTACA
TGCCTGTGTGTGTGGATGCATGCATGTGCTGTGAAGCAGGGAGACCAGCTTTCCGTTCCTTCTTGTTTTC
TCTCTGCAGTGACCTGGGAGCTGTCATTTCCCTCCTGCTCTGGGGACGACAGCTTTTTGCACTGTACTCA
GGCAATGATGTCACGGATATTTCAGGTCAGTCTTTGAGTTGGGTAGGAGCATGCATCCCTGGCACTGCCA
CATCCCACTCTGACTCTCTCGGGTCTGCATTCTTTCTTTGAGAACACACAGTTCCCCATTCTTATCCTGA
CCTCTGGGCTTTCAGGACTGCCAAGAACATTGGGGATCCTGCCAGAAATGAGGGGAGCTTGAGCTTTCGT
TGGCTTCTATTTGGGGTAGAAGGAGATTGATATGCAGAGCAGCTTCCACTCATCTGACTTTTCATGGGTC
TCTGGGAACAATTTCCAAATGGTAAACTCTCTGGCTTCTCTCTCTTTGCAGATGACCGCTTCCCGAAGCC
CCCTGAGATTGCAAATGGCTATGTGGAGCACTTGTTTCGCTACCAGTGTAAGAACTACTACAGACTGCGC
ACAGAAGGAGATGGTAAGACCTGGACAACTATCTCCTGTGCTCTACCTACAACCCCTGCTCTGACATTTC
CATGATGGGTGGTGCTGAGGTGATTTGCCAGAAAGTTCGTTGCTCTCCTTGGAGCCAGGAGATTTAGATT
CTGATAAGCGTTTTGTCGCCAGTAGCCATGGCCCTTTGGGCAGACTAACTTTTGTCAGCCTCAAGTTTTC
TGTTTTGTTAAGGGGAGGTGATGCCATGCAGCCTACCTCATGTAAATCTCAGAGTCAGATTTACATCTCC
AGCAGATGTGGGAAAAGAAGGAATGCTGATGATGATGTCACCCTCACCTAGTGAGTCTTGCTGTCCTGGC
ACTGCTCTAAGGGCTTTATATTTATTTGCTCACTTAGTCCTTACAGTTTACCTCTGAACAGAGGTTATTA
TTCTCACTTTGCCGATAAGGAAACAGAGGCACCGACAGGTTGAGTATCTTGCCCAAATTCAGGCGGCCTG
TAAGAGGCAGAGTCAGGATTTGAACCCTGAGCCCTCCCTGTACTGCTTGGCTGTGACCGCCATCACCACA
GTGTGTTCTGCTGGGCTTAACTGGTGTCCAGGCACTTGGCTTCCAGCACAGCACTCTTTCCCTTCCTCCT
TCTCGTATTCTCTCTCCTTTCTCCCTTCCTGTCTGCCTCCTTTCTTCTTCTTCTTTTTAATTCTTCTCCT
TAAATGCCTTCTCACTCTGCTCTGGGTGCAGACTTGACTTTTCCTTTGGCTCATTTCTTGCCTTTTGTTT
CAGGAGTATACACCTTAAATGATAAGAAGCAGTGGATAAATAAGGCTGTTGGAGATAAACTTCCTGAATG
TGAAGCAGGTGGGTGCTGAGCACTGAGCACTTAAGAGAGCAGGCAGGCGTCCAGCGGGGAACGTCCTAGA
GGCACAGCCTTCCAGTGCGGCTTCCTCTGAGCACACAAGAGCCAGGAGGAGGGATGTGGGAGAACCGCAG
CTGGCCAGGGAGAGACTTAAGCAGTTAGGTGATGACTCCCTAAGGATCACCAGGGTCTTGTTCATTGGGG
CCTGAAGGGCACTGGCTGAATCCACTGTCGGCATTGCCACAGATCAGGAGAGCCTGTGCATACAGAGAGC
CTGCTAGAGAGCCCTGGGTCTAAGGAGAAGCAAGCTCCAGGGAGAACAAGTCAAGGGATAACATAAAATC
TTAATCCATGGAAGCCTAGCAGGAGGCTGGACATGGGCTGGAACTCCTGCTTCTCGTTATTAGGAGGAAC
TGTTGCTCTCTCCTTTCCATCTCAGAACCAGAGGCAAAGGCCCAGCCTCTTCTGCTCTTACTGGTGTGGA
AAGGCCAGCCTCTTCTGCTGTGAGTGGTGTGGAACTACCAACCTGCCTCGTATTAACTGCAACATCTATA
AAGTATGAGCTCCAGCCAATGCTGCTCTAGATTCCTCTTTCTTCAGAGATGATGAATTATTGTAGCTCCT
AGCCCTTTCTTTTTTCTTTCTTTCTTTTTTTTTTTTTTTGAGACAGAGTTTTGCTCTCGTCGCCTAGGCT
GGAGTGCAGTGGTGCTAATTCCTGACCTCAGGTGATCCAACTGCCTCAGCCTCCCAAAGTGCTGGGATTA
CAGGCGTGAGCCACCGCATCTGGCCCCTAGCCCTTTCAATGAATTTCAGGGAATTGTGAAAATTCCTTTG
TTGAGATAATTGTTTAAATATAATATAGTTCACCAGCCAGGCCTCAAAAAACTCAGTATTTCTCATTTCC
TTATTTAGAAATAGAGCTTTTTGTAATGTAAACAATTAAAAAAATTATTTTAAAACTGCAACTATTGGAA
ATGAGATCAGCAGGTGGTAAGGACAAAGCATTTAAATCTTTCCAGTTTATGCAGCAGTGACAGCCGCCAA
TGCTTTCACCCCTTTCTCAGATGGAAAGGCTCTTGCACATTTCCACTCACGAGTGTCTTGCTCTCCTTGA
CAGTATGTGGGAAGCCCAAGAATCCGGCAAACCCAGTGCAGCGGATCCTGGGTGGACACCTGGATGCCAA
AGGCAGCTTTCCCTGGCAGGCTAAGATGGTTTCCCACCATAATCTCACCACAGGGGCCACGCTGATCAAT
GAACAATGGCTGCTGACCACGGCTAAAAATCTCTTCCTGAACCATTCAGAAAATGCAACAGCGAAAGACA
TTGCCCCTACTTTAACACTCTATGTGGGGAAAAAGCAGCTTGTAGAGATTGAGAAGGTGGTTCTACACCC
TAACTACCACCAGGTAGATATTGGGCTCATCAAACTCAAACAGAAGGTGCTTGTTAATGAGAGAGTGATG
CCCATCTGCCTACCTTCAAAGAATTATGCAGAAGTAGGGCGTGTGGGTTACGTGTCTGGCTGGGGACAAA
GTGACAACTTTAAACTTACTGACCATCTGAAGTATGTCATGCTGCCTGTGGCTGACCAATACGATTGCAT
AACGCATTATGAAGGCAGCACATGCCCCAAATGGAAGGCACCGAAGAGCCCTGTAGGGGTGCAGCCCATA
CTGAACGAACACACCTTCTGTGTCGGCATGTCTAAGTACCAGGAAGACACCTGCTATGGCGATGCGGGCA
GTGCCTTTGCCGTTCACGACCTGGAGGAGGACACCTGGTACGCGGCTGGGATCCTAAGCTTTGATAAGAG
CTGTGCTGTGGCTGAGTATGGTGTGTATGTGAAGGTGACTTCCATCCAGGACTGGGTTCAGAAGACCATA
GCTGAGAACTAATGCAAGGCTGGCCGGAAGCCCTTGCCTGAAAGCAAGATTTCAGCCTGGAAGAGGGCAA
AGTGGACGGGAGTGGACAGGAGTGGATGCGATAAGATGTGGTTTGAAGCTGATGGGTGCCAGCCCTGCAT
TGCTGAGTCAATCAATAAAGAGCTTTCTTTTGACCCATTTCTGTGTTGTGTTCAGTCTTGAGTCTTTTTT
ATTTGCTCCTTTATGGTCCAGGGTAGTCAGAAGGTATAGAGTCTACTGGGAGTATGGCAGAAAACACCCT
AAACCCACTGGAAATCCCGAAGGTGATACAAACTCTTCCACCTTAGGGAATCATGCTCACTGATTGAGTG
CCTATTGAATGCTAGGTCCCAGAAAGTTAACTGTTGTCCTTGTTTTACAGACAAGGAAACAGAGACTCAG
AGATGGTAAGTGAGTTGCTTAAGGTTACATAGCTATGAAACAGGGAAGCAGAACTTTGAACCCAGGTCTG
TTTGATACAAACTCAGAGGTCCTTTCACTGCATGCTGTTGCCTCTTTAAAGCGAATTAGGAGAAAAGGCA
TGGGCCTTGGTGAGGAAGAGGCTAGCTCAAAATGGGATGGGGAAAAAGTGTTTTAACACAGACAGTACTT
CAGAGTTTGGGATCTAACCTACCAGCACTCCAGAAAACAAAAGATGATGATGTAAAGGCAGGATCTCTGG
ACTTGCTGAGTCCAAATCATGGACTTGGCCCAAACATTTGCTAAGTAATTTAGGCTTCCTGGCCATTAGT
TCCTGGTCTCTTCCATGAAGGACTTGAGCCAGGGTAACGTCCCTATCAGCAATAAGCTTCTGGGACTCTG
TAAAGAGTGGGGAGGTAGAGTCCCTGCCTCAGTAGAAGCTGCTTTTCCTACTTGGAACAAACCCACCCTT
CACCAGGAATCAAAAGCAAGCTGTCTGAGCCTCCTCTCCTGATGGCAGGTCATGCTATTAAGGGTCTCCC
AGAAGAAAGCTTCTGGAGCTCTGAGAAACAGAAGCTCTTCTAACAGAACTTTTTCTTGCTATAAGCCTTC
GACACTGATGTCACTCTCTCTCACCAGGGTCTGGCCATCTCTGTGAAGCTCTCCTCACAGGCACCCTGCA
GATTCATCAATAAACTTCAACAGGTTAGCGCTAGTAGGCCAATAAGACCTATATTCTAGAACCTGTGAAA
GAGATAATAGCCAGAAACTGGCAAACACCAGTCAAACTCTTTAAAAAAATGCCTCTTAGAACACAGTCAC
TTACACATTTTCCTAGATCTCTTCAGCACCAAGTTTCTTGGAAGAACAAGGCACTCTGCAGTTTTCTTAA
AGAATTGTCTGATTGTATTAAGTGGCTTCTGGTAAAACAACAGTAAGAGAAAAGCTCTGGCATTCTCTTG
ATCACTCATCTTCTCTGCCTTCCTCCCCAGCACCACCTCCTGGCCTCAGCCTGTGGGCTTCTGCAAAGTC
AGGTATCAGGACTAATTAGCTAGCATCAAATTCCTGTTAATTCCAGAAGCAGGGACAAACCTGTTGGGGA
GGGACTTAATGACAAAATTAGGTATAGGTCTATACGTTAATCAAGGAAAATTCTCAACTTCCTTAAACTT
ACTTACCACCCTAGAGGAGAGCCAAATCCACTCTGATGACGTTTGGTCATAGGAAGGAAATTGGGGAAAA
TTACAAGTTTCTCCAATCCATGTAAAATTAAGAAATCCTGGGGAAGTTGTAAAGAGAAAACAGTACCCTA
TTCCCTTGAAAGGCAGAATAGGTTTATAACTTATAATCGAAGGTCTCCTTCAGGAAGGACTTCTTGAACC
CCATATGTCCTCTTACAACACTCCAATATTGCCTGTAAAAAAGTCGGATGGGTCATAGCGGCTACTGCGA
GACCTTCGAGCCATTAACCAAATAGTCCAGACCACCCACCCTGTCATTCCTAACCCTTACACCATTATCA
AGCAAGATCCCTCATGACTGCCAGTGGTTCACAGTAATAGACCTCAATGATGACTTCTGGGCTTGCCCCT
TAGCTGTGGATAGCTGGGACATATTTGCTTTTGAATGGGAAGACCCTCATTCCAGTTGGAAGCAATAGTA
GTGATGGACAGTTCTACCCCAAGGGTTTACAGACTCTCCAAACCTATTAGTCAAAGTTTGGAACAAGTCC
TAGAGAATTTCCCTCTTTCATCATCCTTATGTCTACTCCAATATGTGGATGACCTGCTCATTTCCAGAGG
CACCAAAGACCAAGTAACCGCAATTTTAATTAGCTTTCTAAATTTCCTAAGGGAACAATGGTTACGGGTC
TCAAAAAGTAAACTCCAGCTTGTAGAACCTGAGGTAAAACACATGGGGCACTTAATAAGCAAAGGTAAGC
GGAAGATAGGGCCCGAACAAATGGAAGGGATCATATCCCTGCCACTGCCTGAGACAAAACAAGAACTTAG
AAAATTCTTCAGGCTAGCTGGAAATTATTGCCTGTGGATTGACTCTTATTCTTAAAAACAAAACCTCTAT
ATCTAAAACTTACCCAAGAAGGGCCTGACCCCCTTCTTTGGACCCCACAAGAAGTCCAGCAAGTTGAGGA
ACTAAAACATCTACTTATAACTGCCCCTGTCTTAGCTCTGCCATCCTTAGAACAGCCATTTCACTTTTTT
GTTAATATAAGCAATGGGGCAGCTATAGGAGTACTCACTCAAAAGCATGGGGCCATTGCCAGCCCACAGC
CTTTCTGTCAAACATTCTTGACGTGGTAACCTGTGGGTGGCCCGAATGTATCCAATCTACAGCAGCAACT
GTTTTATTGACAGAGGAAAGTAGAAAAATAACCTTTGGGGGAAGTCTCATCCTAAGCACACCCCATCAGG
TTAAAACCATTCTTAGCCAAAAAGCAGGGAGATGGCTTACAGATTCAAGAATCCTAAAATATGAAGCTAT
CCTATTAGAAAAAGGTGATTTAACCCTAACCACTGACAATGTGCTCAACCCCGCCACCTTCCTTTTTTTT
TTTTTTTTTTGAGATGGAGTTTCACTCTTATTGCCCATGCTAGAGTGCAATGGCACAATCTCGGCTTATC
GTAATCTTTGCCTCCTGGGTTCAAGCAATTCTCCTGCCTCAGCCTATTAAGTAGCTGGGATTACAGGCAT
GCTACCATGCCTGGCTAATTTTTTTGTATTTTTAGTAGAGATGGGGTTTCTCCATGTTGGTCAGGCTGGT
CTTGAACTCCTGTCCTCAGGTGATCTGCCTGCCTTGGCCTCCCAAAGTGTTGGGATTACAGGCGTGAGCC
ACTGTGCCCGGCCTACCCCGCCACCTTCCTGACGGAAATCCAAACGCTGAAGACCCCAAGCACAAATGTT
TGAACTTAATCATCAAACTAAAGTAAGGCCTGATCTAAGTGAGACCCCTTTAAAAACAGGGCACCATCTC
TTTGTAGACAGCTCCTCTCGGGTAATTGAAGAGAAAAAACATAATGGCTACTCAGTGGTTGATGGAGAAA
CTCTCACAGAAGTAGAATCAGGGAGACTTCCCAATAACTGGTCTAACCAAACATGTGAGTTGTTCGCACT
AAATCAAGCCTTAAAATAGCTGCAGAATCAAGAAGGGACTATTTACACCAATTCCAAATATGCTTTTGGA
GTGGCCCATACCTTTGGGAAGATTTGGACTGAACAAGGCCTTATTACTAGCAAGGGCCAAAACCTTGTCC
ACAAAGAGTTAATCATGCAAGTATTAGAAAACCTTCAGCTACCAGAGGACATAGCCATTGTTCATGTCCC
AGGACACCATAAAGGTCTCTCTTTCAAGAGTTACTTCCCAAGAAGCACCCATTTTCCACTGAACCCCTTG
TCTCCCTTCCCCATCTGCAACCCCCATCTTCTCCCAAGCAGACAAAGAAAAATTAAAGAAAATAGGGGCT
AACAAAAACTCAGAAGAGAAACGGATACTGCCAGATAACAAAGAAATGTTGTCCAAACCCCTAATGACAG
AAATCTTAACACAACTTCACCAAGGGACCCATTGGGGTCCCCAAGCCATGTGTGATGCAGTCCTTAGAAC
CTATGGGTGTACAGGGATTTATACCCTCACCAGGCAAGTCACAGACAGTTGTATGGTATGCAGAAAAACC
AATAAACTTTAAGAAAACTGCCCTTCGGCGGAAGAAATCCTGGGCTAAGGCCATTTCCAAAGTGTCCATA
TTGACTTATACCGAAATGCCCCAAATAGGCTGCCTCAAATGTTTGTCAGTTATAGTGGACCATCTCACTC
ACTGGGTGGAAGCCATTCACCTGCCAAGTGCAACTGCCAATAATGTAGTTAAAGTACTAATAGAAGACTC
CATACCCAGATTCAGATTAATAGAGAACATTGACTCAGACAATGGAACCCATTTTACTGCACAGGTCATT
AAAGGATTAACCCAGACACTAGAGATAAAATGGGAATATCATACTCCCTGGCACCCACCCTCATCAGGAA
AAGCAGAAAGAATGAATCAAAATAAATAAATCACCTAACTAAACTAATCTTGGAAACCCGACTGCCATGA
ACTAAGTACCTCCCTGTCGCCTTACTAAGAATTCTCATGTTTGTCCTTATCATCGATAAG
(hMxA) (Accession No. NP_001171517)
SEQ ID NO: 10
MVVSEVDIAKADPAAASHPLLLNGDATVAQKNPGSVAENNLCSQYEEKVRPCIDLIDSLRALGVEQDLAL
PAIAVIGDOSSGKSSVLEALSGVALPRGSGIVTRCPLVLKLKKLVNEDKWRGKVSYQDYEIEISDASEVE
KEINKAQNAIAGEGMGISHELITLEISSRDVPDLTLIDLPGITRVAVGNQPADIGYKIKTLIKKYIQRQE
TISLVVVPSNVDIATTEALSMAQEVDPEGDRTIGILTKPDLVDKGTEDKVVDVVRNLVFHLKKGYMIVKC
RGQQEIQDQLSLSEALQREKIFFENHPYFRDLLEEGKATVPCLAEKLTSELITHICKSLPLLENQIKETH
QRITEELQKYGVDIPEDENEKMFFLIDKVNAFNQDITALMQGEETVGEEDIRLFTRLRHEFHKWSTIIEN
NFQEGHKILSRKIQKFENQYRGRELPGFVNYRTFETIVKQQIKALEEPAVDMLHTVTDMVRLAFTDVSIK
NFEEFFNLHRTAKSKIEDIRAEQEREGEKLIRLHFQMEQIVYCQDQVYRGALQKVREKELEEEKKKKSWD
FGAFQSSSATDSSMEEIFQHLMAYHQEASKRISSHIPLIIQFFMLQTYGQQLQKAMLQLLQDKDTYSWLL
KERSDTSDKRKFLKERLARLTQARRRLAQFPG
(hMxA) (Accession No. NM_001178046)
SEQ ID NO: 11
ATCTGATTCAGCAGGCCTGGGTTCGGGCCCGAGAACCTGCGTCTCCCGCGAGTTCCCGCGAGGCAAGTGC
TGCAGGTGCGGGGCCAGGAGCTAGGTTTCGTTTCTGCGCCCGGAGCCGCCCTCAGCACAGGGTCTGTGAG
TTTCATTTCTTCGCGGCGCGGGGCGGGGCTGGGCGCGGGGTGAAAGAGGCGAAGCGAGAGCGGAGGCCGC
ACTCCAGCACTGCGCAGGGACCGGAATTCTGTGGCCACACTGCGAGGAGATCGGTTCTGGGTCGGAGGCT
ACAGGAAGACTCCCACTCCCTGAAATCTGGAGTGAAGAACGCCGCCATCCAGCCACCATTCCAAGCTTAC
TTTGCAAAGAAGGAAGATGGTTGTTTCCGAAGTGGACATCGCAAAAGCTGATCCAGCTGCTGCATCCCAC
CCTCTATTACTGAATGGAGATGCTACTGTGGCCCAGAAAAATCCAGGCTCGGTGGCTGAGAACAACCTGT
GCAGCCAGTATGAGGAGAAGGTGCGCCCCTGCATCGACCTCATTGACTCCCTGCGGGCTCTAGGTGTGGA
GCAGGACCTGGCCCTGCCAGCCATCGCCGTCATCGGGGACCAGAGCTCGGGCAAGAGCTCCGTGTTGGAG
GCACTGTCAGGAGTTGCCCTTCCCAGAGGCAGCGGGATCGTGACCAGATGCCCGCTGGTGCTGAAACTGA
AGAAACTTGTGAACGAAGATAAGTGGAGAGGCAAGGTCAGTTACCAGGACTACGAGATTGAGATTTCGGA
TGCTTCAGAGGTAGAAAAGGAAATTAATAAAGCCCAGAATGCCATCGCCGGGGAAGGAATGGGAATCAGT
CATGAGCTAATCACCCTGGAGATCAGCTCCCGAGATGTCCCGGATCTGACTCTAATAGACCTTCCTGGCA
TAACCAGAGTGGCTGTGGGCAATCAGCCTGCTGACATTGGGTATAAGATCAAGACACTCATCAAGAAGTA
CATCCAGAGGCAGGAGACAATCAGCCTGGTGGTGGTCCCCAGTAATGTGGACATCGCCACCACAGAGGCT
CTCAGCATGGCCCAGGAGGTGGACCCCGAGGGAGACAGGACCATCGGAATCTTGACGAAGCCTGATCTGG
TGGACAAAGGAACTGAAGACAAGGTTGTGGACGTGGTGCGGAACCTCGTGTTCCACCTGAAGAAGGGTTA
CATGATTGTCAAGTGCCGGGGCCAGCAGGAGATCCAGGACCAGCTGAGCCTGTCCGAAGCCCTGCAGAGA
GAGAAGATCTTCTTTGAGAACCACCCATATTTCAGGGATCTGCTGGAGGAAGGAAAGGCCACGGTTCCCT
GCCTGGCAGAAAAACTTACCAGCGAGCTCATCACACATATCTGTAAATCTCTGCCCCTGTTAGAAAATCA
AATCAAGGAGACTCACCAGAGAATAACAGAGGAGCTACAAAAGTATGGTGTCGACATACCGGAAGACGAA
AATGAAAAAATGTTCTTCCTGATAGATAAAGTTAATGCCTTTAATCAGGACATCACTGCTCTCATGCAAG
GAGAGGAAACTGTAGGGGAGGAAGACATTCGGCTGTTTACCAGACTCCGACACGAGTTCCACAAATGGAG
TACAATAATTGAAAACAATTTTCAAGAAGGCCATAAAATTTTGAGTAGAAAAATCCAGAAATTTGAAAAT
CAGTATCGTGGTAGAGAGCTGCCAGGCTTTGTGAATTACAGGACATTTGAGACAATCGTGAAACAGCAAA
TCAAGGCACTGGAAGAGCCGGCTGTGGATATGCTACACACCGTGACGGATATGGTCCGGCTTGCTTTCAC
AGATGTTTCGATAAAAAATTTTGAAGAGTTTTTTAACCTCCACAGAACCGCCAAGTCCAAAATTGAAGAC
ATTAGAGCAGAACAAGAGAGAGAAGGTGAGAAGCTGATCCGCCTCCACTTCCAGATGGAACAGATTGTCT
ACTGCCAGGACCAGGTATACAGGGGTGCATTGCAGAAGGTCAGAGAGAAGGAGCTGGAAGAAGAAAAGAA
GAAGAAATCCTGGGATTTTGGGGCTTTCCAGTCCAGCTCGGCAACAGACTCTTCCATGGAGGAGATCTTT
CAGCACCTGATGGCCTATCACCAGGAGGCCAGCAAGCGCATCTCCAGCCACATCCCTTTGATCATCCAGT
TCTTCATGCTCCAGACGTACGGCCAGCAGCTTCAGAAGGCCATGCTGCAGCTCCTGCAGGACAAGGACAC
CTACAGCTGGCTCCTGAAGGAGCGGAGCGACACCAGCGACAAGCGGAAGTTCCTGAAGGAGCGGCTTGCA
CGGCTGACGCAGGCTCGGCGCCGGCTTGCCCAGTTCCCCGGTTAACCACACTCTGTCCAGCCCCGTAGAC
GTGCACGCACACTGTCTGCCCCCGTTCCCGGGTAGCCACTGGACTGACGACTTGAGTGCTCAGTAGTCAG
ACTGGATAGTCCGTCTCTGCTTATCCGTTAGCCGTGGTGATTTAGCAGGAAGCTGTGAGAGCAGTTTGGT
TTCTAGCATGAAGACAGAGCCCCACCCTCAGATGCACATGAGCTGGCGGGATTGAAGGATGCTGTCTTCG
TACTGGGAAAGGGATTTTCAGCCCTCAGAATCGCTCCACCTTGCAGCTCTCCCCTTCTCTGTATTCCTAG
AAACTGACACATGCTGAACATCACAGCTTATTTCCTCATTTTTATAATGTCCCTTCACAAACCCAGTGTT
TTAGGAGCATGAGTGCCGTGTGTGTGCGTCCTGTCGGAGCCCTGTCTCCTCTCTCTGTAATAAACTCATT
TCTAGCAGACAAAAAAAAAAAAAAAAAA
(hMxA) (Accession No. NG_027788)
SEQ ID NO: 12
ATCATTCTTCACAGAACTAGAAAAAACATTCTTAACATTCATATGGAACCAAAAAAGGAGCCCGCATAGC
CAAAGCAAAACTAAGCAAAAAGAATAAACCTGGAGGCATCATATTACCCAACTTCAAACTATACTACAAG
GCTATAGTCCCCAAAACAGCATGATACTGATATAAAAATAGGCACATAGACCAATGGAACAGAATACATA
ACCCAGAAATAAAGCCAAATACTTATAGCTGACTGAACTTTGACAAAGCAAACAAAAGCATAAAGTGGGG
AAAGGACACCCTATTCAACACATGGTGCTGGGATAATTGGCAAGCTACATGTAGAAGAATGAAACTGGAT
CCTCATATCTTGCCTTATACAAAAGTCAACTCAAGATGGATCAAAGACTTAAATCTAGGACCTAAAACCA
TAAAAATTCTAGAAGATAACATTGGAAAAACCCTTGTAGACATTGGCTTAGGCAAAGAGTTCATGACCAA
GAACCCAAAAGCAAATGCAACAAAAATGAAGACAAATGGATCGGACTTAATTAAGCTGAAAAGCTTCTAC
ACAGCAAAAGAAATAATTAGCAGAGTAAATAGACAACATACAGAGTGGGAGAAAATTTTGCAAACTATGC
AGCCAACGAAGGGCTAATATCCAGAATCTACAAGGAACTCAAACAAATCAGCAAGAAGAAAACAAATACT
CCCTCACAAAGTGGGCTAAGGATGTGAATAGACAATTCTCAAAAGAAGATATACAAATGGCCAACATATA
AAAAAAAATCAACATCACTAATTATTAGGAAAATGCAAATCAAAACCACAATGGGATACCACCTTACTCC
TGCAAGAAGGGCCACAATCAAGAAATCAAAAAATAATAGATGTTGGCATTAATGTGGTGAAAGGGATCAC
TTTTACACTGCTGGCAGGAATGTAAACTAGTACAACCAATGGAAAACAGTAGAGTTTCCTTAAATAACTG
AAAGTAGATCTACCATTTGATCCAACAATCCCACTAGTGGGTATTTAGCCAGTGGAAAATAAGTCATTAT
ATGAAAAAGACACTTGCACACACATGTTTATAGCAGCACAATTCCCCAAGTGCCCACCAATCAACAAGTG
GACAAAATGTTATATATATATGTGTATATATATGTATATATATATAATGTGATATATATGTATATATATG
TATATATATATAATGTGATATATATGTATATATATAATGTGATATATATGTATATATAATAATGTGATAT
ATATGTGTATATATAATGTGATATATATGTATATATATATAATGTGATATATATGTATATATATATAATG
TGATATATATGTGTGTGTGTATATATATAATGTGATATATATATGTGAATACTACTACTACTACTACTAC
TACTACTAATACTCAGCCATAAAAAGGAATGAAATAATGGCATTCACAGCAACCTGGATGGAGTTGGAGA
CCATTATTCTAAGTGAAGTAACTCGGGAATAAAAACCAAACATTGTATATTCTCACTCATAAGTGAGATT
ATACAAGCTATGAGGATGCAAAGTCATAAGAATGATACAATAAACATTGGGAACTCAGGGGGAAGGGTGA
GGGTGGGGTGAGGGATAAAAGACTAGACATCGGGTACAGTATACACTGCTTGGGTGATGGGTGCACCAAA
ATCCCAGAAATCACGATTAAAGAACTTGTCAATGTAACCAAACACCACCTGTTCCTCAAAAACTATTGAA
ATAAAAAATAAATACATAAAAGAAGTTGAGGTTAAATTAATCTAAGTAGGGAGTGCATTTGGGCCAAGCT
TGAGGATTGCAACGTTGGAGCACAGATTTAAGTTGCCCTGAATCTACACTCTGATAAGTGGCATTTATAA
GTGGATTTTTAAAGGCAAAAGGGGGAGACAGGGAGAGGGCTGATACAAAGCTGTTTGTCAGGAATTCTTA
TTGGTTTACAGAAATAACATTGATAAGTGCTTATTAGTCCATTTTCATGCTGCTGATAAAGACATACCTC
AGACTGGGCAATTTACAAAAGAAAGAGGTTTATTGGACCTATAGTTCCACATGGCTGAGGAGGCCTCACA
ATCATGGTGGAAGGCAAGGAGGAGCAAGTCACATCTTACATGGATGACAGCAGGAAAAAAGAGAGAGCTT
GTGCAGAGAAACTCCCTTTTTTAAAACCATCAGATCTTGTGAGACCCGTTCAGGATCATGAGAACAGCAC
AGGAAAGACCCACCCCCAAGATTCAATCATCTCCCACCGGGTCTCTCCCACAACATGAGTGAATTATGGG
AGCTACAGGATGAGATTTGGGTGAGGACACAGAGCCAAACCATATCAGAGTGATTGACTATACATTGTTA
AGCTACAGGGTGTGGATTACAGCGTCCAGTGTGGCATTATTAGGTTAATCCATAGCTATTAATACCTGTG
GCAATAGTAAGAGGTTTCAAGAGATGAACACACAGCTGAAAGTGGGGAGTAGGGAGTGGTTGCTGTCTTA
TTTTAATGTCTCTCTGGGCCTGATAATTTTAAAAGCCTTGCATTCTTCGGATACAAGTTCCTTTCTTTTT
TTCAAGAACAACAGAGAAAGGCTGCCTGGCTACCTTTACCTGCAGCACCTCTGATAGTGGCTGCTTGGCA
GCTGGTTAGGTAAACCCAATTTCTACTGGATAAACTTCATGGACATTCACCCATGTTAGTACTGGGGAAT
GCTCTGAAATAGCAAACTTGTCCATACAGCTCCCTTGCTGCTCAGAGGGTCTGGAGAGGAACAGCAGAGG
CCCAGCCCCAAATACTTCTTGTACCGGCTTCTATTATGAGAGGTGATGGCACCTTTAGGCACTTATATTT
CAGGTAGAAGGAGCAGGAATAAAGAAGAGAGGGCCACAATCTGAAATATCAGTTCTGGATCTCCCTGGGG
GCAAGACACTGGCCCTTAGCTGAGCTATTCTGTCATCACTGTGAGCCCTCATGCTCGACCTCACCGGAGC
ACCCCTCGATCATGTGCTGAATGCCTAGCACGCGCCTGGCACTGTGGGGTACAGAAATCTAGAGCACAGA
GCTGAGTTTTCAGGGCTTAGCTGTCCTCACATCTGTGACACAAACAAGGATCAAGGAAAGTTTTGTTTTT
GTTATTTTTATTTTTTTTTAAGTTGGGGCCTTACTCTGTAGCCCAAGCTAGAGTGCAGTGGTGCAAACAT
AGCTCATTGCACCCTCGAACTCCTGGGCTCAAGTCATCCTCCCACCTTGGCCTCCAGAGTAGCTGGAAGC
CCAGCTCAAGGAAAGCATTAACATGTTCTACTTTTCACCATAACTCCTGTGTATTGGGAAATGGAAAATG
AGCTTTTGAAGTGGCCTATTCGCTTCCTCCATGAGTACTTCCTGAACACTTTCCTCTTGGCCACTGCTCC
CAAGACCCCCTCAAAGTCTCTCTCCTGGACTTCCAGCTTCTGGAATGAGCCCCAAGAGTGGCACTTAGCT
CCAGCTCCTAGTAGCTCACTCCTTCCTTGCTCACCTGTGCCCCCCACCTGTCTCCTTAGGGGCAGTGGCT
TTGTCTTACTCATTTGCATATCACAAATGTCCAGCATAGAAGCCAGCATGGGGCCCAGTAAGTGTTTGTG
GAGAAAGAAAGGGAAGAGGTGAAGAAGGGAGGAGACGGAGGGAGAAAAGGAGGGCAAGAGAGGGGATGTC
ATGTCATGTCATGTCATGTCATGTCAGTTCTCCCTTCCTAGATCTGGACAAGGGAAAGAAAAGCAAGCAA
GCCTGACCGCCTCAGTGCAGACCCTCTAAGATGCAAGCCTTCCCCAGGTAAGACAGTTCCGCAGGGTGCT
TCTGTGGGCTGGCTTTCTGAGCAGCCATCTCAAAGTATGCGAAAGAAATAGATTTTGGGCCGGGCACGGT
GGCTCATGCCTGTAATCCCAGCACTTTAGGAGGCCGAGGCGTGTGGATCACCTGAAGTCAGGAGTTCAAG
ACCAGCCTGGCCAACATGGTGAGACCTTGTATCTACTAAAAATACAAAAACTAGCCGGGCTTGGTGGCGC
ATGCCTGTAGTCCCAGCTACTCAGGAGGCTGAGGCAGGAGAATCGCTTGAACCCAGGAGGTAAATGTTGC
AGTAAGCCGAGATTGCCCCACTGCACTCCAGCCTGGATGACAAGAGTGAGACTCCATCTCAAAAAAAAAA
AAAAAAAAAGATTTTAGGGGTAAAATATTTTGCTTTCCTTCAGTGGCTGGGCCACCTGTACACAATGCCT
GAACCCAGGTCCTGCAGCCCCCAGCTTGGCTCTCCAAAGCTCACCAGTATCAAAGGATTTCAGCTTCTGC
TCCTCTCCGCAGGGCTCTCTGGTGGGCCCCAGACAGAGATGAAGGAAAAGTCTGGCCTTGACCTTGAGGA
CCAAAAGCGACCACGTCAGACGCCAGCAGTCTCGTGCCCAGTTGCCAAGGGATCACCTGAGGAAGGCCCT
TAAAATACAGGTGGTAATGCAGGGGTCTTGGGATGGCCCCGGACCGTTCATTTCTAATAATTTCCCAGGT
GAAGCCAATGCTGCTGTTCCAGGAACCACCCTTTGAGAAGCACGGGTCTATACCACACGCACAGAAGAGG
AAACGAAACGAGGCAAAGGAAACCAGTCCCTTTACTCGCGCCCAGGCAGGACAAAGCGGGCAAGGGTGGG
GGCGGGGGCGCGGGCGCGGGCGCGGGCAGGCGGACGGACTTGCAGGAAATGCAGCCGGGAGGATGGGCAG
GTTGTGGGATGCTCCGCCGTTCCGCGACCACCCGCCTCCCCGGCCCAGCAGAACCGGGTCCAGGCTGGGG
GAGGTGCTAGCGGTGTATGTGAGTAACACTGCCCCCTCGTCGTGGCACCGCCCCCTTCTTCCCTGCCCCC
TTCCCCGCCCCCTGGATTCTGAAGTCTGAAGTCATGCAATTTGACTTTTCCTTTCTGGAAACCAGCGAGC
AGAAATGAAACCGAAACTGAATTGTCCGGGAAATTCGCGGTGGGGGCGGAGAGCGCAGGGAGAAGTAAGC
CCAGTGCAGGATCCTGAGGCCCGTGTTTGCAGGACCAGGGCCGGCCTTCCGATTCCCCATTCATTCCAGA
AGCACCGAACCACGCTGTGCCCGGATCCCAAGTGCAGCGGCACCCAGCGTGGGCCTGGGGTTGCCGGTTG
ACCCGGTCCTCAGCCTGGTAGCAGAGGCCAGGCCAGTGCCACAAGGTCAGGCTCCACTGGGCCCTGCCCG
CCCAGGAGCCTGTGGTGTCAGGCACCTGAAGGGGTGGGTTGCCCCTCCACACCTGTGGGTGTTTCTCGTT
AGGTGGAAGGAGAGACTTGGAAAAGAAAAAGACACAGAGACAAAGTATAGAGAAAGAAATAAGGGGACCC
AGGGAACCAGCGTTCAGCATATGGAGGATCCCGCCAGCCTCTGAGTTCCCTTAGTATTTATTGATCATTT
GTGGGTGTTTCTCCGAGAGGGGGATGTGTCAGGGTCACAAGACAATAGTGGGGAGAGAGTCAGCAGACAA
ACACGTGAACAAAGGTCTTTGCATCATAGACAGGGTAAAGAATCAAGTGCTGTGCTTTTAGATATGCATA
CACATAAACGTCTCAATGCTTTACAAAGCAGTATTGCTGCCTGCATGTCCCACCTCCAGCCTTAAAGTGG
TTTTTCCCTATCTCAGTAGATGGAACGTACAATCGGGTTTTATACCGAGACATTCCATTGCCCAGGGACG
GGCAGGAGACAGATGCCTTCCTCTTGTCTCAACTGCAAGAGGCATGCCTTCCTCTTATATTAATCCTCCT
CAGCACAGACCCTTTATGGGTGTCGGACTGGGGGACGGTCAGGTCTTTCCCTTCCCACGAGGCCGTATTT
CAGACTATCACATGGGGAGAAACCTTGGACAATACCTGGCTTTCCTAGGCAGAGGTCCCTGCGGCCTTCC
GCAGTGTTTGTGTCCCTGGGTACTTGAGATTAGGGCGTGGTGATGACTCTTAAGGAGCATGCTGCCTTCA
AGCATCTGTTTAACAAAGCACATCTTGCACCGCCCTTAATCCATTTAACCCTGAGTGGACACAGCACATG
TTTCAGAGAGCATGGGGTTGGGGGTAAGGTTATAGATTAACAGCATCCCAAGGCAGAAGAATTTTTCTTA
GTACAGAACAAAATGGAGTCTCCTATGTCTACTTCTTTCTACACAGACACAGTAACAATCTGATCTCTCT
TGCTTTTCCCCACAGGCACCTAAGTCCACCTGGGCCTGGAGCAGGACAGGTTGCAAAAGAAAATATCTCG
GGACCCCCAAACTCCTTATGCTAAGGGAAACATCGAGCCTGGGAACTGAGCCATCAACGCTGCCATTCTT
TTTCCCAAACAGAACCCTGTTGTCAGAGGTACACCCAGAGCAACTCCACACCGGGTGCATGCCACAGCAA
CTCCATCTTAAATAGGAGCTGGTAAAACGAGGCTGATACCTACTGGGCTGCATTCCCAGACGGGTAAGGC
ATTCTAAGTCACAGGGTGAGATAGGAGGTCAGCACAAGGTCATAAAGGTCATAAAGACCTTGCTCATAAA
ACAGGTTGCAATAAAGAAGCTGGTTAAAACTCACCACAACCAAGATGGCCACGAGAGTGACCTGTGGTTG
TCCTCATTTTTACACTCCCACCAGCGCATGACAGTTTACAAATGCCATGGCAATGTCAGGAAGTTACCCT
ACATGTATGTCTAAAACGGGGAGGCATGAATAATCCACCCCGTGTTTAGCATATCATCAAGAATAACCAC
AAAAATGGGCAACCAGCAGCCCTCAGGGTTGCTCTGTGGAGTAGCCATTCTCTTACTCCTTCACTTCAGG
CAGGGGAGATGAATTTCCCTTTGAGCCTGTGGACAGAGGGCAGCCTGGCAGAGGGACAGGCATCAACAAA
GCCTCTGAGTGTGACCTGGGTCCTGAGTTCAGGGATGGCAATGGGGTAACTGAGACTGGATCAGAAGTTG
CCTGGAAGAAGAGAGGGCCATTATCAGAGTATAAGAGTGTGAAACTTATCTGGTAGGAAAAAAAAAAGAG
TAACTGCTGTTCGTTTAATAGAGCACACATCCTCTGCAATTCAGGTGGGAGGTTGTGAGCTTCTTAAGTA
CAGAAGCTGCCTCTTGATTTCAGCATAGCGAGGAGGTGCTGAAGAGCGCAGGTTTGGAGAATGATCACCT
GGATTGGAACCATAGCTCTACCAATATGGAACCCAGCTCCTTAGGCCTCGGTCTTCTCATGGAGAACATG
GTGTGATAATCCTACTCCTCTGGGAGGGTGGCTGTTAAGGTGAGTGATTGCACACGAGTCCCTCTAGCTG
ATGTTACCGAGCGGTCCTTGTTTTTAGAGCTCCCAAGAGGGTGGCGGGCCACTTCCAAGATGGCGGCAAG
CCTCTTGTTCTCTGACCTGGGGTTCTTGGCCTCACAGATTCCAAGGAATGGAATCTTGGGGCATGTGGTG
AGTGTTCTCATTAGGACGAACCCAGGCACTTAGACATGCGGGAACAATGGCGAGCCTTTAGCCCGAATGG
GAGCGGCAATGGGTGCCTCGCTGGATCAGGAGCACAGCGGACACACTGCCGGATCCAGAGGGGTGGACAT
CAGTGGTGGGTCTGTGATGGCGGCAAACAACAGCAGACAGCAAATGGCAGTGGTGGATGGCAAGCGAAAG
CTCAGCTCCAGCCATAACAAACACGGACCAGAAGAGTGTGCAGTTGCAAGATTTAACAGAGTGAAAACAG
ACGTCCCATACAAAGGGAGGGAACCCAAAGGGGGTTGCCATTGCTGGGTCGAATGCCTGGGTTTCTGTCT
CAATCACTGTCCCTCCCCCTGTGCTCTCAGGCAATAGATGATTGGCTATTTCTTTACTTCCTGTTTTTGC
CTAATTAGTATTTTAGTGAGCTCTCTTTACTACCTGATTGGTCGGGTGTGAGCTAAGTTGCAAGCCCTGT
GTTTAAAGGTGGATGTGGTCACCTTCCCAGCTAGGCTTAGGGATTCTTAGTCGGCCTGGGACATCCAGCT
CGTCCCGTCTCTCACTGACACCTAACACATCCTACATGCTCAGTAGACAGGAGCTATTATTGTATGACTG
ACTGCTGAATTACACCCCAAAACAACCACCTATACTCCCCAAGGCCCTGCCATATCATATGCATATTAGA
GGCAATGAAGCACCAAAATCTAAGGCCAGGGAAGGACTGCGGTGGACAAGGGAAGTGCCTAGGGCGTTAA
AACTTAAGGAGGCATTGCACCAACTGGGAGTGAGCTGTGTGTGCTGTGTTTCCTGGGCAGGGCGCCTCAC
TGCCCTTCCGAGTCCTGCCCTGGCTGAGGTTGTGCCTCTATCTGGTTATCAGTTGCCTGCAGCTCTTTCT
GATTTTGTTCGTGAACAAGGGCCCTTAGGGAAGCCTCCCGCTGTTGGAAACAGCAGCAGCATCACCCAGA
GTGAAGTCTCTGTTCACATGAGTGCCAGCTCTGCTCACACCTACCCTGCGTTCCCAAGGCACTGTTCCTG
CTTCAGAAAGCATCAGCCCAGCCTGCTCACAGTCCCCCAGGATGTGCTGTTGCCTGGGTTCTTGACTAGG
AAGTAAAAGGCCCAGAGGGGTTGAGTGTGTGTGTGTGTGTGTGTGTGTGTGTGTGTGTGTTAAAGTGAGG
TCATTGCTAATTCCTGGGAGAACTTGTGTTCCCCTTGGATGAACCCCTTGGATGAAGAACCCCTTGGATG
GGGTTGCCCAGCCCTAGGAGTGGATCCTGCAAAGCAAAATAGATTTCAGCACAGGGAGCTGGCTGGGGGG
CTCCTCCCCCTCTTGTAGGTGCAGCAGTATTGAATTTGATTCCATCCAGGTTTCCCCTGACCCCAGGACC
TAAAGCTGGGGCAGGTTCTGGGCCTTGAGGTTGGTTGGGGGTGACCCTTCCTAATCATGGAAGTCTTTAG
CGGGGGCTTGTAAGGGACTGTGAGCTGAGCTAAGGAGAATGGAGGTGGGGTGCCAACCGCTCCCAAAGGG
AAATGCCATCTCCCACTCACAGCCAGTTAGCCGAGAATGGGAGCTCCCAAAGGGAGGACCACCCATGGGT
CTGCTTGACTCAGCCCTCCCCAACCCCTTTCACCTTTGCAGTAAAACTCTAGCCAAGGAAGACAAAGAGA
CCTTTGGAGACCAAAACAGAACTTTTAATTCGGGCCAACAGCAGGCTCATGCCCAAAATGGCTGCCAACC
CCAACAAAGAAAGCAGCTAGCTTATATGTCGTTTGAGATGGGAAAAACAAGGTAGGATACAGGTTTCAGA
CAAAGACAGTAAATTACTTAACCCGTGACAATTCTGAGGAAACTGGCAATTCAGTTATATTGACTAGTCA
TCCTCCAAGCTGGACTAGGGTTGGAGGCTGGGGTCCCGAGGCAGGTGATAAGCTTTTGAGATAAGCTTGC
ATCTGCAACTTGTTACAATGCTGGGAGGGGCTGCTTAAAATTTTAGCCTATGTGTTACTTCTAAATAGCT
TATACTAAATGTTAACTGTTTTCATGTGCGTCCATGTGAAGAGACCACCAAACAGGCTTTGTGTGAGCAA
CATGGCTGTGTATTTCACCTGGGTGCAGGCGGGCTGAGTCCGAAAAGAGAGTCAGCGAAGGGAGATAGGG
GTGGGGCCGTTTTATAGGATTTGGGAAGGTAATGGAAAATTACAGTCAAAGGGGGTTGTTCTCTGGTGGG
CAGGGGTGAATCTCACAAAGTACATTCTCAAGGGTGGGGAGAATTACAAATAACCTTCTTAAGGGTGGGG
AAGATTACAAAGTACATTGATCAGTTAGGGTGGGGCAGGAACAAATCACAATGGTGGAATGTCATCAGTT
AAGGCTGTTTTTACTTCTTTTGTGGATCTTCAGTTACTTTAGGCCATCTGGATGTATACCTGCAAGTCAC
AGGGGATGCGATGGCCTGGCCTGGGATGCGATGGCCTGGCCTGACAACTATTACCTATGTTATGTTTATT
ATTTTAAGCTTTATTATTACTATTTTATTTATTTTATTTTATTTTCCTTCCACACACCCGTTTCCACCCT
GGAGAGGCCAGATGAGCCAGACTCCAGGGAGGCCTAGAAGTGGGCAAGGGGAAACGGGAAAGGAGGAAGA
TGGTATGGGTGTGCCTGGTTAGGGGTGGGAGTGCTGGACGGAGTTCGGGACAAGAGGGGCTCTGCAGCCA
TTGGCACACAATGCCTGGGAGTCCCTGCTGGTGCTGGGATCATCCCAGTGAGCCCTGGGAGGGAACTGAA
GACCCCCAATTACCAATGCATCTGTTTTCAAAACCGACGGGGGGAAGGACATGCCTAGGTTCAAGGATAC
GTGCAGGCTTGGATGACTCCGGGCCATTAGGGAGCCTCCGGAGCACCTTGATCCTCAGACGGGCCTGATG
AAACGAGCATCTGATTCAGCAGGCCTGGGTTCGGGCCCGAGAACCTGCGTCTCCCGCGAGTTCCCGCGAG
GCAAGTGCTGCAGGTGCGGGGCCAGGAGCTAGGTTTCGTTTCTGCGCCCGGAGCCGCCCTCAGCACAGGG
TCTGTGAGTTTCATTTCTTCGCGGCGCGGGGCGGGGCTGGGCGCGGGGTGAAAGAGGCGAAGCGAGAGCG
GAGGCCGCACTCCAGCACTGCGCAGGGACCGGTGAGTGTCGCTTCTGGGGGCAGCGCCCAGTAACCGCGC
TAGGAGCGCGGAGAAGGGCATTGGGAGAGCGGCGTTCGTGGCGGAGACTAGCGCTCCGGAGCACGGGCAC
GACGGGGGCACCTTCTCGGCTGCTAGTAACTAACAATAATAATAATCATAATCATAGCAAGGGCGCTGAT
GGGCGGGCTCGGAGCACGCCTGATTCTGGTTCCCACCAGGCTGCCCAGGCTCCTGATGACGCATCAGAAA
CATCCCCCTAACCCGCGGCCTTCCTGCAGGAGAGGTTGGGAAGGGGTGGGGGACGGGGCTCGGGGGAGGT
CTCCGAGGGACTCTAGTAAGCGGGGAAGGGCGCCGGGAAAGTTTCAGATCCACGGCTGCGCGGGCCACGA
GCCCACCCGAACGCCGACCACTGCTTTCCGTCGACTTCTATTTCCTGGGAACGCGCGAAAGCAAACCCAA
GTCAGACTGCGGAGGTCGCTGGGGAGGGAAGGTTCAAGGAGTTCTCGCCGATCCTGCTGAATAAAGGGGG
TTCCGAGCTGGGCCGAGATGGGGCATGCGCGGGAAGACCCCTGCCCGCTGTTCCCCCCCACCGCCCCAGT
GGATGCCATGCCTGGGGCCTCCCCGGCGCGTGGGGCTGACGCACCCTCGGGGTCCATCGTAGTTGGCCGG
GATCGTGGAGTGGGTGCGGTGGACGAAGGGAGGCAGGACAGTCCCGGGGGTGGCAGAAGGAGCCCGGGCA
CAGCTGAGACCTGCGCTCCCATCCCACCAACACTCACAGCAGGTGCTGCCGAGCTGGGCAATTGGGATGG
CCCAAGTTATTTGGTTAAATTTTAAATCACGTTTGTTACTGGGAAGTAGAGTCCAGTGATGCTAACCGCG
CCTCTACCTCCACCACCGGTGTCAGTCCCAAAGGGCTCCTAAAATGGCTGTGTCATCTTTCAGCCTTGGA
CCGCAGTTGCCGGCCAGGAATCCCAGTGTCACGGTGGACACGCCTCCCTCGCGCCCTTGCCGCCCACCTG
CTCACCCAGCTCAGGGGCTTTGGTAGGTAGCAGTGCATTTGGTCTAAAGGGCAAGATGTTCTCTCTTTTA
TTCATAACAAATTTAAATACCAGCAGGGTTTGGGGGGAAAAACGCTTTCAGAAGAAAAGGTGAATGTCAG
TCCTGCAAGAGTTAGTTTTAAAACTAGACTGAATTGGCACATGTATACCTATGTAACAAACCTGCACGTT
CTGCACATGTACCCCAGAACTTAAAAGCTTATAAAAAAAGAAAAAACTAGACTGGATTATGTTGGGAAAG
TGTAGCCTCTTCCATCTTAGGCATTTCCTAGAACGTAGGCAGTAGGTGGTCCTTATTAGGAGTTTTGGGA
GAGGAAGGGGGCTGAATCCTACCTCCCATCCCTGCTCCTCTATGGGGTCTGAGCTGAGGAAGCTTCACCA
CAAGGAGAGAACCCCCTGACAACCCTGGATGCCACCTTTACCCTCACTGCAGGAATTCTGTGGCCACACT
GCGAGGAGATCGGTTCTGGGTCGGAGGCTACAGGAAGACTCCCACTCCCTGAAATCTGGAGTGAAGAACG
CCGCCATCCAGCCACCATTCCAAGGTAAGGCAGAAATGAAGTGGGCCGTTGGGTTCTTTCTTTTCTTTCT
TTCTTTTTTTTGAGACAAGGTCTCACTCTGTCGCCCAGGCTGGAGTGCAGTGGCGCCATCTCAGCTCACT
GCAACCCCCGCCTCCCAGGTTCAAGCGATTCTGGTGCCTCAGTCTCCTGAGTAGCTGGGATTACAGGCAC
ACATCACCACACCTGGCTAATTTGTATGTGTTTAGTAGAGACAGCATTTCACCATATTGGCCAGGCTGGT
TTCAAACTCCTGGCCTCAAATGATCCACCCGCCTTGGCTTCCCAAAGTACTGGGATTACAGGCACGAGCC
ACTGCACCCAGTCAGGTTCATTTTAGTTGTTATGTTAACCAGGTTTCCTGCACCTGTGCGCTAACTTTCA
CTTTCCCAAAAGGTTTCAGGGTGACCCAGCAGGCAATGAGTGATTCTCAAATTCAGGATTTATTGTGAGA
GATTCACACACACAATTGAGCAGACATTCACAGTACAATGATTAAAGGGAGTGATAGGGTAAGGACCCAC
AGTGGAGGCTCTGGAGGCCAGCCCACTGACAGCCACTCCAGGGAGTCCAGAAGTCCCGCTCTAGTGCTGG
GTGGTGGAGGGAAATCTGTTCCTCCAGGGACCTCGTCCTCGGCTGCCCAGCTGCCAAAGTCAGGAATAAG
CTTTCAGAAATCTCACTGCCAAGATTCCGAAAACGCTTCAGACATTGCTAGTCCCTTGTCGCTTTTGCGA
TCCTCCACAGGTGTGCGTGCCACTGGGTCCTTATTCACTGGGGTCTCTGGTGGCATTGGGCCACAGCAAG
TGTTCCCTCATCCCCTTAGTCTACCACACACATGCTTACCACTTTGAAGAAAAACCCCTTTACTATGAGC
GAAAGTGAGAAACACGTATGTTTATTGTTTCTAAAGAAAGAAACTTAATATGGGCTTAATGCTACCTAGT
GAGTGCCTCCATTTTGAGACATTAGGGTCACAAGTCATTATTATATATCATGGGCACAAACCTGCCCTGG
GCAGGGACGGAAGGAAGCCCCTGCACAGGGGCAGTTGCTCAGGATGTGAGAAGAGCCTGGTGCATAACCC
CATCCATGCCCACCTAACATCTCAGGCTCTGACCAGTGGGGCTGTGCAGTAGCGAGTGGATGGAGGGCTG
GAACCCTGCAGCCTCCTCTCCAAACACAGGGTGCAGCCAAGACATTTTAGGAGCAATTTGGGATGGAGAG
CTAGGAGTCGCCACCTCTTGGCTCTTCCAAGGCCGGAACTGGTGCCTGCACTCAGTTCAGTTTGAAGACT
GCAGCTGGATGCCAAGTTCCATGGAGGAGTAAGAAACCGGTTTGAACTCCCGAGATTGCCCTGCCCCTGA
AATCCAAACTGATGTTCCGAATGATCAGGGAAAGGTACAAACGTTTATGGTTTACAGACAAAACCCATAA
GGTTTAGCTTTCAGAGAATCTCATTTTATGAAGCAAATTAGGGAAGGGAATCTACTCACCAAGTCCTGTT
TCAGCTGATTGAGTGGAACCTGTGGTCATGTGGTACAAGTCCTGGTCTCAATGATGCTCCTTATCTGGCT
GCAGAAAGGCCAACTGAGGCAACCATAGCCCAGAAGACTGGTACTCCTGAGAGGCAGATGAAGTGGTGGT
CTTTGATATCGAGCCTGGGATGCCCTGGGCACATGAGGTATTTCCAAAGGCATGGGAGTTTTAGGGAATA
AATTCCCAGATTGTCAGACTCCATAAGTACCGTTTACAATGGATTACCTTTTATAACCATCCCAATCCTA
CCTGACAAAAGAGGTGGGCAGATTACGAGGTCAGGAAATTGAGACCATCCTGGCTAACACTGTGAGACCC
CATCTCTACTAAATAAATACAAAAAATTAGCCGGGTCTGGTGGCGTGCACCTGTAGTCCCAGCTACTTGG
GAGCCTGAGGCAGGATAATCGCTTGAACCCGGGAGGTGGAGGTTGCAGTGAGCCAAGATTGCGCCACTGC
TCTCCAGCCTGGTGACAAAGCGAGACTGTCTCAAAAAAAAAAAAAAAAAGAAAGAAATACATCCCTTTCT
TCCCTTCCAAATCGAGCAAGGATGCCTGCCCTGGAAGTGTATAAACCCGGGGAAGGGAGACAGAAAAGGA
TAGTTTTAAGTATTGGTGTTGGGGACGTGTTCTTTAGCCAAGGCAGCATGAACCCATGGCAGCACTTCCC
AACCTTCCTGACATGGGCGTTTCTGTGAACTCCAGTGTGATGGAGAAATGGCATTGGCTCAGGTGTGCAG
CTAGATATGTTACAGAGCAGGGTGACAGGCAGGGGTGATGAGTTTTGTTTTAACAACCTGTCCCTTCAAC
CCTCATGGTACTGACAAAGATCACATGGCTCTCGGGGGAGATTCCTGCGAGGGGAAGCAAGGAGAGCATC
CTTACATATTATTGATCCAGGCAGCAGATTTGCAGCAAAGCTCTGTGCTTTATTCATCTGTTAAAATAGT
TAAAATAGTCAAAACATAGGAAAAGGATTCTGGGAAGTCAGAATCGGCTTCAGAGCACACCCCTCCTGCA
CTTGCCCGGTTCTCAGACTTGGGAATGGGACTGGGTGGGTGGGTACTCTCGGGTGTTCCGCGGGTTTGGG
TCTTACTTGTACACTTTGCTTGATTTCAAGGAGGTGCAGGAGAACAGCTCTGTGATACCATTTAACTTGT
TGACATTACTTTTATTTGAAGGAACGTATATTAGAGGTAAGTTGGTGCATGCTATTTTCTGTAACATTTA
TTTTGAGTCATAGGAGAAAGATTTTCAGTTACTTTTATCCAAGATTATTAGACACTGTAAAATTTCATAT
TTAGGCACTTGTCCTACAACATTTTAAAAATGAATTTCAAATACATACGTGTGTATTTGTAATGCAGACA
AGTATAAGGCAGTCAGTTACATGCTTTCAAGAGTAAAATGAATGACATTTCATTTCCCCCATTTGTGGGA
GTAAAAGAATGACAATATGAAATTGATGATCAAAAGAAAGAGCATAAAAGATTTAGAGCTCACGTGTTTT
TTAAACTAAAGGTTTGGGTATCAAATTACCGTAATATTTGGATTCTCTTGGCTACATTGGAAACAGTTCT
ATAACAATTTTATTTTTAAATGTAAAGTTTTTGTTTGTTGTTGTTTTTAAGACGGGGTCTCGCTCTGTCG
CCCAGGCTGGAGTGCAGTGGTGCGATCTCGGCTCACTGCAACCTTCACCTCCCAGGTTCAAGTGATTCTC
CTGCCTCAGCCTCCCAAGTAGCTGGGACTACAAACACACACCACCACTTCCAGCTAATTTTTGTATATTT
AGTAGAGATGGGGTTTCACTATGTTGGCCAAGCTCGCTTCGAACTCCTGACCTCAGGTGATCCACTCACC
TTGGTCTCCCAAAGTGCTGGGTGGGATACAGGCGTGAGCTACTGTGCCCAGCCTTTAAATGTAAACTTTT
TAATTGTATTACAACTGCATCAGAAGTTGTATACTTTGCAACTATTCAAATTTATACTGAAAACGTTTTT
GAAGTTCAACCTAAAATTATGACAGGAGATAGTTTTAGAAAATATTTTGGGGAACAGAGGCATATTCTAT
TTTTTTTTTTTGAGACAGAGTCTTGCTCTGTTGCCCAGACTGGGGTGCAGTGGCGTGATCTCAGCTGACT
GCATCCTCTGCCTGCCAGGTTCAAGCAATTCTTTGCCTCAGCCTCCTGAGTAGTTGGGATTACAGGTGCC
CGCCACCATGCCTGGCTAATTTTTTTGTATTTTTAGTAGAGACAAGTTTTACCATCTTGGCCAGGGTGGT
ATTGAACTCCTGACCTCATGATCTACCTGCCTCAGCCTCCCAAAGTGCTGAGATTACAGGCGTGAGCCCC
GGGGCCCGGCCTGGAGGGATATTCTTGAAGCGCCTTGAGCAGGGAGGCAGCGTTTGTCTTTATCTGACCT
TGGCTTCTTTGGGTCACTCTGTTTCTCTTTCCGTGAATAAAAAGCCAGTGAGCACACACTGTGTCCCAGG
CACTCTTCTACGCTCTGGGGACATCACCATGAACAACTAGTCAGAGTCCCCACCTCCAGGGGCCTTCCGT
TCTGGTGGTGGGTTTGGTTCCATGGTTGAATGCACCCAGCTGCTTATCTGTTCAATAGGCATCTGCTTTA
TTTTAAGCTTACTTTGCAAAGAAGGAAGATGGTTGTTTCCGAAGTGGACATCGCAAAAGCTGATCCAGCT
GCTGCATCCCACCCTCTATTACTGAATGGAGATGCTACTGTGGCCCAGAAAAATCCAGGCTCGGTAAGTT
GCTCTCTGAAAGTCGCTATCCATGTGACATGAGCCATGCCCATTCGAGGCTGCCCTTTTCTACAGCGGTG
CTACTGCTGCCCAGATATGCCTGCTCTCTCGCCTCTCCTGTGCCAGGACGCAGATCCTGACCCTCTACTT
GCCAGCTGACAACCATGTATAGACTCGTTGCCTTAGCTCACCGTGGGTGGAGGTGCTGCTGGGGTTGGGG
ACACTGGCTGAGCTGTCGATGGGTTCAGCTCATCTATACTAGAAGGAACTGGCCCAGGCCCTGGGTTCAA
GGAGCACTAGGTTTGTTTTTCCTGCCCACATCATGATTCAGTCTCAAGCTACAAACCCTGGGGCATCATT
AAGATATTATCTTTGTAGGGAAGCCACGTGGTGAATTTTTTGCCCAGAATCAAGACATATTTTTGGTGGG
AACCAATCATGGCCCCTGGAATCAGTCCAACCTCAGTTGGTCCCGACGCTGACACTCTCAGCTGTGTGAT
TGTGGGTAGTTATTCCTCCTGTTATAAGCCTGGTTTTCCATATCTAAAATGAGAATAATGGTCTTTGCTT
TATATGGCTGAGAGCAAAGTGCAGGGAGTGTCCAGGTACTCAGAGTGCTCAGTTTCTTATTACCGTGGAT
CACTGGTCTGTTTCAGGCTGGCTCTGTTTTCCTAGGCAATGTTAAACAATTTTTCAAACAATATTCAAAA
AACATTGAAGCGTTTAGAAAAATACAGAGGACCACCACCTTTCCACCACCCTGATACCACCGTTTACATT
TTTCTGCATTTCCGTCCCTGGTGCGTCTCTTGTCTGGCTGTAAGAGATGTAATTATGTCAGGGTATGGGG
CAGGAGGAACTCTCACCCACTCATACTTGCGTGCTTTGGAAGCAGCTTGGCCTTTTCCAGTCAAGGAGAA
CAGAGTGTGTCTCGTGATCTGGCAGTTCCACTTCTGGGGATAGACTGTGGTTCTCAAAGTGTGGCCCCCA
GCCCAGCCCATTAGCATCACCTGGGAAGTTGACTGAAATGCAAATGACCAGCCCCTCCTCCCACTCTTAG
ACCTGGTGAATTGGACACTCTGGGGCAGGGCCCACCCCCTGTGCTTTACCAGGCTCTCCAGGTGATTCTG
CTGTGTGCTGGAGTTTGAGGACCATGTGTAAACCCTTGCACATCCCCCTGGGAGCCACATGCATAGCAGC
ACTGTTTATAACAGCAAAACTCAGACCCTGTCTACGTGCCTGTCATGGTGGAATGGATACTGGGAGTGTG
GTATGTTCATGTAGCAGAATTCTATACAGTAGTAAAGAGGAATGAACTAGAGTTCCAGGTATCAAAATGG
CTGCATCAAATGAACAACGTTGAACCAAAGAATCAAGTTACAGGAAATATACAAAATGATTCCACTTACG
TAAAATTCCCCAACAGGCAGCAATAGGCAATATACCATTGTGGGAAAAACATATAGGTGGCAAAACTATA
AAGAAAAGCAAGGATGTGATGATTGCAAGCCTCAGGATAGAGGGACCCTCTGGAGGTGAAGGAGTTGACC
CAGGGAGGTGTGTGCAATGTCCTAAGTACTGGGAGTGTTTGTGTTCTTAACCCAAGTGGTTGGTACATGC
ATATTTGCTTTATTATGTTTGACACACTTTTGTAAATAGATAGTATAAATAAATGAAAACAAACAAAAAG
TTATAAGGTGAACTAAGACCGAGGCTACCAACTGTATTCATGCATTTGGTAAGGCTGTGGTTCTTTCTCA
GTCAGGGCCCATTTTGCTCCCTGGAACTTGTGGCCAGGTCTAGAGACATCTTGGTTGTCACAACTCAGGG
TGAATAGTGAATAGAGGCCAGGTATTTGGCTAAACCTCCTACAATGTGTAGGGTAGCCCCTGCAACAAAC
AATCTTCTAACCCCAAATATGAATAGCTTCTTGTCCTGTTATAAAGAAGCTATTCTAGTAAAAACGTCTG
TCTATGATGAAGCATGCACAAAAATAGTCATTAGAAAGAGGTAAAAGACAAAATGATTTTCTCATATTTT
CTTCCTGAACCTCAATCAGCCCACTTTAGGAAAATTGCACCCAGCTGCTGGTAGGTAGGCAGGACCGAGT
GTGAAGTCTGCTGCTTTCTCTGTTTTTATGCAAGTACTTCACTATTTTGATTACTTTAGTTTTATAGTAA
TTATGGAAATTAGGTAATGCTAGTCCTCTGACTTTGTTCTTCTTTTTCAAAGTCATTTTGACTAAGAATA
TTTAGATCATTTCTATTTAATGTGACTGGTAATATAATTAGATTTGAGAATATCATCTTGCTATTTGTTT
TCTATTTGTCCCGTCTGTTCTTCGTTCCCCCTTTCGTCTTTTTCTGCCTTCTCTTGGATTATTTTTTATG
ATTCCATCATGTCTCCTTTGTTGTCTTATTAAGTATAACTCTTGGTGTTTTTAGTATATATCTTTAACTT
AATAAGTCAACCTTCAAGTGATAGTCTGTCACTTCATGTATAGTGCGAGAACCTTAGAATAGTGTATTTC
CATCTCTCTGCTCTGAGCCTTCATACTATTTCTATCATGCATCTTTTACATACATCATAAACCCCACAAT
ACATTGTTATTATTGATGTTCAAACATTCAATTATCTTTTAAATAAAGATAGCAAAGGAATACAAAAAAG
TGTAGTAGTTACCCCTTCTAGTATAGACTCCTTTGTATAGATCCAGATTTCCATTCAGTATCATTTTCCT
TCTACCTAAAGAACTTCTTTAACATTTCCTGTAGTGCAGGTCTGCTGGTAATGAATTAGTTAAGCTTTTG
AATGGCTAAAAAAGTCTTTGTTTTGCCTTCATTTTTAAAAGTTATTTTTGCTGGGTATAGAATTCTAGAT
TGATGGTGTTTTTCAGTACTTTAAAAATACTGCTTCACTGTCTTCTCGCTTGTTATTGCTTCTGATAAGA
TTGACAGCAGATTTCTCATTTGTGTCCCTCTGCTCACACTGTATCATTCTCTGGCTGCTCCTAACATTTT
CTCTTTATTACTGGTTTTGAGCAATTTGACCCTCTTATGGCTTGATGATGTTTATGTTTGCTGTGCTTAA
TGTCTGTTGAGTTTCTGGGATCTTTGGGTTTATGGTTTTCATTAAGTTTGAGGGAATTGTATGTATTATT
TCTTCAAATATTTTTTTCTGTCTCTCTTCCATTCTCTTTTGGGGATTCCAGTAACCTGTGTATTAGACTT
ATTGAAGTTGGCCGTCTTTAATGGAGTGTATTGGTTCATTCTCACACTGCTATAAAGAACTGCCTGAAAC
TAGGTAATTTATAAAGAAAAGAGGTTTAATTGACTCACAGTCCACATGGCTGGGGAGGCCTCAGGAAACT
TACAATCATGGAGGAAGGCATCTCTTCACAAGGTGGCAGGAGAGAGAATGACTGAAGGAGGAACTTGCCA
AACACTTATAAAACCATCAGACCTCATGAAAACTCACTATCATGAGAACAGCATGGGGGAAACCTCCCCC
ACAATCCAATTACCTCCACCTGGTCTCTCCCTTGACACGTAGGGATTATGGGGATTACAATTCGAGATGA
GATTTGGGTAGGGACACAGAACCAAACCATATCATGAGCATGATTTGCAGGCCATGAAGAATTCTCCATT
TTTGTTTCCTCCAGGTGGCTGAGAACAACCTGTGCAGCCAGTATGAGGAGAAGGTGCGCCCCTGCATCGA
CCTCATTGACTCCCTGCGGGCTCTAGGTGTGGAGCAGGACCTGGCCCTGCCAGCCATCGCCGTCATCGGG
GACCAGAGCTCGGGCAAGAGCTCCGTGTTGGAGGCACTGTCAGGAGTTGCCCTTCCCAGAGGCAGCGGTA
AGAACTTACATTCTGTGTTAGTCTGCTCAGGCTGCCATAACAAAATACCACAGACAGGGTGGCTTATACA
ACAAAAGTTTATTTTCTCACAGTTCTGGAGACTGGAAGTCCAAAATCAGGGTTTAGCTTCTCCTGAGGCC
TTTCTCCATGGCTTGCAGATGGCCACCTCCTCACCGCTCCCCCATGCGGCCTTCCTTCCACACACAAGCA
TCTCTGCTGTCTCTTCCCCTTCTTCATAAGGGCACCAGTCATATTGGATTGGGGCCTACCCTAATGACCT
CATTTAACCTTAATTGCCTCTTTAAAGGCCTTATCTTCAAATACAGTCCCATTAGGGGTTAGGGCTTCAA
CATAGGAATTTGGAGGGAACACCATTICATAACGCTATCTCATTGCACATTTTTTTCACATAAGTCATAT
GTAATCTACAGTTTGAGGAGAATCTGAATAAACACATTTGGGTCCCCCAGTTCAGAACTATATCAGTGAG
GTCTCAAAGAGTTCAGGCCTGGGACGGGTCTTGCAATACAGATCAGGTGTGGTAGGAAGAATATGGAAGG
AGTTTACAGTAGGAGGACTGTTGTAAGGTGGCCTGGTAGCAGCAGGATGCTTTCCTAATGGGGGTAGAGT
GTGTATGCTGGGGGGATAATGGAAGATGTTCGGATGAGTTTCGGGGGATTCCCAATGTGGTCTGCCACCT
GAGCTGATGGCAGAACACTGGGATGAGGCAGGAAGCCAAAAGTGGTGGCTTTCAAGCGTTAGTAAGCAAA
AACTCACCTGGGTTACATACTCAGAATGCATGTTCTTGAGGTCACCCAGACACAGTGCGATGTCCCCGCA
TATCAGAGGGTAAGACCAGAAAGTTTCCAGTTTTAAATGTCTCCCCATATGATTGTATAAAAGTTTGAGA
ACCATGGGCCTAAGGCGCTATGTAGGTCTTTTAAGAGCAAAGTGGAGCACTGATGTGGGCGTGGCCTCCT
AGGGATCGTGACCAGATGCCCGCTGGTGCTGAAACTGAAGAAACTTGTGAACGAAGATAAGTGGAGAGGC
AAGGTCAGTTACCAGGACTACGAGATTGAGATTTCGGATGCTTCAGAGGTAGAAAAGGAAATTAATAAAG
GTGAGTACCCCCTGTTTGGATGCCTGGTCAAGCCTTCTGACATGCATGGGGTCTGTTTGTAACTGTTCAT
ACTCCCACCTCCCTGGGCCTGTGCTGTCAGGACACCTTTCTCCTGCACATCAGGCCACGGTTCCTTCTAC
TTCTTTTACCTCATTATGACCAGCACGCTTGGATATCAGCATCTGATAGCAATCATTTATTTCTGGCCAG
GCACAGTGGCTTGTGCCTGTAATCCCAGCTCTTTGGGAGGCTGACACGGGGGGATTGCTTGAGCCTAGGA
GTTCAAGACCAGCCAGGGCAACATAGTGAGACCCCGTCTCTTAAAAAAAAAAAATTAAAATAGCTGGGCA
GGGTGGCATGCACCTGTGGTCCCAGCTATTCAGGAGGCTGAGGTGGGAGAGTTGCTTGAGCCTGGGAAGT
CAAGGCTGCAGTGAGCCGTGATCTTGCTACGGCACTCTAGCCTGACAACAGAGTGAGACCCTGTCTCAAA
AACACATGTATTGCTTATTATGTAAGTATCTAGAATAATGTGAATTTTAAAATGTCCCCACATATGGATG
ATCTGTCCCTTATTCAAGGGCTTCCCTACTTAGATCTGGCAGGAAGAGGAGCCAGATATGGGGGTGAGGG
AGCTCCTCCCCCTGTTCCTTTGTACAAGGAACTCCACATTGTGGACAGGATCGTCACTGAACCCCACTCA
GAACCAGCACCCTTTTCTAAGAAAGAGGAGTGACTGTGTTTGCATAATCCCAGCTTAGGCTAATTCATGG
CAGGCCTCCATAAATGCAAACCACAAGGATCAATTTGAAGTGTCTGCAAGGGGAAATGACACCAGCAGTG
TAGACAGGGTAGAGTAGCTGACAAAGAACAGCCTCTGTGAGATGCATGGATAACATCTTCCTATCGACCT
TCATGTTTTTCTGGCATGTCACATGTTTAAGTTTCATTCACACTGGGAAGGTACTGAAGAGACATGAACT
AATGCCCAGCAGTAGGAAGGGACGGGTTTAGCATTTGTAAAGATGGAGCATTAATCACATTTGTTGACTG
TTTAAAGAAAGATAAATAATGTTATTGACAAACCGTGATTTTGAATTAGTTGGGATTAGGTTGGCTGCTT
GCAGCAGAAAACTCAAAATAACTGTGTGCCTTAGCCCTTGCTGTATAACAAACCCATCTTAAAACTAATG
GCTTGACCACTTTTATTTCTCATGATTTGATGGACCAGCTGGGCAGTTCTTCTCTGGGCTAGCTGGGCTG
GGGCTGATGGTCCAGGATGGCCCTTGGCTGGAGTGACCGGGCCTTCTGTCTGTGTGGTCTCTCACCCTCC
AGAAGGCTAAGCTGGACTTATCAATGTGGGTGGAGGGGTTCCCAGCAGCAAGAGAGGGCAAGACCCGACA
TCTAACACCTTTCGGTCTCTCCTGACGTGGCACTGTGTAACATCCCCTTAGCCAGAGAAAGTCACGTGGC
CAAGCCCAATTTCAAGGGACCGATTTTCTCTCTCTCCATGGAAGTGACGAAGGCACCCTGTAAAGTAGCG
TGCATACAGGGATGGAAGGGAATGTGGATGCCATTGTGCCAGCAAGTTGCTGAGACAGCGGTCTAAACAA
TGTAGAGGCTTTCTGTCCCTCTCATATAAGTCTGAAGGCGGGCAGACCAGAGCTGATTGGGGATTCCAAT
GTCAGGAACCAAGATTTATTCTTCTCCCATTTCTTCATTGGGTGTGGCCTCTGTTTCCAAGGCCCCCCCG
TGACTTAGTCAGCAAGTCCGCTTTGAAGCCAGCTGGACCATGGCAAGGGGCATATCTCTGCCTTTGTAAG
CACACTTTCTGGAAGTTGCACATAACATTTTCACATGGCCCATTGGCCAGAACCCGATCTCATGACCACA
TGGCAGGTACATGGATATTGGGGGAACAATTAGTGGACCATAACCACTGATATTTCCTAAGTTCTAAATT
GATATCAAACATCCCAAAAAGGCATTCTAGATTTAGAAAAGAGTAAAGTGGTGTTAGCCAACAATTTGAT
GAAACAAATTCATATCCTAAAATTCATTAAGGAGGAAGGAGCAAAATAAAATCTCTTAATGGGATGTTAA
CAGCCAGTGCTTATCTTAGCTAAAATAAGCACATTTCCCCATATAATTTTCCAGTTTATATTTTAGGCAT
TTCCATATATTTTTATTTGTTTTTATTTTGCTTGGTTGCTAATTTCCTACTGACATCAATGAGAAGGATT
TAGGAATGCTACCAGGAAGAACTTCTTGCCTCCGCCCAGCTTTGGACTGGTCTAAGTGGGTGTCACTCAT
GGTGACGTTCTCACAAGGTCTCTCTACACACAGTGCTGGCCAACAGCAGGGAAAATACTGAGTTATCCTT
TGAGATCTCTTTTATCCCAATCACAGAAAATTGAATCTGCTCCAAATATGCTTTTATCCATGACTCGCAG
AGAGGAGAAGATGCTTTCAGAGTATTCACCATCATGAGATCCGTTTATCCTAAGCTCTGTTTGGGTTTGA
TTTTCCCTGTCTCTTTTCTAGCCCAGAATGCCATCGCCGGGGAAGGAATGGGAATCAGTCATGAGCTAAT
CACCCTGGAGATCAGCTCCCGAGATGTCCCGGATCTGACTCTAATAGACCTTCCTGGCATAACCAGAGTG
GCTGTGGGCAATCAGCCTGCTGACATTGGGTATAAGGTCAGACTTCAGACCCATTCTGACCTTGGCCGTG
GCGTGGGGATGGGGGAGTGGAGGGGTGGGAGGAGAAAGAGGGTACTGTATTAGAGTAACCGTGAGTCCAG
AGCTGAGTTTTGGAGTTAGTATTTGGAGGTGTGAGTGGGGAATTTAGAGAGCCCGTTGGTCACAGTCTGT
TCTGTCAAGTTGAATGGAAGCTTCTTTGGAGAAAGTGAGGCCAGTGGGCACAGTTGGAAATGTGTTCTGT
GTATTTGTTTTATGTTTTATGCAATGACTTGTTTTTGGTTATATACATTTTGCAGCATATCTAAAGTGCT
GTGTATTAGGAAGGGGTCTTATGTGGGAAGAGAGCATTAAAAATAAGTATAATGGGCCACACACAGTGGC
TCAGTCCTATAATCCCAGCACTTTGGGAGGCTGAGGCAGGAGGATTCCTTGAGCCCAAGAGTTTGACAGA
AACCTGAGCAACATAGTGAGACCCCCTTCTCTATAAAAGAAAGGTTAAAAAATTAGCCAGGTATGGTGGC
GTGCACCTGTCAGCTACTAGGAGGATTGCTTGAACCAGGGAGGCTGTGATGAGCCGTGATTGTGCCACTG
CACTCCAGCCTGGGCAACAGAGCAAGAATCTGTCTCAAAACAAAAAACAAAACAAAACAAGCAAGAAAGA
AATAGGTATAATGATATTTTAGTATCAGTGAATCTCACTTTACAGATTAAAGATTTAGGGGTGAAGTGGG
GTTTTTTGGCCACCATTTTTCATTGTGACCATCAGATCTGAGGTCTTAGGGGTTAATTATCTGAAACTTC
ATGGTTTTCCCTGAGCCTATAGCTCTGCTTCTGCCACAGATAATTTATTTTCTCATAATTCCAGCTTGGT
ACCTCCAGGGTTGTGTTTGTGGGTTCATTTCTCCAAAGTTACTTCTTTTGGGGGAAATACCCCTGGGACT
CTTAGGGCCTAAAGCAAGTGCAAGGTCAGGACTTGTCTCACCTCTCACTTGCCTTTGCCATACTCACGAG
TCACCTCCTCTCATTTCCTTACAGATCAAGACACTCATCAAGAAGTACATCCAGAGGCAGGAGACAATCA
GCCTGGTGGTGGTCCCCAGTAATGTGGACATCGCCACCACAGAGGCTCTCAGCATGGCCCAGGAGGTGGA
CCCCGAGGGAGACAGGACCATCGGTGAGAGTGGGGGAGCCCCACTGTGCTCAGTGAGAATGGGGGAGCCC
GCCTGTGCTCGGTGAGAATGGGGGAGCCCACCTGTGCTCGGTGAGAATGGGGGAGCCCGCCTGTGCTCGG
TGAGAATGGGGGAGCCCGCCTGTGCTCGGTGGTCTGCCAGTGGGCAAGCGTCCCTCCAGTCTCCATGGGC
TTTGCTCAGTGGGGACCTGCCTCCACTAAGACCTGCTAAGGGAGCAGGTTTGGTGCCCACCAAGGCCAAG
TGAAATGAGCTGCTTTTGACTCTCACTGGCTAGGTTGCCTTGTAAGCCTTATCTACTTGCTCAGAAAGGC
ACAGTGGGCTCGGAAGCAGGTCAAACTCAGGAGGCACATGGTACTCATTAAGAATGCATTTGAGATGGGA
TGTCCATAACTCAAGGGATAAACAAAACGTGGCGTGTTCTACAGTGGACCCGGGTGAAGGAGCTTGGGGA
GAGCCACATGCTGTTCTGGGAGGCATCCCTGCCTTCACGCGGCTTGTCGTGGAGTTCTTTTCTGGAGCGG
GGCTCCACTGCCCCCATGGTTCTGCAGGGGCTATGGCCTGTCCTCAAGCAAGGATGGGAGGAAACCCTGG
GAGGCCGGGGGCGTGAGCAGTTGTTCGTTCACCTCTGCCTCGTGACTGAGCACGTTCTCTCCCCAAATAC
ATCTGGCTCGCAGGAATCTTGACGAAGCCTGATCTGGTGGACAAAGGAACTGAAGACAAGGTTGTGGACG
TGGTGCGGAACCTCGTGTTCCACCTGAAGAAGGGTTACATGATTGTCAAGTGCCGGGGCCAGCAGGAGAT
CCAGGACCAGCTGAGCCTGTCCGAAGCCCTGCAGAGAGAGAAGATCTTCTTTGAGAACCACCCATATTTC
AGGTGCGCTTGCCTGGGTTTCATCATGGATCAGTCCAAGCCCAGGATGTCAGGCCTTCCAGGGGACAGTG
GCAGCCGTCCCACAGATGTGTGGAGTGTGTGTGTGTGTGTGTGCGTGTGTGTGTGTGCGCGTGTGTGTGT
GACTATGCTTGTTCCCCAACAAGGACTATGGAATTCACCTAGAAGAATAGGAAGGGGATTACAAAATACT
GCCAAGAAAAAAAAAACTAAAAACCAATCAAAATAGGGAGAGAACAATGTACAATAATTTACGTAGCATG
GTGCTGGAACCATATTTTATAAAAACATAAATAGAAGAGAATAGGAAAAAAGTAGAAAGCCCAGAAATAG
ACCTAGATATATATATTTGACACATGATAAATGCAGCATTTCAAATCAAATAGTGGACTATATCAGCTTG
AGTATCTATTAGTGGTGTTAGGATAATTAATATTTGGGAAAAAACTAAAATACTGCCCTTACTTATCTCA
TTATACCAAAATAGTTAAAGTTTAAAGTTAAATATTAAGAGGAAGCAATAAGCATCTCAGAAGAAAGGTA
GGTGAATAGTTTATAAGATTTTTCTATCCCTCCTACCAAAAGTGACATTTTTTAAAAGGAAAAGACTGAC
AAATTGGTAAGATTTAAAATGATGAGACTATGTAGAGTTGTAAACATTCTTACATTCAGTTCTCCCAGAA
GCCTACAGAGAGCCATTACTCAGAATTCCAGGAATATCAAATGGAAACTTACATCCTGTTCTGCACATTC
ACAATTGCCAGAAGATGAGATGATTCAGTGTCCATTGATGGATGGATGCAGAAAGCAATGTGGTCTGTAC
AAAAACATGGAATACTTTCAGCCTTAGAAAGGAAAGACATTCTGACACATGCTACAACATGGATGAAGCT
TGGGAACATTCTACTAAGTGAAAGAACCCAGTCGTAAGAGGACGGATACTGTCTGATTCCACTTAGCTGA
GGTCCCTGGAGTAGTCAGATTCATAGAGACAGAAAGAATGATGGGCACCAGGGGCTGGGAGAGAGAGAAT
GGGCAGGTAGTGTTTAATGGGGCCATTGTTTCAGTTTGGGAATATGAAAAGTTCTGAAGACAGACAGTGG
TGATGGTTGCATAATAGTGTGAATGTACTTAATGCCACTCAAGTGTACTCTTAAAGATGGTTAAATGGTC
AATTTTATATGTAGTTTACCACAATTTAGAAAAATTGACAGAGAAACTGAAGCTTAGGTATGAGTATACT
CACAAAAAGGCACAGAAACTCATGCTTCACTGCTGCCTTTATCCTAAAATGTCCTATAAAATGTGGGAAA
CCCTGTAATAACTCACTCTGTGAGCACAAATTTGGATCGAGTGAGAAGATACTTGACTTCCTTCCTCCAG
GCAGCCCATGGTTTAAGTTTTTATCTTGGACAAGATATCTTGTGTCTCTTCTCCTCAGTGTTCTGCTACC
CATTTATCTCAATATGCTTCAATGTATTTGTATGAAGATATGTCTGTATCCATTATGATCACCTACACAT
ATTACACATAGAAGGGGGTATGTGTTATAAAAACATATCTATACATGTCTGTGTATTTTTGTGATGACCA
AGTCTATAGTCAGACACCATGATACACATTTATTATATCAGCTGGAAGAGCTCATTCCATATCATTGTGG
AAATATCCTAGATTGCTAAAATTCAGTCATAATCCTATTCAATCCAGTTCTGAGTATTTGTTGGGTACCA
ACTGCAAGACATTCCATCCAGTTGTAAGCAACTGAAATTTGCCTTGACTTTCCCCAACAGCAAAAAGGCA
GACATGCGTGTTCTGGCTACATCAAGGTGGAAATCGGTCCTGTGTTCTCTTCTAGGGATCTGCTGGAGGA
AGGAAAGGCCACGGTTCCCTGCCTGGCAGAAAAACTTACCAGCGAGCTCATCACACATATCTGTGTAAGC
ACGGGCAGAGCTGTGGGTTCTCTAAAAAGAATACTACGACCGCAGAGCTGAACCTTGCTGGCTTCTTAAA
CATCACTGTACACACAGATCTTCTGAGGATCTTGTTAAGATGCAGTTTCAGATTCTGTGGGTCTGGAGTG
GGGCCTAGAATTCTGCATTTCCAGCAAGCCCCCAGACAATGTGGATATTCCTTTTCAGGGGACCACAGTC
AGGGGGAATGCTGATAGACTATATCTACTGGGCCAAAATAAAAATTAAAATCTTATGCACAAGCTACTAA
CTCTTCCTTTCTCATTGACAACCACTATTATAATGTCTTAGTCATTCTAATGAACATATTTTTTAACTTC
TAAAAGCTTTGTAAAAGCTCTCTGTGGTTCTTTTTAAAAGTCTGCCTGAATATAGTGTCTTCCTTTTTCA
AATTTTCTTTTCTTTTCTTTTCTTTCTTTTTTTTTTTTTTTTTTTTTTTTTTGAGACAGAGTCTCACTCT
GTTGCCCAGGCTGGAGTGCAGTGGTGTGATCTCAGCTCACTGCAACCTCTGCCTCCTGGGTTCAAGCAAT
TCTCCTACCTCAGCCTCCTGAGTAGCTGGGATTACAGGTGCCCACCACCATGCCTGGCTAATTTTTGTAT
TTTTAGTAGAGACAGGGTTTCACCATGTTGACCAGACTGGCCTTTTTCAAATTTTCAACTCAGCACCAGA
GTGCAAGGTCTTCCACGTGGTCCCCAGGAATGCGGGTGCATAACAGGGTTGTTTCCAGCCGACCATGATG
AGTGCAGAGCTCTCTGGGGTCCCACTGTATGCAGAAAGAGGATGCTTCCTTATTAGATTCCCCACCTCGA
GCAAGCCCATGGGGATTGATTTTTTGCCTCTGCACCAAGTCAGGTTCATAAGTTCCCGTTCGAATTTTCT
TACCTAGACAGATGCCCTTGTGGCTGAGCCGGGCTTCATTGCTGCCTTCTCCTTGAGCCCCTGCCTGGCC
ACTGTTACTGGGGCTGGCCTCTGACTACCCCTCACTAACTTGTGAGTCCACCGATACATTTAAAGGTGCA
GCTTTCACATGTCAGCTGGCATTTTTAGATGTTTGCCGTGGAAGGGTGAGCCAGCATATGGCGTCAACCG
TATTGTTAAAAACATAAGTCTCTGATCACTTTTTATTGATTGCAAGCAACATAAAAGTTGTTGAATCTCA
AATTGCTCCAAATGCCACTTTTTCAGAACCTACTAGACAAGTGGATCTCTCCAGTCTCCCTCCAGAGAGT
TTACCTAATATGACCACAGAGGAACTGCTCCCGGGTCACTCTGCCGGGGCCTAGGACCCATGCACAGTGG
GTGCCACAGTGCTGCTCATGAGGCTGCTGTCGCAGGAGTGGGGAAGGGGGAAGACCTGGGCAGAAAACAG
TGCCCCCAGTGTGTGCCCCCCTGCACCTCCCCCGGGTCTGGAAAAGCTTCCTTTTAGAGGAAGCCAGGAA
GTCAAATGGCCCACACAACTCCTCTGCAGAGGGAGGCCCGGGACCTCCTTTTCATTCTCTGTTCATCTTT
ACACATTTCCATTATTTTCTCTCCATTTTCCTCAGAAATCTCTGCCCCTGTTAGAAAATCAAATCAAGGA
GACTCACCAGAGAATAACAGAGGAGCTACAAAAGTATGGTGTCGACATACCGGAAGACGAAAATGAAAAA
ATGTTCTTCCTGATAGATGTGAGTGTTGCCAGCTGCATGGAGCTGGAGAAGCACATGTCATGGTCAAAAA
AGGGACCCTGGGCCTTATGCACTTCCTTCTTCACTCCCCCAAGGCTGATCCAAAGACATCTGGCCCGTAG
CACTCAAAGGGTGGACAGGGCTGAGGGAGGCAGGGCAGGGAGTGCAGATGTGGGGGTGGAGTCAGCAGCG
AGGGATGCTCAGGCTGCGTTGTGCCTACTCTGTCACGAGCATCACCCAGATCCCTAAGGCAGTAAGGGGT
GGGATAGGATTTTCTAGTGCCAAAACCTCTTCTTTCCCCTGATCCACAGTGTCCCATAAGAAAGCAAAGA
ATGACTCCCCACCCTCCACATAGGCACGGCCTCCAAATGACCTTGACACTTGGATTTGAAGTCTATCCAC
TTATACTGATGTTTTTCTTCTTGACAGAAAGTTAATGCCTTTAATCAGGACATCACTGCTCTCATGCAAG
GAGAGGAAACTGTAGGGGAGGAAGACATTCGGCTGTTTACCAGACTCCGACACGAGTTCCACAAATGGAG
TACAATAATTGAAAACAATTTTCAAGAAGGTGAGTGTCTTAGTCCCTTCTTTTGGGCTGCTACAACCGAA
TACCTGAGACTGGGTCATTTATAAACAGTAGAAACTTATTGCTCATTGTTCTGGAGGTGAGAAATCTATT
CTTAAGGAATCAGGAAATTTGGTGTCTGGTGAGAGCTTGTTCTCTGCTTCAAAGATGGCACCTTCTAGCT
GTGTCCTCTCATGGGATAAGGGACGAACAAGCTTCCTCGGACCTCTTTTTTACAGGGGTACCACAGGCAT
ACCTCAGAGATATTGTGGGTTCAGTTCCAGACAAAAAGAATATTGCAATAATGCAAGTCATATAAACTTC
TTGGTTCCTGGTGCATAAACAGTTCATTTATGCCCTACTGCAGTCTATTAAGTATGTAATAGCATTAGGC
CTAAAAAATATGTATGTACCTTAGTTTAAAACACCTTATTGCTAAAAAATTGCTGGTACAGAAACAAAAA
GTGAGCATGTGCTACTGGAAAAAAATGGTGCTGATTTGCTTGACATGGGATTGCCACAGACTTTCAATTT
GTAAAAAATACGGTATCAGTGAAGTGCAATAAAACAAGATATGCCTGGAATGCCATTATGCGGGCAGAGT
GCTCATAACCCAATCCTTCCTAAAGGTCTCCTCTGTTGATACCATCACACTGGGGATTAAGTTTCAACAT
AGGAATTTTTAGGGGACACCAACATGTAGACCATAGCAATGAGTCAATACCGTGGTAAACCTGATACGTT
GGCTTAAGACAGAGAAGAGTGGGGCAGTTGGGGAGGATGGTCAGGATAAGGAGCTAGTGACAACTAAAGC
CATGTTTGCTCTCTTCTATATCACTGAACCCAAATGACCATCCACTGATGAATTGATAAACCAACTGGCT
TGTGTCTGTGTGTAGCGGTTGGCTTTGGCTGTCATAACAAAGTACCACAGACTTGGGGGGGCTTAAACAG
TAGAAATTTATTTTCTTACAGTTCTGGAGGCTGGAAGTCCAAGATCAAGATGTTGGTGGAGCTGGTTCCT
TCTAAGGCCTCTCTCCTTGCCTTGCAGATGGCCTCTTCTCATTGGGTCCTCATGTGGTTGTCCCTCAGTG
TGTGTGTCCTCATCTCCTCCCACAAGGACACTAGGCAGATGGGATGAGGGCCCACCCTAGTGACCTGATT
TCAATTTAATTACCTCTTTGCCTGTCTCCAAACACAGTCAGATTCTGAGTTTCTGAGGGTTAGGACTTCA
ACATTGGAGTTTGAAGAGGTCACAACTCAAGCTCAGCCCGTAACACCAAGTCCTGGAATATTTCCAGCCA
CAGACAGGCACAGAGTGCTGGTCCACAGCACCCCATGGATGAACCTTGAAAATGTCATGCTGAGTGAAAG
AAGCCAGCCACAAAGGCCACACGGTCTATGATTCCATTGATAGAAAATGGCTAGAACAGGCAAACCCAGG
CAGGCAGAAAGCAGAATAGTGGCTGCCAGGGGCTGGGGAGGGAAAAGTGGGAAGTTATCACTGATGGGTG
ATGGATGTGGGGTTTGGGAGTTATGTCTGGGGATGGTCGCACAACTTTGTGAATATACTAAAATTCACTC
ACCCATACACTTTTTTTTTTCTTTTTCTTTGGAGACAGGGTCTCACTCTGTTGCCCAGGCTGGGGTGCAG
TGGCTCAGTCTCGGCTCACTGCACCCTCTGCCTCCCAGATTCAAGCGATTCTCCTGCTTCAGCCTCCACC
TCCCTAGTAGCTGGGATTATAGGCACCTGTCTAATTTTTGTATTTTTAGTAGAGATGGGGTTTCACCATG
TTGGCCAGGCTGGTCTCGACCTCCTGACCTCAGGTGATCACTGGCCTCAGCCTCCCAGAGTGTTGGGATT
ACGGGCGTGAGCCACTGTGCCTGGCCTGAACCATATATTTTTAACAGAGTGAATGTTATACTATGTAAAT
GACATCTCAATTAGAAAAATCCTTATGGGAAAATATTTCCTGACTAAAAAAAGTGTTCTAGATTACCACT
CAAAAAGGAACTCAAACCCTCTGAACTTCTGATGGGGCTAACTCTCTCTAGTGTGGATTGTTGGGAGTAC
AAATCATTCCAAAAGTTTAAAGAAAAATGCAGCATCTTACACAGTGAACAGTGCTACTGTATCACATTCA
TACAAGTTGATGTGCCTGGTTTACTCTGTTATCCCATTTGACTTGTAAACACTTTCTACACATGGCAATA
CTTTCGACACATGAATATGTGATGTATTGTTAATTCCAGAAAGTGTTCATGCTCATTTCTAATGGGCATG
CAGTTGAGGGCAAGGAGTGTATTATGGTACAATTTCTTTGGTAAAACTAAAATTGGATTCACAAAACTTC
ATACTCGAGTACGTTTTTAAGAAGGGGTCTTGGCCGGGCACGGTGGCTCACGCCTGTAATCCCAGCACTT
TGGGAGGCCGAGGCAGGTGGATCATGAGGTCAGGAAATTGAGACCATCCTGGCTAACACGGTGAAACCCC
GTCTCTACTAAAAATACAAAAAAATTAGCCGGGCGTGGTGGCGGGTACCTGTAGTCCCAGCTACTTGGGA
GGCTGAGGCAGAAGAATGGCGTGAACCCGGGAGGCAGAGCTTGCAGTGAGCTGAGATCACGCCACTGCAC
TCCAGCCTGGGTGACAGAGCGAGACTCTGTCACAAACAAACAAATGAAAAAAAAGGGTCTTACTCGAAGT
TTCTGCGTATGTGGGTTCCTGGCATCGTACCTGGCTCTGCACTCCCCTTCCTGAGATGACTAAGGAAAAT
TATCTTCAGATCTGGTTTTGTGTGTGTGTGTGTGTGTGTGTGTGTGTGTGTGTGTGTGTGTGTGTGTAAT
CCCTGGATATTTTTAGTTTACCAGTTAGATTTGATTTGATACCACTTTTTCTTGCCATTTATATTTTCAG
AAAATTTAGAATGGTATTGTGTTTAGAAAAATGTGCAAGATTATTTTTGTAAAATAATTTAGAGGGTTTT
TTTTCCTGCTATAGGCCATAAAATTTTGAGTAGAAAAATCCAGAAATTTGAAAATCAGTATCGTGGTAGA
GAGCTGCCAGGCTTTGTGAATTACAGGACATTTGAGACAATCGTGAAACAGCAAATCAAGGCACTGGAAG
AGCCGGCTGTGGATATGCTACACACCGTGACGGGTGAGTGCTCAGTTTCACCTCTGAGCATTGATTTCTA
AAGAAAGGAAAGGTTCGAACCAAAGCCAGCACCAAACTTCAGCACTTTCCTCCTGGGGTGCATCCCACAC
CAACGAGCAAACCTCTCATTCTCCAGATGCCAAGTTGGTATTCAACAATTCAATTCAATTCTGACACTAA
CTACCCTCAGTCAGTGTGGACCCCATAGCTTAAGGGCTCAGTTCCACAACACTGGCCCCAACTACAAATG
CCGGTCACAAGTCCCAGACCTCCTATTCTTCTGATGGACTGTTTATAAATCAAGGTTCTTGCGACCCATT
CCTCAGGTCAACCAAGAACTCTGGAACACACTTCACTGACATTTACTGGTCTATTAGAAAGGATTTGATA
AGGGGCACAAATGAAGCTGTTGGAGAGGCACATAGTAGGGGCCTGAACACAGAAGCTTCTGTCCCCACGG
GGTTGGGGGCACCATCCTCATGGCACAGAGATGTGGTCATCAACCAGGGAGCTCTTGGAACCTCACCGCG
GAGAAGGTTTTATGGAGGCCTCATCATGAAGGCATGATGGAGGATTGACTCAATCTCCAGGCCCTCCCTC
CTCTGTGGAGCTGGAAGTTCTAAGTTTCTAGCCAAGGCTTGGTCTTTCTAGTGCCCGGCCCCAATCCTGA
AGCTATGTAGGGGCCCACCAGGCATCATCTCTTCAAAACACGAGATACTCCTAAGGCTGGACATTCCAAG
AGATGTAGGGGCTCTATGTTAGGAAATGGGGACAAAGACAAAATATTTATATTTTTCCTATCACACCACA
CCTCCCCCCTGCTCCACTGCTATGCTGGCTTCACACTCAAAATCGGCTGTTTATTTGAAATCTCCGAGGA
GTAAAGCCAATGGTTCCATAACTGCACGTGTAGATGTGTTTGGAACCTTTTGGAGTGCTGTAGGAATCTA
GGTGTGTCACGGATAGGTAGGAAACTAGATCCTACTGTGGATCCACTCCCTTCTTGAAATGCTTTGCTTT
CTTGGTTTTCCAGGTATTAAATCTCTATTCTTCATCCTCTCCTTGACTGACAGTATCCTTACTCACACTT
CAGCTGCCTCATCTTAGCAGTAATTAATAATCACTCATGGATCCATGAACTAAGGAGCTGGAGATAGCCT
CAGAACAGCTCATTCAGAGGTGTATTTCCAGTAAAATTGACCTTTTAGCCCTGATAATCATATACCAAAA
CCTGCAATCATGTTGTTTTGGTCCATTGTAGACTCTTAACTCATTCCAGAGGAAAGTTTATAATACTTAG
AGCCTTATAGTCATAAAAATCAACATAGATATACCTATTTCTTTTTCAGAAATGTATGACATGGAGATCA
ATAAGAGGTTTTCAATCATAAAGATACTATACCTTGTATTACAATAAAATTCTGTGAGGAAGTAGAATAG
AAATGAGTTTCAAAAATAAAAGATAAATAATATAAATTTTTTAATCTAAGAGCTTGTTCTTGTATTTTTT
TCAAATGGATAATGTAGACACTCAAATTCCATTGATATATTTAAGAGTGATTTGACTTATATTAAGAGTT
GTATTATAAAATATTAATATTTATAATTTAAAAGAAATTACATTCTTTGCAGCTATTTAGGATAAAAAGT
TTAAATATCAAATAAATGTATGCCAGGGGTCATTTGCTTTTAAGATTCTTCCAGCAAATTATTAAGCAAA
AAGAGCATGCCTTGCTTTTTCATGGTAAAGAGAAGAAGGGAGCGGGGAGAGGGGAAACTTTACTTCATAC
CATTTGATCCTCATATTTTTTTGCATCTTAAGAAGAGAACAAATGATCCTACCAATATTGAACTATTTTT
CTCTCTTTGATTAGATATGGTCCGGCTTGCTTTCACAGATGTTTCGATAAAAAATTTTGAAGAGTTTTTT
AACCTCCACAGAACCGCCAAGGTAAAACCAACCATGTGTTGTTTAAAAAAAAAAAAGAAAAGAAATTAAG
CTTGACACTAGAAAATAGATTTCTTGGATGAGGATTATTTCAACTTTATTGTATACTTTTAGAACAGCAA
ATAACATCACTCACTAGTGCTTCTTCTGATGTTACCGGTGATGTCTGGTTAAAAGCAATAAAGGAGGGAG
TGCTTAAACGCACAGAACAAGAGATCCACAGTTAGCGGAGAAGATTATCACATCTAAGGGCAATGGCTCC
AAATCCAGAAACTCACTGAGGAAACTACATATAAAAATAGAATATTTCTGGCCCGAGTGGGCATGATGAG
CCTGTAATCCCAGCACTTTGGGAGGCTGAGGCCGGTAGATGACTTAAAGCCAGGAGTTTGAGACCAGCCT
GGCCCACATGGCAAAACCCCATCTCTACTAAAAATACAAAAAAGTAGCTGGACGTGGTGGTGCATGCCTG
TAATCCCAGCTACTTGGGAGGCTGACACTTTAGAATTGATTGAGCCCAGGAGGTGGAAGTTGCAGTGAGC
CAACATTGCATCACTGCACTCTAGCCTAGGCGATGGAGCGAGACCCCGTCTCAAAAAAAAAAAAAAAAAC
AAACAAAAAAACTTTCCATCCAGAGTGAGGAAAGAGCCTACAGGAAATGAGCCTGGGGGACAGACTGGGC
CAAGAGACCAGACTTAGCCACTCTTAGAAATAGGIGTCCCCGGCACAGATGAGGAGCCTGGCCCCATGAT
TCACCAGCTGGAGGCCTTGGGATGTGCCACTTCCAGCCTGTGCCCCTGACTCCTCATTCATAAAAGAAGA
CTGATAAGGCCTTCCTCAGAAGGTTGAGATGGACGTGGAGTAAGATGTTTAGGATGCACCTGCCACTGTG
CACTGTGCCTCTCCTCAAGGCCTGGAGGGTCCAGGGGTGAAGTTTCTCCTCCTCAGGTTTTGGCAACCAG
TTTCTCTAAACCCCGGGAACATAAAACATAATTTTCTGACTTAAACATGGCTTTCCTGCTCATCCCTGTG
GATTATCTGATGGATATGACAATCCTCGCCATCAGATATAGAAGCCCCTAAAAGAGAAAGGAAAGAAGCT
GAGTTACGGGGCCTGAAAGCAAGCCTGTGCAGGTCCCCAGGCCCCGGGATGGGGGTCCGGCCCATCTGTG
GCTCAAGCCTCCTGGGAAGCTCTGACCCTCAGCCAGGGCTAGAAACCTGCCTTAGATACACCAGGGCGCG
GCCCAGAGGGCTGTTCCAGGAAACGTGCTGTTTCACTCACGTTGGGTAACCTGGTATTTACGGACTTCTT
ACCTACTTTCCTGTGACTCAGGAATTTGTGTCTTGAGGGAAACTGTATTTATTTATTTTTTACTGTAGTC
CAAAATTGAAGACATTAGAGCAGAACAAGAGAGAGAAGGTGAGAAGCTGATCCGCCTCCACTTCCAGATG
GAACAGATTGTCTACTGCCAGGACCAGGTATACAGGGGTGCATTGCAGAAGGTCAGAGAGAAGGAGCTGG
AAGAAGAAAAGAAGAAGAAATCCTGGGATTTTGGGGCTTTCCAGTCCAGCTCGGCAACAGACTCTTCCAT
GGAGGAGATCTTTCAGCACCTGATGGCCTATCACCAGGTACGTCTTCGCGTGGTTCAGGATGCCAGCTTC
CATTCTTTCCTTTTCTTCTGAACGCCTCTCTCTTTAGTCTTGCTCTCTCTGTAGGTGACGTTGGTCAGCT
CTGTCGTTTACCTCCTTGTTAGCCTCCTGTATTAGTCCATTTTCATGCTGCTGATAAAGACATACCTGAG
ACTGGGCAATTTACAAAAGAAAGAGGTTTAACGGACTTACAGTTCCACATGGCTGGGGAGGCCTTCTACC
ATCACGGCAGAAGGCAATGGGCACTTCTTACCTGGCGGCGGTGGCAAGAGAGAGAATGAGAGCCAAGTGA
AAGGGGTTTCCCCTTATCAAACCATCAGATCTCATGAGAGTTACTCACTACCATGAGAACAGTATTGGAG
AAACTGCCCCCATGATTCAGTTATCTCCCCCTGAGTCCCTCCCACAACAGGTGGGAATTATGGGAGTACA
ATTCAAGATGAGATTTGGGTGGGGACACAGAGCCAAACCATATCGCCTTCGTAGAAGCAGCTCAACCTCA
GACAGAGAGATGGTGGCTTAGAGCCAGTGACATCTGGTTTTGATGGCTGTCTAGCTCTGGCCAAGTTACT
TAACCTCTCTGAGCCTCAGCTTTCTTTGTAAAATGGTGTCTCCTCATAGATTCTAGTGCATATTCCAGGA
GACGAGTGTGGATGATGATAATGGATTGCTAATGGAAAAACCAAACTCTGTTAAAATATTTGAAAGAGGT
TTATTCTGAGCCAAATATGAGGGACCATGGCTCTGGGAACAGTCTCAGGAGGTCCTGAGGAAGTGTGCCT
GAGGCTGTCAGGATGCAGTTTGATTTTATACATTTCAGAGAGGCAGGAATTGTAGGTAAAATCATAAATC
AATACATGGGAGGTGTACTTGCCCTCCCTAAAGAGGCAGGACACCTTGAAGGAGGGGGAGCTTACCGGTC
ATAGGTGGGTTCAGAGATTTTCTGGTTGACGATTGCTTGAAAGAGTTAAACTTTGTCTACAAACTTGACA
TCAATAGAAAGAAATGCCTGAGTTAAGGCAGTGTTAGAGGCCAAAGGTATGTAGATGAAGACTCTGGGTA
GCAGCCTTCAGAGAGAATAAATGGTAAATGTTTCTTTTCAGGCCTTAGAGGCAGCAGGCTCTCAGTTAAT
CTCTCCTAGATTCAGGGAAGGCCTAGAAGGGGAGAGGTCTGACTGCATTAATGGAGATTCTCTACAGGTG
CAAATTCCCCCCCCACAAAACATGGCAGGGCCATTTCAATCTGTTGGTCCTGTTACAGCCGTTTCAAAAT
ATGTCCACAAAATATATTTTTAGGTAAAATATTTGTATTTCCTTTAGGGTCTGCAATCTGTCTTGTGATG
CTATACCAGAGTCGGGTTGGAAAGTAAGCCATTTTATACTGAGTTCATGGAAACTCATCCAAGGAGATTT
CATGGTTTGTGGGGTGTGTGTGACTTAACCCCTGCCTCACATGACTTTATAATATGGTATCTTACTACTC
CAGAGTCTTTTTGGCCAACCTTATGATCTCAATTTCAACCTAAACTCCAAAAGGGCCTGGCTTCTCTTCC
TGTTACGGCCAGGAATTCAGATTTTCAGGTTTCTCTGGGGTCCACTTGGCCAAGAGGGGGTCTGTTGAGT
TGGCTGGAAGGCATAGGATTTTATTTCTGGTTTACAACAATTTCCTTAGTGCAGCATTGGAATGCAATGG
TAGCAGACTAAATGGAAGCTATCGCGTAGACACATGCTTTGGTTGATACTGCACGATTCAGTTAACCTGA
AGTACAATCTAATTCATCCTAGGGAAGGAGGCAGTGAACACAGACACAACTCAGGTAGAGCCCTTGGGAT
GTGTAAACACCTGAGGAGGTAAAGCAAATTGTAATCTCTCGGTTTATCAGATGTCCCCATTGCCTTACTA
TTTGGATGCTTTAAAGCAGGGCCTCTCAAACTCCACCCAGCACAGAGGCCTCCTGGGATCTTGTGGAAAT
GCAGATGCTGATTGCAGGTCAGGATGAAGCTGAGATTCTGCCTTTCTTTTTTTTTTTTTTTTGAAACCGA
GTCTCACTCCATTACCCAGGCTGGAGTGCAGTGGCACAATCTCAGCTCACTGCAACCTCTACATCCTGGG
TTCAAGCAATTCACCTGCCTCAGCCTCCCAAGTAGCTGGGATTACAGGCTTACCTTGCCACCATGCCTAG
CTATTTTTTCTATTTTTAGTAGAGATGGACTTTTACCATGTTGGCCAGGCTGGTCTTGAACTCCTGACCT
CAAGTGATCTGCCTGCCTCGGCCTCCCAAAGTGCTGGGATTACAGGCGTGAGCCACCATGCCTGGCCTCT
GCATTTCTAACGGGCTCTCAGGGGTCACCATACTACTGGATAGAGGCCACACTTGGAGGAGCAAGGCTCT
AAACCGAGGGTCAACATCCATTCCTCCAGACACTGGGAGCTGCATGCACGTGAGTGAAGCCAGTTAAGGG
GAAGACAGGCATGCACATCAGCTTCTCCTGCAGCCAAGCTCACACCTGTCCGCTGCTTCCACTGCCTCCT
AGAATGAACAGTTACCTTGAGAGTAGGTGAGGCATATACATGCACAGAATCCAAACAATAGGATGAGTGA
CAATGGCAGAGGAGTCTCCGAGCCAAGCAGCTCCCTGGACAGAAGCAGCCCTTCTCCGGGTTCATTTCTG
TCCTCCGAGGCTGACTCATGCACTCAAAAGCTCCCATGCATATACATTTTATAATGGTTTTTACACAAAG
GTTAGCAGAGGAGTGGACGTGCTGCTCTGTACCCTGCCTCTTTTGCTGTACCTGGGAGATTGTTCTGCCT
CAGTTCTGATGGGGCTGCCTTGTTCTTTTCAATGGCTGCTGAGTATCCCATTTTATGGATGTGGTATATT
GAGCCAGCTCCCTTTAAGCGAACAGTTTGTTTGCAGTCTTTTGCTAATGCAGGTGTGTTGCTGTGAATAG
GTTTGTTTGTATATCATGTATCTGGAAGCATCAATTCCTAGAAATGAGATTCCTGGTATATTAGGATTGT
GCAGGGAAACAGAACCACAGATATATGTATGTAAAGAAGTATATTTCAGCCAGGCATGGTGGCTCATGCC
TGTAATCCCGGCACTCTGGAAGGCTGAGGTGGGTGGATCTCTTGAGGCCAGGAGTTTGAGACCAGCCTGG
CCAACATGGCGAAACCCGGTCTCTACTAAAAATACAAAAAAATTAGCTGTGCATGGTGGCCCATGCCTAT
AGTCCCAGCTACTTGGGAAGCTGAGATATGAGAATTGCTCGAACCTGGGAGGCAGAGGTTGCAGTGAGCC
AAGATCACACCACTGCACTCCAGCCTGGGTGACAGAGCGAGACTCCATCTCAAAACAAACAAACAAACAA
AAAACAAAACAAAACAAAAAACAGAAGGAAAGAAAGAAATATATGTATATTTCAAGGAATTGGCTTCTGC
CATTTTGGGAGCTGGCAAGTCCAAAATCCCAGGGCAGGCCAGCAGGAAGAGCAGGCCAGAAATTGCAGCA
GGAGCTAAGGCTGAGTCCACAGGCGGAATTTCTTCTTTTTAGGGAAACCTCATTTTTCTTCTTAAGACCC
TCAACTGATTGGATGAGGCCCATCCACATCATTGAGAATGGTCTCCTTCACTTAAAGTCAGTGGGTTACA
CATGTTACCCACATCTACAGAATACCTCCGCAGCAATACCTAGATTCGTGTTTGATGGAATCACTGGGGA
CTCGAGCCTAGCCAAGCTAACACATGAAACACACCATCACAGCTGGGGAAAGGATGGCTTATTTTAGACT
GATAAAGATGACCCAGAGAAGGCCTGCTCCATCCACACTGGCCGCTTTAGTCTGCACTAAAGTTGTTGGT
TTTTTTTGTTTGTTTGGTTTTTTTTTGGTGACAGAGTCTCACTCTGTCGCCCAGGCTGGAGTGCAGTGGC
GCAGTCTCAGCTCACTGCAACCTCTGCATCCTGAGATCAAGCGATTCTCCTGCCTCAGCCTCCTGAGTAG
CTGGGACTACAGGCACGTGCCACCACACTCGGCTAATTTTTGTTTTTTCAGTAGAGACGGGGTTTCACCA
TATTGGCCAGGCTGGTCTTGAACGCCTGACCATGTGATCCATCCGCCTCAGTCTACCAAAGTGCTGGGTT
TACAGGCGTGAGCCACCACGCCCGGCCTTGTTGGGGTTTTTTGACAGCCTAATAGGTGAAAATGACATCT
CATTACAATCTTAATTGGCATTCTCTTATGACAACAAGCTGGTACATCTTTTTGTGTGTTGAGGGTTATT
TCTATTTCTTGCTCAGCAAACAGTTCATCCAGGAAGAGCTTCTTGGTGAGATAGTAGACCTCTGCGATTT
CTGTTGCAGACGATCTACATTTTGTCATTTGCTTTGTCATTTTTGTCTATGGTGGTTTTAGACTATGCGT
AAGTTTTCTAGAGCAGAAACTCAAGTTGGATTTGGGCCTCAGTGGTTATTGCCATACTTTAAAAGGACTT
TGTCTCCCTGAGATGATAAATGAGGTGGACAATATTTTCTTTAAGTAATTTCTTATTTTAACTGTTACAT
GATACCTTTGGCCCATTTGGAGTTCTTTGATGTCAAGAATGAGGCAGGATCCAGATGGCAGCAGAGGTCC
CAGTCCCATCCTGGAAGGGTCGTCTAGTTCCCACTGGTACTCCACACGCCCACTCAGGCACTCACTTCCC
CTCTGCGTTGGGTCTTGTCTGCAAGACTCTCTTATGTTTTACCATCTAGTGCAGCCAGCACCCCCACATC
ACCCTCACTTTTTCTTTCTTTAAATTGTGCAGAAATATTCATCATGTCTATTTTGCCATCTTAACCATTT
GGGGGTACATAGTTCAGTGGCATTAAGTACATTCATATTGTGCCAGCATCACCAGCAGCCATCTCCAGGA
CCCTATCACCTTCCCACACTGAAACTCTGTCCCCATTAAACACATTCCCCATTCCCCGCCCCTGAATCCC
TGACAGCTACCATCCTACTGTCTGTCTCTGTGAATTCAACTAACCTAAGTACCTCATAGGAGTTGTGACT
GGCTTGTTTCATGCAGTATGATGTCCTCATCCAGGTGGTAGCAAGTGTCAGAGTTTCACGCCTATTTATT
TATTATTATGAGACAGAGTCTTGCTCTGTCGCCCAGCCTGGAGTACAATGGCGCGATCCCAGCTCACTGC
AGCCTCCCCCTGCCTGGGTTCAAACAATTCTCCTGCCTCAGCCTCCCATGGTGTGCCGCCACACCTGGCT
ATTTTTTGTATTTTTAGTAGAGACGCGGTTTCACCACGTTGACCAGGCTGGTCTGGAAATGCAGTTTTTG
CACTGTCTGCCTGCTTACCTTTATAGAGCATATTTTGCCCTCTTCCATCAGAATTACCCATTTAATGGTC
AGGAAAAGCTGCTGGGAATATGACTCATAGCTGGGACATTCTCTGCACTGTGCATAGTTCCTCTCTGCCA
CCACCATGGAGGAGATTGATGGGTTTGAAACCCAGGGGAAGGTCATTGCCCTGCGAGGGTCTCCCTCATT
GAGAATCTGGATCCCCTCATGTGCACATGGTGAGGTCAGAGTCCCCTCCTCACAGTGTCCCCTCCACCCT
CCCGTGAACTGTTCTTTCCTTCCAGGAGGCCAGCAAGCGCATCTCCAGCCACATCCCTTTGATCATCCAG
TTCTTCATGCTCCAGACGTACGGCCAGCAGCTTCAGAAGGCCATGCTGCAGCTCCTGCAGGACAAGGACA
CCTACAGCTGGCTCCTGAAGGAGCGGAGCGACACCAGCGACAAGCGGAAGTTCCTGAAGGAGCGGCTTGC
ACGGCTGACGCAGGCTCGGCGCCGGCTTGCCCAGTTCCCCGGTTAACCACACTCTGTCCAGCCCCGTAGA
CGTGCACGCACACTGTCTGCCCCCGTTCCCGGGTAGCCACTGGACTGACGACTTGAGTGCTCAGTAGTCA
GACTGGATAGTCCGTCTCTGCTTATCCGTTAGCCGTGGTGATTTAGCAGGAAGCTGTGAGAGCAGTTTGG
TTTCTAGCATGAAGACAGAGCCCCACCCTCAGATGCACATGAGCTGGCGGGATTGAAGGATGCTGTCTTC
GTACTGGGAAAGGGATTTTCAGCCCTCAGAATCGCTCCACCTTGCAGCTCTCCCCTTCTCTGTATTCCTA
GAAACTGACACATGCTGAACATCACAGCTTATTTCCTCATTTTTATAATGTCCCTTCACAAACCCAGTGT
TTTAGGAGCATGAGTGCCGTGTGTGTGCGTCCTGTCGGAGCCCTGTCTCCTCTCTCTGTAATAAACTCAT
TTCTAGCAGACACTGCTCTGCCATGTTTTGTATTTTGGCGAGAAGCCTGAAACTAGCAGGTAGGGTGCAG
TGGAGCAGTGGACGTAAAGCTGCCCTCTGTGGCGGGGCCAGGTAGGAGCAAGCAAAAGAACAGGGTCTGA
TGATTTCCTAGAGACTGGAGGGAAATGGGGGAACACACTTTAAAAAATGAAACAATATGAAGCCAACGGT
GCTAACTTAATAATGTGCATTGATCAAATGTCCAATTTGTTGAGTCACTGGGCACCTGGCATGGAGTTGA
AAAGCACAAGGCCCAGTCAGGAAGCGGTTGCCAGGCCAGGTGCGATGGCTCACGCTGTAATCCCTTCACT
TTGGGAGGCCGAGGCGGGTGGATCACCTGAGGTCAGGAGTTCGGGACCAGCCTGACCAATATGGTGAAAC
CCTGTCTCTACTAAAAATACAAAAATTAGCTGGGTGTGGTGGCGTGTGCCTATAGTCCCAGCTACTCAGG
AGGCTGAGACATGAGAATTGCTTAAACCCGGGAGGTGGAGGTTGCAGTGAGCCCAGATCGTGCCACTGCA
CTCCAGCCTGGGCTACAGAGCAAGACTCCATCTCAGGGAAAAAAAAAGGTTTCCCAGTGGGACTTTACCT
GCAGAGGCTGTGGCTGGGGGAGCGGTCTGGGAAGCAGAGAAGGCGGAACACTAACCTTGGAGGAGAACCT
GACTGAAGTTCTCTGGTCTAGCCTGGTTGGGGGAGACTCGAGCCACACTCCTACGCCCTGACTCTGAGAA
CCATGGCTCCCAATCAGTGCTGCTGCTGGGCCTCCTCACCATGAGGCCAGGGCAGGCTCAAGGGCAGGAA
ATCGAACAGTGGACTAGATGGATGGGGCAGGAGAGGACCGGACACTCTGTCCCTTAAGGGGAGAGTTGAA
GGATCAGGATTGCCCCAGCCTAGGAAAGAGAAGGCCAAAAGGCAACAAGAGAGATGAAGTGTGAGCACCA
AGGTGTGACATCCTCTGGGGAGGTATATTCTAGATGATCAGTGTGGGGAATGGGGGTACTTTCTACAGAG
GGGCCCCCGGATGGCATGGCTTCCTCAAGAGCTGTGAGGTCCCAGGCAGTGGGGGCATCAGGCCAAGCTG
CACAGCTGCTTGTCAGGTTGGGGGTGGGGGCTGGACCAGCAACACCATCCTCTCCCCTCTGGACCACGCC
CCCCCATCCCAGATCTCTTCTGACTCTTGGGACTGTGTAATTTTCAATTCTTCGGGAAGTTTGAGAATAC
ATTTATAAGAGAGGATGTCCGGAGAGCAGTGGACATTTCACAAGCCCAGGTCAGCAGCTGCCGCTGGGCA
GCAACTTTATGGTTCCCCCAGGTGCTGAAGTGCACCTGCAGGTTGACTTGATCTACTGTTAACAGAGGGT
GATCAGTTTGACTCATGCTTAAGGGAGAGACGACACCAACACACACACAGAGGTTTGCAAGAGTAAGGCA
GGGTGGATCCAAACATTCTTAATTCAATACTAGGCACGCGTTCTCGCAGTGGACATGGCCCAGGTCCTCT
GGCTGTGTTGGTGCATCAGACAGCTCGGCCCTTCCATGAGGCAGGTGCCCTCTCGTGGCAGGCCTGGGAA
ACTTCTCCACTACAAGACGTCCCTTCCTTCCACCACCACCCAAATCAGTCTTCCACTTGCCTTAAGTTTC
TGCTGCACGGGAAACAGAGTCAGAGGTGTCAAAGTGTTGCACAGGATTCTATTTTGCTGTTGTTAGAATT
TTGCTAGTGATAACCAGAGAGGATGGAGGAAGAAAGTTGACAGTATGTGTCCCAGAGAACAGCAATTTCA
ATTTCCCCAGCAGAGTGAACCTGCTGGGGATGCCCCAAGGTGCCCGCAAGGATGTTCCTTTCAGCCAAAA
TGAACATGTGTGGTGATCCCCCAGGCCTGTCCTAACTAGTCATCCAGAGCCGAGAGGGGAGTGGCCCCAG
CGGCCAGAACTGGGAGAGAGCCCTGGAGACCACTTTGTGTGACTCCTTCCTG

### BRIEF DESCRIPTION OF THE DRAWINGS

The invention will be more clearly understood from the following description of an embodiment thereof, given by way of example only, with reference to the accompanying drawings, in which:—

FIG. 1 illustrates distribution plots of (A) SAA, (B) CRP and (C) Hp for healthy, infectious and inflammatory disease.

FIG. 2 illustrates mean SAA, CRP and Hp trends over time for septic arthritis.

FIG. 3 illustrates a receiver operating characteristic (ROC) curve showing the area under the curve (AUC) plot for Hp when evaluating biomarker ability to classify healthy and non-healthy patients.

FIG. 4 illustrates a ROC curve showing an AUC for SAA when evaluating biomarker ability to classify healthy and non-healthy patients.

FIG. 5 illustrates a ROC curve showing an AUC for CRP when evaluating biomarker ability to classify healthy and non-healthy patients.

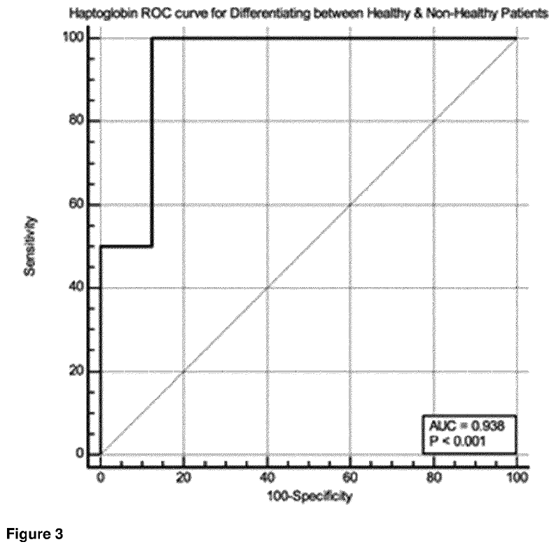

FIG. 6 illustrates a ROC curve showing an AUC for SAA when evaluating biomarker ability to classify healthy and Inflammation groups.

FIG. 7 illustrates a ROC curve showing an AUC for SAA when evaluating biomarker ability to classify infectious and non-infectious groups.

FIG. 8 illustrates a ROC curve showing an AUC for Hp when evaluating biomarker ability to classify Infectious and Non-Infection groups.

FIG. 9 illustrates a ROC curve showing an AUC for CRP when evaluating biomarker ability to classify Infection and Non-infection groups.

FIG. 10 illustrates a ROC curve showing an AUC for SAA when evaluating biomarker ability to classify Infectious and Inflammatory groups.

FIG. 11 illustrates a ROC curve showing an AUC for Hp when evaluating biomarker ability to classify Infectious and Inflammatory groups.

FIG. 12 illustrates a ROC curve showing an AUC for CRP when evaluating biomarker ability to classify Infectious and Inflammatory groups.

FIG. 13 illustrates a ROC curve showing an AUC for Hp when differentiating between healthy and inflammatory conditions in an individual.

FIG. 14 illustrates a ROC curve showing an AUC for MxA when evaluating biomarker ability to differentiate bacterial infection from non-bacterial infection.

FIG. 15 illustrates a ROC curve showing an AUC for Hp when evaluating biomarker ability to differentiate bacterial infection from non-bacterial infection.

FIG. 16 illustrates a ROC curve showing an AUC for CRP when evaluating biomarker ability to differentiate bacterial infection from non-bacterial infection.

FIG. 17 illustrates a ROC curve showing an AUC for SAA when evaluating biomarker ability to differentiate bacterial infection from non-bacterial infection.

### DETAILED DESCRIPTION OF THE DRAWINGS

### Materials and Methods

In this study SAA, CRP and Hp levels were retrospectively assessed in repository equine serum samples (n=162) with clinical records available. Also in this study SAA, CRP, Hp and MxA levels were retrospectively assessed in repository human serum samples (n=60) with clinical records available. Samples were included in the study if they had a healthy, infectious or inflammatory clinical record at the time of collection and divided into five groups: Healthy, Inflammation, Infection, Non-Healthy (inflammation and Infection combined) and Non-Infection (Healthy and Inflammation combined). The Kruskal Wallace test was engaged on all samples in the Healthy, Inflammatory and Infectious groups to determine differences in levels across the groups for each biomarker, where P<0.05 was considered statistically significant. Receiver operating curves were used on a subset of data across groups where data for all three biomarkers was available for each sample to identify the diagnostic ability of the biomarkers to differentiate between healthy and disease states. Diagnostic ranges were determined by Youden index (a single statistic that captures the performance of a dichotomous diagnostic test) and in Microsoft Excel using diagnostic value statistics, sensitivity, specificity, positive predicative values and negative predictive values. The biomarkers were also assessed in cases where serial sampling across a time course was available to compare performance across time.

Protein levels were determined using immunoassays. CRP and Hp levels were measured using species appropriate enzyme linked immunosorbent assays (ELISA). SAA concentrations were determined using a commercial lateral flow test. MxA levels were measured using species appropriate enzyme linked immunosorbent assays (ELISA). Statistical manipulations were performed using Microsoft Excel and MedCalc Software. Other methods can be used to detect nucleic acids, such as PCR, quantitative (or real time) PCR (qPCR), reverse transcriptase (RT) PCR (RT-PCR), RT-qPCR, Photometry, Fluorescence, lateral flow methods, and those methods described above. Such techniques can give quantitative outputs such as molar, ng/ul, ng/ml, copies/ul, and optical density (OD) readings.

### C Reactive Protein (CRP)

Serum CRP protein concentrations were determined using a CRP Horse ELISA kit and performed using antibodies and standards obtained from Abcam, Cambridge, UK. All reagents were equilibrated to room temperature. Serum samples were diluted before use with 1× Diluent (supplied as 5× diluent concentrate and diluted 1/5 using deionized water). 100 μL of each standard, including zero control, and each sample was pipetted, in duplicate or dilution, into predesignated wells on a 96 well micro titer plate and covered with an adhesive plate cover. The plate was left to incubate at room temperature for thirty (30±2) minutes. The plate was then aspirated and washed four times with wash buffer (supplied as 20×concentrate and diluted 1/20 with deionized water). Following this, 100 μL CRP Horse HRP conjugate (10 μL enzyme-antibody conjugate to 990 μL of 1× diluent per test strip) was added to each well and the plate covered with an adhesive plate cover. The plate was left to incubate at room temperature for thirty (30±2) minutes in the dark. The plate was then aspirated and washed four times with wash buffer. Following this, 100 μL of TMB Substrate was added to each well, the plate covered with an adhesive plate cover and incubated in the dark at room temperature for precisely ten minutes. After ten minutes, 100 μL of Stop Solution was added to each well. Absorbance was then read at 450 nm on a microplate reader (Benchmark microplate reader, Bio-Rad, US). CRP levels were extrapolated from standard curve of CRP level (ng/mL) versus optical density at 450 nm (Excel).

### C Reactive Protein (CRP) (Human Sample Analysis)

Serum CRP protein concentrations were determined using a CRP human turbi latex immunoturbidimetric assay and performed using reagents and calibrator from Spectrum Diagnostics, Obour City, Egypt. The reagents and spectrophotometer were brought to 37° C. and the reagents were shaken gently before mixing. The working reagent was prepared with 1 part of Latex Reagent and 4 parts of Buffer Reagent. The calibrator was reconstituted with distilled water, mixed gently and incubated at room temperature for 10 minutes before use. The spectrophotometer was adjusted to zero with distilled water. Following this, 500 μl working reagent and 5 μl calibrator or sample were pipetted into a cuvette and mixed. Absorbance was read at 540 nm immediately (A1) and after 2 minutes (A2) of the sample addition. The concentration of CRP was calculated using the following calculation:

(A2−A1)sample×calibrator concentration=mg/L CRP (A2−A1)calibrator

### Haptoglobin (Hp)

Serum samples were diluted before use with 1× Diluent (supplied as 5× diluent concentrate and diluted 1/5 using deionized water). 100 μL of each standard, including zero control, and each sample was pipetted, in duplicate or dilution, into pre-designated wells on a 96 well micro titer plate and covered with an adhesive plate cover. The plate was left to incubate at room temperature for thirty (30±2) minutes. The plate was then aspirated and washed four times with wash buffer (supplied as 20× concentrate and diluted 1/20 with deionized water). Following this, 100 μL Haptoglobin Horse HRP conjugate (10 μL enzyme-antibody conjugate to 990 μL of 1× diluent per test strip) was added to each well and the plate covered with an adhesive plate cover. The plate was left to incubate in the dark at room temperature for thirty (30±2) minutes. The plate was then aspirated and washed four times with wash buffer. Following this, 1004 of TMB Substrate was added to each well, the plate covered with an adhesive plate cover and incubated in the dark at room temperature for precisely ten minutes. After ten minutes, 100 μL of Stop Solution was added to each well. Absorbance was then read at 450 nm on a microplate reader (Benchmark microplate reader, Bio-Rad, US). Haptoglobin levels were extrapolated from standard curve of Haptoglobin level (ng/mL) versus optical density at 450 nm (Excel).

### Haptoglobin (Hp) (Human Sample Analysis)

Serum Hp protein concentrations were determined using a Hp human immunoturbidimetric assay and performed using reagents and calibrator from Kamiya Biomedical Company, Seattle, Wash., USA. All reagents and specimens were allowed to come to room temperature and mixed gently before using. The spectrophotometer was adjusted to zero with distilled water prior to sample testing. In a cuvette, 6 μl calibrator/control/sample was added to 500 μl buffer reagent and incubated at 37° C. for 5 minutes. A reading was taken at 600 nm, after which 140 μl antiserum reagent was added to the cuvette, mixed with the sample and buffer reagent and incubated at 37° C. for 5 minutes. A second reading was then taken at 600 nm. The difference between reading 1 and reading 2 was calculated and the concentration of Hp was extrapolated from standard curve of Hp level (mg/dL) versus absorbance at 600 nm (Excel).

### Serum Amyloid A (SAA)

Serum SAA samples were analysed utilising a commercial lateral flow immunoassay test, Equine SAA, Stablelab. Assay comprised of immunoassay strip housed in a cartridge, mix solution for dilution and propriety EQ1 reader device for SAA quantitation. Samples were allowed to come to room temperature and allowed to mix before use. Samples required a dilution whereby 5 μL of sample (serum/plasma) was pipetted into a mix solution. 4 drops of sample (approx. 100 μL) was then added to the sample well of the lateral flow cartridge. A timer was then set for a total run time of ten minutes, following which the cartridge was inserted into associated reader device for SAA quantitation. Readout were shown in μg/ml.

### Serum Amyloid a (SAA) (Human Sample Analysis)

Serum SAA samples were analysed utilising a lateral flow immunoassay test. The assay comprised of an immunoassay strip housed in a cartridge, mix solution for dilution and \handheld reader device for SAA quantitation. Samples were allowed to come to room temperature and mixed before use. Samples required a dilution whereby 5 μL of sample (serum/plasma) was pipetted into a mix solution. 4 drops of sample (approx. 100 μL) was then added to the sample well of the lateral flow cartridge. A timer was then set for a total run time of ten minutes, following which the cartridge was inserted into associated reader device for SAA quantitation. Readouts were shown in ug/ml.

### Myxovirus Resistance Protein A (MxA)

Serum samples were diluted before use with 1× Diluent. 100 μL of each standard, including zero control, and each sample was pipetted, in duplicate or dilution, into pre-designated wells on a 96 well micro titre plate. The plate was left to incubate at room temperature for 1 hour, shaking at ca. 300 rpm on an orbital microplate shaker. The plate was then aspirated and washed three times with wash buffer (supplied as 10× concentrate and diluted 1/10 with deionized water). Following this, 100 μL Biotin Labelled Anti-MxA Antibody Solution was added to each well and left to incubate at room temperature for 1 hour, shaking at ca. 300 rpm on an orbital microplate shaker. The plate was then aspirated and washed three times with wash buffer. Following this, 100 μL of Streptavidin-HRP Conjugate was added to each well and the plate incubated at room temperature for 30 minutes, shaking at ca. 300 rpm on an orbital microplate shaker. The plate was then washed five times with wash buffer. 100 μL of Substrate Solution was added to each well, the plate covered with tin foil and incubated in the dark for 20 minutes at room temperature (no shaking carried out during this incubation). 100 μL of Stop Solution was then added to each well to stop colour development. Absorbance was then read at 450 nm on a microplate reader (Benchmark microplate reader, Bio-Rad, US). MxA levels were extrapolated from standard curve of the log of MxA level (ng/mL) versus optical density at 450 nm (Excel).

Myxovirus Resistance Protein A (MxA) (Human Sample Analysis) 100 μL of each standard, including zero control, was pipetted in duplicate into pre-designated wells on a 96 well micro titre plate. Samples were diluted before use with sample diluent. One part sample was added to 4 parts sample diluent. 100 μL HRP-conjugate reagent was added to each well, the plate covered with an adhesive strip and incubated for 60 minutes. Each well was then aspirated and washed for a total of five washes. To Wash, each well was filled with wash solution (400 μL) and then all liquid removed. After the last wash, the plate was inverted and blotted against clean paper towels to ensure complete liquid removal. 50 μL chromogen solution A and 50 μL chromogen solution B was added to each well, gently mixed and incubated for 15 minutes at 37° C. 50 μl stop solution was then added to each well and the optical density read at 450 nm using a microplate reader (Benchmark microplate reader, Bio-Rad, US). MxA levels were extrapolated from standard curve of MxA level (ng/mL) versus optical density at 450 nm (Excel).

### Results

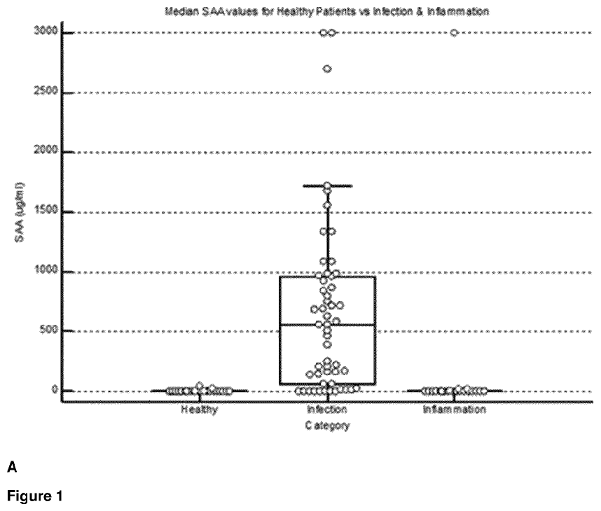

In single time point analysis levels of SAA (n=99) and CRP (n=39) there was a significant difference in biomarker values between the Healthy, Inflammation and Infection groups, p<0.000001 and p=0.010325, respectively (see FIG. 1A, 1B). SAA had a median value of 558 ug/ml for infection compared to median values of 0 ug/ml for both inflammatory disease and healthy controls. There was no significant difference between the Healthy group and the Inflammation group for SAA. CRP demonstrated a median value of 19.45 ug/ml for the infection group, approximately three times normal reference levels compared to 5.45 ug/ml and 2.98 ug/ml for the Healthy and Inflammation groups respectively which are within normal limits. CRP was slightly more elevated in the Healthy group than in the Inflammation group, but this was not considered statistically significant. There was a statistically significant distribution of Hp levels (n=24) across all three groups (p=0.000345) (FIG. 1C). The median Hp value was 2153 ug/ml for the Infection group, 731 ug/ml for the inflammation group and 397 ug/ml for the Healthy group.

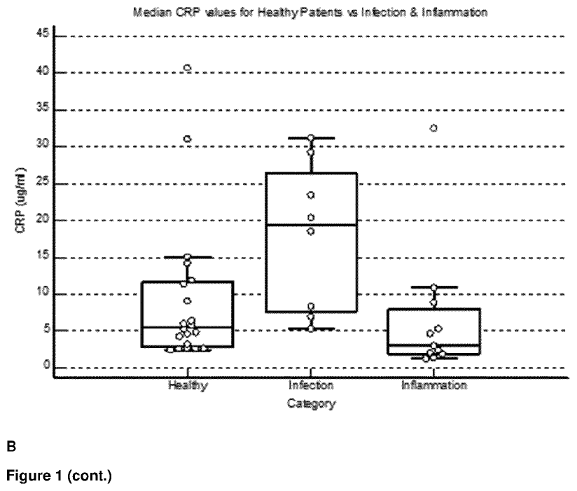

In a time course assessment for patients with a confirmed infection (n=3) (see FIG. 2), CRP was elevated from baseline levels within 12 hours of infectious challenge. SAA started to elevate rapidly at 24 hours for cases of septic arthritis. Hp levels started to elevate immediately to just above the reportedly normal reference level (200-1000 ug/ml) within 10 hours, reaching a peak at 30 hours. None of the biomarkers returned to normal levels during the time course (FIG. 2).

ROC analysis was undertaken on a subset of data for which SAA, CRP and Hp (n=24) levels were available to identify the diagnostic ability of the biomarkers to differentiate between healthy and disease states.

### Distinguishing Healthy and Non-Healthy (Infection and Inflammation Combined)

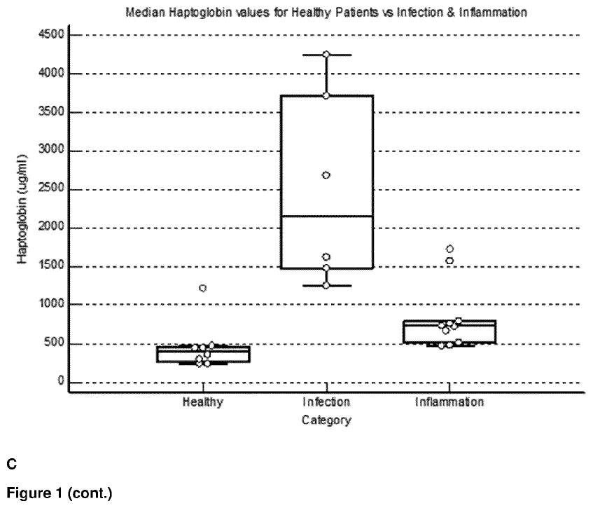

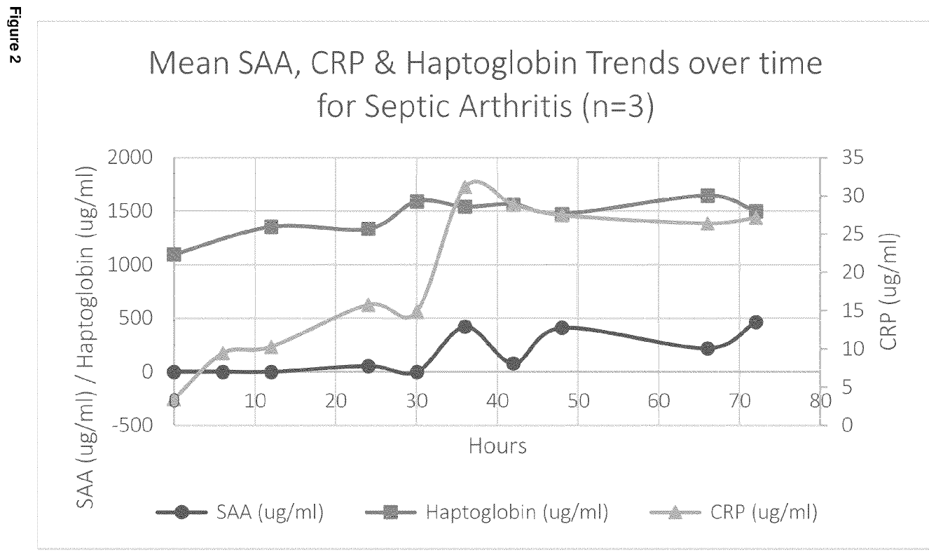

The most significant area under the curve (AUC) for the Healthy and Non-Healthy Group was observed for Hp (see FIG. 3) with an optimum cut off value of >473 ug/ml reported by Youden Index. Sensitivity was 100%, demonstrating all Non-Healthy patients are differentiated from the Healthy group (FIG. 3 and Table 3 below) and specificity was 87.5% with an overall accuracy of 95.8%. AUC value for SAA was not significant for the same group at 0.656 and p=0.118 (see FIG. 4). Sensitivity was reported at 37.5% and specificity was 100%. The AUC for CRP was also not significant at 0.520 and P=0.8992.

TABLE 3
showing diagnostic performance statistics for a range of Hp cut-off values,
including Youden Index associated criterion of greater than 473 ug/ml, to distinguish
between healthy and non-healthy patients (infection and inflammation combined).

| Cut-off (ug/ml) -> | 400 | 473 | 500 | 600 | 700 |
|---|---|---|---|---|---|
| Sensitivity | 100.0 | 100.0 | 87.5 | 81.3 | 75.0 |
| Specificity | 50.0 | 87.5 | 87.5 | 87.5 | 87.5 |
| Pos. Predictive Value | 80.0 | 94.1 | 93.3 | 92.9 | 92.3 |
| Neg. Predictive Value | 100.0 | 100.0 | 77.8 | 70.0 | 63.6 |
| Accuracy | 83.3 | 95.8 | 87.5 | 83.3 | 79.2 |

### Distinguishing Healthy and Inflammation

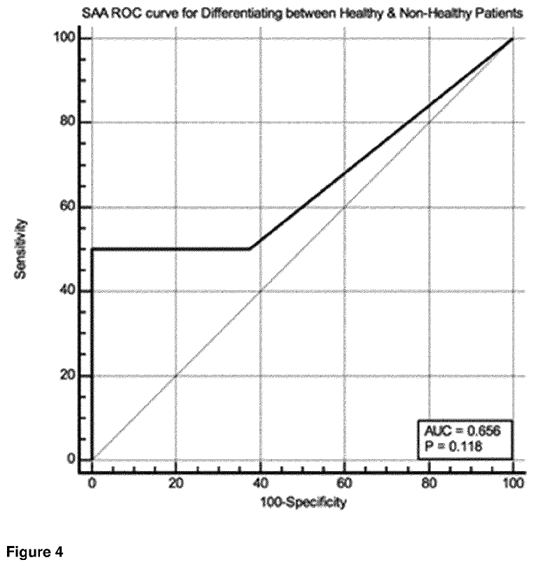

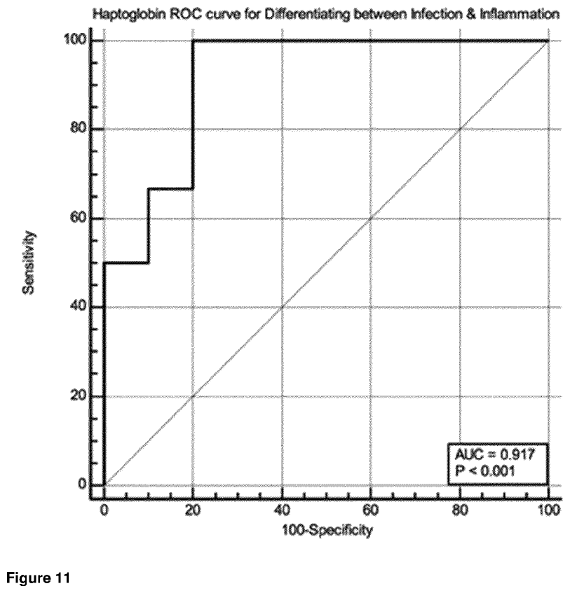

The most significant AUC was observed for Hp at 0.900 and p=0.0001 with a cut off value of 473 ug/ml (FIG. 13 and Table 4) SAA reported an AUC of 0.550, p=0.66 (FIG. 6). CRP had an AUC of 0.800 and P=0.13.

TABLE 4
showing diagnostic performance statistics for a range of Hp cut-off values,
including Youden Index associated criterion of greater than 473 ug/ml,
to distinguish between healthy and inflammation patients

| Cut-off (ug/ml) | 473 | 600 | 796 | 1217 |
|---|---|---|---|---|
| Sensitivity | 100.0 | 70.0 | 20.0 | 20.0 |
| Specificity | 87.5 | 87.5 | 87.5 | 88.9 |
| Pos. Predictive Value | 90.9 | 87.5 | 66.7 | 66.7 |
| Neg. Predictive Value | 100.0 | 70.0 | 46.7 | 50.0 |
| Accuracy | 94.4 | 77.8 | 50.0 | 52.6 |

### Distinguishing Infection and Non-Infection (Healthy and Inflammation)

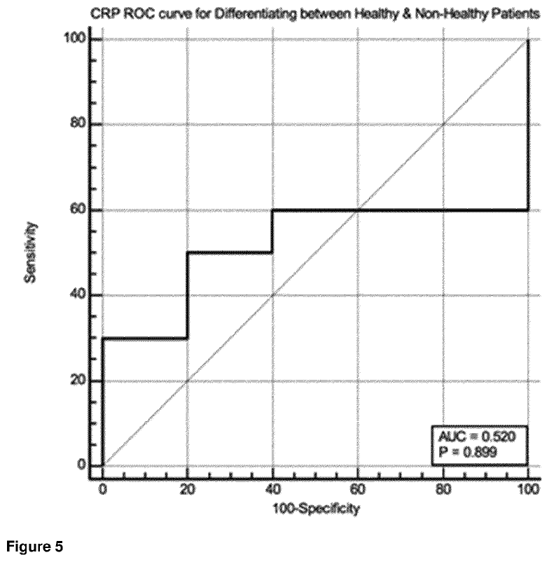

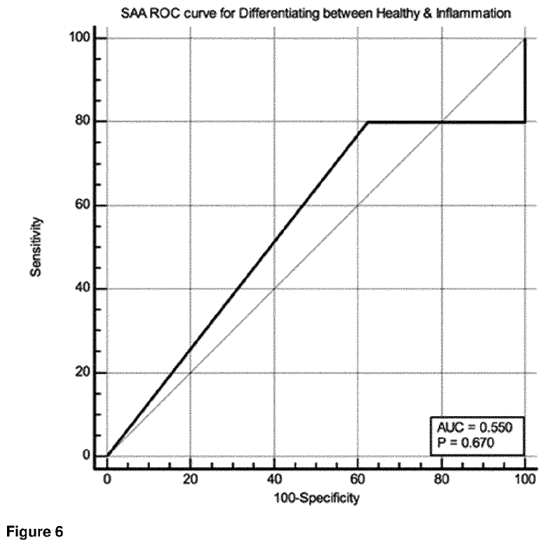

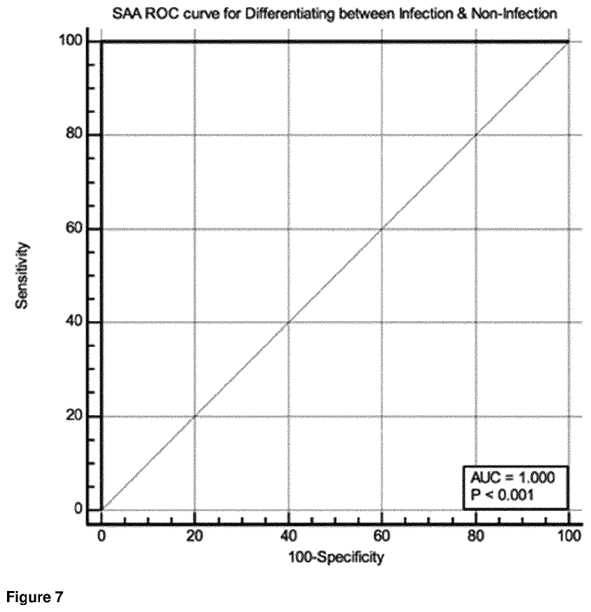

The most significant AUC was observed for SAA when differentiating between the Infection and Non-Infection groups. AUC was 1.000 (P<0.0001) (FIG. 7) and optimum cut off value was identified as 18.5 ug/ml (Table 5). Hp reported an AUC of 0.917, p<0.001 at Youden Index criterion >1217 ug/ml (FIG. 8 and Table 6). AUC for CRP was 0.820 (p=0.0056) at Youden Index criterion >4.7 ug/ml (FIG. 9 and Table 7)

TABLE 5
showing diagnostic performance statistics for a range of SAA cut-off values,
including Youden Index associated criterion of greater than 18.5 ug/ml, to distinguish
between infection and non-infection patients (healthy & inflammation combined)

| Cut-off (ug/ml)-> | 15 | 18.5 | 50 | 100 |
|---|---|---|---|---|
| Sensitivity | 100.0 | 100.0 | 100.0 | 100.0 |
| Specificity | 94.4 | 94.4 | 100.0 | 100.0 |
| Pos. Predictive Value | 85.7 | 85.7 | 100.0 | 100.0 |
| Neg. Predictive Value | 100.0 | 100.0 | 100.0 | 100.0 |
| Accuracy | 95.8 | 95.8 | 100.0 | 100.0 |

TABLE 6
showing diagnostic performance statistics for a range of Hp cut-
off values, including Youden Index associated criterion of
greater than 1217 ug/ml, to distinguish between infection and
non-infection patients (healthy & inflammation combined)

| Cut-off (ug/ml)→ | 400 | 473 | 500 | 600 | 700 | 1217 |
|---|---|---|---|---|---|---|
| Sensitivity | 100.0 | 100.0 | 100.0 | 100.0 | 100.0 | 100.0 |
| Specificity | 22.2 | 38.9 | 50.0 | 55.6 | 61.1 | 88.9 |
| Pos. Predictive Value | 30.0 | 35.3 | 40.0 | 42.9 | 46.2 | 75.0 |
| Neg. Predictive Value | 100.0 | 100.0 | 100.0 | 100.0 | 100.0 | 100.0 |
| Accuracy | 41.7 | 54.2 | 62.5 | 66.7 | 70.8 | 91.7 |

TABLE 7
showing diagnostic performance statistics for a range of CRP cut-off values,
including Youden Index associated criterion of greater than 4.7 ug/ml, to distinguish
between infection and non-infection patients (healthy and inflammation combined)

| Cut-off (ug/ml) | 2 | 4.7 | 6 | 8 | 10 |
|---|---|---|---|---|---|
| Sensitivity | 100.0 | 100.0 | 80.0 | 60.0 | 40.0 |
| Specificity | 20.0 | 70.0 | 80.0 | 80.0 | 90.0 |
| Pos. Predictive Value | 38.5 | 62.5 | 66.7 | 60.0 | 66.7 |
| Neg. Predictive Value | 100.0 | 100.0 | 88.9 | 80.0 | 75.0 |
| Accuracy | 46.7 | 80.0 | 80.0 | 73.3 | 73.3 |

### Infection and Inflammation

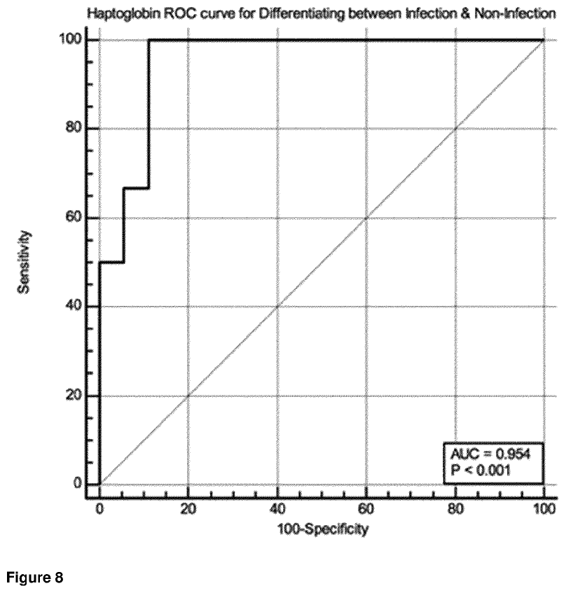

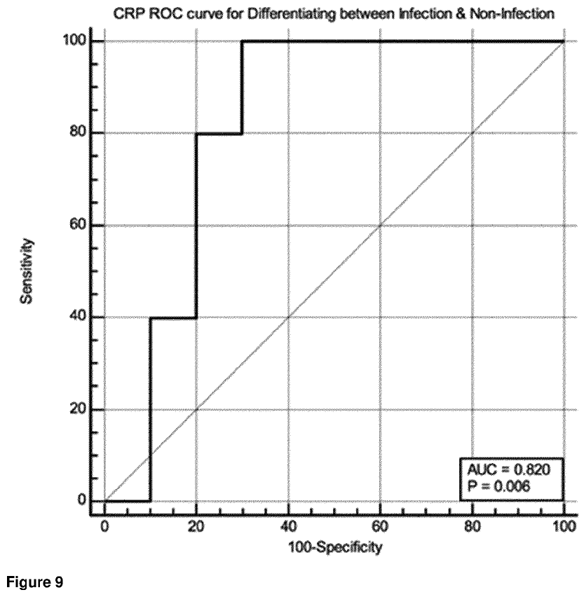

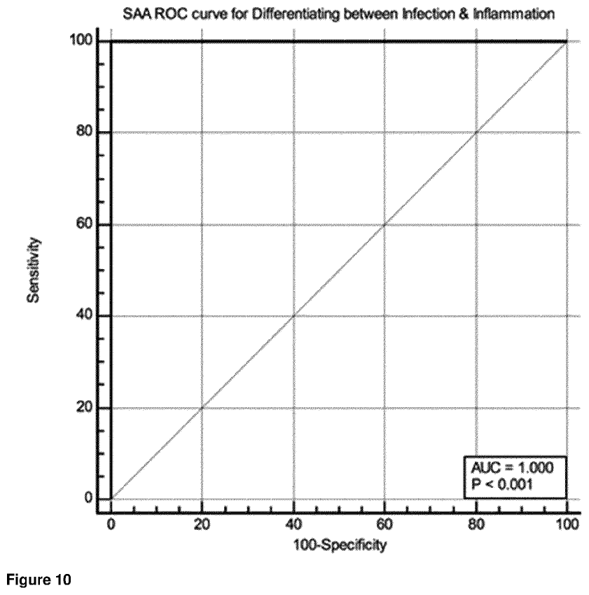

SAA AUC was 1.000, P<0.0001 and Youden Index criterion was >18.5 ug/ml (FIG. 10, Table 8). AUC for Hp was 0.917, P<0.0001 and Youden Index criterion was >796 ug/ml (FIG. 11, Table 9). CRP AUC was 0.800, P=0.1336 and Youden Index criterion >2.456 ug/ml (FIG. 12, Table 10).

TABLE 8
showing diagnostic performance statistics for a range of SAA cut-off values,
including Youden Index associated criterion of greater than 18.5 ug/ml,
to distinguish between infection and inflammation patients

| Cut-off (ug/ml) | 15 | 18.5 | 50 | 100 |
|---|---|---|---|---|
| Sensitivity | 100.0 | 100.0 | 100.0 | 100.0 |
| Specificity | 94.4 | 94.4 | 100.0 | 100.0 |
| Pos. Predictive Value | 85.7 | 85.7 | 100.0 | 100.0 |
| Neg. Predictive Value | 100.0 | 100.0 | 100.0 | 100.0 |
| Accuracy | 95.8 | 95.8 | 100.0 | 100.0 |

TABLE 9
showing diagnostic performance statistics for a range of Hp cut-off values,
including Youden Index associated criterion of greater than 796 ug/ml,
to distinguish between infection and inflammation patients.

| Cut-off (ug/ml)-> | 473 | 500 | 600 | 796 | 1217 |
|---|---|---|---|---|---|
| Sensitivity | 100.0 | 100.0 | 100.0 | 100.0 | 100.0 |
| Specificity | 38.9 | 50.0 | 55.6 | 80.0 | 80.0 |
| Pos. Predictive Value | 35.3 | 40.0 | 42.9 | 75.0 | 75.0 |
| Neg. Predictive Value | 100.0 | 100.0 | 100.0 | 100.0 | 100.0 |
| Accuracy | 54.2 | 62.5 | 66.7 | 87.5 | 87.5 |

TABLE 10
showing diagnostic performance statistics for a range of CRP cut-
off values to distinguish between infection and inflammation patients.

| Cut-off (ug/ml) | 2 | 4.7 | 6 | 8 | 10 |
|---|---|---|---|---|---|
| Sensitivity | 100.0 | 100.0 | 80.0 | 60.0 | 40.0 |
| Specificity | 20.0 | 70.0 | 80.0 | 80.0 | 90.0 |
| Pos. Predictive Value | 38.5 | 62.5 | 66.7 | 60.0 | 66.7 |
| Neg. Predictive Value | 100.0 | 100.0 | 88.9 | 80.0 | 75.0 |
| Accuracy | 46.7 | 80.0 | 80.0 | 73.3 | 73.3 |

### Single and Combined Analysis Between Healthy, Infection and Inflammation Groups in Human Sample Sets

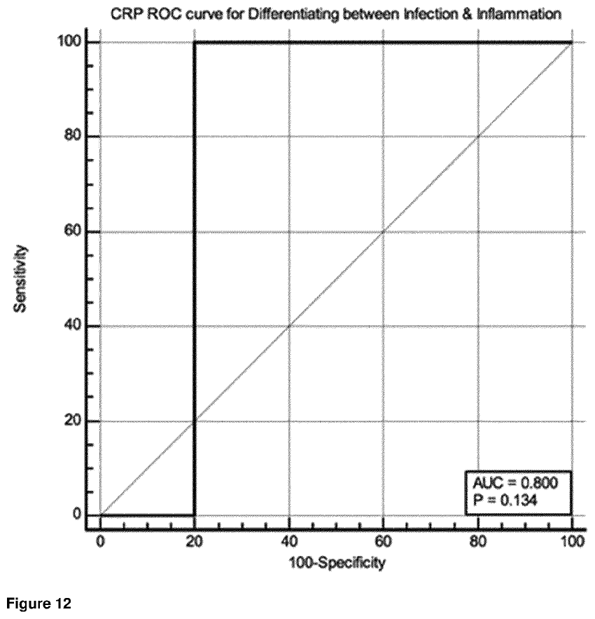

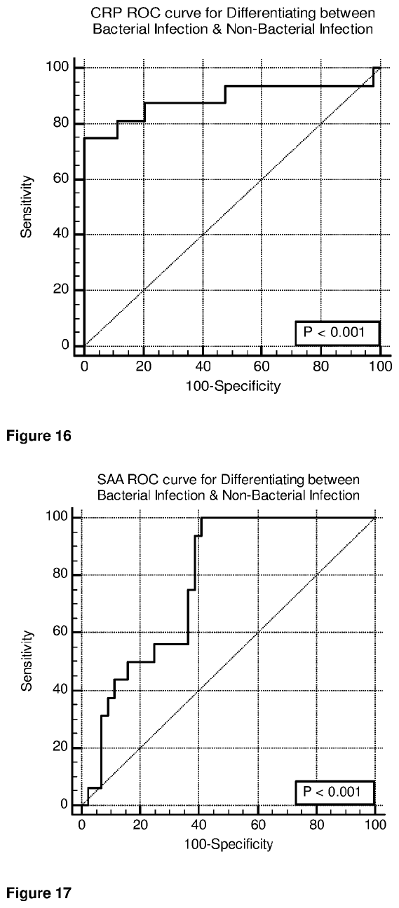

Threshold values were assessed for each of SAA, CRP, MxA and Hp (Table 11 below). The literature reports a wide range of normal values for Hp. The upper threshold in the literature was selected for inclusion at 220 ug/ml. MxA demonstrated the poor performance on ROC analysis for differentiating between the Healthy vs Non-Healthy, Infection vs Non-Infection and Healthy vs Inflammation and Infection vs Inflammation groups. MxA did demonstrate utility as a biomarker for differentiating between Viral and Bacterial groups (see FIG. 14). SAA demonstrated no utility for differentiating between Viral and Bacterial groups (see FIG. 17).

TABLE 11
Threshold values were assessed for each of SAA, CRP, MxA and Hp

| | Cut-off values of ROC analysis | | | |
|---|---|---|---|---|
| | SAA | CRP | Hp | MxA |
| Differentiation | (ug/ml) | (ug/ml) | (mg/dL) | (ng/ml) |
| Healthy vs Non-Healthy | 6 | 29.2 | 159.5 | n/a |
| Infection vs Non-Infection | 10.8 | 50.5 | 193.2 | n/a |
| Healthy vs Inflammation | 6 | 29.2 | 114.2 | n/a |
| Healthy vs Infection | 6 | 29.2 | 159.5 | n/a |
| Infection vs Inflammation | 10.8 | 50.5 | 190 | n/a |
| Bacterial vs Viral | 6 | 97.2 | 182.1 | 174 |

### Healthy and Inflammation

In an analysis to distinguish Healthy and Inflammation groups from each other (Table 11 and Table 12), where those with an infection were not included, CRP at a cut off value of 5 ug/ml performed with an accuracy of 65%. Analysis using a cut off value of 10 ug/ml from the literature yielded accuracy results at 52%. Sensitivity for identifying the individuals from the inflammation group (those categorised based on clinical notes which included an inflammatory condition with no infection) yielded significant false positive results. The interpretation of these results in a clinical setting may lead to a significant portion of patients being treated unnecessarily for inflammation, which may be associated with negative outcomes, e.g. in the case of steroidal treatments. This finding is surprising. The common thinking on CRP is that it is a good biomarker of inflammation. However, this finding also elucidates that CRP alone is not a good differentiator within Healthy and Inflammatory groups, and leads to false positive diagnosis for inflammation with as many as 7 of 10 healthy individuals.

For the same groups, SAA was assessed using cut off values of 10 ug/ml (as per the literature) and 20 ug/ml (as per the data reported here) (Table 12 and Table 13). SAA performed with an accuracy of 52% for differentiating between the two groups. A sensitivity of 15% was recorded when identifying inflammation within the two groups and a specificity of 100% meaning SAA identified all healthy individuals. High specificity came at the expense of sensitivity and accuracy with 11 of 13 cases of inflammation from the Inflammation group reported as negative. In a clinical setting, such results may lead to undertreatment of the individual through withholding of suitable treatment due to the false negative report. As with CRP, SAA is not an accurate differentiator when distinguishing between Healthy and Inflammation groups. This finding is surprising as SAA and CRP are commonly discussed as being biomarkers of inflammation.

Hp has high specificity but low sensitivity at 15% meaning it could not differentiate between Healthy Individuals with Inflammation accurately.

TABLE 12
Performance statistics for SAA and CRP at different
cut-off values to distinguish between Healthy
Individuals and Individuals with Inflammation.

| Description (ug/ml) | sensitivity | specificity | PPV | NPV | accuracy |
|---|---|---|---|---|---|
| Inflammation vs Healthy, | 8 | 100 | 100 | 45 | 48 |
| SAA >= 20 | | | | | |
| Inflammation vs Healthy, | 15 | 100 | 100 | 48 | 52 |
| SAA >= 10 | | | | | |
| Inflammation vs Healthy, | 92 | 30 | 63 | 75 | 65 |
| CRP >= 5 | | | | | |
| Inflammation vs Healthy, | 69 | 30 | 56 | 43 | 52 |
| CRP >= 10 | | | | | |
| Inflammation vs Healthy, | 15 | 100 | 100 | 48 | 52 |
| Hp >= 220 | | | | | |

TABLE 13
Further performance statistics for SAA and CRP at
different cut-off values to distinguish between
Healthy Individuals and Individuals with Inflammation.

| | | | | false | actual | |
|---|---|---|---|---|---|---|
| Description | true | false | true | neg- | pos- | actual |
| (ug/ml) | positive | positive | negative | ative | itive | negative |
| Inflammation vs | 1 | 0 | 10 | 12 | 13 | 10 |
| Healthy, SAA >= 20 | | | | | | |
| Inflammation vs | 2 | 0 | 10 | 11 | 13 | 10 |
| Healthy, SAA >= 10 | | | | | | |
| Inflammation vs | 12 | 7 | 3 | 1 | 13 | 10 |
| Healthy, CRP >= 5 | | | | | | |
| Inflammation vs | 9 | 7 | 3 | 4 | 13 | 10 |
| Healthy, CRP >= 10 | | | | | | |
| Inflammation vs | 2 | 0 | 10 | 11 | 13 | 10 |
| Healthy, Hp >= 220 | | | | | | |

### Inflammation and Infection

In an analysis to distinguish between Inflammation and Infection groups wherein Healthy were omitted from the analysis (see Table 14 and Table 15), CRP at a reference value 10 ug/ml had 80% accuracy. At this range, CRP demonstrated a very good sensitivity of 97% for identifying infection from inflammation within the two groups when healthy individuals were not included in the analysis. Specificity was not good however, and CRP incorrectly assigned a diagnosis of infection to 9 of 13 individuals from the Inflammation group who were experiencing a non-infectious inflammation. Such a diagnosis in a clinical setting could potentially lead to the administration of an inappropriate treatment to individuals who are not experiencing an infection.

In the same analysis, SAA was assessed at 10 ug/ml to distinguish between the Inflammation and Infection groups. SAA demonstrated an accuracy of 86% and specificity and sensitivity of 86% and 85% respectively. SAA accurately categorised 32 of 37 individuals from the Infection group and 11 of 13 individuals from the Inflammation group when the Healthy group was omitted. This data supports the clinical utility of SAA as a biomarker for differentiating between infection and inflammation. However, since the Healthy group were omitted from the analysis and the findings of Table 11 and Table 12 show SAA as a poor differentiator of Healthy and Inflammation groups, SAA alone is not a suitable biomarker for identifying between the three groups Healthy, Inflammation and Infection, though it does differentiate well between Inflammation and Infection or Healthy and Infection.

Hp had a specificity of 85% but low sensitivity at 24% meaning it could not differentiate between Healthy Individuals with Inflammation accurately.

TABLE 14
Performance statistics for SAA and CRP at different
cut-off values to distinguish between
Individuals with Infection and Individuals with Inflammation

| Description/(ug/ml) | sensitivity | specificity | PPV | NPV | accuracy |
|---|---|---|---|---|---|
| Infection vs Inflammation, | 84 | 92 | 97 | 67 | 86 |
| SAA>= 20 | | | | | |
| Infection vs Inflammation, | 86 | 85 | 94 | 69 | 86 |
| SAA >= 10 | | | | | |
| Infection vs Inflammation, | 97 | 8 | 75 | 50 | 74 |
| CRP>=5 | | | | | |
| Infection vs Inflammation, | 97 | 31 | 80 | 80 | 80 |
| CRP>= 10 | | | | | |
| Infection vs Inflammation, | 24 | 85 | 82 | 28 | 40 |
| Hp >= 220 | | | | | |

TABLE 15
Further performance statistics for SAA and CRP at different
cut-off values to distinguish between Individuals
with Infection and Individuals with Inflammation

| | | | | | | actual |
|---|---|---|---|---|---|---|
| Description | true | false | true | false | actual | neg- |
| (ug/ml) | positive | positive | negative | negative | positive | ative |
| Infection vs | 31 | 1 | 12 | 6 | 37 | 13 |
| Inflammation, | | | | | | |
| SAA >= 20 | | | | | | |
| Infection vs | 32 | 2 | 11 | 5 | 37 | 13 |
| Inflammation, | | | | | | |
| SAA >= 10 | | | | | | |
| Infection vs | 36 | 12 | 1 | 1 | 37 | 13 |
| Inflammation, | | | | | | |
| CRP >= 5 | | | | | | |
| Infection vs | 36 | 9 | 4 | 1 | 37 | 13 |
| Inflammation, | | | | | | |
| CRP >= 10 | | | | | | |
| Infection vs | 9 | 2 | 11 | 28 | 37 | 13 |
| Inflammation, | | | | | | |
| Hp >= 220 | | | | | | |

### Healthy Vs Non-Healthy (Infection and Inflammation)

In an analysis differentiating between Healthy and Non-Healthy groups, CRP at the cut off value of 10 ug/ml distinguished non-healthy individuals within the group with a sensitivity of 90%. However, specificity was poor meaning many healthy individuals were incorrectly assigned a non-healthy status (Table 16 and Table 17), i.e., false positives.

SAA distinguished between the Healthy and Non-Healthy group with sensitivity at 68%. There were false negative findings, and 16 of 50 non-healthy individuals were assigned a Healthy status despite being clinically non-healthy (Table 16 and Table 17), i.e., false negatives. Hp has high specificity but low sensitivity at 22% meaning it could not differentiate between Healthy Individuals with Inflammation accurately.

TABLE 16
Performance statistics for SAA and CRP at different cut-off values to distinguish
between Healthy Individuals and Non-Healthy Individuals (Inflammation and Infection).

| Description/(ug/ml) | sensitivity | specificity | PPV | NPV | accuracy |
|---|---|---|---|---|---|
| Non-healthy vs | 64 | 100 | 100 | 36 | 70 |
| Healthy, SAA >= 20 | | | | | |
| Non-healthy vs | 68 | 100 | 100 | 38 | 73 |
| Healthy, SAA >= 10 | | | | | |
| Non-healthy vs | 96 | 30 | 87 | 60 | 85 |
| Healthy, CRP >= 5 | | | | | |
| Non-healthy vs | 90 | 30 | 87 | 38 | 80 |
| Healthy, CRP >= 10 | | | | | |
| Non-healthy vs | 22 | 100 | 100 | 20 | 35 |
| Healthy, Hp >= 220 | | | | | |

TABLE 17
Further performance statistics for SAA and CRP at different cut-off
values to distinguish between Healthy Individuals and Non-Healthy
Individuals (Inflammation and Infection).

| | | | | | actual | actual |
|---|---|---|---|---|---|---|
| Description | true | false | true | false | pos- | neg- |
| (ug/ml) | positive | positive | negative | negative | itive | ative |
| Non-healthy vs | 32 | 0 | 10 | 18 | 50 | 10 |
| Healthy, SAA >= 20 | | | | | | |
| Non-healthy vs | 34 | 0 | 10 | 16 | 50 | 10 |
| Healthy, SAA >= 10 | | | | | | |
| Non-healthy vs | 48 | 7 | 3 | 2 | 50 | 10 |
| Healthy, CRP >= 5 | | | | | | |
| Non-healthy vs | 45 | 7 | 3 | 5 | 50 | 10 |
| Healthy, CRP >= 10 | | | | | | |
| Non-healthy vs | 11 | 0 | 10 | 39 | 50 | 10 |
| Healthy, Hp >= 220 | | | | | | |

### Infection Vs Non-Infection (Healthy and Inflammation)

CRP at the cut off value of 10 ug/ml distinguished Infection from Non-Infection groups. However, specificity was poor as previously seen in single biomarker analysis, resulting in significant false positives (Table 19), which may lead to inaccurate treatment decisions and unnecessary clinical investigations affecting the patient and economic burden. SAA performed better when distinguishing between the Infection and Non-Infection group with good sensitivity and very good specificity.

TABLE 18
Performance statistics for SAA and CRP combined
for distinguishing between Individuals with
Infection and Non-Infection (Healthy and Inflammation).

| Description/(ug/ml) | sensitivity | specificity | PPV | NPV | accuracy |
|---|---|---|---|---|---|
| Infection vs Non- | 84 | 96 | 97 | 79 | 88 |
| infection, SAA >= 20 | | | | | |
| Infection vs Non- | 86 | 91 | 94 | 81 | 88 |
| infection, SAA >= 10 | | | | | |
| Infection vs Non- | 97 | 17 | 65 | 80 | 67 |
| infection, CRP >= 5 | | | | | |
| Infection vs Non- | 97 | 30 | 69 | 88 | 72 |
| infection, CRP >= 10 | | | | | |

TABLE 19
Further performance statistics for SAA and CRP combined
for distinguishing between Individuals with Infection
and Non-Infection (Healthy and Inflammation).

| Description | true | false | true | false | actual | actual |
|---|---|---|---|---|---|---|
| (ug/ml) | positive | positive | negative | negative | positive | negative |
| Infection vs | 31 | 1 | 22 | 6 | 37 | 23 |
| Non- | | | | | | |
| infection, | | | | | | |
| SAA >= 20 | | | | | | |
| Infection vs | 32 | 2 | 21 | 5 | 37 | 23 |
| Non- | | | | | | |
| infection, | | | | | | |
| SAA >= 10 | | | | | | |
| Infection vs | 36 | 19 | 4 | 1 | 37 | 23 |
| Non- | | | | | | |
| infection, | | | | | | |
| CRP >= 5 | | | | | | |
| Infection vs | 36 | 16 | 7 | 1 | 37 | 23 |
| Non- | | | | | | |
| infection, | | | | | | |
| CRP >=10 | | | | | | |

### Combined Healthy Vs Non-Healthy (Infection and Inflammation)

Combination of CRP and SAA to differentiate between Healthy and Non-Healthy groups shows improvement over the individual biomarker assessments with improved accuracy across all combinations (Table 20). However, while sensitivities increased, specificities were not improved. The combination allowed better detection of true positives than individual analysis, meaning non-healthy statuses are being detected more accurately and therefore the combination reduced the incidence of false negatives (see Table 21).

The clinical outcome of the combination is better ability to identify non-heathy individuals compared to individual analysis of SAA or CRP. However, there was an elevation of false positives at some combinations whereby individuals in the Healthy groups were categorised as Non-Healthy, i.e., false positives. In a clinical setting this may lead to Individuals being treated incorrectly or unnecessarily undergoing further clinical investigative work.

TABLE 20
Performance statistics for SAA and CRP combined for
distinguishing between Healthy and Non-Healthy Individuals.

| | Cut off | | | | | |
|---|---|---|---|---|---|---|
| Description | (ug/ml) | Sensitivity | Specificity | PPV | NPV | Accuracy |
| Non-healthy | (5, 10) | 30 | 98 | 75 | 88 | 87 |
| vs Healthy | | | | | | |
| Non-healthy | (5, 20) | 30 | 98 | 75 | 88 | 87 |
| vs Healthy | | | | | | |
| Non-healthy | (10, 10) | 30 | 92 | 43 | 87 | 82 |
| vs Healthy | | | | | | |
| Non-healthy | (10, 20) | 30 | 92 | 43 | 87 | 82 |
| vs Healthy | | | | | | |

TABLE 21
Further performance statistics for SAA and CRP combined for
distinguishing between Healthy and Non-Healthy Individuals.

| | Cut off/ | | | | | |
|---|---|---|---|---|---|---|
| Description | (ug/ml) | Sensitivity | Specificity | PPV | NPV | Accuracy |
| Non-healthy | (5, 10) | 49 | 7 | 3 | 1 | 50 |
| vs Healthy | | | | | | |
| Non-healthy | (5, 20) | 49 | 7 | 3 | 1 | 50 |
| vs Healthy | | | | | | |
| Non-healthy | (10, 10) | 46 | 7 | 3 | 4 | 50 |
| vs Healthy | | | | | | |
| Non-healthy | (10, 20) | 46 | 7 | 3 | 4 | 50 |
| vs Healthy | | | | | | |

### Combined Infection Vs Non-Infection (Healthy and Inflammation)

The combination of SAA and CRP in the differentiation of Infection and Non-Infection groups shows improvement over single biomarker analysis with improved specificities for all combinations (Table 22). The improvement in specificity is associated with a reduction in false negatives for individuals in the Infection group observed in single biomarker analysis particularly for CRP.

TABLE 22
Performance statistics for SAA and CRP
combined for distinguishing between Individuals with
Infection and Non-Infection (Healthy and Inflammation).

| | Cut off | | | | | |
|---|---|---|---|---|---|---|
| Description | (ug/ml) | Sensitivity | Specificity | PPV | NPV | accuracy |
| Infection vs | (5, 10) | 86 | 91 | 94 | 81 | 88 |
| Non-infection | | | | | | |
| Infection vs | (5, 20) | 84 | 96 | 97 | 79 | 88 |
| Non-infection | | | | | | |
| Infection vs | (10, 10) | 86 | 91 | 94 | 81 | 88 |
| Non-infection | | | | | | |
| Infection vs | (10, 20) | 84 | 96 | 97 | 79 | 88 |
| Non-infection | | | | | | |

TABLE 23
Further performance statistics for SAA and CRP combined
for distinguishing between Individuals with
Infection and Non-Infection (Healthy and Inflammation)

| | Cut off | true | false | true | false | actual |
|---|---|---|---|---|---|---|
| Description | (ug/ml) | positive | positive | negative | negative | positive |
| Infection vs | (5, 10) | 10 | 12 | 35 | 3 | 13 |
| Non-infection | | | | | | |
| Infection vs | (5, 20) | 11 | 13 | 34 | 2 | 13 |
| Non-infection | | | | | | |
| Infection vs | (10, 10) | 7 | 12 | 35 | 6 | 13 |
| Non-infection | | | | | | |
| Infection vs | (10, 20) | 8 | 13 | 34 | 5 | 13 |
| Non-infection | | | | | | |

### Combined Non-Infectious Inflammation Vs Other (Healthy and Infection)

Using SAA or CRP alone, it is not possible to identify individuals with non-infectious inflammation from Healthy and Infectious groups. In combination however it has been shown below to be possible to differentiate between the three possible health status outcomes (Healthy, Infection and Inflammation). This is an improvement over existing methods wherein neither CRP or SAA can accurately categorize individuals experiencing non-infectious inflammatory conditions.

TABLE 24
Performance statistics for SAA and CRP combined for
distinguishing between Individuals with Inflammation and
those with either Healthy or Infection conditions (Other)

| | Cut off | | | | | |
|---|---|---|---|---|---|---|
| Description | (ug/ml) | Sensitivity | Specificity | PPV | NPV | accuracy |
| Non-Infectious | (5, 10) | 77 | 74 | 45 | 92 | 75 |
| Inflammation | | | | | | |
| vs Other | | | | | | |
| Non-Infectious | (5, 20) | 85 | 72 | 46 | 94 | 75 |
| Inflammation | | | | | | |
| vs Other | | | | | | |
| Non-Infectious | (10, | 54 | 74 | 37 | 85 | 70 |
| Inflammation | 10) | | | | | |
| vs Other | | | | | | |
| Non-Infectious | (10, | 62 | 72 | 38 | 87 | 70 |
| Inflammation | 20) | | | | | |
| vs Other | | | | | | |

TABLE 25
Further performance statistics for SAA and CRP combined for
distinguishing between Individuals with Inflammation and those
with either Healthy or Infection conditions (Other)

| | Cut off | true | false | true | false | actual |
|---|---|---|---|---|---|---|
| Description | (ug/ml) | positive | positive | negative | negative | positive |
| Non- | (5, 10) | 10 | 12 | 35 | 3 | 13 |
| Infectious | | | | | | |
| Inflammation | | | | | | |
| vs Other | | | | | | |
| Non- | (5, 20) | 11 | 13 | 34 | 2 | 13 |
| Infectious | | | | | | |
| Inflammation | | | | | | |
| vs Other | | | | | | |
| Non- | (10, 10) | 7 | 12 | 35 | 6 | 13 |
| Infectious | | | | | | |
| Inflammation | | | | | | |
| vs Other | | | | | | |
| Non- | (10, 20) | 8 | 13 | 34 | 5 | 13 |
| Infectious | | | | | | |
| Inflammation | | | | | | |
| vs Other | | | | | | |

Analysis of SAA and CRP in combination allow differentiation between three groups: Healthy, Inflammation and Infection. This is not possible using a single biomarker and a single cut-off value. This is because there three outcomes, i.e., healthy, infection or inflammation, and a single biomarker at a single threshold can only provide two outcomes. While there is improvement over single biomarker analysis when SAA and CRP are combined, there remains a need for a more accurate combination to reduce false negative and false positive results, and correctly determine whether an individual is healthy, has an infection, or has an inflammation. One of the issues with the combination is an elevation of false positives, which in a clinical setting may lead to individuals being treated incorrectly or unnecessarily undergoing further clinical investigative work which creates a burden on the patient, the clinician and an economic burden. SAA and CRP perform well to differentiate between Infection and Non-Infection groups. However, to correctly differentiate between Healthy, Inflammation and Infection, improvements are required in distinguishing between Healthy and Non-Healthy groups and between Healthy and Inflammation groups. Improvement in differentiating the Inflammatory group from both Healthy and Infection groups would also be of significant benefit when clinical decisions are being made relating to detection, treatment, and monitoring.

### Combination of SAA, CRP and Hp

A study was undertaken including the biomarker Haptoglobin to assess diagnostic improvement in differentiating the three groups (Healthy, Infection and Inflammation). Owing to the wide range of normal values reported in the literature for Hp (50-220 mg/dl), and the findings by the inventors, the upper was not suitable and so the inventors undertook a study to identify optimal cut off values. The values determined via ROC analysis for the differentiation of the following groups was 114 ug/ml (healthy vs inflammation), 159 ug/ml (healthy vs non healthy) and 193 ug/ml (infection vs non-infection), respectively.

### Healthy Vs Non Healthy (Infection and Inflammation) SAA, CRP, Hp

SAA, CRP and Hp lead to an overall improvement in true and false negative rates within the Healthy and Non-Healthy groups when compared to a dual analysis of SAA and CRP and false positives appear to reduce significantly (Table 26 and Table 27).

TABLE 26
Performance statistics for SAA, CRP and Hp combined for
distinguishing between Healthy and Non-Healthy Individuals.

| | Cut off | | | | | | |
|---|---|---|---|---|---|---|---|
| | (ug/ml) or | Positive | | | | | |
| Description | (mg/dl) | type | Sensitivity | Specificity | PPV | NPV | accuracy |
| Healthy vs | (5, 10, 114) | Healthy | 60 | 94 | 67 | 92 | 88 |
| Non-healthy | | | | | | | |
| Healthy vs | (5, 10, 159) | Healthy | 70 | 84 | 47 | 93 | 82 |
| Non-healthy | | | | | | | |
| Healthy vs | (5, 10, 220) | Healthy | 100 | 72 | 42 | 100 | 77 |
| Non-healthy | | | | | | | |
| Healthy vs | (5, 20, 114) | Healthy | 60 | 94 | 67 | 92 | 88 |
| Non-healthy | | | | | | | |
| Healthy vs | (5, 20, 159) | Healthy | 70 | 84 | 47 | 93 | 82 |
| Non-healthy | | | | | | | |
| Healthy vs | (5, 20, 220) | Healthy | 100 | 70 | 40 | 100 | 75 |
| Non-healthy | | | | | | | |
| Healthy vs | (10, 10, 114) | Healthy | 60 | 88 | 50 | 92 | 83 |
| Non-healthy | | | | | | | |
| Healthy vs | (10, 10, 159) | Healthy | 70 | 84 | 47 | 93 | 82 |
| Non-healthy | | | | | | | |
| Healthy vs | (10, 10, 220) | Healthy | 100 | 72 | 42 | 100 | 77 |
| Non-healthy | | | | | | | |
| Healthy vs | (10, 20, 114) | Healthy | 60 | 88 | 50 | 92 | 83 |
| Non-healthy | | | | | | | |
| Healthy vs | (10, 20, 159) | Healthy | 70 | 84 | 47 | 93 | 82 |
| Non-healthy | | | | | | | |
| Healthy vs | (10, 20, 220) | Healthy | 100 | 70 | 40 | 100 | 75 |
| Non-healthy | | | | | | | |

TABLE 27
Further performance statistics for SAA, CRP and Hp combined for distinguishing
between Healthy and Non-Healthy Individuals.

| | Cut off | | | | | | | |
|---|---|---|---|---|---|---|---|---|
| | (ug/ml) or | positive | true | false | true | false | actual | actual |
| Description | (mg/dl) | type | +ve | +ve | −ve | −ve | +ve | −ve |
| Healthy vs | (5, 10, 114) | Healthy | 6 | 3 | 47 | 4 | 10 | 50 |
| Non-healthy | | | | | | | | |
| Healthy vs | (5, 10, 159) | Healthy | 7 | 8 | 42 | 3 | 10 | 50 |
| Non-healthy | | | | | | | | |
| Healthy vs | (5, 10, 220) | Healthy | 10 | 14 | 36 | 0 | 10 | 50 |
| Non-healthy | | | | | | | | |
| Healthy vs | (5, 20, 114) | Healthy | 6 | 3 | 47 | 4 | 10 | 50 |
| Non-healthy | | | | | | | | |
| Healthy vs | (5, 20, 159) | Healthy | 7 | 8 | 42 | 3 | 10 | 50 |
| Non-healthy | | | | | | | | |
| Healthy vs | (5, 20, 220) | Healthy | 10 | 15 | 35 | 0 | 10 | 50 |
| Non-healthy | | | | | | | | |
| Healthy vs | (10, 10, 114) | Healthy | 6 | 6 | 44 | 4 | 10 | 50 |
| Non-healthy | | | | | | | | |
| Healthy vs | (10, 10, 159) | Healthy | 7 | 8 | 42 | 3 | 10 | 50 |
| Non-healthy | | | | | | | | |
| Healthy vs | (10, 10, 220) | Healthy | 10 | 14 | 36 | 0 | 10 | 50 |
| Non-healthy | | | | | | | | |
| Healthy vs | (10, 20, 114) | Healthy | 6 | 6 | 44 | 4 | 10 | 50 |
| Non-healthy | | | | | | | | |
| Healthy vs | (10, 20, 159) | Healthy | 7 | 8 | 42 | 3 | 10 | 50 |
| Non-healthy | | | | | | | | |
| Healthy vs | (10, 20, 220) | Healthy | 10 | 15 | 35 | 0 | 10 | 50 |
| Non-healthy | | | | | | | | |

### Infection Vs Non Infection (Healthy and Inflammation):SAA, CRP, Hp

SAA, CRP and Hp demonstrated a significant improvement in the detection of true positives and a reduction in the false negative rate compared to SAA and CRP alone or combined.

TABLE 28
Performance statistics for SAA, CRP and Hp combined for
distinguishing between Infection and Non-Infection

| | Cut off | | | | | |
|---|---|---|---|---|---|---|
| | (ug/ml) or | Sensi- | Spec- | | | |
| Description | (mg/dl) | tivity | ificity | PPV | NPV | accuracy |
| Infection vs | (5, 20, 114) | 84 | 96 | 97 | 79 | 88 |
| Non-infection | | | | | | |
| Infection vs | (5, 10, 114) | 62 | 86 | 91 | 94 | 81 |
| Non-infection | | | | | | |
| Infection vs | (5, 20, 159) | 84 | 96 | 97 | 79 | 88 |
| Non-infection | | | | | | |
| Infection vs | (5, 20, 220) | 84 | 96 | 97 | 79 | 88 |
| Non-infection | | | | | | |
| Infection vs | (10, 20, 114) | 84 | 96 | 97 | 79 | 88 |
| Non-infection | | | | | | |
| Infection vs | (10, 10, 114) | 86 | 91 | 94 | 81 | 88 |
| Non-infection | | | | | | |
| Infection vs | (10, 20, 159) | 84 | 96 | 97 | 79 | 88 |
| Non-infection | | | | | | |
| Infection vs | (10, 20, 220) | 84 | 96 | 97 | 79 | 88 |
| Non-infection | | | | | | |

TABLE 29
Further performance statistics for SAA, CRP and Hp combined for distinguishing
between Infection and Non-Infection

| | Cut off | | | | | | |
|---|---|---|---|---|---|---|---|
| | (ug/ml) or | positive | true | false | true | false | actual |
| Description | (mg/dl) | type | +ve | +ve | −ve | −ve | +ve |
| Infection vs | (5, 20, 114) | 31 | 1 | 22 | 6 | 37 | 23 |
| Non-infection | | | | | | | |
| | (5, 10, 114) | 32 | 2 | 21 | 5 | 37 | 23 |
| Infection vs | (5, 20, 159) | 31 | 1 | 22 | 6 | 37 | 23 |
| Non-infection | | | | | | | |
| Infection vs | (5, 20, 220) | 31 | 1 | 22 | 6 | 37 | 23 |
| Non-infection | | | | | | | |
| Infection vs | (10, 10, 114) | 32 | 2 | 21 | 5 | 37 | 32 |
| Non-infection | | | | | | | |
| Infection vs | (10, 20, 114) | 31 | 1 | 22 | 6 | 37 | 23 |
| Non-infection | | | | | | | |
| Infection vs | (10, 20, 159) | 31 | 1 | 22 | 6 | 37 | 23 |
| Non-infection | | | | | | | |
| Infection vs | (10, 20, 220) | 31 | 1 | 22 | 6 | 37 | 23 |
| Non-infection | | | | | | | |

### Non-Infectious Inflammation Vs Other (Healthy and Infection): SAA, CRP, Hp

The combination of Hp surprisingly improved the differentiation of the Inflammation group within the three groups, Improved sensitivity and specificity and overall accuracy were observed compared to CRP and SAA. The false negative rate was maintained and there was a decrease in false positives.

TABLE 30
Performance statistics for SAA, CRP and Hp combined for
distinguishing between Non-Infectious Inflammation and Other
(Healthy and Infection)

| | Cut off | | | | | |
|---|---|---|---|---|---|---|
| | (ug/ml) or | Sensi- | Spec- | | | accu- |
| Description | (mg/dl) | tivity | ificity | PPV | NPV | racy |
| Non-Infectious | (5, 10, 114) | 77 | 85 | 59 | 93 | 83 |
| Inflammation | | | | | | |
| vs Other | | | | | | |
| Non-Infectious | (5, 10, 159) | 46 | 89 | 55 | 86 | 80 |
| Inflammation | | | | | | |
| vs Other | | | | | | |
| Non-Infectious | (5, 10, 220) | 15 | 100 | 100 | 81 | 82 |
| Inflammation | | | | | | |
| vs Other | | | | | | |
| Non-Infectious | (5, 20, 114) | 85 | 83 | 58 | 95 | 83 |
| Inflammation | | | | | | |
| vs Other | | | | | | |
| Non-Infectious | (5, 20, 159) | 54 | 87 | 54 | 87 | 80 |
| Inflammation | | | | | | |
| vs Other | | | | | | |
| Non-Infectious | (5, 20, 220) | 15 | 98 | 67 | 81 | 80 |
| Inflammation | | | | | | |
| vs Other | | | | | | |
| Non-Infectious | (10, 10, 114) | 54 | 85 | 50 | 87 | 78 |
| Inflammation | | | | | | |
| vs Other | | | | | | |
| Non-Infectious | (10, 10, 159) | 46 | 89 | 55 | 86 | 80 |
| Inflammation | | | | | | |
| vs Other | | | | | | |
| Non-Infectious | (10, 10, 220) | 15 | 100 | 100 | 81 | 82 |
| Inflammation | | | | | | |
| vs Other | | | | | | |
| Non-Infectious | (10, 20, 114) | 62 | 83 | 50 | 89 | 78 |
| Inflammation | | | | | | |
| vs Other | | | | | | |
| Non-Infectious | (10, 20, 159) | 54 | 87 | 54 | 87 | 80 |
| Inflammation | | | | | | |
| vs Other | | | | | | |
| Non-Infectious | (10, 20, 220) | 15 | 98 | 67 | 81 | 80 |
| Inflammation | | | | | | |
| vs Other | | | | | | |

TABLE 31
Further performance statistics for SAA, CRP and Hp combined for distinguishing
between Non-Infectious Inflammation and Other (Healthy and Infection)

| | Cut off | | | | | | |
|---|---|---|---|---|---|---|---|
| | (ug/ml) or | positive | true | false | | false | actual |
| Description | (mg/dl) | type | +ve | +ve | true −ve | −ve | +ve |
| Non-Infectious | (5, 10, 114) | 10 | 7 | 40 | 3 | 13 | 47 |
| Inflammation | | | | | | | |
| vs Other | | | | | | | |
| Non-Infectious | (5, 10, 159) | 6 | 5 | 42 | 7 | 13 | 47 |
| Inflammation | | | | | | | |
| vs Other | | | | | | | |
| Non-Infectious | (5, 10, 220) | 2 | 0 | 47 | 11 | 13 | 47 |
| Inflammation | | | | | | | |
| vs Other | | | | | | | |
| Non-Infectious | (5, 20, 114) | 11 | 8 | 39 | 2 | 13 | 47 |
| Inflammation | | | | | | | |
| vs Other | | | | | | | |
| Non-Infectious | (5, 20, 159) | 7 | 6 | 41 | 6 | 13 | 47 |
| Inflammation | | | | | | | |
| vs Other | | | | | | | |
| Non-Infectious | (5, 20, 220) | 2 | 1 | 46 | 11 | 13 | 47 |
| Inflammation | | | | | | | |
| vs Other | | | | | | | |
| Non-Infectious | (10, 10, 114) | 7 | 7 | 40 | 6 | 13 | 47 |
| Inflammation | | | | | | | |
| vs Other | | | | | | | |
| Non-Infectious | (10, 10, 159) | 6 | 5 | 42 | 7 | 13 | 47 |
| Inflammation | | | | | | | |
| vs Other | | | | | | | |
| Non-Infectious | (10, 10, 220) | 2 | 0 | 47 | 11 | 13 | 47 |
| Inflammation | | | | | | | |
| vs Other | | | | | | | |
| Non-Infectious | (10, 20, 114) | 8 | 8 | 39 | 5 | 13 | 47 |
| Inflammation | | | | | | | |
| vs Other | | | | | | | |
| Non-Infectious | (10, 20, 159) | 7 | 6 | 41 | 6 | 13 | 47 |
| Inflammation | | | | | | | |
| vs Other | | | | | | | |
| Non-Infectious | 10, 20, 220) | 2 | 1 | 46 | 11 | 13 | 47 |
| Inflammation | | | | | | | |
| vs Other | | | | | | | |

Combination of SAA, CRP, Hp and MxA were Assessed Using ROC Analysis to Determine Cut Off Values of Interest.

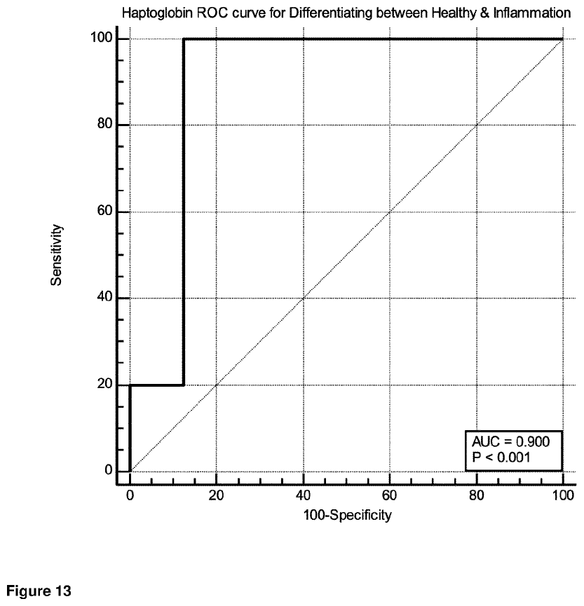

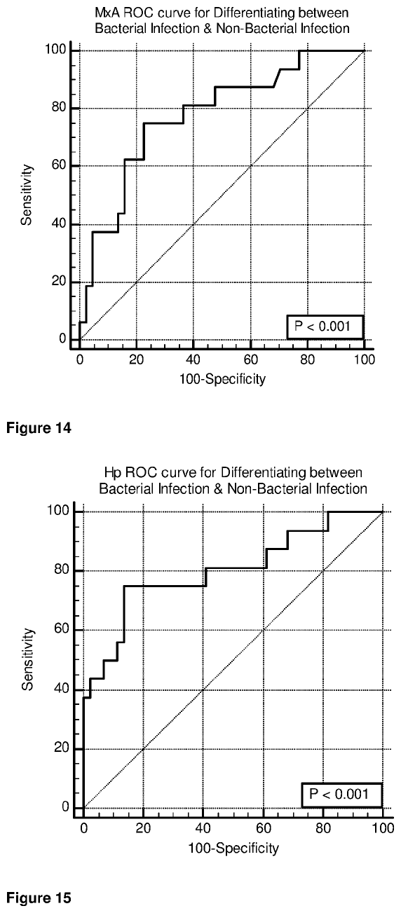

SAA, CRP, MxA and HP were combined using ROC analysis cut-off values of interest as shown in Table 11 and in FIGS. 14 to 17. Such values may be used in combination with the methods of the invention to further improve performance statistics and may be selected from in some examples of the invention to optimise performance in certain groups, e.g., depending on the setting which may be an infection screening centre, a clinical practice, or a chronic disease centre.

### Healthy Vs Non Healthy (Infection and Inflammation): Hp

TABLE 32
Performance statistics for SAA, CRP and Hp combined for
distinguishing between Healthy and Non-Healthy groups.

| | Cut off | | | | | |
|---|---|---|---|---|---|---|
| | (ug/ml) or | Sensi- | Spec- | | | |
| Description | (mg/dl) | tivity | ificity | PPV | NPV | Accuracy |
| Healthy | (5, 6, 114) | 40 | 94 | 57 | 89 | 85 |
| vs Non-healthy | | | | | | |
| Healthy | (5, 6, 159) | 50 | 86 | 42 | 90 | 80 |
| vs Non-healthy | | | | | | |
| Healthy | (10, 6, 114) | 40 | 88 | 40 | 88 | 80 |
| vs Non-healthy | | | | | | |
| Healthy | (10, 6, 159) | 50 | 86 | 42 | 90 | 80 |
| vs Non-healthy | | | | | | |
| Healthy | (29, 6, 114) | 50 | 82 | 36 | 89 | 77 |
| vs Non-healthy | | | | | | |
| Healthy | (29, 6, 159) | 60 | 80 | 38 | 91 | 77 |
| vs Non-healthy | | | | | | |
| Healthy | (29, 10, 114) | 70 | 80 | 41 | 93 | 78 |
| vs Non-healthy | | | | | | |
| Healthy | (29, 10, 159) | 80 | 76 | 40 | 95 | 77 |
| vs Non-healthy | | | | | | |
| Healthy | (29, 20, 114) | 70 | 80 | 41 | 93 | 78 |
| vs Non-healthy | | | | | | |
| Healthy | (29, 20, 159) | 80 | 76 | 40 | 95 | 77 |
| vs Non-healthy | | | | | | |

TABLE 33
Further performance statistics for SAA, CRP and Hp combined for distinguishing
between Healthy and Non-Healthy groups.

| | Cut off | | | | | | |
|---|---|---|---|---|---|---|---|
| | (ug/ml) or | true | false | true | false | actual | actual |
| Description | (mg/dl) | +ve | +ve | −ve | −ve | +ve | −ve |
| Healthy vs | (5, 6, 114) | 4 | 3 | 47 | 6 | 10 | 50 |
| Non-healthy | | | | | | | |
| Healthy vs | (5, 6, 159) | 5 | 7 | 43 | 5 | 10 | 50 |
| Non-healthy | | | | | | | |
| Healthy vs | (10, 6, 114) | 4 | 6 | 44 | 6 | 10 | 50 |
| Non-healthy | | | | | | | |
| Healthy vs | (10, 6, 159) | 5 | 7 | 43 | 5 | 10 | 50 |
| Non-healthy | | | | | | | |
| Healthy vs | (29, 6, 114) | 5 | 9 | 41 | 5 | 10 | 50 |
| Non-healthy | | | | | | | |
| Healthy vs | (29, 6, 159) | 6 | 10 | 40 | 4 | 10 | 50 |
| Non-healthy | | | | | | | |
| Healthy vs | (29, 10, 114) | 7 | 10 | 40 | 3 | 10 | 50 |
| Non-healthy | | | | | | | |
| Healthy vs | (29, 10, 159) | 8 | 12 | 38 | 2 | 10 | 50 |
| Non-healthy | | | | | | | |
| Healthy vs | (29, 20, 114) | 7 | 10 | 40 | 3 | 10 | 50 |
| Non-healthy | | | | | | | |
| Healthy vs | (29, 20, 159) | 8 | 12 | 38 | 2 | 10 | 50 |
| Non-healthy | | | | | | | |

### Infection Vs Non-Infection (Healthy and Inflammation): Hp

TABLE 34
Performance statistics for SAA, CRP and Hp combined for
distinguishing between Infection and Non-Infection groups

| | Cut off | | | | | |
|---|---|---|---|---|---|---|
| | (ug/ml) or | Sensi- | Spec- | | | |
| Description | (mg/dl) | tivity | ificity | PPV | NPV | accuracy |
| Infection vs | (29, 20, 114) | 84 | 96 | 97 | 79 | 88 |
| Non-infection | | | | | | |
| Infection vs | (29, 20, 159) | 84 | 96 | 97 | 79 | 88 |
| Non-infection | | | | | | |

TABLE 35
Further performance statistics for SAA, CRP and Hp combined
for distinguishing between Infection and Non-Infection groups

| | Cut off | | | | | |
|---|---|---|---|---|---|---|
| | (ug/ml) or | false | true | | actual | actual |
| Description | (mg/dl) | +ve | −ve | false −ve | +ve | −ve |
| Infection vs | (29, 20, 114) | 1 | 22 | 6 | 37 | 23 |
| Non-infection | | | | | | |
| Infection vs | (29, 20, 159) | 1 | 22 | 6 | 37 | 23 |
| Non-infection | | | | | | |

Non-Infectious Inflammation vs Other (Healthy and Infection): Hp

TABLE 36
Performance statistics for SAA, CRP and Hp combined for
distinguishing between Individuals with Inflammation and those
with either Healthy or Infection conditions (Other)

| | Cut off | | | | | |
|---|---|---|---|---|---|---|
| | (ug/ml) or | Sensi- | Spec- | | | |
| Description | (mg/dl) | tivity | ificity | PPV | NPV | Accuracy |
| Non-Infectious | (5, 6, 114) | 46 | 87 | 50 | 85 | 78 |
| Inflammation | | | | | | |
| vs Other | | | | | | |
| Non-Infectious | (5, 6, 159) | 15 | 89 | 29 | 79 | 73 |
| Inflammation | | | | | | |
| vs Other | | | | | | |
| Non-Infectious | (10, 6, 114) | 23 | 87 | 33 | 80 | 73 |
| Inflammation | | | | | | |
| vs Other | | | | | | |
| Non-Infectious | (10, 6, 159) | 15 | 89 | 29 | 79 | 73 |
| Inflammation | | | | | | |
| vs Other | | | | | | |
| Non-Infectious | (29, 6, 114) | 8 | 91 | 20 | 78 | 73 |
| Inflammation | | | | | | |
| vs Other | | | | | | |
| Non-Infectious | (29, 6, 159) | 0 | 94 | 0 | 77 | 73 |
| Inflammation | | | | | | |
| vs Other | | | | | | |
| Non-Infectious | (29, 10, 114) | 31 | 89 | 44 | 82 | 77 |
| Inflammation | | | | | | |
| vs Other | | | | | | |
| Non-Infectious | (29, 10, 159) | 23 | 94 | 50 | 81 | 78 |
| Inflammation | | | | | | |
| vs Other | | | | | | |
| Non-Infectious | (29, 20, 114) | 38 | 87 | 45 | 84 | 77 |
| Inflammation | | | | | | |
| vs Other | | | | | | |
| Non-Infectious | (29, 20, 159) | 31 | 91 | 50 | 83 | 78 |
| Inflammation | | | | | | |
| vs Other | | | | | | |

TABLE 37
Further performance statistics for SAA, CRP and Hp combined for distinguishing
between Individuals with Inflammation and those with either Healthy or Infection conditions
(Other)

| | Cut off | | | | | | |
|---|---|---|---|---|---|---|---|
| | (ug/ml) or | true | false | true | false | actual | actual |
| Description | (mg/dl) | +ve | +ve | −ve | −ve | +ve | −ve |
| Non-Infectious | (5, 6, 114) | 6 | 6 | 41 | 7 | 13 | 47 |
| Inflammation | | | | | | | |
| vs Other | | | | | | | |
| Non-Infectious | (5, 6, 159) | 2 | 5 | 42 | 11 | 13 | 47 |
| Inflammation | | | | | | | |
| vs Other | | | | | | | |
| Non-Infectious | (5, 10, 114) | 3 | 6 | 41 | 10 | 13 | 47 |
| Inflammation | | | | | | | |
| vs Other | | | | | | | |
| Non-Infectious | (5, 10, 159) | 2 | 5 | 42 | 11 | 13 | 47 |
| Inflammation | | | | | | | |
| vs Other | | | | | | | |
| Non-Infectious | (5, 20, 114) | 1 | 4 | 43 | 12 | 13 | 47 |
| Inflammation | | | | | | | |
| vs Other | | | | | | | |
| Non-Infectious | (5, 20, 159) | 0 | 3 | 44 | 13 | 13 | 47 |
| Inflammation | | | | | | | |
| vs Other | | | | | | | |
| Non-Infectious | (10, 6, 114) | 4 | 5 | 42 | 9 | 13 | 47 |
| Inflammation | | | | | | | |
| vs Other | | | | | | | |
| Non-Infectious | (10, 6, 159) | 3 | 3 | 44 | 10 | 13 | 47 |
| Inflammation | | | | | | | |
| vs Other | | | | | | | |
| Non-Infectious | (10, 10, 114) | 5 | 6 | 41 | 8 | 13 | 47 |
| Inflammation | | | | | | | |
| vs Other | | | | | | | |
| Non-Infectious | (10, 10, 159) | 4 | 4 | 43 | 9 | 13 | 47 |
| Inflammation | | | | | | | |
| vs Other | | | | | | | |

### Differentiation Between Healthy, Inflammation, and Viral Vs Bacterial Groups

A companion study was carried out to assess the performance of the biomarkers Hp, SAA, MxA and CRP in differentiating between viral and bacterial infection based on the studies presented above. CRP, MxA and Hp emerged as potential candidates for differentiating between viral and bacterial infection with all three demonstrating diagnostic capability with P<0.001 in ROC analysis (see Table 38 and Table 39).

TABLE 38
Performance statistics for SAA, CRP, Hp and MxA combined for
distinguishing between Healthy, Inflammation, Bacterial and Viral
groups simultaneously.

| | positive | Sensi- | | | | |
|---|---|---|---|---|---|---|
| Description | type | tivity | Specificity | PPV | NPV | Accuracy |
| Infection vs | Infection | 81 | 96 | 97 | 76 | 87 |
| Non-infection | | | | | | |
| Bacterial vs | Bacterial | 100 | 100 | 100 | 100 | 100 |
| Viral | | | | | | |
| Bacterial vs | Bacterial | 88 | 100 | 100 | 96 | 97 |
| Non-bacterial | | | | | | |
| Viral vs Non- | Viral | 76 | 97 | 94 | 88 | 90 |
| viral | | | | | | |
| Healthy vs | Healthy | 100 | 76 | 45 | 100 | 80 |
| Non-healthy | | | | | | |
| Inflammation | Inflamma- | 38 | 96 | 71 | 85 | 83 |
| vs Non- | tion | | | | | |
| inflammation | | | | | | |

Note that the cut off values used for SAA, CRP, CRP, Hp, MxA, SAA, Hp were 20 ug/ml, 10 ug/ml, 97 ug/ml, 182 mg/dl, 0.174 ug/ml, 6 ug/ml, and 114 mg/dl, respectively.

TABLE 39
Further Performance statistics for SAA, CRP Hp and MxA combined for distinguishing
between Healthy, Inflammation, Bacterial and Viral groups simultaneously.

| | | true | false | true | false | actual | actual_ |
|---|---|---|---|---|---|---|---|
| Description | +ve type | +ve | +ve | −ve | −ve | +ve | ve |
| Infection vs | Infection | 30 | 1 | 22 | 7 | 37 | 23 |
| Non- | | | | | | | |
| infection | | | | | | | |
| Bacterial vs | Bacterial | 14 | 0 | 16 | 0 | 14 | 16 |
| Viral | | | | | | | |
| Bacterial vs | Bacterial | 14 | 0 | 44 | 2 | 16 | 44 |
| Non- | | | | | | | |
| bacterial | | | | | | | |
| Viral vs Non- | Viral | 16 | 1 | 38 | 5 | 21 | 39 |
| viral | | | | | | | |
| Healthy vs | Healthy | 10 | 12 | 38 | 0 | 10 | 50 |
| Non-healthy | | | | | | | |
| Inflammation | Inflammation | 5 | 2 | 45 | 8 | 13 | 47 |
| vs Non- | | | | | | | |
| inflammation | | | | | | | |

Note that the cut off values used for SAA, CRP, CRP, Hp, MxA, SAA, Hp were 20 ug/ml, 10 ug/ml, 97 ug/ml, 182 mg/dl, 0.174 ug/ml, 6 ug/ml, and 114 mg/dl, respectively.

### DISCUSSION

The main objective of this study is to evaluate serum amyloid A (SAA), C reactive protein (CRP), haptoglobin (Hp) and myxovirus resistance protein A (MxA) to assess their usefulness in determining an individual's health status by distinguishing between healthy, infectious, and inflammatory conditions. It is also an objective to use the expression levels of these proteins (or the genes encoding said proteins) to differentiate between viral and bacterial infection.

The evaluation of CRP, SAA, Hp, and MxA in this study demonstrated a number of surprising findings. SAA, CRP and Hp and MxA are all elevated in the presence of infection. However, only Hp is consistently elevated in the presence of non-infectious inflammation. Elevated SAA and CRP are not associated with healthy controls or non-infectious inflammatory disease but are positively correlated with the presence of infection. This is a departure from previous findings in the literature which report SAA and CRP as being non-specific markers associated with inflammatory processes. Elevated Hp is positively correlated with both inflammatory conditions and infectious conditions. In time course analysis, SAA and CRP are earlier, more dynamic indicators of the onset of infection and can be used to monitor disease progression and response to therapy.

These findings are meaningful and present the possibility of a unique method for the differentiation of healthy, inflammatory and infectious groups by combining the biomarker levels at particular reference points. This could be extended to biomarker levels at particular reference points over particular periods of time. SAA is shown here to be a sensitive and specific marker of infection which can classify groups into infection vs non-infectious, thereby identifying infectious cases which may be candidates for antimicrobial treatment. However, SAA is not a good indicator of healthy or inflammatory groups by itself, and so those patients in inflammatory groups who have an inflammatory disease, which may progress to a more aggressive or infectious condition, or those with sterile inflammatory conditions, are not classified/detected when SAA is used, alone and so are at risk of being either untreated or mistreated. This poses a significant problem as in the absence of a method to definitively differentiate healthy, inflammatory, and infectious conditions, clinicians may feel the need to treat a patient to avoid the risk associated with withholding treatment.

The data promotes SAA as a definitive marker of infection which could be used to differentiate/identify infection from inflammatory conditions. The data also strongly indicates that SAA in combination with Hp can be used to both detect infection and differentiate non-infectious inflammation from a healthy status.

Though the biomarkers are useful as single biomarkers for detecting groups i.e. individual groups, alone they lack the required sensitivity and specificity to differentiate between all groups in the study. However, when considered together it is possible to correctly identify the groups to a high degree of accuracy. The combination of the biomarkers as described here is also useful to monitor response to therapy in both inflammatory and infectious cases, whereby a decrease in the relevant biomarkers is associated with an improved health status. The method may also be engaged to assess the efficacy of anti-inflammatory or anti-microbial therapies. It may also be engaged as an ongoing screen in chronic inflammatory cases.

CRP demonstrated a good ability to correctly identify inflammation within combined Inflammation and Healthy groups. However, it has poor specificity, misclassifying many healthy samples as inflammation in the analysis. SAA demonstrated a poor ability to identify inflammation within the two groups at just 15% specificity. Specificity was 100%, meaning that it correctly categorised all healthy individuals but also misclassified a significant number of individuals with inflammation as healthy. In an analysis where the biomarkers were assessed for their ability to distinguish between non-infectious inflammation and infection, when the Infection and Inflammation groups were combined, CRP was demonstrated to be good at correctly identifying the presence of infection. However, specificity was poor, and it could not correctly categorise individuals in the Inflammation group leading to false positives. SAA showed good performance when identifying infection and differentiating within the Infection and Inflammation groups combined. However, the results from Table 12 herein SAA was unable to accurately differentiate between Healthy and Inflammation groups means that the Inflammation group, if presented alongside Healthy individuals as is the normal course of events in clinical settings (e.g., non-inflammatory conditions and disease screening), may not be correctly diagnosed as having an inflammation and may be misclassified as apparently healthy individuals.

The outcome of a dichotomous assessment, if applied to a clinical setting, may have serious health consequences for individuals being assessed, since it is not possible to achieve three outcomes using this assessment. Individuals presenting for health status assessment are generally healthy (non-infectious) or non-healthy (infectious or non-infectious), where non-healthy is either infection or inflammation and non-infectious is either healthy or inflammation. Infection can further be differentiated into Viral and Bacterial. The study presented here further assessed the biomarkers individually and then combined the biomarkers for each of the three main groups on this basis, i.e., Healthy vs Non-Healthy (where the Non-Healthy group is a combination of both Inflammation and Infection groups) and Infectious vs Non-Infectious (where the Non-Infectious group is a combination of the Healthy and Inflammatory group) and Inflammation vs Other (a combination of Healthy and Infectious groups).

Combining SAA and CRP to determine the health status of an individual allows for differentiation between the Healthy, Inflammation and Infection groups, which is a significant improvement over existing methods, where neither CRP nor SAA can accurately identify individuals within the Inflammation group. Inflammation can now be distinguished from Healthy and Infection Groups in a health status assessment, whereas previously this was not possible as testers could not use the biomarkers CRP and SAA individually.

### Summary of CRP, SAA, HP Combination Findings

The combination of SAA, CRP and HP provide a new and improved method for the identification and differentiation of the three main groups that make up a health status assessment, i.e., Healthy, Inflammation, and Infection. Haptoglobin is primarily associated with hemolytic anemia. It has a wide range of normal values and to date it has been difficult to determine appropriate cut off values in the literature. Haptoglobin is not reported as a major human acute phase protein when compared to SAA and CRP and appears to have received limited interest. These studies show, however, that Hp, as part of a three outcome analysis using SAA and CRP, improves differentiation between the groups which ultimately leads to better identification of health status and more accurate selection of treatment for Individuals.

In practical use, when SAA (and/or CRP) values are below the threshold levels reported here, the presence of an elevated Hp is a positive indicator for inflammatory disease without an infectious process. This is a significant improvement upon existing methods of differentiating infectious and inflammatory conditions.

A testing method sequence is proposed for assessing the efficacy of the biomarkers together when differentiating between the three groups (healthy, inflammatory, and infectious) (see Table 40).

This method could be further developed through the addition of MxA into the sequence to further improve one or more of the parameters of sensitivity, specificity, positive predictive value and negative predictive value.

CRP is the biomarker most frequently associated with the identification of bacterial infection. CRP on its own however cannot be used to distinguish between the multiple groups associated with a health status assessment. The sensitivity and specificity and overall accuracy for CRP as a biomarker for bacterial infection varies in the literature. Studies carried out by the inventors identified a cut off value of 97 ug/ml associated with a sensitivity of 75% and a specificity of 100% for identifying individuals within the Bacterial Infection Group. Similar reasoning may be applied to SAA and HP. SAA, though a good biomarker for differentiating between infection and non-infection, demonstrated no ability to differentiate between viral and bacterial groups as seen in FIGS. 14-17.

The inventors developed a combination of biomarkers, namely at least two, three or all from SAA, CRP, HP and MxA, and cut off values to differentiate between Healthy, Inflammation, and Infection groups, and further between Viral groups, simultaneously. The preferred combination determined is as follows: SAA, CRP, CRP, HP, MxA, SAA, Hp at the following thresholds 20 ug/ml, 10 ug/ml, 97 ug/ml, 182 mg/dL, 0.174 ug/ml, 6 ug/ml, 114 mg/dL, with the following logic:
if (‘SAA’>20) and (‘CRP’>10), then the determination is an Infection;
if (‘CRP’>97) or ((‘Hp’>182) and (‘MxA’>0.174)), then the determination ise Bacterial Infection;
otherwise determine Viral Infection;
else of (‘SAA’<6) or (‘Hp’<114) determine Healthy;
otherwise return “Inflammation”

The combination of the biomarkers according to the cut off values herein demonstrate a very significant and surprising improvement over current methods that are unable to accurately differentiate simultaneously between Healthy, Inflammatory and Infection groups including Bacterial and Viral groups. The logical parameters above demonstrate the excellent sensitivity and specificity with significant improvement over CRP for the identification of Bacterial Infection Groups wherein the combination was able to detect all groups simultaneously with an accuracy ranging from 83% to 100% relating to the group identified with average accuracy for the method.

Put another way, in one combination there is SAA, CRP and Hp. In the other combo MxA is added to differentiate between viral and bacterial groups, for example. If the logic for Mxa and CRP at the 97 ug/ml cut off is removed (see text/comments on Table 38 and 39) you get “Infection” instead of “Viral or “Bacterial”.

The health status assessment of subjects can be carried out simultaneously or sequentially on all three or four biomarkers to determine the health status of the subject. If an infection status is identified in the subject, the assessment can be carried out in a single and/or in multiple step algorithm logic analysis, e.g., determine infection followed by differentiation of viral and bacterial infection. Additional biomarkers of viral or bacterial infection may also be assessed, such as procalcitonin, calprotectin, biomarkers associated with the coagulation cascade, and interleukin 1, 6, 8, and 11. If an infection (bacterial or viral) or an inflammation status is determined in the sample from the subject, then the subject is selected for appropriate treatment, as detailed in, for example Table 1. Subjects selected for treatment can be further monitored using the same method to monitor changes in health status include severity of the ailment and a return to healthy status.

The analysis of the sample from the subject may be interpreted by eye through the use of colorimetric methods and/or through electronic devices where algorithm logic can be employed according to the preferred combination herein to determine health status.

The study has found that SAA, CRP and Hp when combined can accurately distinguish between three groups in a health status assessment. The combination of biomarkers may be interpreted simultaneously, and the study has shown greater diagnostic accuracy compared to single or dual biomarker evaluations. The study has also found that SAA, CRP and Hp when in combination can accurately distinguish an outcome method of health assessment as detailed in the steps above, providing for a further outcome by differentiating viral and bacterial individuals accurate within a group that also comprises of healthy and inflammation groups.

These studies surprisingly found that despite all the biomarkers assessed being reportedly pro-inflammatory markers, they do not all behave in the same way in response to inflammation and are elevated or at normal levels based on the type of inflammation involved. The Applicant also presents a method of combining the biomarkers at particular reference levels to achieve greater levels of accuracy than currently available methods. This method can differentiate between healthy and inflammatory patients as well as inflammatory and infectious patients potentially leading to a reduction in the number of non-infectious cases treated unnecessarily with antimicrobials and an increase in the number of infections identified and treated correctly. This method also identifies inflammatory conditions as distinct from infectious conditions, and so patients with inflammatory symptoms without infection can also be treated and/or monitoring appropriately. Furthermore, the study shows that the biomarkers may be engaged together in a system whereby infection and/or inflammation can be detected early and corresponding treatment efficacy can be measured using the biomarkers over hours and/or days.

The identification of a biomarker or a panel of biomarkers which provides additional information on whether a condition is infectious or inflammatory in nature would present a number of positive outcomes, including a foreseeable reduction in unnecessary use of the already limited arsenal of antimicrobials available, rapid intervention, better clinical decision making and improved health management at home and in a clinical setting through monitoring regimes.

SAA, Hp, CRP, and MxA have been studied here as potential diagnostic and prognostic indicators that provide superior differentiation than currently available methods and which will aid clinicians in the treatment decision making process.

In the specification the terms “comprise, comprises, comprised and comprising” or any variation thereof and the terms “include, includes, included and including” or any variation thereof are considered to be totally interchangeable and they should all be afforded the widest possible interpretation and vice versa.

The invention is not limited to the embodiments hereinbefore described but may be varied in both construction and detail.

1. A method of (a) determining a health status of an individual, (b) of monitoring the effectiveness of treatment of an infectious disease in an individual with an infectious disease, or (c) of identifying an individual with an infection or an infectious disease that is suitable for treatment with a therapy for treating the infectious disease, the methods comprising the steps of assaying a biological sample from the individual for the positive expression of at least three genes, or proteins encoded by said genes, selected from SAA, CRP, Hp and MxA, and wherein the positive expression of the at least three genes, or proteins encoded by said genes, above or below a specified threshold level (a) correlates with the health status of the individual, (b) correlates with the effectiveness of treatment of the infectious disease in the individual, and (c) correlates with identifying the treatment of the infection or the infectious disease in the individual.

2. (canceled)

3. (canceled)

4. A method according to claim 1, wherein the at least three genes, or proteins encoded by said genes, are SAA and two selected from CRP, Hp and MxA.

5. A method according to claim 1, wherein all four genes, or proteins encoded by said genes are selected.

6. A method according to claim 1, wherein the at least three genes, or proteins encoded by said genes, are SAA and two selected from CRP, Hp, and MxA and wherein when the protein expression levels of SAA are less than 6 ug/ml, the protein expression levels of Hp are less than 114 mg/dl, the protein expression levels of CRP are less than 10 ug/ml, and the protein expression levels of MxA are less than 0.174 ug/ml, the individual is healthy.

7. A method according to claim 1, wherein the at least three genes, or proteins encoded by said genes, are SAA and two selected from CRP, Hp, and MxA and wherein when the protein expression levels of SAA are less than 20 ug/ml, when the protein expression levels of Hp are greater than 114 mg/dl, when the protein expression levels of CRP are greater than 5 ug/ml, and when the protein expression levels of MxA are greater than 0.174 ug/ml, the individual is experiencing an inflammatory event or is suffering from an inflammatory condition.

8. A method according to claim 1, wherein the at least three genes, or proteins encoded by said genes, are SAA and two selected from CRP, Hp, and MxA and wherein when the protein expression levels of SAA are greater than 20 ug/ml, when the protein expression levels of Hp are greater than 114 mg/dl, when the protein expression levels of CRP are greater than 10 ug/ml, and when the protein expression levels of MxA are greater than 0.174 ug/ml, the individual is experiencing an infection.

9. A method according to claim 1, wherein the at least three genes, or proteins encoded by said genes, are SAA and two selected from CRP, Hp, and MxA and wherein when the protein expression levels of SAA are about 18 ug/ml to about 21 ug/ml, when the protein expression levels of Hp are about 50 mg/dl to 220 mg/dl, when the protein expression levels of CRP are about 3 ug/ml to 29 ug/ml, and when the protein expression levels of MxA are greater than 0.174 ug/ml, the individual is at risk of onset of an autoimmune and/or an autoinflammatory condition.

10. A method according to claim 1, wherein the at least three genes, or proteins encoded by said genes, are SAA and two selected from CRP, Hp, and MxA and wherein when the protein expression levels of SAA are less than 20 ug/ml, when the protein expression levels of Hp are less than 160 mg/dl, when the protein expression levels of CRP are less than 5 ug/ml, and when the protein expression levels of MxA are less than 0.174 ug/ml, the individual is responding to treatment; and wherein when the protein expression levels of SAA are greater than 20 ug/ml, when the protein expression levels of Hp are greater than 160 mg/dl, when the protein expression levels of CRP are greater than 5 ug/ml, and when the protein expression levels of MxA are greater than 0.174 ug/ml, the individual still has the infection and is not responding to treatment.

11. A method of determining the progression of treatment of an individual with an inflammatory disease, the method comprising the step of assaying a biological sample from the individual with the inflammatory disease for the positive expression of at least two genes, or proteins encoded by said genes, selected from CRP, Hp and MxA, and negative expression of SAA, wherein the positive expression of the at least two genes, or proteins encoded by said genes, above or below a specified threshold level, and the negative expression of SAA, correlates with the effectiveness of treatment of the inflammatory disease or disorder in the individual.

12. A method according to claim 11, wherein the threshold levels for Hp protein expression levels are optionally 114 mg/dl, the threshold levels for CRP protein expression levels are 5 ug/ml, and the threshold levels for MxA are 0.174 ug/ml.

13. A method according to claim 11, wherein when the protein expression levels of Hp are less than 114 mg/dl and the protein expression levels of CRP are less than 5 ug/ml, and when the protein expression levels of MxA are less than 0.174 ug/ml, the individual is responding to treatment; and wherein when the protein expression levels of Hp are greater than 114 mg/dl, the protein expression levels of CRP are greater than 5 ug/ml, and when the protein expression levels of MxA are greater than 0.174 ug/ml, the individual is experiencing an inflammatory response and is not responding to treatment.

14. (canceled)

15. (canceled)

16. (canceled)

17. (canceled)

18. (canceled)

19. A method of treating an infection, an infectious disease, an inflammatory condition, or an autoinflammatory condition in a subject, the method comprising: receiving the results of an assay that indicates that the level of at least three genes, or proteins encoded by said genes, selected from SAA, CRP, Hp and MxA, in a sample obtained from the subject is above or below a threshold level; and administering a treatment for the disease or condition, wherein the at least three genes, or proteins encoded by said genes, are SAA, CRP and Hp.

20. The method of claim 19, wherein the treatment comprises administering a suitable antibiotic to the subject determined to have an infection; administering an anti-inflammatory drug if the subject is determined to have an inflammatory condition; or administering a suitable treatment as set out in Table 1.

21. (canceled)

22. (canceled)

23. The method of claim 1 wherein the disease or condition is selected from those set out in Table 1.

24. A method for differentiating whether an individual is healthy, has an inflammatory condition or has a viral or bacterial infection, the method comprising the steps of assaying a biological sample from the individual for the positive expression of at least three genes, or proteins encoded by said genes, selected from SAA, CRP, Hp and MxA, and wherein the positive expression of the at least three genes, or proteins encoded by said genes, above or below a specified threshold level correlates with the health status of the individual, wherein if the concentration of SAA is >20 ugf/ml, and the concentration of CRP is >10 ug/ml, the individual is determined to have an infection; wherein if the concentration of CRP>97 ug/ml or the concentration of Hp>182 mg/dl and the concentration of MxA>0.174 ug/ml, the individual has a bacterial infection or a viral infection; and wherein if the concentration of SAA<6 ug/ml or the concentration of Hp<114 mg/dl, the individual is determined to be healthy and wherein if the conditions of the above are not met the individual is determined to have inflammation.

25. (canceled)

26. A kit for determining the health status of a subject, the kit comprising a control oligonucleotide and a set of oligonucleotides for detecting SEQ ID NO: 2, 3, 5, 6, 8, 9, 11 and 12, wherein detection of at least three of SEQ ID NO: 2, 3, 5, 6, 8, 9, 11 or 12 above a threshold in the sample is indicative of the health status of the subject, and wherein the sensitivity of the assay for detecting the at least three sequences from SEQ ID NO: 2, 3, 5, 6, 8, 9, 11 or 12 is at least 80%, wherein the at least three oligonucleotides are selected from one of SEQ ID NO: 2 or 3, one of SEQ ID NO: 5 or 6, and one of SEQ ID NO: 8 or 9.

27. A kit according to claim 25, wherein the support further comprises having at least four or more oligonucleotides anchored thereon selected from one or both of SEQ ID NO: 2 or 3, one or both of SEQ ID NO: 5 or 6, and one or both of SEQ ID NO: 8 or 9.

28. A kit according to claim 25, wherein the support further comprises at least one or both oligonucleotides anchored thereon selected from SEQ ID NO: 11 and 12.

29. A method according to claim 1, wherein when a negative expression of the at least three genes encoding SAA, CRP, and Hp, or proteins encoded by said genes, are below the threshold level, the result indicates a healthy status in the subject, and the treatment, if being administered, can be stopped.

30. A method according to claim 11, wherein when a negative expression of the at least three genes encoding SAA, CRP, and Hp, or proteins encoded by said genes, are below the threshold level, the result indicates a healthy status in the subject, and the treatment, if being administered, can be stopped.

31. A method according to claim 19, wherein when a negative expression of the at least three genes encoding SAA, CRP, and Hp, or proteins encoded by said genes, are below the threshold level, the result indicates a healthy status in the subject, and the treatment, if being administered, can be stopped.
